SEQUENCE LISTING (177 sequences)

SEQ ID NO: 1 - protein, 6 aa, synthetic construct
  REGION 1..6: note = Synthetic Construct
SGYHWS

SEQ ID NO: 2 - protein, 16 aa, synthetic construct
  REGION 1..16: note = Synthetic Construct
RIDWDDDKYYSTSLKS

SEQ ID NO: 3 - protein, 7 aa, synthetic construct
  REGION 1..7: note = Synthetic Construct
SYVYFDY

SEQ ID NO: 4 - protein, 12 aa, synthetic construct
  REGION 1..12: note = Synthetic Construct
RASQSVRGRFLA

SEQ ID NO: 5 - protein, 7 aa, synthetic construct
  REGION 1..7: note = Synthetic Construct
DASNRAT

SEQ ID NO: 6 - protein, 9 aa, synthetic construct
  REGION 1..9: note = Synthetic Construct
QQSSSWPPT

SEQ ID NO: 7 - protein, 5 aa, synthetic construct
  REGION 1..5: note = Synthetic Construct
DYAIH

SEQ ID NO: 8 - protein, 17 aa, synthetic construct
  REGION 1..17: note = Synthetic Construct
IISPSSGSTNYAQKFQG

SEQ ID NO: 9 - protein, 15 aa, synthetic construct
  REGION 1..15: note = Synthetic Construct
DIHSGSSGYYYGFDV

SEQ ID NO: 10 - protein, 15 aa, synthetic construct
  REGION 1..15: note = Synthetic Construct
RASESVDFFGISFLA

SEQ ID NO: 11 - protein, 11 aa, synthetic construct
  REGION 1..11: note = Synthetic Construct
QHYTSSPPVYT

SEQ ID NO: 12 - protein, 6 aa, synthetic construct
  REGION 1..6: note = Synthetic Construct
SGYYWA

SEQ ID NO: 13 - protein, 17 aa, synthetic construct
  REGION 1..17: note = Synthetic Construct
SISGSGSTTYYADSVKG

SEQ ID NO: 14 - protein, 8 aa, synthetic construct
  REGION 1..8: note = Synthetic Construct
DGFGYFDY

SEQ ID NO: 15 - protein, 10 aa, synthetic construct
  REGION 1..10: note = Synthetic Construct
SASSSVSYVY

SEQ ID NO: 16 - protein, 7 aa, synthetic construct
  REGION 1..7: note = Synthetic Construct
DASSLES

SEQ ID NO: 17 - protein, 9 aa, synthetic construct
  REGION 1..9: note = Synthetic Construct
VQGLQTPWT

SEQ ID NO: 18 - protein, 5 aa, synthetic construct
  REGION 1..5: note = Synthetic Construct
DYGIH

SEQ ID NO: 19 - protein, 16 aa, synthetic construct
  REGION 1..16: note = Synthetic Construct
EIYHSGSTYYSPSLKS

SEQ ID NO: 20 - protein, 15 aa, synthetic construct
  REGION 1..15: note = Synthetic Construct
DVAPGSSGYYDGFDF

SEQ ID NO: 21 - protein, 11 aa, synthetic construct
  REGION 1..11: note = Synthetic Construct
RASQGIGSSLA

SEQ ID NO: 22 - protein, 10 aa, synthetic construct
  REGION 1..10: note = Synthetic Construct
QQYDQWPPWT

SEQ ID NO: 23 - protein, 6 aa, synthetic construct
  REGION 1..6: note = Synthetic Construct
SGYHWD

SEQ ID NO: 24 - protein, 17 aa, synthetic construct
  REGION 1..17: note = Synthetic Construct
GISGYGGSTYYADSVKG

SEQ ID NO: 25 - protein, 11 aa, synthetic construct
  REGION 1..11: note = Synthetic Construct
HSYYGSGNFDY

SEQ ID NO: 26 - protein, 15 aa, synthetic construct
  REGION 1..15: note = Synthetic Construct
RASESVDFFGKSFLH

SEQ ID NO: 27 - protein, 7 aa, synthetic construct
  REGION 1..7: note = Synthetic Construct
DASNLET

SEQ ID NO: 28 - protein, 9 aa, synthetic construct
  REGION 1..9: note = Synthetic Construct
QQSYSWPPT

SEQ ID NO: 29 - protein, 5 aa, synthetic construct
  REGION 1..5: note = Synthetic Construct
DYWIH

SEQ ID NO: 30 - protein, 17 aa, synthetic construct
  REGION 1..17: note = Synthetic Construct
WISPSGGGTKYAQKFQG

SEQ ID NO: 31 - protein, 7 aa, synthetic construct
  REGION 1..7: note = Synthetic Construct
GAYEFDY

SEQ ID NO: 32 - protein, 12 aa, synthetic construct
  REGION 1..12: note = Synthetic Construct
RASQSVSSRFLA

SEQ ID NO: 33 - protein, 9 aa, synthetic construct
  REGION 1..9: note = Synthetic Construct
QQSYPTPLT

SEQ ID NO: 34 - protein, 7 aa, synthetic construct
  REGION 1..7: note = Synthetic Construct
TGGVAVS

SEQ ID NO: 35 - protein, 10 aa, synthetic construct
  REGION 1..10: note = Synthetic Construct
RIATATYFDY

SEQ ID NO: 36 - protein, 12 aa, synthetic construct
  REGION 1..12: note = Synthetic Construct
RASQTVFSRYLA

SEQ ID NO: 37 - protein, 10 aa, synthetic construct
  REGION 1..10: note = Synthetic Construct
QQSYYWPPWT

SEQ ID NO: 38 - protein, 7 aa, synthetic construct
  REGION 1..7: note = Synthetic Construct
TGGVAVG

SEQ ID NO: 39 - protein, 17 aa, synthetic construct
  REGION 1..17: note = Synthetic Construct
AISGYGSTTYYADSVKG

SEQ ID NO: 40 - protein, 10 aa, synthetic construct
  REGION 1..10: note = Synthetic Construct
LPYSAYAFDY

SEQ ID NO: 41 - protein, 11 aa, synthetic construct
  REGION 1..11: note = Synthetic Construct
RASQGVSSYLA

SEQ ID NO: 42 - protein, 7 aa, synthetic construct
  REGION 1..7: note = Synthetic Construct
AASTLQS

SEQ ID NO: 43 - protein, 9 aa, synthetic construct
  REGION 1..9: note = Synthetic Construct
QHHYGTPLT

SEQ ID NO: 44 - protein, 5 aa, synthetic construct
  REGION 1..5: note = Synthetic Construct
GYAIH

SEQ ID NO: 45 - protein, 17 aa, synthetic construct
  REGION 1..17: note = Synthetic Construct
IISPSGGGTKYAQKFQG

SEQ ID NO: 46 - protein, 5 aa, synthetic construct
  REGION 1..5: note = Synthetic Construct
HPFAY

SEQ ID NO: 47 - protein, 15 aa, synthetic construct
  REGION 1..15: note = Synthetic Construct
RASQSVDFYGISFLD

SEQ ID NO: 48 - protein, 11 aa, synthetic construct
  REGION 1..11: note = Synthetic Construct
QQYVSSPPEYT

SEQ ID NO: 49 - protein, 5 aa, synthetic construct
  REGION 1..5: note = Synthetic Construct
GYGIH

SEQ ID NO: 50 - protein, 7 aa, synthetic construct
  REGION 1..7: note = Synthetic Construct
RIDAFDI

SEQ ID NO: 51 - protein, 15 aa, synthetic construct
  REGION 1..15: note = Synthetic Construct
RASQSVDFDGFSFLH

SEQ ID NO: 52 - protein, 9 aa, synthetic construct
  REGION 1..9: note = Synthetic Construct
QQRDSWPYT

SEQ ID NO: 53 - protein, 6 aa, synthetic construct
  REGION 1..6: note = Synthetic Construct
LYDVAY

SEQ ID NO: 54 - protein, 15 aa, synthetic construct
  REGION 1..15: note = Synthetic Construct
RASQSVDFHGKSFLH

SEQ ID NO: 55 - protein, 9 aa, synthetic construct
  REGION 1..9: note = Synthetic Construct
EQSLEVPFT

SEQ ID NO: 56 - protein, 17 aa, synthetic construct
  REGION 1..17: note = Synthetic Construct
IISPSGGSTKYAQKFQG

SEQ ID NO: 57 - protein, 8 aa, synthetic construct
  REGION 1..8: note = Synthetic Construct
LGYGYFDV

SEQ ID NO: 58 - protein, 15 aa, synthetic construct
  REGION 1..15: note = Synthetic Construct
RASQSVDFYGISFLH

SEQ ID NO: 59 - protein, 9 aa, synthetic construct
  REGION 1..9: note = Synthetic Construct
VQALQLPLT

SEQ ID NO: 60 - protein, 6 aa, synthetic construct
  REGION 1..6: note = Synthetic Construct
SGHYWS

SEQ ID NO: 61 - protein, 16 aa, synthetic construct
  REGION 1..16: note = Synthetic Construct
DISHSGSTYYSQSLKS

SEQ ID NO: 62 - protein, 9 aa, synthetic construct
  REGION 1..9: note = Synthetic Construct
GSRTGYFDY

SEQ ID NO: 63 - protein, 11 aa, synthetic construct
  REGION 1..11: note = Synthetic Construct
RASQSISSYLN

SEQ ID NO: 64 - protein, 124 aa, synthetic construct
  REGION 1..124: note = Synthetic Construct
EVQLVESGGGLVQPGGSLRLSCAASGFTFSDYAIHWVRQAPGKGLEWIGIISPSSGSTNYAQKFQGRVTISRDNSKNTLYLQLNSLRAEDTAVYYCARDIHSGSSGYYYGFDVWGQGTLVTVSS

SEQ ID NO: 65 - protein, 114 aa, synthetic construct
  REGION 1..114: note = Synthetic Construct
DIQLTQSPSSLSASVGDRVTITCRASESVDFFGISFLAWYQQKPGKAPKLLIYDASNRATGIPSRFSGSGSGTDFTLTISSLQPEDFATYYCQHYTSSPPVYTFGQGTKVEIKR

SEQ ID NO: 66 - protein, 118 aa, synthetic construct
  REGION 1..118: note = Synthetic Construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSS

SEQ ID NO: 67 - protein, 107 aa, synthetic construct
  REGION 1..107: note = Synthetic Construct
DIQLTQSPSSLSASVGDRVTITCSASSSVSYVYWYQQKPGKAPKLLIYDASSLESGVPSRFSGSGSGTDFTLTISSLQPEDFATYYCVQGLQTPWTFGQGTKVEIKR

SEQ ID NO: 68 - protein, 123 aa, synthetic construct
  REGION 1..123: note = Synthetic Construct
EVQLVESGGGLVQPGGSLRLSCAASGFTFSDYGIHWVRQAPGKGLEWIGEIYHSGSTYYSPSLKSRVTISRDNSKNTLYLQLNSLRAEDTAVYYCARDVAPGSSGYYDGFDFWGQGTLVTVSS

SEQ ID NO: 69 - protein, 109 aa, synthetic construct
  REGION 1..109: note = Synthetic Construct
DIQLTQSPSSLSASVGDRVTITCRASQGIGSSLAWYQQKPGKAPKLLIYDASNRATGIPSRFSGSGSGTDFTLTISSLQPEDFATYYCQQYDQWPPWTFGQGTKVEIKR

SEQ ID NO: 70 - protein, 121 aa, synthetic construct
  REGION 1..121: note = Synthetic Construct
EVQLVESGGGLVQPGGSLRLSCAASGYSISSGYHWDWIRQAPGKGLEWVSGISGYGGSTYYADSVKGRFTISRDNSKNTLYLQLNSLRAEDTAVYYCARHSYYGSGNFDYWGQGTLVTVSS

SEQ ID NO: 71 - protein, 112 aa, synthetic construct
  REGION 1..112: note = Synthetic Construct
DIQLTQSPSSLSASVGDRVTITCRASESVDFFGKSFLHWYQQKPGKAPKLLIYDASNLETGVPSRFSGSGSGTDFTLTISSLQPEDFATYYCQQSYSWPPTFGQGTKVEIKR

SEQ ID NO: 72 - protein, 116 aa, synthetic construct
  REGION 1..116: note = Synthetic Construct
EVQLVESGGGLVQPGGSLRLSCAASGFTFSDYWIHWVRQAPGKGLEWIGWISPSGGGTKYAQKFQGRVTISRDNSKNTLYLQLNSLRAEDTAVYYCARGAYEFDYWGQGTLVTVSS

SEQ ID NO: 73 - protein, 109 aa, synthetic construct
  REGION 1..109: note = Synthetic Construct
DIQLTQSPSSLSASVGDRVTITCRASQSVSSRFLAWYQQKPGKAPKLLIYDASNRATGIPSRFSGSGSGTDFTLTISSLQPEDFATYYCQQSYPTPLTFGQGTKVEIKR

SEQ ID NO: 74 - protein, 116 aa, synthetic construct
  REGION 1..116: note = Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSS

SEQ ID NO: 75 - protein, 109 aa, synthetic construct
  REGION 1..109: note = Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKR

SEQ ID NO: 76 - protein, 120 aa, synthetic construct
  REGION 1..120: note = Synthetic Construct
EVQLVESGGGLVQPGGSLRLSCAASGFSLSTGGVAVSWIRQAPGKGLEWIGEIYHSGSTYYSPSLKSRVTISRDNSKNTLYLQLNSLRAEDTAVYYCARRIATATYFDYWGQGTLVTVSS

SEQ ID NO: 77 - protein, 110 aa, synthetic construct
  REGION 1..110: note = Synthetic Construct
DIQLTQSPSSLSASVGDRVTITCRASQTVFSRYLAWYQQKPGKAPKLLIYDASNRATGIPSRFSGSGSGTDFTLTISSLQPEDFATYYCQQSYYWPPWTFGQGTKVEIKR

SEQ ID NO: 78 - protein, 121 aa, synthetic construct
  REGION 1..121: note = Synthetic Construct
EVQLVESGGGLVQPGGSLRLSCAASGFSLSTGGVAVGWIRQAPGKGLEWVSAISGYGSTTYYADSVKGRFTISRDNSKNTLYLQLNSLRAEDTAVYYCARLPYSAYAFDYWGQGTLVTVSS

SEQ ID NO: 79 - protein, 108 aa, synthetic construct
  REGION 1..108: note = Synthetic Construct
DIQLTQSPSSLSASVGDRVTITCRASQGVSSYLAWYQQKPGKAPKLLIYAASTLQSGVPSRFSGSGSGTDFTLTISSLQPEDFATYYCQHHYGTPLTFGQGTKVEIKR

SEQ ID NO: 80 - protein, 114 aa, synthetic construct
  REGION 1..114: note = Synthetic Construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSS

SEQ ID NO: 81 - protein, 114 aa, synthetic construct
  REGION 1..114: note = Synthetic Construct
DIQLTQSPSSLSASVGDRVTITCRASQSVDFYGISFLDWYQQKPGKAPKLLIYDASNRATGIPSRFSGSGSGTDFTLTISSLQPEDFATYYCQQYVSSPPEYTFGQGTKVEIKR

SEQ ID NO: 82 - protein, 115 aa, synthetic construct
  REGION 1..115: note = Synthetic Construct
EVQLVESGGGLVQPGGSLRLSCAASGYTFSGYGIHWVRQAPGKGLEWIGEIYHSGSTYYSPSLKSRVTISRDNSKNTLYLQLNSLRAEDTAVYYCARRIDAFDIWGQGTLVTVSS

SEQ ID NO: 83 - protein, 112 aa, synthetic construct
  REGION 1..112: note = Synthetic Construct
DIQLTQSPSSLSASVGDRVTITCRASQSVDFDGFSFLHWYQQKPGKAPKLLIYDASSLESGVPSRFSGSGSGTDFTLTISSLQPEDFATYYCQQRDSWPYTFGQGTKVEIKR

SEQ ID NO: 84 - protein, 115 aa, synthetic construct
  REGION 1..115: note = Synthetic Construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSS

SEQ ID NO: 85 - protein, 112 aa, synthetic construct
  REGION 1..112: note = Synthetic Construct
DIQLTQSPSSLSASVGDRVTITCRASQSVDFHGKSFLHWYQQKPGKAPKLLIYDASSLESGVPSRFSGSGSGTDFTLTISSLQPEDFATYYCEQSLEVPFTFGQGTKVEIKR

SEQ ID NO: 86 - protein, 117 aa, synthetic construct
  REGION 1..117: note = Synthetic Construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSS

SEQ ID NO: 87 - protein, 112 aa, synthetic construct
  REGION 1..112: note = Synthetic Construct
DIQLTQSPSSLSASVGDRVTITCRASQSVDFYGISFLHWYQQKPGKAPKLLIYDASSLESGVPSRFSGSGSGTDFTLTISSLQPEDFATYYCVQALQLPLTFGQGTKVEIKR

SEQ ID NO: 88 - protein, 118 aa, synthetic construct
  REGION 1..118: note = Synthetic Construct
EVQLVESGGGLVQPGGSLRLSCAASGYSITSGHYWSWIRQAPGKGLEWIGDISHSGSTYYSQSLKSRVTISRDNSKNTLYLQLNSLRAEDTAVYYCARGSRTGYFDYWGQGTLVTVSS

SEQ ID NO: 89 - protein, 108 aa, synthetic construct
  REGION 1..108: note = Synthetic Construct
DIQLTQSPSSLSASVGDRVTITCRASQSISSYLNWYQQKPGKAPKLLIYDASNLETGVPSRFSGSGSGTDFTLTISSLQPEDFATYYCQHHYGTPLTFGQGTKVEIKR

SEQ ID NO: 90 - protein, 215 aa, Homo sapiens
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 446 aa, Homo sapiens
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 16 aa, synthetic construct
  REGION 1..16: note = Synthetic Construct
EVGSYVLCAKFPFCIT

SEQ ID NO: 93 - protein, 17 aa, synthetic construct
  REGION 1..17: note = Synthetic Construct
EVGSYPPCVGFYPYCKT

SEQ ID NO: 94 - protein, 17 aa, synthetic construct
  REGION 1..17: note = Synthetic Construct
EVGSYSSCKAPYPYCKG

SEQ ID NO: 95 - protein, 18 aa, synthetic construct
  REGION 1..18: note = Synthetic Construct
EVGSYALCEPFATSVCRP

SEQ ID NO: 96 - protein, 18 aa, synthetic construct
  REGION 1..18: note = Synthetic Construct
EVGSYTLCEPFATSVCRP

SEQ ID NO: 97 - protein, 13 aa, synthetic construct
  REGION 1..13: note = Synthetic Construct
ESSCKAPYPYCKG

SEQ ID NO: 98 - protein, 14 aa, synthetic construct
  REGION 1..14: note = Synthetic Construct
ETLCEPFATSVCRP

SEQ ID NO: 99 - protein, 20 aa, synthetic construct
  REGION 1..20: note = Synthetic Construct
SGRSAGGGGTPLGLAGSGGS

SEQ ID NO: 100 - protein, 12 aa, synthetic construct
  REGION 1..12: note = Synthetic Construct
SGRGPLGLAGGS

SEQ ID NO: 101 - protein, 12 aa, synthetic construct
  REGION 1..12: note = Synthetic Construct
SGGGPLGLAGGS

SEQ ID NO: 102 - protein, 11 aa, synthetic construct
  REGION 1..11: note = Synthetic Construct
GGGPLGLAGGS

SEQ ID NO: 103 - protein, 11 aa, synthetic construct
  REGION 1..11: note = Synthetic Construct
GGGPLGLAGGS

SEQ ID NO: 104 - protein, 446 aa, synthetic construct
  REGION 1..446: note = Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 446 aa, synthetic construct; identical to SEQ ID NO: 91
  REGION 1..446: note = Synthetic Construct

SEQ ID NO: 106 - protein, 251 aa, synthetic construct
  REGION 1..251: note = Synthetic Construct
EVGSYVLCAKFPFCITSGRSAGGGGTPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 252 aa, synthetic construct
  REGION 1..252: note = Synthetic Construct
EVGSYPPCVGFYPYCKTSGRSAGGGGTPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 252 aa, synthetic construct
  REGION 1..252: note = Synthetic Construct
EVGSYSSCKAPYPYCKGSGRSAGGGGTPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 252 aa, synthetic construct
  REGION 1..252: note = Synthetic Construct
EVGSYALCEPFATSVCRPSGRSAGGGGTPLGLAGSGGSDIQLTQSPSSLSASVGDRVTITCRASQVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 253 aa, synthetic construct
  REGION 1..253: note = Synthetic Construct
EVGSYTLCEPFATSVCRPSGRSAGGGGTPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 244 aa, synthetic construct
  REGION 1..244: note = Synthetic Construct
EVGSYSSCKAPYPYCKGSGRGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 244 aa, synthetic construct
  REGION 1..244: note = Synthetic Construct
EVGSYSSCKAPYPYCKGSGGGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 243 aa, synthetic construct
  REGION 1..243: note = Synthetic Construct
EVGSYSSCKAPYPYCKGGGGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 240 aa, synthetic construct
  REGION 1..240: note = Synthetic Construct
ESSCKAPYPYCKGSGGGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 245 aa, synthetic construct
  REGION 1..245: note = Synthetic Construct
EVGSYTLCEPFATSVCRPSGRGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 245 aa, synthetic construct
  REGION 1..245: note = Synthetic Construct
EVGSYTLCEPFATSVCRPSGGGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 241 aa, synthetic construct
  REGION 1..241: note = Synthetic Construct
ETLCEPFATSVCRPSGGGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 244 aa, synthetic construct
  REGION 1..244: note = Synthetic Construct
EVGSYTLCEPFATSVCRPGGGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 240 aa, synthetic construct
  REGION 1..240: note = Synthetic Construct
ETLCEPFATSVCRPGGGPLGLAGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 241 aa, synthetic construct
  REGION 1..241: note = Synthetic Construct
ETLCEPFATSVCRPGGGPLGLAGGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 241 aa, synthetic construct; identical to SEQ ID NO: 120
  REGION 1..241: note = Synthetic Construct

SEQ ID NO: 122 - protein, 5 aa, synthetic construct
  REGION 1..5: note = Synthetic Construct
EVGSY

SEQ ID NO: 123 - protein, 5 aa, synthetic construct
  REGION 1..5: note = Synthetic Construct
SGRSA

SEQ ID NO: 124 - protein, 6 aa, synthetic construct
  REGION 1..6: note = Synthetic Construct
PLGLAG

SEQ ID NO: 125 - protein, 7 aa, synthetic construct
  REGION 1..7: note = Synthetic Construct
ENLYFQG

SEQ ID NO: 126 - protein, 5 aa, synthetic construct
  REGION 1..5: note = Synthetic Construct
GGGGS

SEQ ID NO: 127 - protein, 4 aa, synthetic construct
  REGION 1..4: note = Synthetic Construct
SGGS

SEQ ID NO: 128 - protein, 4 aa, synthetic construct
  REGION 1..4: note = Synthetic Construct
GGSG

SEQ ID NO: 129 - protein, 5 aa, synthetic construct
  REGION 1..5: note = Synthetic Construct
GGSGG

SEQ ID NO: 130 - protein, 5 aa, synthetic construct
  REGION 1..5: note = Synthetic Construct
GSGSG

SEQ ID NO: 131 - protein, 5 aa, synthetic construct
  REGION 1..5: note = Synthetic Construct
GSGGG

SEQ ID NO: 132 - protein, 5 aa, synthetic construct
  REGION 1..5: note = Synthetic Construct
GGGSG

SEQ ID NO: 133 - protein, 5 aa, synthetic construct
  REGION 1..5: note = Synthetic Construct
GSSSG

SEQ ID NO: 134 - protein, 120 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE

SEQ ID NO: 135 - protein, 13 aa, synthetic construct
  REGION 1..13: note = Synthetic Construct
FSLSTGGVGVGWI

SEQ ID NO: 136 - protein, 20 aa, synthetic construct
  REGION 1..20: note = Synthetic Construct
LALIDWADDKYYSPSLKSRL

SEQ ID NO: 137 - protein, 15 aa, synthetic construct
  REGION 1..15: note = Synthetic Construct
ARGGSDTVIGDWFAY

SEQ ID NO: 138 - protein, 11 aa, synthetic construct
  REGION 1..11: note = Synthetic Construct
RASQSIGSYLA

SEQ ID NO: 139 - protein, 9 aa, synthetic construct
  REGION 1..9: note = Synthetic Construct
DASNLETGV

SEQ ID NO: 140 - protein, 10 aa, synthetic construct
  REGION 1..10: note = Synthetic Construct
YCQQGYYLWT

SEQ ID NO: 141 - protein, 123 aa, synthetic construct
  REGION 1..123: note = Synthetic Construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 142 - protein, 107 aa, synthetic construct
  REGION 1..107: note = Synthetic Construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 143 - protein, 5 aa, synthetic construct
  REGION 1..5: note = Synthetic Construct
EVGSY

SEQ ID NO: 144-145 - skipped (2 entries)

SEQ ID NO: 146 - protein, 11 aa, synthetic construct
  REGION 1..11: note = Synthetic Construct
VLCAKFPFCIT

SEQ ID NO: 147 - protein, 12 aa, synthetic construct
  REGION 1..12: note = Synthetic Construct
PPCVGFYPYCKT

SEQ ID NO: 148 - protein, 12 aa, synthetic construct
  REGION 1..12: note = Synthetic Construct
SSCKAPYPYCKG

SEQ ID NO: 149 - protein, 13 aa, synthetic construct
  REGION 1..13: note = Synthetic Construct
ALCEPFATSVCRP

SEQ ID NO: 150 - protein, 13 aa, synthetic construct
  REGION 1..13: note = Synthetic Construct
TLCEPFATSVCRP

SEQ ID NO: 151 - protein, 13 aa, synthetic construct
  REGION 1..13: note = Synthetic Construct
TLCEPFATSVCRP

SEQ ID NO: 152 - protein, 5 aa, synthetic construct
  REGION 1..5: note = Synthetic Construct
SGRSA

SEQ ID NO: 153 - protein, 4 aa, synthetic construct
  REGION 1..4: note = Synthetic Construct
SGRG

SEQ ID NO: 154 - protein, 6 aa, synthetic construct
  REGION 1..6: note = Synthetic Construct
PLGLAG

SEQ ID NO: 155 - protein, 5 aa, synthetic construct
  REGION 1..5: note = Synthetic Construct
GGGGT

SEQ ID NO: 156 - skipped

SEQ ID NO: 157 - protein, 4 aa, synthetic construct
  REGION 1..4: note = Synthetic Construct
SGGG

SEQ ID NO: 158 - skipped

SEQ ID NO: 159 - protein, 12 aa, synthetic construct
  REGION 1..12: note = Synthetic Construct
GGGGSGGSGGGS

SEQ ID NO: 160 - protein, 4 aa, synthetic construct
  REGION 1..4: note = Synthetic Construct
SGGS

SEQ ID NO: 161 - skipped

SEQ ID NO: 162 - protein, 36 aa, synthetic construct
  REGION 1..36: note = Synthetic Construct
EVGSYVLCAKFPFCITSGRSAGGGGTPLGLAGSGGS

SEQ ID NO: 163 - protein, 37 aa, synthetic construct
  REGION 1..37: note = Synthetic Construct
EVGSYPPCVGFYPYCKTSGRSAGGGGTPLGLAGSGGS

SEQ ID NO: 164 - protein, 37 aa, synthetic construct
  REGION 1..37: note = Synthetic Construct
EVGSYSSCKAPYPYCKGSGRSAGGGGTPLGLAGSGGS

SEQ ID NO: 165 - protein, 38 aa, synthetic construct
  REGION 1..38: note = Synthetic Construct
EVGSYALCEPFATSVCRPSGRSAGGGGTPLGLAGSGGS

SEQ ID NO: 166 - protein, 38 aa, synthetic construct
  REGION 1..38: note = Synthetic Construct
EVGSYTLCEPFATSVCRPSGRSAGGGGTPLGLAGSGGS

SEQ ID NO: 167 - protein, 29 aa, synthetic construct
  REGION 1..29: note = Synthetic Construct
EVGSYSSCKAPYPYCKGSGRGPLGLAGGS

SEQ ID NO: 168 - protein, 29 aa, synthetic construct
  REGION 1..29: note = Synthetic Construct
EVGSYSSCKAPYPYCKGSGGGPLGLAGGS

SEQ ID NO: 169 - protein, 28 aa, synthetic construct
  REGION 1..28: note = Synthetic Construct
EVGSYSSCKAPYPYCKGGGGPLGLAGGS

SEQ ID NO: 170 - protein, 25 aa, synthetic construct
  REGION 1..25: note = Synthetic Construct
ESSCKAPYPYCKGSGGGPLGLAGGS

SEQ ID NO: 171 - protein, 30 aa, synthetic construct
  REGION 1..30: note = Synthetic Construct
EVGSYTLCEPFATSVCRPSGRGPLGLAGGS

SEQ ID NO: 172 - protein, 30 aa, synthetic construct
  REGION 1..30: note = Synthetic Construct
EVGSYTLCEPFATSVCRPSGGGPLGLAGGS

SEQ ID NO: 173 - protein, 26 aa, synthetic construct
  REGION 1..26: note = Synthetic Construct
ETLCEPFATSVCRPSGGGPLGLAGGS

SEQ ID NO: 174 - protein, 29 aa, synthetic construct
  REGION 1..29: note = Synthetic Construct
EVGSYTLCEPFATSVCRPGGGPLGLAGGS

SEQ ID NO: 175 - protein, 25 aa, synthetic construct
  REGION 1..25: note = Synthetic Construct
ETLCEPFATSVCRPGGGPLGLAGGS

SEQ ID NO: 176 - protein, 26 aa, synthetic construct
  REGION 1..26: note = Synthetic Construct
ETLCEPFATSVCRPGGGPLGLAGGGS

SEQ ID NO: 177 - protein, 26 aa, synthetic construct
  REGION 1..26: note = Synthetic Construct
ETLCEPFATSVCRPGGGGSGGSGGGS

(19) United States
(12) Patent Application Publication
(10) Pub. No.: US 2026/0258131 A1
(43) Pub. Date: Sep. 3, 2026

(54) ANTI-CTLA4 ANTIBODIES AND METHODS OF MAKING AND USING THE SAME

(71) Applicant: ADAGENE PTE. LTD., Singapore (SG)

(72) Inventors: Peter Peizhi LUO, Morgan Hill, CA (US); Fangyong DU, Suzhou, Jiangsu (CN); Yan LI, San Jose, CA (US); Guizhong LIU, Fort Lee, NJ (US)

(73) Assignee: ADAGENE PTE. LTD., Singapore (SG)

(21) Appl. No.: 19/474,632

(22) PCT Filed: Apr. 12, 2024
(86) PCT No.: PCT/CN2024/087455

§ 371 Date: Oct. 10, 2025

(30) Foreign Application Priority Data
Apr. 13, 2023 (WO) PCT/CN2023/088000

(51) Int. Cl. C07K 16/28; A61K 39/00; A61K 45/06; A61P 35/00

(52) U.S. Cl. CPC C07K 16/2818; A61K 45/06; A61P 35/00; A61K 2039/505; C07K 2317/14; C07K 2317/41; C07K 2317/52; C07K 2317/565; C07K 2317/732; C07K 2317/92

(57) ABSTRACT

Provided herein are antibodies (or antigen binding fragments thereof) that bind to human CTLA4 (e.g., antibodies that bind to human CTLA4) and have enhanced effector functions, masked antibodies (e.g., activatable antibodies) that bind to human CTLA4, nucleic acid molecules encoding the same, pharmaceutical compositions thereof, and methods of their therapeutic use (e.g., for treatment of cancer).

### CROSS REFERENCE TO RELATED APPLICATIONS

The application claims the priority benefit of International Application No. PCT/CN2023/088000, filed Apr. 13, 2023, the contents of which are incorporated by reference in their entirety.

### SUBMISSION OF SEQUENCE LISTING ON ASCII TEXT FILE

The contents of the electronic sequence listing (695402001840SEQLIST. xml; Size: 223, 607 bytes; and Date of Creation: Apr. 12, 2023) is herein incorporated by reference in its entirety.

### FIELD OF THE INVENTION

The present disclosure relates to antibodies that bind to human Cytotoxic T-lymphocyte Protein 4 (CTLA4), masked antibodies (e.g., activatable antibodies) that bind to human CTLA4, nucleic acids encoding the same, pharmaceutical compositions thereof, and their therapeutic use.

### BACKGROUND

CTLA4 is a member of the immunoglobulin (Ig) superfamily of proteins that acts to downregulate T-cell activation and maintain immunogenic homeostasis. It has been shown that in vivo antibody-mediated blockade of CTLA4 enhanced anti-cancer immune responses in a syngeneic murine prostate cancer model (Kwon et al. (1997) Proc Natl Acad Sci USA, 94 (15): 8099-103). In addition, blockade of CTLA4 function was shown to enhance anti-tumor T cell responses at various stages of tumor growth in tumor-bearing mice (Yang et al. (1997) Cancer Res 57 (18): 4036-41; Hurwitz et al. (1998) Proc Natl Acad Sci USA 95 (17): 10067-7). However, the development of antibody-based therapeutics suitable for human use remains difficult, as translation from pre-clinical animal models to human safety is often poor. Accordingly, a need exists for anti-CTLA4 antibodies that are cross-reactive among different species, such as humans and experimental animals (e.g., mouse, monkey, rat, etc.), to concurrently enable animal model studies and provide suitable human therapeutic candidates. In addition, because CTLA4 is expressed on healthy cells, such as T effector cells, CTLA4 can have negative impacts on healthy cells and cause dangerous side effects, such as immune checkpoint inhibitor-associated diabetes (Akturk, H. K., et al. “Immune checkpoint inhibitor-induced type 1 diabetes: a systematic review and meta-analysis.” Diabetic Medicine 36.9 (2019): 1075-1081). Therefore, a need exists for the development of safer anti-CTLA4 antibodies that are only active in certain contexts, such as in the protease-rich tumor microenvironment.

### BRIEF SUMMARY

In one aspect, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering. In some embodiments, the isolated antibody induces antibody-dependent cell cytotoxicity (ADCC) against a CTLA4-expressing human cell or a human Treg cell, wherein the ADCC activity of the isolated antibody is higher than that of a control antibody comprising a human IgG1 Fc region that does not comprise the substitutions, and/or wherein the ADCC activity of the isolated antibody is higher than the ADCC activity of ipilimumab.

In some embodiments of this aspect, the VH comprises a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3, and the VL comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6. In certain embodiments, the VH comprises the amino acid sequence of SEQ ID NO: 74, and/or the VL comprises the amino acid sequence of SEQ ID NO: 75. In one embodiments, the heavy chain comprises the amino acid sequence of SEQ ID NO: 91, and the light chain comprises the amino acid sequence of SEQ ID NO: 90. In another embodiment, the heavy chain comprises the amino acid sequence of SEQ ID NO: 91 without the C-terminal lysine, and the light chain comprises the amino acid sequence of SEQ ID NO: 90. In other embodiments of this aspect, the VH comprises a CDR-H1 comprising the amino acid sequence of SEQ ID NO: 12, a CDR-H2 comprising the amino acid sequence of SEQ ID NO: 13, a CDR-H3 comprising the amino acid sequence of SEQ ID NO: 14, and the VL comprises a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17. In certain embodiments, the VH comprises the amino acid sequence of SEQ ID NO: 66, and/or the VL comprises the amino acid sequence of SEQ ID NO: 67. In still other embodiments of this aspect, the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 53, and the VL comprises a CDR-L1 comprising the amino acid sequence of SEQ ID NO: 54, a CDR-L2 comprising the amino acid sequence of SEQ ID NO: 16, and a CDR-L3 comprising the amino acid sequence of SEQ ID NO: 55. In certain embodiments, the VH comprises the amino acid sequence of SEQ ID NO: 84, and/or the VL comprises the amino acid sequence of SEQ ID NO: 85. In yet other embodiments of this aspect, the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 46, and the VL comprises a CDR-L1 comprising the amino acid sequence of SEQ ID NO: 47, a CDR-L2 comprising the amino acid sequence of SEQ ID NO: 5, and a CDR-L3 comprising the amino acid sequence of SEQ ID NO: 48. In certain embodiments, the VH comprises the amino acid sequence of SEQ ID NO: 80, and/or the VL comprises the amino acid sequence of SEQ ID NO: 81.

In some embodiments of this aspect, the antibody comprises two heavy chains and two light chains, wherein each human IgG1 Fc domain of the heavy chain comprises an S298A substitution, an E333A substitution, and a K334A substitution. In some embodiments of this aspect, the antibody comprises two heavy chains and two light chains, wherein each human IgG1 Fc domain of the heavy chain comprises an S298A substitution, an E333A substitution, a K334A substitution, and a K326A substitution. In some embodiments of this aspect, the antibody comprises two heavy chains and two light chains, wherein each human IgG1 Fc domain of the heavy chain comprises an F243L substitution. In some embodiments of this aspect, the antibody comprises two heavy chains and two light chains, wherein each human IgG1 Fc domain of the heavy chain comprises an F243L substitution, an R292P substitution, a Y300L substitution, and a P396L substitution. In some embodiments of this aspect, the antibody comprises two heavy chains and two light chains, wherein each human IgG1 Fc domain of the heavy chain comprises an F243L substitution, an R292P substitution, a Y300L substitution, a V305I substitution, and a P396L substitution. In some embodiments of this aspect, the antibody comprises two heavy chains and two light chains, wherein each human IgG1 Fc domain of the heavy chain comprises an F243L substitution, an R292P substitution, a Y300L substitution, a L235V substitution, and a P396L substitution. In some embodiments of this aspect, the antibody comprises two heavy chains and two light chains, wherein each human IgG1 Fc domain of the heavy chain comprises a S239D substitution, an I332E substitution, and an A330L substitution. In some embodiments of this aspect, the antibody comprises two heavy chains and two light chains, wherein each human IgG1 Fc domain of the heavy chain comprises a S239D substitution and an I332E substitution.

In another aspect, provided herein is a masked antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises, from N-terminus to C-terminus, a masking unit (MU) and a linkage unit (LU), wherein the MU comprises an amino acid sequence selected from the list consisting of SEQ ID NOs: 146-151. In some embodiments of this aspect, the VH comprises a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3, and the VL comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6. In certain embodiments, the VH comprises the amino acid sequence of SEQ ID NO: 74, and/or the VL comprises the amino acid sequence of SEQ ID NO: 75. In other embodiments of this aspect, the VH comprises a CDR-H1 comprising the amino acid sequence of SEQ ID NO: 12, a CDR-H2 comprising the amino acid sequence of SEQ ID NO: 13, a CDR-H3 comprising the amino acid sequence of SEQ ID NO: 14, and the VL comprises a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17. In certain embodiments, the VH comprises the amino acid sequence of SEQ ID NO: 66, and/or the VL comprises the amino acid sequence of SEQ ID NO: 67. In still other embodiments of this aspect, the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 53, and the VL comprises a CDR-L1 comprising the amino acid sequence of SEQ ID NO: 54, a CDR-L2 comprising the amino acid sequence of SEQ ID NO: 16, and a CDR-L3 comprising the amino acid sequence of SEQ ID NO: 55. In certain embodiments, the VH comprises the amino acid sequence of SEQ ID NO: 84, and/or the VL comprises the amino acid sequence of SEQ ID NO: 85. In yet other embodiments of this aspect, the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 46, and the VL comprises a CDR-L1 comprising the amino acid sequence of SEQ ID NO: 47, a CDR-L2 comprising the amino acid sequence of SEQ ID NO: 5, and a CDR-L3 comprising the amino acid sequence of SEQ ID NO: 48. In certain embodiments, the VH comprises the amino acid sequence of SEQ ID NO: 80, and/or the VL comprises the amino acid sequence of SEQ ID NO: 81. In some embodiments, the masked antibody is a Fab fragment, an scFv, a F (ab′)₂ fragment, or an Fv fragment.

In some embodiments of this aspect, the masking peptide (MP) further comprises an N-terminal unit (NU) linked to the N-terminal of the MU. In some embodiments, the N-terminal unit is about 1-10 amino acid residues long. In certain embodiments, the N-terminal unit comprises an amino acid sequence selected from the group consisting of SEQ ID NOs: 143-145.

In some embodiments of this aspect, the linkage unit (LU) does not comprise a cleavage site. In some embodiments, the LU comprises a linker. In certain embodiments, the linker comprises an amino acid sequence selected from the group consisting of SEQ ID NOs: 155-161. In certain embodiments, the linker comprises the amino acid sequence of SEQ ID NO: 159.

In some embodiments of this aspect, the masked antibody is an activatable antibody. In some embodiments wherein the antibody is an activatable antibody, the LU comprises at least a first cleavage site (C₁). In some embodiments, the first cleavage site (C₁) is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In certain embodiments, the first cleavage site (C₁) comprises an amino acid sequence selected from the group consisting of SEQ ID NOs: 152-154. In some embodiments wherein the antibody is an activatable antibody, the LU further comprises a second cleavage site (C₂). In some embodiments, the second cleavage (C₂) site is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In certain embodiments, the second cleavage site (C₂) comprises an amino acid sequence selected from the group consisting of SEQ ID NOs: 152-154. In certain embodiments, the second cleavage site (C₂) comprises an amino acid sequence selected from the group consisting of SEQ ID NOs: 152-154. In some embodiments, the first and second cleavage sites are different. In some embodiments wherein the antibody is an activatable antibody, the LU further comprises a first linker. In certain embodiments, the first linker comprises an amino acid sequence selected from the group consisting of SEQ ID NOs: 155-161. In some embodiments, the LU further comprises a second linker (L₂). In certain embodiments, the second linker comprises an amino acid sequence selected from the group consisting of SEQ ID NOs: 155-161. In some embodiments, the LU comprises, from N-terminus to C-terminus the first cleavage site (C₁), the first linker (L₁), the second cleavage site (C₂), and the second linker (L₂) (i.e., a C₁-L₁-C₂-L₂ configuration). In other embodiments, the LU comprises, from N-terminus to C-terminus the first linker (L₁), the first cleavage site (C₁), and the second linker (L₂) (i.e., a L₁-C₁-L₂ configuration). In some embodiments, the LU comprises an amino acid sequence selected from the group consisting of SEQ ID NOs: 99-103. In certain embodiments, the MU comprises the amino acid sequence of SEQ ID NO: 146, and the LU comprises the amino acid sequence of SEQ ID NO: 99. In certain embodiments, the MU comprises the amino acid sequence of SEQ ID NO: 147, and the LU comprises the amino acid sequence of SEQ ID NO: 99. In certain embodiments, the MU comprises the amino acid sequence of SEQ ID NO: 148, and the LU comprises the amino acid sequence of SEQ ID NO: 99. In certain embodiments, the MU comprises the amino acid sequence of SEQ ID NO: 149, and the LU comprises the amino acid sequence of SEQ ID NO: 99. In certain embodiments, the MU comprises the amino acid sequence of SEQ ID NO: 150, and the LU comprises the amino acid sequence of SEQ ID NO: 99. In certain embodiments, the MU comprises the amino acid sequence of SEQ ID NO: 148, and the LU comprises the amino acid sequence of SEQ ID NO: 100. In certain embodiments, the MU comprises the amino acid sequence of SEQ ID NO: 148, and the LU comprises the amino acid sequence of SEQ ID NO: 101. In certain embodiments, the MU comprises the amino acid sequence of SEQ ID NO: 148, and the LU comprises the amino acid sequence of SEQ ID NO: 102. In certain embodiments, the MU comprises the amino acid sequence of SEQ ID NO: 148, and the LU comprises the amino acid sequence of SEQ ID NO: 101. In certain embodiments, the MU comprises the amino acid sequence of SEQ ID NO: 151, and the LU comprises the amino acid sequence of SEQ ID NO: 100. In certain embodiments, the MU comprises the amino acid sequence of SEQ ID NO: 151, and the LU comprises the amino acid sequence of SEQ ID NO: 101. In certain embodiments, the MU comprises the amino acid sequence of SEQ ID NO: 150, and the LU comprises the amino acid sequence of SEQ ID NO: 101. In certain embodiments, the MU comprises the amino acid sequence of SEQ ID NO: 150, and the LU comprises the amino acid sequence of SEQ ID NO: 102. In certain embodiments, the MU comprises the amino acid sequence of SEQ ID NO: 150, and the LU comprises the amino acid sequence of SEQ ID NO: 102. In certain embodiments, the MU comprises the amino acid sequence of SEQ ID NO: 150, and the LU comprises the amino acid sequence of SEQ ID NO: 103. In certain embodiments, the MU comprises the amino acid sequence of SEQ ID NO: 150, and the LU comprises the amino acid sequence of SEQ ID NO: 103. In some embodiments, the masking peptide comprises an amino acid sequence selected from the group consisting of SEQ ID NOs: 162-177.

In certain embodiments of this aspect, the masked antibody comprises two heavy chains and two light chains, wherein each human IgG1 Fc domain of the heavy chain comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering. In some embodiments of this aspect, the masked antibody comprises two heavy chains and two light chains, wherein each human IgG1 Fc domain of the heavy chain comprises an S298A substitution, an E333A substitution, and a K334A substitution. In some embodiments of this aspect, the masked antibody comprises two heavy chains and two light chains, wherein each human IgG1 Fc domain of the heavy chain comprises an S298A substitution, an E333A substitution, a K334A substitution, and a K326A substitution. In some embodiments of this aspect, the masked antibody comprises two heavy chains and two light chains, wherein each human IgG1 Fc domain of the heavy chain comprises an F243L substitution. In some embodiments of this aspect, the masked antibody comprises two heavy chains and two light chains, wherein each human IgG1 Fc domain of the heavy chain comprises an F243L substitution, an R292P substitution, a Y300L substitution, and a P396L substitution. In some embodiments of this aspect, the masked antibody comprises two heavy chains and two light chains, wherein each human IgG1 Fc domain of the heavy chain comprises an F243L substitution, an R292P substitution, a Y300L substitution, a V305I substitution, and a P396L substitution. In some embodiments of this aspect, the masked antibody comprises two heavy chains and two light chains, wherein each human IgG1 Fc domain of the heavy chain comprises an F243L substitution, an R292P substitution, a Y300L substitution, a L235V substitution, and a P396L substitution. In some embodiments of this aspect, the masked antibody comprises two heavy chains and two light chains, wherein each human IgG1 Fc domain of the heavy chain comprises a S239D substitution, an I332E substitution, and an A330L substitution. In some embodiments of this aspect, the masked antibody comprises two heavy chains and two light chains, wherein each human IgG1 Fc domain of the heavy chain comprises a S239D substitution and an I332E substitution. In some embodiments, the heavy chains each comprise an amino acid sequence selected from the group consisting of SEQ ID NOS: 104-105, and the light chains each comprise an amino acid sequence selected from the group consisting of SEQ ID NOS: 106-121. In certain embodiments, the heavy chains each comprise the amino acid sequence of SEQ ID NO: 105, and the light chains each comprise the amino acid sequence of SEQ ID NO: 121. In some embodiments, the masked antibody induces antibody-dependent cell cytotoxicity (ADCC) against a CTLA4-expressing human cell or a human Treg cell, wherein the ADCC activity of the isolated antibody is higher than that of a control antibody comprising a human IgG1 Fc region that do not comprise the substitutions, and/or wherein the ADCC activity of the isolated antibody is higher than the ADCC activity of ipilimumab.

In another aspect, provided herein is an isolated polynucleotide encoding one or more polypeptide chains of any of the isolated antibodies or masked antibodies described herein. In yet another aspect, provided herein is a vector comprising said polynucleotide. In some embodiments, the vector is an expression vector and/or a display vector. In still another aspect, provided herein is a host cell comprising said polynucleotide or said vector. In some embodiments, the host cell is a eukaryotic cell. In certain embodiments, the host cell is a Chinese Hamster Ovary (CHO) cell. In yet another aspect, provided herein is a method of making any of the antibodies or masked antibodies described herein, the method comprising culturing said host cell under conditions suitable for producing the antibody or activatable antibody. In some embodiments, the method further comprises recovering the antibody or masked antibody produced by the cell.

In still another aspect, provided herein is a pharmaceutical composition comprising any of the isolated antibodies or masked antibodies described herein, and a pharmaceutically acceptable carrier.

In yet another aspect, provided herein is a method of treating or delaying progression of a cancer in a subject in need thereof, the method comprising administering to the subject an effective amount of any of the isolated antibodies or masked antibodies described herein, or a pharmaceutical composition comprising any of the isolated antibodies or masked antibodies described herein.

In still another aspect, provided herein is a method of reducing size of a solid tumor in a subject in need thereof, wherein the solid tumor has a size of about 400-1000 mm³, the method comprises administering to the subject an effective amount of any of the isolated antibodies or masked antibodies described herein, or a pharmaceutical composition comprising any of the isolated antibodies or masked antibodies described herein.

In yet another aspect, provided herein is a method of reducing on-target off-tumor toxicity in a subject having a cancer in need thereof, wherein the method comprises administering to the subject an effective amount of any of the isolated antibodies or masked antibodies described herein, or a pharmaceutical composition comprising any of the isolated antibodies or masked antibodies described herein. In some embodiments, the tumor is a colorectal tumor or a lung tumor. In some embodiments, the tumor is a cold tumor. In certain embodiments, the cold tumor is selected from the group consisting of pancreatic cancer, ovarian cancer, and head and neck cancer.

In some embodiments of any of the preceding methods, the method further comprises administering to the subject an effective amount of at least one additional therapeutic agent. In some embodiments, the at least one additional therapeutic agent is selected from the group consisting of viral gene therapy, immune checkpoint inhibitors, target therapies, radiation therapies, vaccination therapies, and chemotherapies. In certain embodiments, the at least one additional therapeutic agent is an immune checkpoint inhibitor. In certain embodiments, the at least one additional therapeutic agent is an inhibitor of PD-1. In some embodiments of any of the preceding methods, the method comprises administering to the subject an effective amount of the antibody, the masked antibody, or the pharmaceutical composition prior to a surgery or after a surgery to remove a solid tumor in the subject.

In yet another aspect, provided herein is a method inducing phagocytosis of cells expressing CTLA4, the method comprising contacting the cells expressing CTLA4 with an effective amount of any of the isolated antibodies or masked antibodies described herein. In some embodiments, the phagocytosis is antibody-dependent cellular phagocytosis (ADCP). In certain embodiments, the antibody, or the masked antibody, has a half maximal effective concentration (EC50) of less than 3 nM for inducing ADCP of cells expressing CTLA4.

It is to be understood that one, some, or all of the properties of the various embodiments described above and herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIG. 1 show functional display of scFvs targeting CTLA4 on yeast, as determined by flow cytometry.

FIG. 2 shows an exemplary selection process for activatable antibodies targeting human CTLA4. A yeast library displaying fusion proteins were subjected to several rounds of FACS-based screening.

FIG. 3 shows binding affinity of CTLA4 activatable antibody clones in the scFv format, including the CTLA4 activatable antibody clone BC3864, with the masking peptide intact, or with the masking peptide cleaved by the TEV protease, as compared to the scFv fragment of the target antibody with no masking peptide.

FIGS. 4A-4D show the masking efficiency of exemplary CTLA4 activatable antibodies against recombinant human CLTA4-hisFc, as compared to the parental antibody TY21580, as determined by enzyme-linked immunoassay (ELISA).

FIGS. 5A-5B show activity of CTLA4 activatable antibodies TY24649 and TY24652 upon removal of the masking peptide. FIG. 5A shows reduced CE-SDS results of activatable antibody TY24649 and TY24652 with no treatment, treated with the protease MMP9. FIG. 5B shows binding of activatable antibodies TY24649 and TY24652 treated with the protease MMP9, as compared to the parental antibody TY21580, as determined by ELISA.

FIGS. 6A-6B show the size-exclusion chromatography (SEC) profiles of exemplary activatable antibodies under accelerated stress conditions. FIG. 6A shows the SEC profiles of activatable antibody TY24649 and TY24652 after six cycles of freezing and thawing, as compared to the control condition. FIG. 6B shows the SEC profiles of the exemplary activatable antibodies after storage at 40° C. for up to 28 days, as compared to the control condition.

FIG. 7 shows the hydrophobic interaction chromatography (HIC) profiles of exemplary activatable antibodies, as compared with the parental antibody TY21580, and the commercial drug Tecentriq.

FIG. 8 shows the effect on IL-2 secretion from SEA induced human peripheral blood mononuclear cell (PBMC) activation of isotype control antibody, parental antibody TY21580 and TY24040, or exemplary target activatable antibodies TY24649, TY24652 and TY22404 before and after cleavage, as measured by ELISA.

FIG. 9 shows the antibody-dependent cell-mediated cytotoxicity (ADCC) activity of isotype control antibody, the parental antibody TY21580 and TY24040, or exemplary activatable antibodies TY24649, TY24652 and TY22404 before and after cleavage, on HEK293F cells transiently overexpressing human CTLA4, as determined by an ADCC reporter gene assay.

FIG. 10 shows the CLTA4 blockade activity of isotype control antibody, the parental antibody TY21580 and TY24040, or exemplary activatable antibodies TY24649, TY24652 and TY22404 before and after cleavage, as determined by an CTLA4 blockade bioassay.

FIG. 11 shows the binding activities of parental and activatable anti-CTLA-4 antibodies to cell surface human CTLA4 expressed on HEK293F cells. The concentration-dependent binding curves of isotype control antibody, the parental antibody TY21580 and TY24040, or exemplary activatable antibodies TY24649 and TY24652 before and after cleavage, were determined by flow cytometry assay.

FIG. 12 shows the binding affinities of wild type and ADCC enhancing mutant Fc to Fcγ receptor CD16 of human, monkey and mouse origins. The activated TY24649 with wild type Fc and the activated TY24652 with mutant Fc were tested by Biacore.

FIGS. 13A-13C show the in vivo anti-tumor efficacy of parental antibody TY21580, isotype control antibody, or exemplary CTLA4 activatable antibodies TY24649 and TY24652, non-cleavable antibody TY24851 in an MC38 syngeneic mouse colorectal tumor model. FIG. 13A shows the tumor growth curves of different treatment groups and the individual tumor growth curves for the groups of female C57BL/6 mice bearing MC38-established tumors. In the top panel, data points represent group mean; error bars represent SEM. In the lower panels, curves represent tumor growth in individual mice in each treatment group. 10 mg/kg of TY21580, TY24649, TY24652 and TY24851 was administered two times a week for 4 doses. FIG. 13B shows the tumor growth curves of different treatment groups and the individual tumor growth curves for the groups of female C57BL/6 mice bearing MC38-established tumors. In the top panel, data points represent group mean; error bars represent SEM. In the lower panels, curves represent tumor growth in individual mice in each treatment group. 2 mg/kg of TY21580, TY24649, TY24652 and TY24851 was administered two times a week for 4 doses. FIG. 13C shows the tumor growth curves of different treatment groups for the groups of female C57BL/6 mice bearing MC38-established tumors. Data points represent group mean; error bars represent SEM. 0.05, 0.2, or 1 mg/kg of TY21580 and TY24652 was administered two times a week for 4 doses.

FIGS. 14A-14C show the in vivo anti-tumor efficacy of isotype control antibody, parental antibody TY21580, or exemplary CTLA4 activatable antibodies TY24649 and TY24652, non-cleavable antibody TY24851, in a CT26 syngeneic mouse colorectal tumor model. Tumor growth curves of different treatment groups of female C57BL/6 mice bearing CT26-established tumors are shown. Data points represent group mean; error bars represent SEM. FIG. 14A shows the tumor growth curves of 5 mg/kg of TY21580, TY24649, TY24652 and TY24851 administered two times a week for 4 doses, with the top panel showing the average of each group and the lower panels showing the tumor volume of individual mice in each group. FIG. 14B shows the tumor growth curves of 1 mg/kg of TY21580, TY24649, TY24652 and TY24851 was administered two times a week for 4 doses, with the top panel showing the average of each group and the lower panels showing the tumor volume of individual mice in each group. FIG. 14C shows the tumor growth curves of 0.5 mg/kg TY21580 and 0.1, 0.5, and 2.5 mg/kg TY24652 administered two times a week for 4 doses.

FIGS. 15A-B shows the in vivo anti-tumor efficacy of isotype control antibody, parental antibody TY21580 and activatable antibodies TY24649 and TY24652, in a Lewis syngeneic mouse liver tumor model. Tumor growth curves of different treatment groups of female C57BL/6 mice bearing Lewis-established tumors are shown. Data points represent group mean; error bars represent SEM. FIG. 15A shows the tumor growth curves of 10 mg/kg of TY21580, TY24649 and TY24652 was administered two times a week for 4 doses, FIG. 15B shows the tumor growth curves of 2 mg/kg of TY21580, TY24649 and TY24652 was administered two times a week for 4 doses

FIG. 16 shows a pharmacokinetic analysis of parental antibody TY21580 and activatable antibodies TY24649, TY24652 and TY22404.

FIG. 17 shows the plasma concentration-time profile of the activatable anti-CTLA-4 antibodies TY24649 and TY24652 in cynomolgus monkeys following 2 weekly i. v. dose at 30 mg/kg.

FIG. 18 shows the effects of the anti-CTLA4 antibodies on the stimulation of peripheral lymphocyte subtypes. The parental TY21580 and activatable antibodies TY22404, TY24649 and TY24652 were i. v. administered weekly into cynomolgus monkeys at 30 mg/kg for 2 doses. Peripheral blood samples were collected before and during the treatments, subtypes of lymphocytes were examined by flow cytometry with specific markers. Percentages of the central memory CD4+ T cells, proliferative central memory CD4+ T cells, and proliferative central memory CD8+ T cells were significantly affected on Day 14 post-dose by the anti-CTLA4 antibodies.

FIG. 19 shows the effects of parental and activatable anti-CTLA4 antibodies in combination with anti-PD-1 antibody on the induction of diabetes in the NOD/LtJ mice. The blood glucose levels in each mouse were monitored over time and plotted.

FIG. 20 shows the antibody-dependent cellular phagocytosis (ADCP) of cells overexpressing CTLA-4, induced by the anti-CTLA4 antibodies and measured using a luciferase assay.

FIG. 21A shows the in vivo anti-tumor efficacy of activatable anti-CTLA4 antibody TY24652, anti-CD137 antibody AG10131, and a combination thereof in an MC38 syngeneic mouse colorectal tumor model. The tumor growth curves of different treatment groups are shown for the groups of female C57BL/6 mice bearing MC38-established tumors. Data points represent group mean; error bars represent SEM. 0.2 mg/kg TY24652, 10 mg/kg AG10131, and a combination thereof were administered two times a week for 4 doses.

FIG. 21B shows the in vivo anti-tumor efficacy of activatable anti-CTLA4 antibody TY24652, an anti-PD-1 antibody, and a combination thereof in a CT26 syngeneic mouse colorectal tumor model. Tumor growth curves of different treatment groups of female C57BL/6 mice bearing CT26-established tumors are shown. Data points represent group mean; error bars represent SEM. 0.5 mg/kg TY24652, 5 mg/kg anti-PD-1 antibody, and a combination thereof were administered two times a week for 4 doses

FIGS. 22A-22E show Treg cell depletion by activatable anti-CTLA4 antibody TY24652 in tumor-infiltrating lymphocytes (TIL), peripheral lymphocytes (PMBC), and the spleen in a CT26 syngeneic mouse colorectal tumor model. Brackets with one asterisk (*) and brackets with three asterisks (***) indicate a significant difference between TY24652 and the isotype control at p<0.001 and p<0.05, respectively. Brackets with “n. s.” indicate no significant difference between TY24652 and the isotype control. FIG. 22A shows the percent Treg of CD4+ T cells. FIG. 22B shows the CD8+T to Treg ratio. FIG. 22C shows the CTLA4 expression level of Tregs. FIG. 22D shows the percent CD4+ T cells of the total T cells. FIG. 22E shows the percent CD8+ T cells of the total T cells.

FIGS. 23A-23B show plasma concentration-time profiles of total form of anti-CTLA4 antibodies following a single intravenous or subcutaneous injection at 0.5 mg/kg into minipigs. FIG. 23A shows plasma concentration-time profiles for minipigs injected intravenously. FIG. 23B shows plasma concentration-time profiles for minipigs injected subcutaneously.

FIG. 24 shows ADCC reporter activity of anti-CTLA4 antibodies with various Fc mutations as compared to the parental antibody TY21580, Ipilimumab, and an isotype control.

FIG. 25 shows the in vivo anti-tumor efficacy of isotype control antibody, parental antibody TY21580 and antibodies with Fc mutations TY24037, TY24039, and TY24040 in a MC38 colon cancer model. Data points represent group mean; error bars represent SEM.

FIG. 26A-C show the in vivo anti-tumor efficacy of isotype control antibody, ipilimumab, TY21580 and TY24040 in a MC38 colon cancer model. FIG. 26A shows the average tumor volume up to day 20 in mice treated with the antibodies at 0.5 mg/kg or 5 mg/kg; error bars represent the SEM. FIG. 26B shows the tumor volume for individual mice treated with ipilimumab, TY21580 and TY24040 at 0.5 mg/kg vs. the isotype control. FIG. 26C shows the tumor volume for individual mice treated with ipilimumab, TY21580 and TY24040 at 5 mg/kg vs. the isotype control. In FIGS. 26B-26C, the number inset shows the fraction of mice that survived beyond day 40 of the study.

FIGS. 27A-27B show Treg depletion by an isotype control, ipilimumab, TY21580 and TY24040 in the tumor microenvironment of a MC38 colon cancer model. FIG. 27A shows the percentage Treg cells of CD4+ T cells in tumors from the mice. The bracket with asterisks indicates a statistically significant difference in both TY21580 and TY24040 as compared to the isotype control. FIG. 27B shows the number of Tregs per million live cells. The bracket with the asterisk indicates a statistically significant difference in each of ipilimumab, TY21580, and TY24040 as compared to the isotype control. **indicates P<0.01; *indicates P<0.05.

FIG. 28A-C show the in vivo anti-tumor efficacy of isotype control antibody, ipilimumab, TY21580 and TY24040 to suppress the growth of large, established tumors (~1, 500 mm³) in a MC38 colon cancer model. FIG. 28A shows the average tumor volume up to day 20 in mice treated with the antibodies at 0.5 mg/kg or 5 mg/kg; error bars represent the SEM. FIG. 28B shows the tumor volume for individual mice treated with ipilimumab, TY21580 and TY24040 at 0.5 mg/kg vs. the isotype control. FIG. 28C shows the tumor volume for individual mice treated with ipilimumab, TY21580 and TY24040 at 5 mg/kg vs. the isotype control.

FIGS. 29A-29D show a positron emission tomography-computed tomography (PET/CT) scan of mice injected with the indicated antibody. Animals were imaged at the indicated time points following injection. H22-CTLA4 tumors are on the left side of the animal and H22 tumors are on the right side. Signal intensity reflects percentage injected dose per gram of tissue (% ID/g) as indicated in the key on the right. FIG. 29A shows TY21580, FIG. 29B shows TY22404, FIG. 29C shows TY24652, and FIG. 29D shows TY24851.

FIGS. 30A-30D show graphs indicating the mean % ID/g in the indicated tissues in mice injected with the indicated antibody. Different bars within each tissue sample reflect the data at the indicated time point. FIG. 30A shows TY21580, FIG. 30B shows TY22404, FIG. 30C shows TY24652, and FIG. 30D shows TY24851.

FIG. 31 shows the effect of various control and anti-CTLA-4 antibodies on IL-2 secretion from SEA activated human peripheral blood mononuclear cell (PBMC) s. AFU indicates afucosylated variants. Asterisk indicates antibodies that comprise Fc-enhancement mutations.

FIG. 32 shows the experimental design for evaluating various control and anti-CTLA-4 antibodies in a MC38 syngeneic colon cancer tumor mouse model. Mice were dosed twice bi-weekly. AFU indicates an afucosylated variant.

FIG. 33 shows the in vivo anti-tumor efficacy of isotype control antibody, ipilimumab, TY21580 and TY24040, and TY21580-AFU to suppress the growth of established tumors (~50-100 mm3) in a MC38 colon cancer model. FIG. 33A shows the average tumor volume up to day 22 in mice treated with the antibodies at 0.5 mg/kg; error bars represent the SEM. AFU indicates an afucosylated variant. FIG. 33B shows the tumor volume for individual mice treated with ipilimumab, TY21580, TY21580-AFU and TY24040 at 0.5 mg/kg.

FIG. 34 shows the experimental design for evaluating various control and anti-CTLA-4 antibodies in a MC38 syngeneic colorectal cancer tumor mouse model. Mice bearing large, established tumors (~200-300 mm3) were dosed three times a week once every 3 days (q3d×3) with 1 mg/kg isotype or an anti-CTLA-4 antibody. AFU indicates an afucosylated variant.

FIG. 35 show Treg depletion by an isotype control, ipilimumab, TY21580, TY21580-AFU, and TY24040 in the tumor microenvironment of a MC38 colon cancer model. *indicates P<0.05. FIG. 35A shows fluorescence activated cell sorting (FACS) plots of CD4+ cells. FIG. 35B shows the percent Treg cells of the CD4+ T cells in tumors from the mice. The bracket with asterisks indicates a statistically significant difference in TY21580 and TY21580-AFU, and in TY21580, TY21580-AFU, and TY24040 as compared to the isotype control and ipilimumab. FIG. 35C shows the number of Tregs per million cells. The bracket with the asterisk indicates a statistically significant difference in each of ipilimumab, TY21580, TY21580-AFU, and TY24040 as compared to the isotype control. FIG. 35D shows the percentage splenic Treg cells compared to CD4+ T cells from the mice. Error bars represent the SEM. AFU indicates an afucosylated variant

### DETAILED DESCRIPTION

To provide anti-CTLA4 antibodies with powerful anti-tumor activity and reduced off-target effects, the inventors developed a suite of masked anti-CTLA4 antibodies (including activatable anti-CTLA4 antibodies), each comprising an N-terminal masking peptide and an IgG1 Fc region. Without wishing to be bound by any theory or hypothesis, the anti-tumor activity of the antibodies is at least two-fold: 1) blocking CTLA4 sequestration of CD80 and CD86 ligands to stimulate CD28 mediated downstream cell signaling, and 2) target cell killing through enhanced IgG1 Fc-mediated effector functions such as ADCC and ADCP. The masking peptide is designed to compete with the anti-CTLA4 antibody moiety for target binding, thereby inhibiting on-target off-tumor activities in healthy tissues and allowing anti-tumor activities in the tumor microenvironment (TME).

A masked antibody is designed to mask its antigen-binding site with a masking unit in the masking peptide, which prevents the antibody from binding to its target in healthy tissues. Masked antibodies may be activatable, wherein the masking unit is designed to be unmasked to activate the antibody in a TME having certain activation conditions, allowing the antigen-binding site to bind to its target. For example, the masking peptide of an activatable antibody may comprise a cleavage site that, upon cleavage by a protease, results in removal of the masking unit from the antibody and activation of the antibody to bind CTLA4. In normal tissues and in circulation, where the expression level of CTLA4 is low, the masking unit in the activatable anti-CTLA4 antibody functions to block binding to CTLA4, reducing off-target effects associated with anti-CTLA4 antibodies. However, in the TME where the local concentration of CTLA4 is high, the masking antibody is capable of binding to CTLA4 on target cells in the presence of the masking unit (i.e., inactivated state for an activatable antibody), when the masking unit in a portion of the masking antibody is cleaved off (i.e., partially activated state for an activatable antibody), and/or when the masking unit is fully cleaved off (i.e., fully activated state for an activatable antibody). Furthermore, in the TME where protease activity has been reported to be elevated, the masking unit in an activatable antibody is cleaved off, leading to enhanced binding of the activated anti-CTLA4 antibody to CTLA4 on target cells. Thus, the masked anti-CTLA4 antibodies described herein exhibit enhanced anti-tumor activity, both through blocking CTLA4 sequestration of CD80 and CD86 ligands and through enhanced ADCC/ADCP effector functions, with reduced toxicity on normal cells as compared to existing anti-CTLA4 antibodies.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

Before describing the present disclosure in detail, it is to be understood that this present disclosure is not limited to particular compositions or biological systems, which can, of course, vary. It is also to be understood that the terminology used herein is for the purpose of describing particular embodiments only, and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising,” “consisting,” and “consisting essentially of” aspects and embodiments.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The term “amino acid analogs” refers to compounds that have the same basic chemical structure as a naturally occurring amino acid but the C-terminal carboxy group, the N-terminal amino group, or side chain functional group has been chemically modified to another functional group. The term “amino acid mimetics” refers to chemical compounds that have a structure that is different from the general chemical structure of an amino acid, but that functions similarly to a naturally occurring amino acid. As used herein, the twenty conventional amino acids and their abbreviations follow conventional usage. See e.g., Immunology—A Synthesis (2nd Edition, E. S. Golub and D. R. Gren, Eds., Sinauer Associates, Sunderland, Mass. (1991)).

The terms “polypeptide,” “protein,” and “peptide” are used interchangeably herein and may refer to polymers of two or more amino acids.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification (s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides (s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P (O) S (“thioate”), P (S) S (“dithioate”), (O) NR2 (“amidate”), P (O) R, P (O) OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid of interest.

The term “antibody” is used herein in the broadest sense and specifically covers monoclonal antibodies (including full length monoclonal antibodies), polyclonal antibodies, masked antibodies (e.g., activatable antibodies), multispecific antibodies (e.g., bispecific antibodies), and antibody fragments (e.g., a single-chain variable fragment or scFv) so long as they exhibit the desired biological activity.

The term “antibody” is an art-recognized term and may refer to an antigen-binding protein (i. e, immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as VH) followed by a constant region. The heavy chain constant region is comprised of three domains, CH1, CH2 and CH3. Each light chain has, at the N-terminus, a variable region (abbreviated herein as VL) followed by a constant region at its other end. The light chain constant region is comprised of one domain, CL. The VL is aligned with the VH and the CL is aligned with the first constant domain of the heavy chain (CH1). The pairing of a VH and VL together forms a single antigen-binding site. An IgM antibody consists of 5 of the basic heterotetramer units along with an additional polypeptide called J chain, and therefore contains 10 antigen binding sites, while secreted IgA antibodies can polymerize to form

The term “hypervariable region” or “HVR,” as used herein, refers to each of the regions of an antibody variable domain, which are hypervariable in sequence. HVRs may form structurally defined loops (“hypervariable loops”). Generally, native four-chain antibodies comprise six HVRs; three in the VH (H1, H2, H3), and three in the VL (L1, L2, L3). HVRs are interspersed with regions that are more conserved, termed framework regions (FR). Each VH and VL is composed of three HVRs and four FRs, arranged from amino-terminus to carboxy-terminus in the following order: FR1, HVR1, FR2, HVR2, FR3, HVR3, FR4. HVRs generally comprise amino acid residues from the hypervariable loops and/or from the “complementarity determining regions” (CDRs), CDRs being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196: 901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, MD (1991)). With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3 (Almagro and Fransson, Front. Biosci. 13:1619-1633 (2008)).

Table 1 below provides exemplary CDR definitions according to various algorithms known in the art.

TABLE 1
CDR Definitions

| | Kabat¹ | Chothia² | MacCallum³ | IMGT⁴ | AHo⁵ |
|---|---|---|---|---|---|
| CDR-H1 | 31-35 | 26-32 | 30-35 | 27-38 | 25-40 |
| CDR-H2 | 50-65 | 53-55 | 47-58 | 56-65 | 58-77 |
| CDR-H3 | 95-102 | 96-101 | 93-101 | 105-117 | 109-137 |
| CDR-L1 | 24-34 | 26-32 | 30-36 | 27-38 | 25-40 |
| CDR-L2 | 50-56 | 50-52 | 46-55 | 56-65 | 58-77 |
| CDR-L3 | 89-97 | 91-96 | 89-96 | 105-117 | 109-137 |

¹Residue numbering follows the nomenclature of Kabat et al., J. Biol. Chem. 252: 6609-6616 (1977); Kabat et al., U.S. Dept. of Health and Human Services, “Sequences of proteins of immunological interest” (1991).
²Residue numbering follows the nomenclature of Chothia et al., J. Mol. Biol. 196: 901-917 (1987); Al-Lazikani B. et al., J. Mol. Biol., 273: 927-948 (1997).
³Residue numbering follows the nomenclature of MacCallum et al., J. Mol. Biol. 262: 732-745 (1996); Abhinandan and Martin, Mol. Immunol., 45: 3832-3839 (2008).
⁴Residue numbering follows the nomenclature of Lefranc M. P. et al., Dev. Comp. Immunol., 27: 55-77 (2003); and Honegger and Plückthun, J. Mol. Biol., 309: 657-670 (2001).
⁵Residue numbering follows the nomenclature of Honegger and Plückthun, J. Mol. Biol., 309: 657-670 (2001).

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Within light and heavy chains, the variable and constant regions are joined by a “J” region of about 12 or more amino acids, with the heavy chain also including a “D” region of about 10 or more amino acids (see e.g., Fundamental Immunology Ch. 7 (Paul, W., ed., 2^{nd} ed. Raven Press, N. Y). (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “antibody derivative” or “derivative” of an antibody refers to a molecule that is capable of binding to the same antigen (e.g., CTLA4) that the antibody binds to and comprises an amino acid sequence of the antibody linked to an additional molecular entity. The amino acid sequence of the antibody that is contained in the antibody derivative may be a full-length heavy chain, a full-length light chain, any portion or portions of a full-length heavy chain, any portion or portions of the full-length light chain of the antibody, any other fragment (s) of an antibody, or the complete antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional molecular entity by chemical coupling, genetic fusion, noncovalent association, or otherwise. The term “antibody derivative” also encompasses chimeric antibodies, humanized antibodies, and molecules that are derived from modifications of the amino acid sequences of a CTLA4 antibody, such as conservation amino acid substitutions, additions, and insertions.

The term “antigen-binding fragment” or “antigen binding portion” of an antibody refers to one or more portions of an antibody that retain the ability to bind to the antigen that the antibody bonds to (e.g., CTLA4). Examples of “antigen-binding fragments” of an antibody include (i) a Fab fragment, a monovalent fragment consisting of the V_{L}, V_{H}, C_{L} and C_{H1} domains; (ii) a F (ab′)₂ fragment, a bivalent fragment comprising two Fab fragments linked by a disulfide bridge at the hinge region; (iii) a Fd fragment consisting of the V_{H} and C_{H}, domains; (iv) a Fv fragment consisting of the V_{L} and V_{H} domains of a single arm of an antibody, (v) a dAb fragment (Ward et al., Nature 341: 544-546 (1989)), which consists of a V_{H} domain; and (vi) an isolated complementarity determining region (CDR).

The term “masked antibody” refers to an antibody, or an antigen-binding fragment thereof, comprising a peptide that interferes with, obstructs, reduces the ability of, prevents, inhibits, or competes with the target binding moiety (TBM) of the antibody, or antigen-binding fragment thereof, for binding to its target. A masked antibody may be generated by linking a masking peptide to the TBM of an antibody or an antigen-fragment thereof.

The term “activatable antibody” refers to a masked antibody, or an antigen-binding fragment thereof, that exhibits a first binding affinity to a target when in an inactivated (e.g., inhibited, masked, and/or uncleaved) state, and exhibits a second binding affinity to the target in an activated (e.g., uninhibited, unmasked, and/or cleaved) state, where the second binding affinity is greater than the first binding affinity. An activatable antibody may be generated by linking a masking peptide comprising an activatable component (e.g., a cleavage site) to the target-binding moiety (TBM) of an antibody or an antigen-fragment thereof. Activatable antibodies have been described, for example, in U.S. Pat. Pub. No. 2019/0241886 and U.S. Pat. Pub. No. 2021/0207126, the contents of both of which are incorporated herein by reference in their entirety.

A “target binding moiety (TBM)” refers to a structural moiety of an antigen binding portion of an antibody which binds to the target antigen of the antibody. The TBM may comprise a VH and a VL, such as any VH or VL described herein in any combination.

A “masking peptide” refers to a structural moiety of the masked antibody (e.g., activatable antibody) which inhibits binding of the TBM to its target antigen, and typically comprises, from N terminus to C terminus, a masking unit (MU) and a linkage unit (LU). The C terminus of the masking peptide is typically linked to the N terminus of the VH or the VL of a masked antibody (e.g., activatable antibody). In some embodiments, the masking peptide, or a portion thereof (e.g., the masking unit (MU)), interferes with or inhibits binding of the TBM to its target so efficiently that binding of the TBM to its target is extremely low and/or below the limit of detection (e.g., binding cannot be detected in an ELISA or flow cytometry assay). The masked antibodies (e.g., activatable antibodies) described herein may comprise one or more linkers, e.g., within the LU, disposed between MU and LU, LU and VH or VL, or VH and hinge region of an Fc. As used herein, the terms “masking unit” and “masking moiety” are used interchangeably. As used herein, the terms “linkage unit” and “linkage moiety” are used interchangeably.

The LU of the masking peptide may comprise at least a first cleavage site. A cleavage site generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a cleavage site, the terms encompass enzymatic cleavage, e.g., by a protease, as well as disruption of a disulfide bond between a cysteine-cysteine pair via reduction of the disulfide bond that can result from exposure to a reducing agent. The amino acid sequence of the cleavage site may overlap with or be included within the MU. Activatable antibodies may comprise a cleavage site configured to mediate activation of the antibody. For example, when the cleavage site of an activatable antibody is intact (e.g., uncleaved by a corresponding enzyme, and/or containing an unreduced cysteine-cysteine disulfide bond), the masking peptide, or a portion thereof, may interfere with or inhibit binding of the TBM to its target.

The term “masking efficiency” refers to the efficiency with which the masking peptide inhibits binding of the TBM to the target antigen. Masking efficiency may be measured as the difference in, or the ratio of, a characteristic (e.g., binding affinity for the target antigen) or an activity (e.g., blocking binding of the target antigen to a ligand) of a masked antibody (e.g., activatable antibody) having a TBM and a masking peptide, relative to a corresponding unmasked antibody (“parental antibody”) having the same TBM but lacking the masking peptide. In activatable antibodies, masking efficiency may be measured as the difference in, or the ratio of, a characteristic (e.g., binding affinity for the target antigen) or an activity (e.g., blocking binding of the target antigen to a ligand) of the activatable antibody having a TBM and a masking peptide in its inactivated (e.g., inhibited, masked, and/or uncleaved) state, relative to the activatable antibody in its activated (e.g., uninhibited, unmasked, and/or cleaved) state, or relative to a parental antibody having the same TBM but lacking the masking peptide. For example, the masking efficiency may be measured by dividing the EC50 of an activatable antibody for binding a target antigen in its inactivated (e.g., inhibited, masked, and/or uncleaved) state, relative to the EC50 or K_{D} of the activatable antibody to bind to the target antigen in its activated (e.g., uninhibited, unmasked, and/or cleaved) state, or relative to EC50 or K_{D} of the parental antigen to bind to the target antigen. The EC50 values may be measured in an ELISA assay, for example, as described in Example 3, or a Jurkat NFAT reporter assay, for example, as described in U.S. Pat. App. Pub. No. US20210207126 A1. The K_{D} values may be measured by, for example, using surface plasmon resonance using one of the systems described herein.

“Antibody-dependent cell-mediated cytotoxicity” or “ADCC” refers to a form of cytotoxicity in which secreted immunoglobulin bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9: 457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95: 652-656 (1998). As used herein, an “ADCC-enhancing mutation” refers to a mutation, such as an amino acid substitution, in an antibody that enhances the level of ADCC induced by the antibody.

The term “antibody-dependent cellular phagocytosis” or “ADCP” as used herein refers to a mechanism of cell-mediated immune defense whereby an effector cell of the immune system (e.g., a macrophage or a T lymphocyte) phagocytoses a target cell, whose membrane-surface antigens have been bound by specific antibodies. Methods of assessing ADCP activities are known in the art, including, for example, the assay described in Example 14 of the present application. As used herein, an “ADCP-enhancing mutation” refers to a mutation, such as an amino acid substitution, in an antibody that enhances the level of ADCP induced by the antibody.

The term “binding molecule” encompasses (1) antibody, (2) antigen-binding fragment of an antibody, and (3) derivative of an antibody, each as defined herein.

The term “CTLA4” is used in the present application, and includes the human CTLA4 (e.g., UniProt accession number P16410), as well as variants, isoforms, and species homologs thereof (e.g., mouse CTLA4 (UniProt accession number P09793), rat CTLA4 (UniProt accession number Q9Z1A7), dog CTLA4 (UniProt accession number Q9XSI1), cynomolgus monkey CTLA4 (UniProt accession number G7PL88), etc.). Accordingly, a binding molecule (e.g., an antibody or masked antibody (e.g., activatable antibody)), as defined and disclosed herein, may also bind CTLA4 from species other than human. In other cases, a binding molecule may be completely specific for the human CTLA4 and may not exhibit species or other types of cross-reactivity.

The terms “CTLA4 antibody” and “anti-CTLA4 antibody” are used interchangeably herein and refer to an antibody, as defined herein, capable of binding to human CTLA4.

The term “chimeric antibody” refers to an antibody that comprises amino acid sequences derived from different animal species, such as those having a variable region derived from a human antibody and a murine immunoglobulin constant region.

The term “compete for binding” refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope (s).

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants (see e.g., Epitope Mapping Protocols in Methods in Molecular Biology, Vol. 66, G. E. Morris, Ed. (1996)). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “germline” refers to the nucleotide sequences of the antibody genes and gene segments as they are passed from parents to offspring via the germ cells. The germline sequence is distinguished from the nucleotide sequences encoding antibodies in mature B cells which have been altered by recombination and hypermutation events during the course of B cell maturation.

The term “glycosylation sites” refers to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The terms “N-linked” and “O-linked” refer to the chemical group that serves as the attachment site between the sugar molecule and the amino acid residue. N-linked sugars are attached through an amino group; O-linked sugars are attached through a hydroxyl group. The term “glycan occupancy” refers to the existence of a carbohydrate moiety linked to a glycosylation site (i.e., the glycan site is occupied). Where there are at least two potential glycosylation sites on a polypeptide, either none (0-glycan site occupancy), one (1-glycan site occupancy) or both (2-glycan site occupancy) sites can be occupied by a carbohydrate moiety.

The term “host cell” refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; human cells (e.g., HEK293F cells, HEK293T cells; or human tissues or hybridoma cells, yeast cells, insect cells (e.g., S2 cells), bacterial cells (e.g., E. coli cells) and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized antibody” refers to a chimeric antibody that contains amino acid residues derived from human antibody sequences. A humanized antibody may contain some or all of the CDRs or HVRs from a non-human animal or synthetic antibody while the framework and constant regions of the antibody contain amino acid residues derived from human antibody sequences.

The term “illustrative antibody” refers to any one of the antibodies described in the disclosure and designated as those listed in Tables 2 and 3, and any antibodies comprising the 6 CDRs and/or the VH and VLs of the antibodies listed in Tables 2 and 3. These antibodies may be in any class (e.g., IgA, IgD, IgE, IgG, and IgM). Thus, each antibody identified above encompasses antibodies in all five classes that have the same amino acid sequences for the V_{L} and V_{H} regions. Further, the antibodies in the IgG class may be in any subclass (e.g., IgG1 IgG2, IgG3, and IgG4). Thus, each antibody identified above in the IgG subclass encompasses antibodies in all four subclasses that have the same amino acid sequences for the V_{L} and V_{H} regions. The amino acid sequences of the heavy chain constant regions of human antibodies in the five classes, as well as in the four IgG subclasses, are known in the art.

An “isolated” antibody or binding molecule (e.g., masked antibody) is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see e.g., Flatman et al., J. Chromatogr. B 848: 79-87 (2007).

The term “Kₐ” refers to the association rate constant of a particular binding molecule-antigen interaction, where the term “k_{d}” refers to the dissociation rate constant of a particular binding molecule-antigen interaction.

The term “K_{D}” refers to the equilibrium dissociation constant of a particular antibody-antigen interaction. It is obtained from the ratio of k_{d} to kₐ (i.e., k_{d}/kₐ) and is expressed as a molar concentration (M). K_{D} is used as a measure for the affinity of an antibody's binding to its binding partner. The smaller the K_{D}, the more tightly bound the antibody is, or the higher the affinity between antibody and the antigen. For example, an antibody with a nanomolar (nM) dissociation constant binds more tightly to a particular antigen than an antibody with a micromolar (M) dissociation constant. K_{D} values for antibodies can be determined using methods well established in the art. One method for determining the K_{D} of an antibody is by using surface plasmon resonance, typically using a biosensor system such as a Biacore®system. For example, an assay procedure using the BIACORE™ system (BIAcore assay) is described in at least Example 3 of the present disclosure.

The term “prevent” or “preventing,” with reference to a certain disease condition in a mammal, refers to preventing or delaying the onset of the disease, or preventing the manifestation of clinical or subclinical symptoms thereof.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. 183: 63-98 (1990); Pearson, Methods Mol. Biol. 132: 185-219 (2000); Altschul et al., J. Mol. Biol. 215: 403-410 (1990); Altschul et al., Nucleic Acids Res. 25: 3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

As used herein, the term “binds”, “binds to”, “specifically binds” “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “cold tumor” refers to a tumor in which little to no T-cell infiltration occurs, causing them to be resistant to both natural anti-tumor immune responses and immunotherapy. The lack of T-cell infiltration in cold tumors can be caused by several factors, including, but not limited to, a lack of, or defects in, tumor antigen presentation, and a lack of T-cell activation. Cancers that are considered cold tumors include, but are not limited to, brain cancers (e.g., glioblastoma), breast cancers, head and neck cancers, ovarian cancers, pancreatic cancers, and prostate cancers.

The term “vector” refers to a nucleic acid molecule capable of transporting a foreign nucleic acid molecule. The foreign nucleic acid molecule is linked to the vector nucleic acid molecule by a recombinant technique, such as ligation or recombination. This allows the foreign nucleic acid molecule to be multiplied, selected, further manipulated or expressed in a host cell or organism. A vector can be a plasmid, phage, transposon, cosmid, chromosome, virus, or virion. One type of vectors can be integrated into the genome of a host cell upon introduction into the host cell, and thereby are replicated along with the host genome (e.g., non-episomal mammalian vectors). Another type of vector is capable of autonomous replication in a host cell into which it is introduced (e.g., bacterial vectors having a bacterial origin of replication and episomal mammalian vectors). Another specific type of vector capable of directing the expression of expressible foreign nucleic acids to which they are operatively linked is commonly referred to as “expression vectors.” Expression vectors generally have control sequences that drive expression of the expressible foreign nucleic acids. Simpler vectors, known as “transcription vectors,” are only capable of being transcribed but not translated: they can be replicated in a target cell but not expressed. The term “vector” encompasses all types of vectors regardless of their function. Vectors capable of directing the expression of expressible nucleic acids to which they are operatively linked are commonly referred to “expression vectors.” Other examples of “vectors” may include display vectors (e.g., vectors that direct expression and display of an encoded polypeptide on the surface of a virus or cell (such as a bacterial cell, yeast cell, insect cell, and/or mammalian cell).

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present disclosure, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

### III. Binding Molecules that Bind to Human CTLA4 with Enhanced Effector Function

The present disclosure relates, in part, to isolated binding molecules that bind to human CTLA4, including CTLA4 antibodies, antigen-binding fragments of the CTLA4 antibodies, and derivatives of the CTLA4 antibodies. In some embodiments, the binding molecules that bind to human CTLA4 have enhanced effector functions. In particular, provided herein are isolated antibodies, or antigen-binding fragments thereof, that bind to human CTLA4 and comprise a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering.

S239D and I332E substitutions have been described, for example, in Lazar GA, Dang W, Karki S, et al. Engineered antibody Fc variants with enhanced effector function. Proc Natl Acad Sci USA. 2006; 103 (11): 4005-4010. doi: 10.1073/pnas. 0508123103. As described in Lazar et al., antibodies with S239D and/or I332E substitutions have enhanced binding affinity for Fcγreceptors (FcγRs) and an enhanced ability to promote antibody-dependent cellular cytotoxicity (ADCC) and antibody-dependent cellular phagocytosis (ADCP). The position of the S239D and I332E substitutions are provided according to EU numbering. Both S239 and 1332 are located within the CH2 domain of the Fc region.

Because CTLA4 is expressed on healthy cells, such as T effector cells, known CTLA4 antibodies in the field such as ipilimumab do not have Fc regions with enhanced effector functions (e.g., ADCC and ADCP), in order to avoid toxicity against healthy cells. In order to leverage the ADCC and ADCP effector functions of IgG1 Fc regions, especially those with S239D and I332E substitutions, the inventors created masked antibodies, which selectively bind CTLA4 only in certain environments, such as the tumor microenvironment (TME). Thus, the present disclosure provides antibodies that bind CTLA4 and exhibit enhanced ADCC and ADCP effector functions without sacrificing the safety.

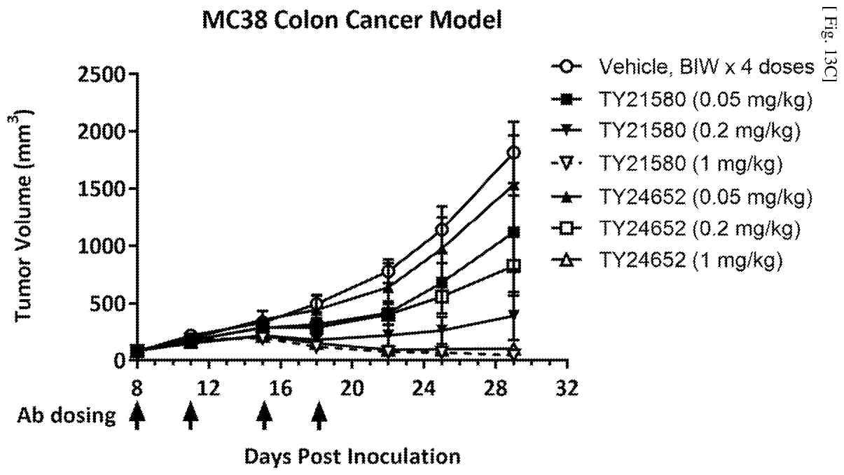

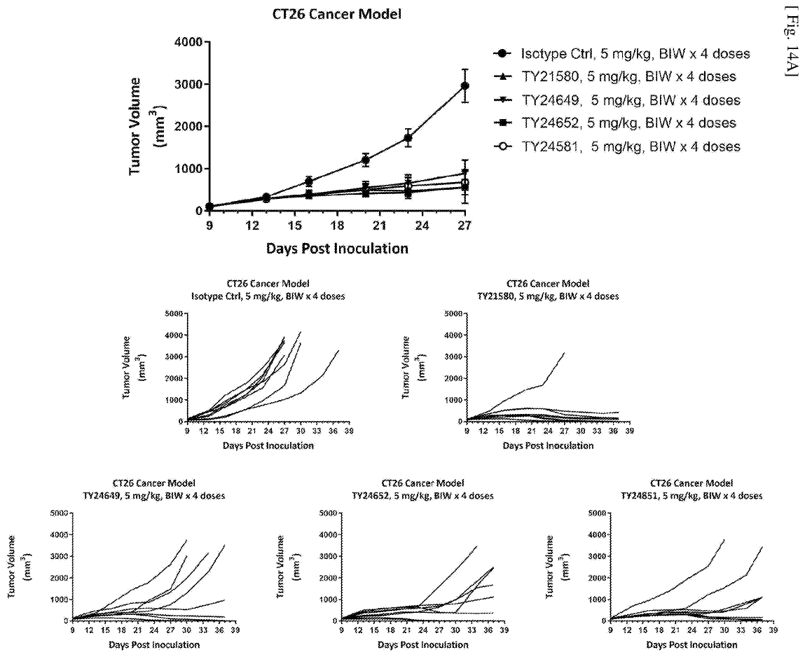

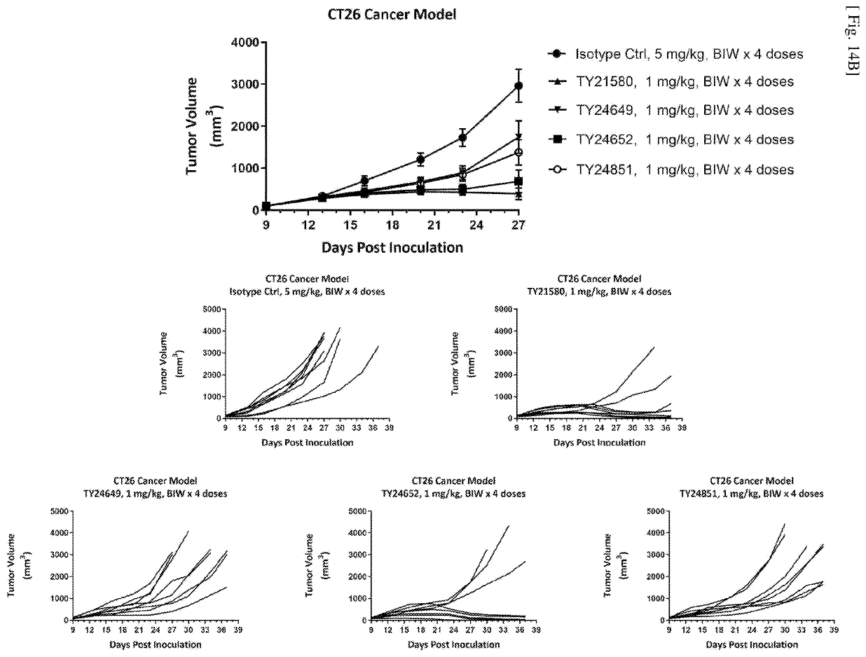

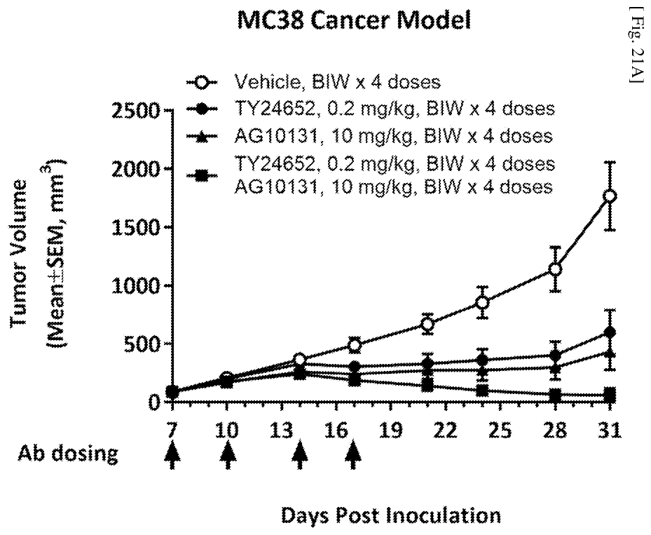

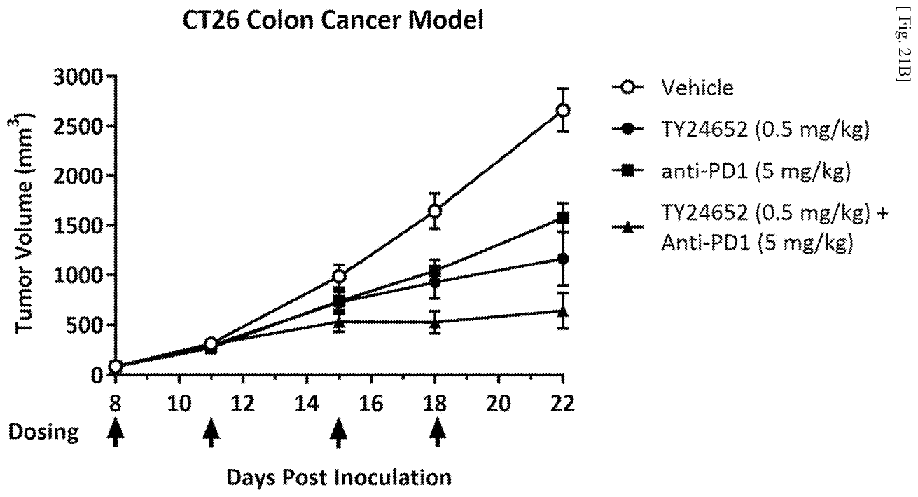

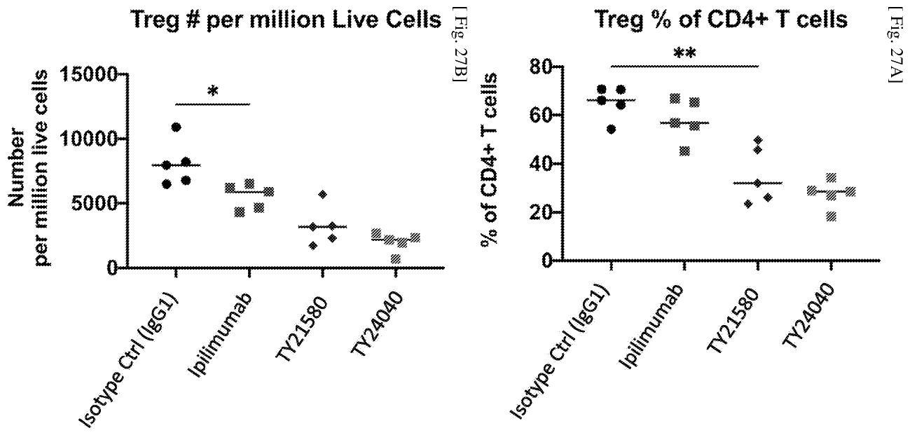

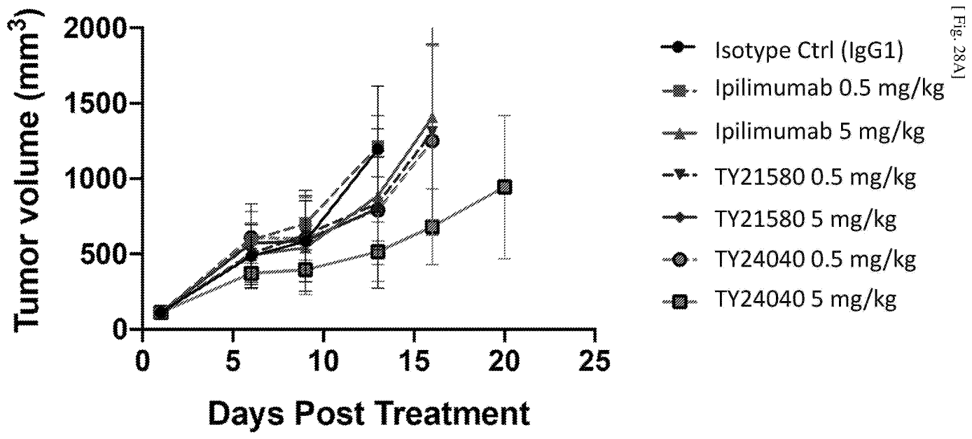

As described in detail in the Examples section herein, IgG1 Fc regions with ADCC- and/or ADCP-enhancing substitutions, including S239D and I332E substitutions, were introduced into antibodies that bind human CTLA4, as well as activatable antibodies that bind human CTLA4. As shown in FIG. 9, FIG. 20, and FIG. 24, introduction of targeted substitutions into the IgG1 Fc regions of parental and activatable antibodies improved their ability to induce ADCC and ADCP over the FDA-approved anti-CTLA4 antibody Ipilimumab. As shown in FIG. 10, introduction of the S239D and I332E substitutions into activatable antibodies also improved their ability to stimulate CD28 mediated downstream cell signaling by blocking CTLA4 sequestration of CD80 and CD86 ligands. In three different animal cancer models, the parental antibody (TY21580), the parental antibody having S239D and I332E substitutions (TY24040), and activatable antibodies having S239D and I332E substitutions (TY24851 and TY24652) significantly inhibited tumor growth (FIGS. 13A-15B and FIG. 25). Anti-CTLA4 antibodies having F243L, R292P, Y300L, V305I, and P396L Fc region substitutions (TY24037) and S239D, I332E, and A330L Fc region substitutions (TY24039) also showed enhanced tumor growth inhibition compared to the parental antibody lacking the Fc region substitutions (TY21580) (FIG. 25). Notably, FIG. 19 shows that, while the parental anti-CTLA4 antibody TY21580 increased immune checkpoint inhibitor-associated diabetes when combined with an anti-PD-1 antibody, the activatable antibodies TY24652 and TY22404 did not. The activatable antibody TY22404 was described in U.S. Publication No. 2021/0207126, which is incorporated by reference in its entirety. These results demonstrate the remarkable efficacy of the anti-CTLA4 antibodies of the present disclosure in the treatment of cancer by leveraging ADCC and ADCP activity without sacrificing safety.

Any one of the antibodies that binds human CTLA4 as described herein may comprise a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering. For example, any one of the anti-CTLA4 antibodies described herein can comprise a human IgG1 Fc region, wherein each IgG1 Fc domain of the human IgG1 Fc region comprises: each human IgG1 Fc domain of the heavy chain comprises: i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution, wherein the residue numbering is according to EU numbering. Exemplary antibodies that bind human CTLA4 are also described, for example, in U.S. Patent Application Publication No. US20210207126A1, which is hereby incorporated by reference in its entirety. In some embodiments, an antibody that binds human CTLA4 as described in U.S. Patent Application Publication No. US20210207126A1 is provided, wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 antibodies described herein are afucosylated.

In some embodiments, an anti-CTLA4 antibody of the present disclosure comprises a first polypeptide and a second polypeptide each comprising a VH (e.g., two heavy chains); and a third polypeptide and a fourth polypeptide each comprising a VL (e.g., two light chains). In certain embodiments, the anti-CTLA4 antibody comprises a first polypeptide comprising a VH and a first Fc domain (i.e., a first heavy chain); a second polypeptide comprising a VH and a second Fc domain (i.e., a second heavy chain); and a third polypeptide and a fourth polypeptide each comprising a VL (e.g., two light chains). In certain embodiments, the first and second Fc domains are the same. In other embodiments, the first and second Fc domains are different. In some embodiments, the first and second Fc domains are both IgG1 Fc domains (e.g., human IgG1 Fc domains). The first and second IgG1 Fc domains may be the same IgG1 Fc domain or different IgG1 Fc domains. In some embodiments, the first and second IgG1 Fc domains each comprise one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In certain embodiments, the first and second IgG1 Fc domains each comprise the same substitutions. In some embodiments, the first polypeptide and second polypeptide are each heavy chains, and/or the third polypeptide and the fourth polypeptide are each light chains.

In some embodiments, an anti-CTLA4 antibody of the present disclosure comprises two heavy chains and two light chains, wherein each human IgG1 Fc domain of the heavy chains comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering. In some embodiments, each human IgG1 Fc domain of the heavy chains comprises an S298A substitution, an E333A substitution, and a K334A substitution, wherein the residue numbering is according to EU numbering. In some embodiments, each human IgG1 Fc domain of the heavy chains comprises an S298A substitution, an E333A substitution, a K334A substitution, and a K326A substitution, wherein the residue numbering is according to EU numbering. In some embodiments, each human IgG1 Fc domain of the heavy chains comprises an F243L substitution. In some embodiments, each human IgG1 Fc domain of the heavy chains comprises an F243L substitution, an R292P substitution, a Y300L substitution, and a P396L substitution, wherein the residue numbering is according to EU numbering. In some embodiments, each human IgG1 Fc domain of the heavy chains comprises an F243L substitution, an R292P substitution, a Y300L substitution, a V305I substitution, and a P396L substitution, wherein the residue numbering is according to EU numbering. In some embodiments, each human IgG1 Fc domain of the heavy chains comprises an F243L substitution, an R292P substitution, a Y300L substitution, a L235V substitution, and a P396L substitution, wherein the residue numbering is according to EU numbering. In some embodiments, each human IgG1 Fc domain of the heavy chains comprises a S239D substitution, an I332E substitution, and an A330L substitution, wherein the residue numbering is according to EU numbering. In some embodiments, each human IgG1 Fc domain of the heavy chains comprises a S239D substitution and an I332E substitution, wherein the residue numbering is according to EU numbering. In certain embodiments, the Fc domains of the two heavy chains are the same. In other embodiments, the Fc domains of the two heavy chains are different. In some embodiments, the anti-CTLA4 antibody is a masked antibody (e.g., activatable antibody), and the two light chains each comprise the structure, from N-terminus to C-terminus, of: masking unit (MU)-linkage unit (LU)-VL.

In some embodiments, an anti-CTLA4 antibody of the present disclosure comprises a first polypeptide and a second polypeptide, wherein one or both of the first polypeptide and the second polypeptide comprise, from N- to C-terminus, an antibody heavy chain variable region (VH) and a heavy chain constant region (e.g., an Fc domain), wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 antibody comprises a first polypeptide and a second polypeptide, each comprising, from N- to C-terminus, an antibody heavy chain variable region (VH) and a heavy chain constant region (e.g., an Fc domain), wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the heavy chain constant region is an IgG1 Fc domain (e.g., a human IgG1 Fc domain). In certain embodiments, the IgG1 Fc domains (e.g., human IgG1 Fc domains) of the first and second polypeptide dimerize to form an IgG1 Fc region (e.g., a human IgG1 Fc region). In certain embodiments, the first and second heavy chain constant regions (e.g., human IgG1 Fc domains) are the same. In other embodiments, the first and second heavy chain constant regions (e.g., human IgG1 Fc domains) are different. In some embodiments, the first and second heavy chain constant regions (e.g., human IgG1 Fc domains) each comprise a S239D substitution and/or an I332E substitution. In some embodiments, the anti-CTLA4 antibody further comprises a third polypeptide and a fourth polypeptide, each comprising a VL. In some embodiments, the anti-CTLA4 antibody is a masked antibody (e.g., activatable antibody) comprising a third polypeptide and a fourth polypeptide, each comprising the structure, from N-terminus to C-terminus, of: masking unit (MU)-linkage unit (LU)-VL. In some embodiments, the first polypeptide and second polypeptide are each heavy chains, and/or the third polypeptide and the fourth polypeptide are each light chains.

In some embodiments, the anti-CTLA4 antibody comprises: a) a CDR-H1 comprising an amino acid sequence selected from SEQ ID NOS: 1, 7, 12, 18, 23, 29, 34, 38, 44, 49, and 60, or a variant thereof comprising up to 5 (e.g., 1, 2, 3, 4, or 5) amino acid substitutions; a CDR-H2 comprising an amino acid sequence selected from SEQ ID NOS: 2, 8, 13, 19, 24, 30, 39, 45, 56, and 61, or a variant thereof comprising up to 5 (e.g., 1, 2, 3, 4, or 5) amino acid substitutions; and a CDR-H3 comprising an amino acid sequence selected from SEQ ID NOS: 3, 9, 14, 20, 25, 31, 35, 40, 46, 50, 53, 57, and 62, or a variant thereof comprising up to 5 (e.g., 1, 2, 3, 4, or 5) amino acid substitutions; and/or b) a CDR-L1 comprising an amino acid sequence selected from SEQ ID NOS: 4, 10, 15, 21, 26, 32, 36, 41, 47, 51, 54, 58, and 63, or a variant thereof comprising up to 5 (e.g., 1, 2, 3, 4, or 5) amino acid substitutions; a CDR-L2 comprising an amino acid sequence selected from SEQ ID NOS: 5, 16, 27, and 42, or a variant thereof comprising up to 5 (e.g., 1, 2, 3, 4, or 5) amino acid substitutions; and a CDR-L3 comprising an amino acid sequence selected from SEQ ID NOS: 6, 11, 17, 22, 28, 33, 37, 43, 48, 52, and 59, or a variant thereof comprising up to 5 (e.g., 1, 2, 3, 4, or 5) amino acid substitutions. In some embodiments, the anti-CTLA4 antibody comprises one, two, three, four, five, or all six of the CDRs shown for any of the exemplary anti-CTLA4 antibodies described in Table 2, below. In some embodiments, the anti-CTLA4 antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

TABLE 2
Anti-CTLA4 Kabat CDR sequences

| Ab | | | | | | |
|---|---|---|---|---|---|---|
| name | CDR-H1 | CDR-H2 | CDR-H3 | CDR-L1 | CDR-L2 | CDR-L3 |
| TY21585 | DYAIH | IISPSSGSTN | DIHSGSSGY | RASESVDFF | DASNRAT | QHYTSSPPV |
| | (SEQ ID NO: | YAQKFQG | YYGFDV | GISFLA | (SEQ ID NO: | YT |
| | 7) | (SEQ ID NO: | (SEQ ID NO: | (SEQ ID NO: | 5) | (SEQ ID NO: |
| | | 8) | 9) | 10) | | 11) |
| TY21586 | SGYYWA | SISGSGSTTY | DGFGYFDY | SASSSVSYV | DASSLES | VQGLQTPW |
| | (SEQ ID NO: | YADSVKG | (SEQ ID NO: | Y | (SEQ ID NO: | T |
| | 12) | (SEQ ID NO: | 14) | (SEQ ID NO: | 16) | (SEQ ID NO: |
| | | 13) | | 15) | | 17) |
| TY21587 | DYGIH | EIYHSGSTY | DVAPGSSGY | RASQGIGSS | DASNRAT | QQYDQWPP |
| | (SEQ ID NO: | YSPSLKS | YDGFDF | LA | (SEQ ID NO: | WT |
| | 18) | (SEQ ID NO: | (SEQ ID NO: | (SEQ ID NO: | 5) | (SEQ ID NO: |
| | | 19) | 20) | 21) | | 22) |
| TY21588 | SGYHWD | GISGYGGST | HSYYGSGNF | RASESVDFF | DASNLET | QQSYSWPPT |
| | (SEQ ID NO: | YYADSVKG | DY | GKSFLH | (SEQ ID NO: | (SEQ ID NO: |
| | 23) | (SEQ ID NO: | (SEQ ID NO: | (SEQ ID NO: | 27) | 28) |
| | | 24) | 25) | 26) | | |
| TY21589 | DYWIH | WISPSGGGT | GAYEFDY | RASQSVSSR | DASNRAT | QQSYPTPLT |
| | (SEQ ID NO: | KYAQKFQG | (SEQ ID NO: | FLA | (SEQ ID NO: | (SEQ ID NO: |
| | 29) | (SEQ ID NO: | 31) | (SEQ ID NO: | 5) | 33) |
| | | 30) | | 32) | | |
| TY21580 | SGYHWS | RIDWDDDK | SYVYFDY | RASQSVRGR | DASNRAT | QQSSSWPPT |
| | (SEQ ID NO: | YYSTSLKS | (SEQ ID NO: | FLA | (SEQ ID NO: | (SEQ ID NO: |
| | 1) | (SEQ ID NO: | 3) | (SEQ ID NO: | 5) | 6) |
| | | 2) | | 4) | | |
| TY21591 | TGGVAVS | EIYHSGSTY | RIATATYFD | RASQTVFSR | DASNRAT | QQSYYWPP |
| | (SEQ ID NO: | YSPSLKS | Y | YLA | (SEQ ID NO: | WT |
| | 34) | (SEQ ID NO: | (SEQ ID NO: | (SEQ ID NO: | 5) | (SEQ ID NO: |
| | | 19) | 35) | 36) | | 37) |
| TY21686 | TGGVAVG | AISGYGSTT | LPYSAYAFD | RASQGVSSY | AASTLQS | QHHYGTPLT |
| | (SEQ ID NO: | YYADSVKG | Y | LA | (SEQ ID NO: | (SEQ ID NO: |
| | 38) | (SEQ ID NO: | (SEQ ID NO: | (SEQ ID NO: | 42) | 43) |
| | | 39) | 40) | 41) | | |
| TY21687 | GYAIH | IISPSGGGTK | HPFAY | RASQSVDFY | DASNRAT | QQYVSSPPE |
| | (SEQ ID NO: | YAQKFQG | (SEQ ID NO: | GISFLD | (SEQ ID NO: | YT |
| | 44) | (SEQ ID NO: | 46) | (SEQ ID NO: | 5) | (SEQ ID NO: |
| | | 45) | | 47) | | 48) |
| TY21689 | GYGIH | EIYHSGSTY | RIDAFDI | RASQSVDFD | DASSLES | QQRDSWPY |
| | (SEQ ID NO: | YSPSLKS | (SEQ ID NO: | GFSFLH | (SEQ ID NO: | T |
| | 49) | (SEQ ID NO: | 50) | (SEQ ID NO: | 16) | (SEQ ID NO: |
| | | 19) | | 51) | | 52) |
| TY21680 | GYAIH | IISPSGGGTK | LYDVAY | RASQSVDFH | DASSLES | EQSLEVPFT |
| | (SEQ ID NO: | YAQKFQG | (SEQ ID NO: | GKSFLH | (SEQ ID NO: | (SEQ ID NO: |
| | 44) | (SEQ ID NO: | 53) | (SEQ ID NO: | 16) | 55) |
| | | 45) | | 54) | | |
| TY21691 | DYAIH | IISPSGGSTK | LGYGYFDV | RASQSVDFY | DASSLES | VQALQLPLT |
| | (SEQ ID NO: | YAQKFQG | (SEQ ID NO: | GISFLH | (SEQ ID NO: | (SEQ ID NO: |
| | 7) | (SEQ ID NO: | 57) | (SEQ ID NO: | 16) | 59) |
| | | 56) | | 58) | | |
| TY21692 | SGHYWS | DISHSGSTY | GSRTGYFDY | RASQSISSYL | DASNLET | QHHYGTPLT |
| | (SEQ ID NO: | YSQSLKS | (SEQ TD NO: | N | (SEQ ID NO: | (SEQ ID NO: |
| | 60) | (SEQ ID NO: | 62) | (SEQ ID NO: | 27) | 43) |
| | | 61) | | 63) | | |

In some embodiments, the anti-CTLA4 antibody comprises: a) a heavy chain variable region comprising an amino acid sequence selected from SEQ ID NOS: 64, 66, 68, 70, 72, 74, 76, 78, 80, 82, 84, 86, and 88; and/or b) a light chain variable region comprising an amino acid sequence selected from SEQ ID NOS: 65, 67, 69, 71, 73, 75, 77, 79, 81, 83, 85, 87, and 89. In some embodiments, the anti-CTLA4 antibody comprises a heavy chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 64, 66, 68, 70, 72, 74, 76, 78, 80, 82, 84, 86, and 88, and/or a light chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 65, 67, 69, 71, 73, 75, 77, 79, 81, 83, 85, 87, and 89. In some embodiments, the anti-CTLA4 antibody comprises a heavy chain variable region and a light chain variable region of any of the exemplary anti-CTLA4 antibodies described in Table 3 below. In some embodiments, the anti-CTLA4 antibody comprises one, two, or all three CDRs of the heavy chain variable region, and/or one, two, or all three CDRs of the light chain variable region shown for any of the exemplary anti-CTLA4 antibodies described in Table 3 below. In some embodiments, the anti-CTLA4 antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In certain embodiments, the first and second IgG1 Fc domains are the same (i.e., comprise the same substitutions). In other embodiments, the first and second IgG1 Fc domains are different.

TABLE 3
Anti-CTLA4 variable region amino acid sequences

| Ab name | VH | VL |
|---|---|---|
| TY21585 | EVQLVESGGGLVQPGGSLRLSCAASGFTFSDY | DIQLTQSPSSLSASVGDRVTITCRASESVDFF |
| | AIHWVRQAPGKGLEWIGIISPSSGSTNYAQKF | GISFLAWYQQKPGKAPKLLIYDASNRATGI |
| | QGRVTISRDNSKNTLYLQLNSLRAEDTAVYYC | PSRFSGSGSGTDFTLTISSLQPEDFATYYCQ |
| | ARDIHSGSSGYYYGFDVWGQGTLVTVSS | HYTSSPPVYTFGQGTKVEIKR |
| | (SEQ ID NO: 64) | (SEQ ID NO: 65) |
| TY21586 | EVQLVESGGGLVQPGGSLRLSCAASGYSITSG | DIQLTQSPSSLSASVGDRVTITCSASSSVSYV |
| | YYWAWIRQAPGKGLEWVSSISGSGSTTYYAD | YWYQQKPGKAPKLLIYDASSLESGVPSRFS |
| | SVKGRFTISRDNSKNTLYLQLNSLRAEDTAVY | GSGSGTDFTLTISSLQPEDFATYYCVQGLQT |
| | YCARDGFGYFDYWGQGTLVTVSS | PWTFGQGTKVEIKR |
| | (SEQ ID NO: 66) | (SEQ ID NO: 67) |
| TY21587 | EVQLVESGGGLVQPGGSLRLSCAASGFTFSDY | DIQLTQSPSSLSASVGDRVTITCRASQGIGSS |
| | GIHWVRQAPGKGLEWIGEIYHSGSTYYSPSLK | LAWYQQKPGKAPKLLIYDASNRATGIPSRF |
| | SRVTISRDNSKNTLYLQLNSLRAEDTAVYYCA | SGSGSGTDFTLTISSLQPEDFATYYCQQYDQ |
| | RDVAPGSSGYYDGFDFWGQGTLVTVSS | WPPWTFGQGTKVEIKR |
| | (SEQ ID NO: 68) | (SEQ ID NO: 69) |
| TY21588 | EVQLVESGGGLVQPGGSLRLSCAASGYSISSG | DIQLTQSPSSLSASVGDRVTITCRASESVDFF |
| | YHWDWIRQAPGKGLEWVSGISGYGGSTYYA | GKSFLHWYQQKPGKAPKLLIYDASNLETG |
| | DSVKGRFTISRDNSKNTLYLQLNSLRAEDTAV | VPSRFSGSGSGTDFTLTISSLQPEDFATYYC |
| | YYCARHSYYGSGNFDYWGQGTLVTVSS | QQSYSWPPTFGQGTKVEIKR |
| | (SEQ ID NO: 70) | (SEQ ID NO: 71) |
| TY21589 | EVQLVESGGGLVQPGGSLRLSCAASGFTFSDY | DIQLTQSPSSLSASVGDRVTITCRASQSVSS |
| | WIHWVRQAPGKGLEWIGWISPSGGGTKYAQK | RFLAWYQQKPGKAPKLLIYDASNRATGIPS |
| | FQGRVTISRDNSKNTLYLQLNSLRAEDTAVYY | RFSGSGSGTDFTLTISSLQPEDFATYYCQQS |
| | CARGAYEFDYWGQGTLVTVSS | YPTPLTFGQGTKVEIKR |
| | (SEQ ID NO: 72) | (SEQ ID NO: 73) |
| TY21580 | EVQLVESGGGLVQPGGSLRLSCAASGYSISSG | DIQLTQSPSSLSASVGDRVTITCRASQSVRG |
| | YHWSWIRQAPGKGLEWLARIDWDDDKYYST | RFLAWYQQKPGKAPKLLIYDASNRATGIPS |
| | SLKSRLTISRDNSKNTLYLQLNSLRAEDTAVYY | RFSGSGSGTDFTLTISSLQPEDFATYYCQQS |
| | CARSYVYFDYWGQGTLVTVSS | SSWPPTFGQGTKVEIKR |
| | (SEQ ID NO: 74) | (SEQ ID NO: 75) |
| TY21591 | EVQLVESGGGLVQPGGSLRLSCAASGFSLSTG | DIQLTQSPSSLSASVGDRVTITCRASQTVFS |
| | GVAVSWIRQAPGKGLEWIGEIYHSGSTYYSPS | RYLAWYQQKPGKAPKLLIYDASNRATGIPS |
| | LKSRVTISRDNSKNTLYLQLNSLRAEDTAVYY | RFSGSGSGTDFTLTISSLQPEDFATYYCQQS |
| | CARRIATATYFDYWGQGTLVTVSS | YYWPPWTFGQGTKVEIKR |
| | (SEQ ID NO: 76) | (SEQ ID NO: 77) |
| TY21686 | EVQLVESGGGLVQPGGSLRLSCAASGFSLSTG | DIQLTQSPSSLSASVGDRVTITCRASQGVSS |
| | GVAVGWIRQAPGKGLEWVSAISGYGSTTYYA | YLAWYQQKPGKAPKLLIYAASTLQSGVPSR |
| | DSVKGRFTISRDNSKNTLYLQLKSLRAEDTAV | FSGSGSGTDFTLTISSLQPEDFATYYCQHHY |
| | YYCARLPYSAYAFDYWGQGTLVTVSS | GTPLTFGQGTKVEIKR |
| | (SEQ ID NO: 78) | (SEQ ID NO: 79) |
| TY21687 | EVQLVESGGGLVQPGGSLRLSCAASGFTFSGY | DIQLTQSPSSLSASVGDRVTITCRASQSVDF |
| | AIHWVRQAPGKGLEWIGIISPSGGGTKYAQKF | YGISFLDWYQQKPGKAPKLLIYDASNRATG |
| | QGRVTISRDNSKNTLYLQLNSLRAEDTAVYYC | IPSRFSGSGSGTDFTLTISSLQPEDFATYYCQ |
| | ARHPFAYWGQGTLVTVSS | QYVSSPPEYTFGQGTKVEIKR |
| | (SEQ TD NO: 80) | (SEQ ID NO: 81) |
| TY21689 | EVQLVESGGGLVQPGGSLRLSCAASGYTFSGY | DIQLTQSPSSLSASVGDRVTITCRASQSVDF |
| | GIHWVRQAPGKGLEWIGEIYHSGSTYYSPSLK | DGFSFLHWYQQKPGKAPKLLIYDASSLESG |
| | SRVTISRDNSKNTLYLQLNSLRAEDTAVYYCA | VPSRFSGSGSGTDFTLTISSLQPEDFATYYC |
| | RRIDAFDIWGQGTLVTVSS | QQRDSWPYTFGQGTKVEIKR |
| | (SEQ ID: NO: 82) | (SEQ ID NO: 83) |
| TY21680 | EVQLVESGGGLVQPGGSLRLSCAASGYTFSGY | DIQLTQSPSSLSASVGDRVTITCRASQSVDF |
| | AIHWVRQAPGKGLEWIGIISPSGGGTKYAQKF | HGKSFLHWYQQKPGKAPKLLIYDASSLES |
| | QGRVTISRDNSKNTLYLQLNSLRAEDTAVYYC | GVPSRFSGSGSGTDFTLTISSLQPEDFATYY |
| | ARLYDVAYWGQGTLVTVSS | CEQSLEVPFTFGQGTKVEIKR |
| | (SEQ ID NO: 84) | (SEQ ID NO: 85) |
| TY21691 | EVQLVESGGGLVQPGGSLRLSCAASGFTFSDY | DIQLTQSPSSLSASVGDRVTITCRASQSVDF |
| | AIHWVRQAPGKGLEWIGIISPSGGSTKYAQKF | YGISFLHWYQQKPGKAPKLLIYDASSLESG |
| | QGRVTISRDNSKNTLYLQLNSLRAEDTAVYYC | VPSRFSGSGSGTDFTLTISSLQPEDFATYYC |
| | ARLGYGYFDVWGQGTLVTVSS | VQALQLPLTFGQGTKVEIKR |
| | (SEQ ID NO: 86) | (SEQ ID NO: 87) |
| TY21692 | EVQLVESGGGLVQPGGSLRLSCAASGYSITSG | DIQLTQSPSSLSASVGDRVTITCRASQSISSY |
| | HYWSWIRQAPGKGLEWIGDISHSGSTYYSQSL | LNWYQQKPGKAPKLLIYDASNLETGVPSR |
| | KSRVTISRDNSKNTLYLQLNSLRAEDTAVYYC | FSGSGSGTDFTLTISSLQPEDFATYYCQHHY |
| | ARGSRTGYFDYWGQGTLVTVSS | GTPLTFGQGTKVEIKR |
| | (SEQ ID NO: 88) | (SEQ ID NO: 89) |

In some embodiments, the antibody that binds human CTLA4 comprises one, two, three, four, five, or six CDRs of antibody TY21580 as shown in Table 2. In some embodiments, the antibody comprises the VH and/or the VL of antibody TY21580 as shown in Table 3. In some embodiments, the antibody comprises a human IgG1 Fe region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 74. In certain embodiments, a VH sequence contains substitutions (e.g., conservative substitutions), insertions, or deletions relative to the amino acid sequence of SEQ ID NO: 74, but retains the ability to bind human CTLA4 as the antibody comprising SEQ ID NO: 74. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 74. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 1, or a variant thereof comprising up to 5 (e.g., 1, 2, 3, 4, or 5) amino acid substitutions, (b) a CDR-H2 comprising the amino acid sequence of SEQ ID NO: 2, or a variant thereof comprising up to 5 (e.g., 1, 2, 3, 4, or 5) amino acid substitutions, and (c) a CDR-H3 comprising the amino acid sequence of SEQ ID NO: 3, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 75. In certain embodiments, a VL sequence contains substitutions (e.g., conservative substitutions), insertions, or deletions relative to the amino acid sequence of SEQ ID NO: 75, but retains the ability to bind human CTLA4 as the antibody comprising SEQ ID NO: 75. In certain embodiments, a total of 1 to 11 (e.g., 1, 2, 3, 4, 5, 6, 7, 8, 9, 10, or 11) amino acids have been substituted, inserted and/or deleted in SEQ ID NO: 75. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 4, or a variant thereof comprising up to 5 (e.g., 1, 2, 3, 4, or 5) amino acid substitutions; (b) a CDR-L2 comprising the amino acid sequence of SEQ ID NO: 5, or a variant thereof comprising up to 5 (e.g., 1, 2, 3, 4, or 5) amino acid substitutions; and (c) a CDR-L3 comprising the amino acid sequence of SEQ ID NO: 6, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 74, or an amino acid sequence having at least 80% (e.g., at least 85%, 90%, 95%, 98%, or 99%; or 100%) sequence identity with SEQ ID NO: 74; and a VL comprising the amino acid sequence of SEQ ID NO: 75, or an amino acid sequence having at least 80% (e.g., at least 85%, 90%, 95%, 98%, or 99%; or 100%) sequence identity with SEQ ID NO: 75, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, and a CDR-H3 comprising the amino acid sequence of SEQ ID NO: 3, or a variant thereof comprising up to 5 (e.g., 1, 2, 3, 4, or 5) amino acid substitutions; and a VL comprising a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 74; and a VL CDR1, a VL CDR2, and a VL CDR3, respectively comprising the amino acid sequences of a CDR1, a CDR2, and a CDR3 of a VL having the sequence set forth in SEQ ID NO: 75, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a light chain and/or a heavy chain of antibody TY24040. In some embodiments, the antibody that binds human CTLA4 comprises a light chain comprising the amino acid sequence set forth in SEQ ID NO: 90, below. In some embodiments, the antibody that binds human CTLA4 comprises a light chain comprising an amino acid sequence having at least 80%, 81%, 82%, 3%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 90. In some embodiments, the antibody that binds human CTLA4 comprises a heavy chain comprising the amino acid sequence set forth in SEQ ID NO: 91, below. In some embodiments, the antibody that binds human CTLA4 comprises a heavy chain comprising an amino acid sequence having at least 80%, 81%, 82%, 3%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 91. In some embodiments, the antibody that binds human CTLA4 comprises a heavy chain comprising the amino acid sequence set forth in SEQ ID NO: 91, without the C-terminal lysine residue.

TY24040 Light Chain
(SEQ ID NO: 90)
DIQLTQSPSSLSASVGDRVTITCRASQSVRGRFLAWYQQKPGKAPK
LLIYDASNRATGIPSRFSGSGSGTDFTLTISSLQPEDFATYYCQQS
SSWPPTFGQGTKVEIKRTVAAPSVFIFPPSDEQLKSGTASVVCLLN
NFYPREAKVQWKVDNALQSGNSQESVTEQDSKDSTYSLSSTLTLSK
ADYEKHKVYACEVTHQGLSSPVTKSFNRGEC
TY24040 Heavy Chain
(SEQ ID NO: 91)
EVQLVESGGGLVQPGGSLRLSCAASGYSISSGYHWSWIRQAPGKGL
EWLARIDWDDDKYYSTSLKSRLTISRDNSKNTLYLQLNSLRAEDTA
VYYCARSYVYFDYWGQGTLVTVSSASTKGPSVFPLAPSSKSTSGGT
AALGCLVKDYFPEPVTVSWNSGALTSGVHTFPAVLQSSGLYSLSSV
VTVPSSSLGTQTYICNVNHKPSNTKVDKKVEPKSCDKTHTCPPCPA
PELLGGPDVFLFPPKPKDTLMISRTPEVTCVVVDVSHEDPEVKFNW
YVDGVEVHNAKTKPREEQYNSTYRVVSVLTVLHQDWLNGKEYKCKV
SNKALPAPEEKTISKAKGQPREPQVYTLPPSRDELTKNQVSLTCLV
KGFYPSDIAVEWESNGQPENNYKTTPPVLDSDGSFFLYSKLTVDKS
RWQQGNVFSCSVMHEALHNHYTQKSLSLSPGK

In some embodiments, the antibody that binds human CTLA4 comprises one, two, three, four, five, or six CDRs of antibody TY21585 as shown in Table 2. In some embodiments, the antibody comprises the VH and/or the VL of antibody TY21585 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 64. In certain embodiments, a VH sequence contains substitutions (e.g., conservative substitutions), insertions, or deletions relative to the amino acid sequence of SEQ ID NO: 64, but retains the ability to bind human CTLA4 as the antibody comprising SEQ ID NO: 64. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 64. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 7, or a variant thereof comprising up to 5 (e.g., 1, 2, 3, 4, or 5) amino acid substitutions, (b) a CDR-H2 comprising the amino acid sequence of SEQ ID NO: 8, or a variant thereof comprising up to 5 (e.g., 1, 2, 3, 4, or 5) amino acid substitutions, and (c) a CDR-H3 comprising the amino acid sequence of SEQ ID NO: 9, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 65. In certain embodiments, a VL sequence contains substitutions (e.g., conservative substitutions), insertions, or deletions relative to the amino acid sequence of SEQ ID NO: 65, but retains the ability to bind human CTLA4 as the antibody comprising SEQ ID NO: 65. In certain embodiments, a total of 1 to 11 (e.g., 1, 2, 3, 4, 5, 6, 7, 8, 9, 10, or 11) amino acids have been substituted, inserted and/or deleted in SEQ ID NO: 65. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 10, or a variant thereof comprising up to 5 (e.g., 1, 2, 3, 4, or 5) amino acid substitutions; (b) a CDR-L2 comprising the amino acid sequence of SEQ ID NO: 5, or a variant thereof comprising up to 5 (e.g., 1, 2, 3, 4, or 5) amino acid substitutions; and (c) a CDR-L3 comprising the amino acid sequence of SEQ ID NO: 11, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 64, or an amino acid sequence having at least 80% (e.g., at least 85%, 90%, 95%, 98%, or 99%; or 100%) sequence identity with SEQ ID NO: 64; and a VL comprising the amino acid sequence of SEQ ID NO: 65 or an amino acid sequence having at least 80% (e.g., at least 85%, 90%, 95%, 98%, or 99%; or 100%) sequence identity with SEQ ID NO: 65, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305T substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 7, or a variant thereof comprising up to 5 (e.g., 1, 2, 3, 4, or 5) amino acid substitutions, a CDR-H2 comprising the amino acid sequence of SEQ ID NO: 8, or a variant thereof comprising up to 5 (e.g., 1, 2, 3, 4, or 5) amino acid substitutions, and a CDR-H3 comprising the amino acid sequence of SEQ ID NO: 9, or a variant thereof comprising up to 5 (e.g., 1, 2, 3, 4, or 5) amino acid substitutions; and a VL comprising a CDR-L1 comprising the amino acid sequence of SEQ ID NO: 10, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 11, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 64; and a VL CDR1, a VL CDR2, and a VL CDR3, respectively comprising the amino acid sequences of a CDR1, a CDR2, and a CDR3 of a VL having the sequence set forth in SEQ ID NO: 65, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21586 as shown in Table 2. In some embodiments, the antibody comprises the VH and/or the VL of antibody TY21586 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 66. In certain embodiments, a VH sequence contains substitutions (e.g., conservative substitutions), insertions, or deletions relative to the amino acid sequence of SEQ ID NO: 66, but retains the ability to bind human CTLA4 as the antibody comprising SEQ ID NO: 66. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 66. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 12, or a variant thereof comprising up to 5 (e.g., 1, 2, 3, 4, or 5) amino acid substitutions, (b) a CDR-H2 comprising the amino acid sequence of SEQ ID NO: 13, or a variant thereof comprising up to 5 (e.g., 1, 2, 3, 4, or 5) amino acid substitutions, and (c) a CDR-H3 comprising the amino acid sequence of SEQ ID NO: 14, or a variant thereof comprising up to 5 (e.g., 1, 2, 3, 4, or 5) amino acid substitutions. In some embodiments, the antibody comprises a human IgG1 Fc region one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 67. In certain embodiments, a VL sequence contains substitutions (e.g., conservative substitutions), insertions, or deletions relative to the amino acid sequence of SEQ ID NO: 67, but retains the ability to bind human CTLA4 as the antibody comprising SEQ ID NO: 67. In certain embodiments, a total of 1 to 11 (e.g., 1, 2, 3, 4, 5, 6, 7, 8, 9, 10, or 11) amino acids have been substituted, inserted and/or deleted in SEQ ID NO: 67. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 15, or a variant thereof comprising up to 5 (e.g., 1, 2, 3, 4, or 5) amino acid substitutions; (b) a CDR-L2 comprising the amino acid sequence of SEQ ID NO: 16, or a variant thereof comprising up to 5 (e.g., 1, 2, 3, 4, or 5) amino acid substitutions; and (c) a CDR-L3 comprising the amino acid sequence of SEQ ID NO: 17, or a variant thereof comprising up to 5 (e.g., 1, 2, 3, 4, or 5) amino acid substitutions. In some embodiments, the antibody comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 66, or an amino acid sequence having at least 80% (e.g., at least 85%, 90%, 95%, 98%, or 99%; or 100%) sequence identity with SEQ ID NO: 66, and a VL comprising the amino acid sequence of SEQ ID NO: 67 or an amino acid sequence having at least 80% (e.g., at least 85%, 90%, 95%, 98%, or 99%; or 100%) sequence identity with SEQ ID NO: 67, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 12, or a variant thereof comprising up to 5 (e.g., 1, 2, 3, 4, or 5) amino acid substitutions, a CDR-H2 comprising the amino acid sequence of SEQ ID NO: 13, or a variant thereof comprising up to 5 (e.g., 1, 2, 3, 4, or 5) amino acid substitutions, and a CDR-H3 comprising the amino acid sequence of SEQ ID NO: 14, or a variant thereof comprising up to 5 (e.g., 1, 2, 3, 4, or 5) amino acid substitutions; and a VL comprising a CDR-L1 comprising the amino acid sequence of SEQ ID NO: 15, or a variant thereof comprising up to 5 (e.g., 1, 2, 3, 4, or 5) amino acid substitutions, a CDR-L2 comprising the amino acid sequence of SEQ ID NO: 16, or a variant thereof comprising up to 5 (e.g., 1, 2, 3, 4, or 5) amino acid substitutions, and a CDR-L3 comprising the amino acid sequence of SEQ ID NO: 17, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 66; and a VL CDR1, a VL CDR2, and a VL CDR3, respectively comprising the amino acid sequences of a CDR1, a CDR2, and a CDR3 of a VL having the sequence set forth in SEQ ID NO: 67, and wherein the antibody comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21587 as shown in Table 2. In some embodiments, the antibody comprises the VH and/or the VL of antibody TY21587 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 68. In certain embodiments, a VH sequence contains substitutions (e.g., conservative substitutions), insertions, or deletions relative to the amino acid sequence of SEQ ID NO: 68, but retains the ability to bind human CTLA4 as the antibody comprising SEQ ID NO: 68. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 68. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 18, or a variant thereof comprising up to 5 (e.g., 1, 2, 3, 4, or 5) amino acid substitutions, (b) a CDR-H2 comprising the amino acid sequence of SEQ ID NO: 19, or a variant thereof comprising up to 5 (e.g., 1, 2, 3, 4, or 5) amino acid substitutions, and (c) a CDR-H3 comprising the amino acid sequence of SEQ ID NO: 20, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 69. In certain embodiments, a VL sequence contains substitutions (e.g., conservative substitutions), insertions, or deletions relative to the amino acid sequence of SEQ ID NO: 69, but retains the ability to bind human CTLA4 as the antibody comprising SEQ ID NO: 69. In certain embodiments, a total of 1 to 11 (e.g., 1, 2, 3, 4, 5, 6, 7, 8, 9, 10, or 11) amino acids have been substituted, inserted and/or deleted in SEQ ID NO: 69. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 21, or a variant thereof comprising up to 5 (e.g., 1, 2, 3, 4, or 5) amino acid substitutions; (b) a CDR-L2 comprising the amino acid sequence of SEQ ID NO: 5, or a variant thereof comprising up to 5 (e.g., 1, 2, 3, 4, or 5) amino acid substitutions; and (c) a CDR-L3 comprising the amino acid sequence of SEQ ID NO: 22, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 68, or an amino acid sequence having at least 80% (e.g., at least 85%, 90%, 95%, 98%, or 99%; or 100%) sequence identity with SEQ ID NO: 68; and a VL comprising the amino acid sequence of SEQ ID NO: 69, or an amino acid sequence having at least 80% (e.g., at least 85%, 90%, 95%, 98%, or 99%; or 100%) sequence identity with SEQ ID NO: 69, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 18, or a variant thereof comprising up to 5 (e.g., 1, 2, 3, 4, or 5) amino acid substitutions, a CDR-H2 comprising the amino acid sequence of SEQ ID NO: 19, or a variant thereof comprising up to 5 (e.g., 1, 2, 3, 4, or 5) amino acid substitutions, and a CDR-H3 comprising the amino acid sequence of SEQ ID NO: 20, or a variant thereof comprising up to 5 (e.g., 1, 2, 3, 4, or 5) amino acid substitutions; and a VL comprising a CDR-L1 comprising the amino acid sequence of SEQ ID NO: 21, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 22, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 68; and a VL CDR1, a VL CDR2, and a VL CDR3, respectively comprising the amino acid sequences of a CDR1, a CDR2, and a CDR3 of a VL having the sequence set forth in SEQ ID NO: 69, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21588 as shown in Table 2. In some embodiments, the antibody comprises the VH and/or the VL of antibody TY21588 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 70. In certain embodiments, a VH sequence contains substitutions (e.g., conservative substitutions), insertions, or deletions relative to the amino acid sequence of SEQ ID NO: 70, but retains the ability to bind human CTLA4 as the antibody comprising SEQ ID NO: 70. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 70. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 23, or a variant thereof comprising up to 5 (e.g., 1, 2, 3, 4, or 5) amino acid substitutions, (b) a CDR-H2 comprising the amino acid sequence of SEQ ID NO: 24, or a variant thereof comprising up to 5 (e.g., 1, 2, 3, 4, or 5) amino acid substitutions, and (c) a CDR-H3 comprising the amino acid sequence of SEQ ID NO: 25, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 71. In certain embodiments, a VL sequence contains substitutions (e.g., conservative substitutions), insertions, or deletions relative to the amino acid sequence of SEQ ID NO: 71, but retains the ability to bind human CTLA4 as the antibody comprising SEQ ID NO: 71. In certain embodiments, a total of 1 to 11 (e.g., 1, 2, 3, 4, 5, 6, 7, 8, 9, 10, or 11) amino acids have been substituted, inserted and/or deleted in SEQ ID NO: 71. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 26, or a variant thereof comprising up to 5 (e.g., 1, 2, 3, 4, or 5) amino acid substitutions; (b) a CDR-L2 comprising the amino acid sequence of SEQ ID NO: 27, or a variant thereof comprising up to 5 (e.g., 1, 2, 3, 4, or 5) amino acid substitutions; and (c) a CDR-L3 comprising the amino acid sequence of SEQ ID NO: 28, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 70, or an amino acid sequence having at least 80% (e.g., at least 85%, 90%, 95%, 98%, or 99%; or 100%) sequence identity with SEQ ID NO: 70; and a VL comprising the amino acid sequence of SEQ ID NO: 71, or an amino acid sequence having at least 80% (e.g., at least 85%, 90%, 95%, 98%, or 99%; or 100%) sequence identity with SEQ ID NO: 71, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 23, or a variant thereof comprising up to 5 (e.g., 1, 2, 3, 4, or 5) amino acid substitutions, a CDR-H2 comprising the amino acid sequence of SEQ ID NO: 24, or a variant thereof comprising up to 5 (e.g., 1, 2, 3, 4, or 5) amino acid substitutions, and a CDR-H3 comprising the amino acid sequence of SEQ ID NO: 25, or a variant thereof comprising up to 5 (e.g., 1, 2, 3, 4, or 5) amino acid substitutions; and a VL comprising a CDR-L1 comprising the amino acid sequence of SEQ ID NO: 26, or a variant thereof comprising up to 5 (e.g., 1, 2, 3, 4, or 5) amino acid substitutions, a CDR-L2 comprising the amino acid sequence of SEQ ID NO: 27, or a variant thereof comprising up to 5 (e.g., 1, 2, 3, 4, or 5) amino acid substitutions, and a CDR-L3 comprising the amino acid sequence of SEQ ID NO: 28, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 70; and a VL CDR1, a VL CDR2, and a VL CDR3, respectively comprising the amino acid sequences of a CDR1, a CDR2, and a CDR3 of a VL having the sequence set forth in SEQ ID NO: 71, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21589 as shown in Table 2. In some embodiments, the antibody comprises the VH and/or the VL of antibody TY21589 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 72. In certain embodiments, a VH sequence contains substitutions (e.g., conservative substitutions), insertions, or deletions relative to the amino acid sequence of SEQ ID NO: 72, but retains the ability to bind human CTLA4 as the antibody comprising SEQ ID NO: 72. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 72. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 29, or a variant thereof comprising up to 5 (e.g., 1, 2, 3, 4, or 5) amino acid substitutions, (b) a CDR-H2 comprising the amino acid sequence of SEQ ID NO: 30, or a variant thereof comprising up to 5 (e.g., 1, 2, 3, 4, or 5) amino acid substitutions, and (c) a CDR-H3 comprising the amino acid sequence of SEQ ID NO: 31, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 73. In certain embodiments, a VL sequence contains substitutions (e.g., conservative substitutions), insertions, or deletions relative to the amino acid sequence of SEQ ID NO: 73, but retains the ability to bind human CTLA4 as the antibody comprising SEQ ID NO: 73. In certain embodiments, a total of 1 to 11 (e.g., 1, 2, 3, 4, 5, 6, 7, 8, 9, 10, or 11) amino acids have been substituted, inserted and/or deleted in SEQ ID NO: 73. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 32, or a variant thereof comprising up to 5 (e.g., 1, 2, 3, 4, or 5) amino acid substitutions; (b) a CDR-L2 comprising the amino acid sequence of SEQ ID NO: 5, or a variant thereof comprising up to 5 (e.g., 1, 2, 3, 4, or 5) amino acid substitutions; and (c) a CDR-L3 comprising the amino acid sequence of SEQ ID NO: 33, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 72, or an amino acid sequence having at least 80% (e.g., at least 85%, 90%, 95%, 98%, or 99%; or 100%) sequence identity with SEQ ID NO: 72; and a VL comprising the amino acid sequence of SEQ ID NO: 73, or an amino acid sequence having at least 80% (e.g., at least 85%, 90%, 95%, 98%, or 99%; or 100%) sequence identity with SEQ ID NO: 73, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 29, or a variant thereof comprising up to 5 (e.g., 1, 2, 3, 4, or 5) amino acid substitutions, a CDR-H2 comprising the amino acid sequence of SEQ ID NO: 30, or a variant thereof comprising up to 5 (e.g., 1, 2, 3, 4, or 5) amino acid substitutions, and a CDR-H3 comprising the amino acid sequence of SEQ ID NO: 31, or a variant thereof comprising up to 5 (e.g., 1, 2, 3, 4, or 5) amino acid substitutions; and a VL comprising a CDR-L1 comprising the amino acid sequence of SEQ ID NO: 32, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 33, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 72; and a VL CDR1, a VL CDR2, and a VL CDR3, respectively comprising the amino acid sequences of a CDR1, a CDR2, and a CDR3 of a VL having the sequence set forth in SEQ ID NO: 73, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21591 as shown in Table 2. In some embodiments, the antibody comprises the VH and/or the VL of antibody TY21591 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 76. In certain embodiments, a VH sequence contains substitutions (e.g., conservative substitutions), insertions, or deletions relative to the amino acid sequence of SEQ ID NO: 76, but retains the ability to bind human CTLA4 as the antibody comprising SEQ ID NO: 76. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 76. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 34, or a variant thereof comprising up to 5 (e.g., 1, 2, 3, 4, or 5) amino acid substitutions, (b) a CDR-H2 comprising the amino acid sequence of SEQ ID NO: 19, or a variant thereof comprising up to 5 (e.g., 1, 2, 3, 4, or 5) amino acid substitutions, and (c) a CDR-H3 comprising the amino acid sequence of SEQ ID NO: 35, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 77. In certain embodiments, a VL sequence contains substitutions (e.g., conservative substitutions), insertions, or deletions relative to the amino acid sequence of SEQ ID NO: 77, but retains the ability to bind human CTLA4 as the antibody comprising SEQ ID NO: 77. In certain embodiments, a total of 1 to 11 (e.g., 1, 2, 3, 4, 5, 6, 7, 8, 9, 10, or 11) amino acids have been substituted, inserted and/or deleted in SEQ ID NO: 77. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 36, or a variant thereof comprising up to 5 (e.g., 1, 2, 3, 4, or 5) amino acid substitutions; (b) a CDR-L2 comprising the amino acid sequence of SEQ ID NO: 5, or a variant thereof comprising up to 5 (e.g., 1, 2, 3, 4, or 5) amino acid substitutions; and (c) a CDR-L3 comprising the amino acid sequence of SEQ ID NO: 37, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 76, or an amino acid sequence having at least 80% (e.g., at least 85%, 90%, 95%, 98%, or 99%; or 100%) sequence identity with SEQ ID NO: 76; and a VL comprising the amino acid sequence of SEQ ID NO: 77, or an amino acid sequence having at least 80% (e.g., at least 85%, 90%, 95%, 98%, or 99%; or 100%) sequence identity with SEQ ID NO: 77, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 34, or a variant thereof comprising up to 5 (e.g., 1, 2, 3, 4, or 5) amino acid substitutions, a CDR-H2 comprising the amino acid sequence of SEQ ID NO: 19, or a variant thereof comprising up to 5 (e.g., 1, 2, 3, 4, or 5) amino acid substitutions, and a CDR-H3 comprising the amino acid sequence of SEQ ID NO: 35, or a variant thereof comprising up to 5 (e.g., 1, 2, 3, 4, or 5) amino acid substitutions; and a VL comprising a CDR-L1 comprising the amino acid sequence of SEQ ID NO: 36, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 37, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 76; and a VL CDR1, a VL CDR2, and a VL CDR3, respectively comprising the amino acid sequences of a CDR1, a CDR2, and a CDR3 of a VL having the sequence set forth in SEQ ID NO: 77, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21686 as shown in Table 2. In some embodiments, the antibody comprises the VH and/or the VL of antibody TY21686 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 78. In certain embodiments, a VH sequence contains substitutions (e.g., conservative substitutions), insertions, or deletions relative to the amino acid sequence of SEQ ID NO: 78, but retains the ability to bind human CTLA4 as the antibody comprising SEQ ID NO: 78. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 78. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 38, or a variant thereof comprising up to 5 (e.g., 1, 2, 3, 4, or 5) amino acid substitutions, (b) a CDR-H2 comprising the amino acid sequence of SEQ ID NO: 39, or a variant thereof comprising up to 5 (e.g., 1, 2, 3, 4, or 5) amino acid substitutions, and (c) a CDR-H3 comprising the amino acid sequence of SEQ ID NO: 40, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 79. In certain embodiments, a VL sequence contains substitutions (e.g., conservative substitutions), insertions, or deletions relative to the amino acid sequence of SEQ ID NO: 79, but retains the ability to bind human CTLA4 as the antibody comprising SEQ ID NO: 79. In certain embodiments, a total of 1 to 11 (e.g., 1, 2, 3, 4, 5, 6, 7, 8, 9, 10, or 11) amino acids have been substituted, inserted and/or deleted in SEQ ID NO: 79. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 41, or a variant thereof comprising up to 5 (e.g., 1, 2, 3, 4, or 5) amino acid substitutions; (b) a CDR-L2 comprising the amino acid sequence of SEQ ID NO: 42, or a variant thereof comprising up to 5 (e.g., 1, 2, 3, 4, or 5) amino acid substitutions; and (c) a CDR-L3 comprising the amino acid sequence of SEQ ID NO: 43, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 78, or an amino acid sequence having at least 80% (e.g., at least 85%, 90%, 95%, 98%, or 99%; or 100%) sequence identity with SEQ ID NO: 78; and a VL comprising the amino acid sequence of SEQ ID NO: 79, or an amino acid sequence having at least 80% (e.g., at least 85%, 90%, 95%, 98%, or 99%; or 100%) sequence identity with SEQ ID NO: 79, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 38, or a variant thereof comprising up to 5 (e.g., 1, 2, 3, 4, or 5) amino acid substitutions, a CDR-H2 comprising the amino acid sequence of SEQ ID NO: 39, or a variant thereof comprising up to 5 (e.g., 1, 2, 3, 4, or 5) amino acid substitutions, and a CDR-H3 comprising the amino acid sequence of SEQ ID NO: 40, or a variant thereof comprising up to 5 (e.g., 1, 2, 3, 4, or 5) amino acid substitutions; and a VL comprising a CDR-L1 comprising the amino acid sequence of SEQ ID NO: 41, or a variant thereof comprising up to 5 (e.g., 1, 2, 3, 4, or 5) amino acid substitutions, a CDR-L2 comprising the amino acid sequence of SEQ ID NO: 42, or a variant thereof comprising up to 5 (e.g., 1, 2, 3, 4, or 5) amino acid substitutions, and a CDR-L3 comprising the amino acid sequence of SEQ ID NO: 43, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 78; and a VL CDR1, a VL CDR2, and a VL CDR3, respectively comprising the amino acid sequences of a CDR1, a CDR2, and a CDR3 of a VL having the sequence set forth in SEQ ID NO: 79, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21687 as shown in Table 2. In some embodiments, the antibody comprises the VH and/or the VL of antibody TY21687 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 80. In certain embodiments, a VH sequence contains substitutions (e.g., conservative substitutions), insertions, or deletions relative to the amino acid sequence of SEQ ID NO: 80, but retains the ability to bind human CTLA4 as the antibody comprising SEQ ID NO: 80. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 80. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 44, or a variant thereof comprising up to 5 (e.g., 1, 2, 3, 4, or 5) amino acid substitutions, (b) a CDR-H2 comprising the amino acid sequence of SEQ ID NO: 45, or a variant thereof comprising up to 5 (e.g., 1, 2, 3, 4, or 5) amino acid substitutions, and (c) a CDR-H3 comprising the amino acid sequence of SEQ ID NO: 46, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 81. In certain embodiments, a VL sequence contains substitutions (e.g., conservative substitutions), insertions, or deletions relative to the amino acid sequence of SEQ ID NO: 81, but retains the ability to bind human CTLA4 as the antibody comprising SEQ ID NO: 81. In certain embodiments, a total of 1 to 11 (e.g., 1, 2, 3, 4, 5, 6, 7, 8, 9, 10, or 11) amino acids have been substituted, inserted and/or deleted in SEQ ID NO: 81. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 47, or a variant thereof comprising up to 5 (e.g., 1, 2, 3, 4, or 5) amino acid substitutions; (b) a CDR-L2 comprising the amino acid sequence of SEQ ID NO: 5, or a variant thereof comprising up to 5 (e.g., 1, 2, 3, 4, or 5) amino acid substitutions; and (c) a CDR-L3 comprising the amino acid sequence of SEQ ID NO: 48, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 80, or an amino acid sequence having at least 80% (e.g., at least 85%, 90%, 95%, 98%, or 99%; or 100%) sequence identity with SEQ ID NO: 80; and a VL comprising the amino acid sequence of SEQ ID NO: 81, or an amino acid sequence having at least 80% (e.g., at least 85%, 90%, 95%, 98%, or 99%; or 100%) sequence identity with SEQ ID NO: 81, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 44, or a variant thereof comprising up to 5 (e.g., 1, 2, 3, 4, or 5) amino acid substitutions, a CDR-H2 comprising the amino acid sequence of SEQ ID NO: 45, or a variant thereof comprising up to 5 (e.g., 1, 2, 3, 4, or 5) amino acid substitutions, and a CDR-H3 comprising the amino acid sequence of SEQ ID NO: 46, or a variant thereof comprising up to 5 (e.g., 1, 2, 3, 4, or 5) amino acid substitutions; and a VL comprising a CDR-L1 comprising the amino acid sequence of SEQ ID NO: 47, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 48, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 80; and a VL CDR1, a VL CDR2, and a VL CDR3, respectively comprising the amino acid sequences of a CDR1, a CDR2, and a CDR3 of a VL having the sequence set forth in SEQ ID NO: 81, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21689 as shown in Table 2. In some embodiments, the antibody comprises the VH and/or the VL of antibody TY21689 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 82. In certain embodiments, a VH sequence contains substitutions (e.g., conservative substitutions), insertions, or deletions relative to the amino acid sequence of SEQ ID NO: 82, but retains the ability to bind human CTLA4 as the antibody comprising SEQ ID NO: 82. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 82. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 49, or a variant thereof comprising up to 5 (e.g., 1, 2, 3, 4, or 5) amino acid substitutions, (b) a CDR-H2 comprising the amino acid sequence of SEQ ID NO: 19, or a variant thereof comprising up to 5 (e.g., 1, 2, 3, 4, or 5) amino acid substitutions, and (c) a CDR-H3 comprising the amino acid sequence of SEQ ID NO: 50, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 83. In certain embodiments, a VL sequence contains substitutions (e.g., conservative substitutions), insertions, or deletions relative to the amino acid sequence of SEQ ID NO: 83, but retains the ability to bind human CTLA4 as the antibody comprising SEQ ID NO: 83. In certain embodiments, a total of 1 to 11 (e.g., 1, 2, 3, 4, 5, 6, 7, 8, 9, 10, or 11) amino acids have been substituted, inserted and/or deleted in SEQ ID NO: 83. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 51, or a variant thereof comprising up to 5 (e.g., 1, 2, 3, 4, or 5) amino acid substitutions; (b) a CDR-L2 comprising the amino acid sequence of SEQ ID NO: 16, or a variant thereof comprising up to 5 (e.g., 1, 2, 3, 4, or 5) amino acid substitutions; and (c) a CDR-L3 comprising the amino acid sequence of SEQ ID NO: 52, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 82, or an amino acid sequence having at least 80% (e.g., at least 85%, 90%, 95%, 98%, or 99%; or 100%) sequence identity with SEQ ID NO: 82; and a VL comprising the amino acid sequence of SEQ ID NO: 83, or an amino acid sequence having at least 80% (e.g., at least 85%, 90%, 95%, 98%, or 99%; or 100%) sequence identity with SEQ ID NO: 83, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 49, or a variant thereof comprising up to 5 (e.g., 1, 2, 3, 4, or 5) amino acid substitutions, a CDR-H2 comprising the amino acid sequence of SEQ ID NO: 19, or a variant thereof comprising up to 5 (e.g., 1, 2, 3, 4, or 5) amino acid substitutions, and a CDR-H3 comprising the amino acid sequence of SEQ ID NO: 50, or a variant thereof comprising up to 5 (e.g., 1, 2, 3, 4, or 5) amino acid substitutions; and a VL comprising a CDR-L1 comprising the amino acid sequence of SEQ ID NO: 51, or a variant thereof comprising up to 5 (e.g., 1, 2, 3, 4, or 5) amino acid substitutions, a CDR-L2 comprising the amino acid sequence of SEQ ID NO: 16, or a variant thereof comprising up to 5 (e.g., 1, 2, 3, 4, or 5) amino acid substitutions, and a CDR-L3 comprising the amino acid sequence of SEQ ID NO: 52, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 82; and a VL CDR1, a VL CDR2, and a VL CDR3, respectively comprising the amino acid sequences of a CDR1, a CDR2, and a CDR3 of a VL having the sequence set forth in SEQ ID NO: 83, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21680 as shown in Table 2. In some embodiments, the antibody comprises the VH and/or the VL of antibody TY21680 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 84. In certain embodiments, a VH sequence contains substitutions (e.g., conservative substitutions), insertions, or deletions relative to the amino acid sequence of SEQ ID NO: 84, but retains the ability to bind human CTLA4 as the antibody comprising SEQ ID NO: 84. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 84. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 44, or a variant thereof comprising up to 5 (e.g., 1, 2, 3, 4, or 5) amino acid substitutions, (b) a CDR-H2 comprising the amino acid sequence of SEQ ID NO: 45, or a variant thereof comprising up to 5 (e.g., 1, 2, 3, 4, or 5) amino acid substitutions, and (c) a CDR-H3 comprising the amino acid sequence of SEQ ID NO: 53, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 85. In certain embodiments, a VL sequence contains substitutions (e.g., conservative substitutions), insertions, or deletions relative to the amino acid sequence of SEQ ID NO: 85, but retains the ability to bind human CTLA4 as the antibody comprising SEQ ID NO: 85. In certain embodiments, a total of 1 to 11 (e.g., 1, 2, 3, 4, 5, 6, 7, 8, 9, 10, or 11) amino acids have been substituted, inserted and/or deleted in SEQ ID NO: 85. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 54, or a variant thereof comprising up to 5 (e.g., 1, 2, 3, 4, or 5) amino acid substitutions; (b) a CDR-L2 comprising the amino acid sequence of SEQ ID NO: 16, or a variant thereof comprising up to 5 (e.g., 1, 2, 3, 4, or 5) amino acid substitutions; and (c) a CDR-L3 comprising the amino acid sequence of SEQ ID NO: 55, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 84, or an amino acid sequence having at least 80% (e.g., at least 85%, 90%, 95%, 98%, or 99%; or 100%) sequence identity with SEQ ID NO: 84; and a VL comprising the amino acid sequence of SEQ ID NO: 85, or an amino acid sequence having at least 80% (e.g., at least 85%, 90%, 95%, 98%, or 99%; or 100%) sequence identity with SEQ ID NO: 85, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 44, or a variant thereof comprising up to 5 (e.g., 1, 2, 3, 4, or 5) amino acid substitutions, a CDR-H2 comprising the amino acid sequence of SEQ ID NO: 45, or a variant thereof comprising up to 5 (e.g., 1, 2, 3, 4, or 5) amino acid substitutions, and a CDR-H3 comprising the amino acid sequence of SEQ ID NO: 53, or a variant thereof comprising up to 5 (e.g., 1, 2, 3, 4, or 5) amino acid substitutions; and a VL comprising a CDR-L1 comprising the amino acid sequence of SEQ ID NO: 54, or a variant thereof comprising up to 5 (e.g., 1, 2, 3, 4, or 5) amino acid substitutions, a CDR-L2 comprising the amino acid sequence of SEQ ID NO: 16, or a variant thereof comprising up to 5 (e.g., 1, 2, 3, 4, or 5) amino acid substitutions, and a CDR-L3 comprising the amino acid sequence of SEQ ID NO: 55, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 84; and a VL CDR1, a VL CDR2, and a VL CDR3, respectively comprising the amino acid sequences of a CDR1, a CDR2, and a CDR3 of a VL having the sequence set forth in SEQ ID NO: 85, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21691 as shown in Table 2. In some embodiments, the antibody comprises the VH and/or the VL of antibody TY21691 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 86. In certain embodiments, a VH sequence contains substitutions (e.g., conservative substitutions), insertions, or deletions relative to the amino acid sequence of SEQ ID NO: 86, but retains the ability to bind human CTLA4 as the antibody comprising SEQ ID NO: 86. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 86. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 7, or a variant thereof comprising up to 5 (e.g., 1, 2, 3, 4, or 5) amino acid substitutions, (b) a CDR-H2 comprising the amino acid sequence of SEQ ID NO: 56, or a variant thereof comprising up to 5 (e.g., 1, 2, 3, 4, or 5) amino acid substitutions, and (c) a CDR-H3 comprising the amino acid sequence of SEQ ID NO: 57, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 87. In certain embodiments, a VL sequence contains substitutions (e.g., conservative substitutions), insertions, or deletions relative to the amino acid sequence of SEQ ID NO: 87, but retains the ability to bind human CTLA4 as the antibody comprising SEQ ID NO: 87. In certain embodiments, a total of 1 to 11 (e.g., 1, 2, 3, 4, 5, 6, 7, 8, 9, 10, or 11) amino acids have been substituted, inserted and/or deleted in SEQ ID NO: 87. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 58, or a variant thereof comprising up to 5 (e.g., 1, 2, 3, 4, or 5) amino acid substitutions; (b) a CDR-L2 comprising the amino acid sequence of SEQ ID NO: 16, or a variant thereof comprising up to 5 (e.g., 1, 2, 3, 4, or 5) amino acid substitutions; and (c) a CDR-L3 comprising the amino acid sequence of SEQ ID NO: 59, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 86, or an amino acid sequence having at least 80% (e.g., at least 85%, 90%, 95%, 98%, or 99%; or 100%) sequence identity with SEQ ID NO: 86; and a VL comprising the amino acid sequence of SEQ ID NO: 87, or an amino acid sequence having at least 80% (e.g., at least 85%, 90%, 95%, 98%, or 99%; or 100%) sequence identity with SEQ ID NO: 87, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 7, or a variant thereof comprising up to 5 (e.g., 1, 2, 3, 4, or 5) amino acid substitutions, a CDR-H2 comprising the amino acid sequence of SEQ ID NO: 56, or a variant thereof comprising up to 5 (e.g., 1, 2, 3, 4, or 5) amino acid substitutions, and a CDR-H3 comprising the amino acid sequence of SEQ ID NO: 57, or a variant thereof comprising up to 5 (e.g., 1, 2, 3, 4, or 5) amino acid substitutions; and a VL comprising a CDR-L1 comprising the amino acid sequence of SEQ ID NO: 58, or a variant thereof comprising up to 5 (e.g., 1, 2, 3, 4, or 5) amino acid substitutions, a CDR-L2 comprising the amino acid sequence of SEQ ID NO: 16, or a variant thereof comprising up to 5 (e.g., 1, 2, 3, 4, or 5) amino acid substitutions, and a CDR-L3 comprising the amino acid sequence of SEQ ID NO: 59, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 86; and a VL CDR1, a VL CDR2, and a VL CDR3, respectively comprising the amino acid sequences of a CDR1, a CDR2, and a CDR3 of a VL having the sequence set forth in SEQ ID NO: 87, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises one, two, three, four, five, or six CDRs of antibody TY21692 as shown in Table 2. In some embodiments, the antibody comprises the VH and/or the VL of antibody TY21692 as shown in Table 3. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antibody that binds human CTLA4 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 88. In certain embodiments, a VH sequence contains substitutions (e.g., conservative substitutions), insertions, or deletions relative to the amino acid sequence of SEQ ID NO: 88, but retains the ability to bind human CTLA4 as the antibody comprising SEQ ID NO: 88. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 88. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 60, or a variant thereof comprising up to 5 (e.g., 1, 2, 3, 4, or 5) amino acid substitutions, (b) a CDR-H2 comprising the amino acid sequence of SEQ ID NO: 61, or a variant thereof comprising up to 5 (e.g., 1, 2, 3, 4, or 5) amino acid substitutions, and (c) a CDR-H3 comprising the amino acid sequence of SEQ ID NO: 62, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 89. In certain embodiments, a VL sequence contains substitutions (e.g., conservative substitutions), insertions, or deletions relative to the amino acid sequence of SEQ ID NO: 89, but retains the ability to bind human CTLA4 as the antibody comprising SEQ ID NO: 89. In certain embodiments, a total of 1 to 11 (e.g., 1, 2, 3, 4, 5, 6, 7, 8, 9, 10, or 11) amino acids have been substituted, inserted and/or deleted in SEQ ID NO: 89. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 63, or a variant thereof comprising up to 5 (e.g., 1, 2, 3, 4, or 5) amino acid substitutions; (b) a CDR-L2 comprising the amino acid sequence of SEQ ID NO: 27, or a variant thereof comprising up to 5 (e.g., 1, 2, 3, 4, or 5) amino acid substitutions; and (c) a CDR-L3 comprising the amino acid sequence of SEQ ID NO: 43, or a variant thereof comprising up to 5 (e.g., 1, 2, 3, 4, or 5) amino acid substitutions. In some embodiments, the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In one embodiment, the antibody that binds human CTLA4 comprises a VH comprising the amino acid sequence of SEQ ID NO: 88, or an amino acid sequence having at least 80% (e.g., at least 85%, 90%, 95%, 98%, or 99%; or 100%) sequence identity with SEQ ID NO: 88; and a VL comprising the amino acid sequence of SEQ ID NO: 89, or an amino acid sequence having at least 80% (e.g., at least 85%, 90%, 95%, 98%, or 99%; or 100%) sequence identity with SEQ ID NO: 89, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH comprising a CDR-H1 comprising the amino acid sequence of SEQ ID NO: 60, or a variant thereof comprising up to 5 (e.g., 1, 2, 3, 4, or 5) amino acid substitutions, a CDR-H2 comprising the amino acid sequence of SEQ ID NO: 61, or a variant thereof comprising up to 5 (e.g., 1, 2, 3, 4, or 5) amino acid substitutions, and a CDR-H3 comprising the amino acid sequence of SEQ ID NO: 62, or a variant thereof comprising up to 5 (e.g., 1, 2, 3, 4, or 5) amino acid substitutions; and a VL comprising a CDR-L1 comprising the amino acid sequence of SEQ ID NO: 63, or a variant thereof comprising up to 5 (e.g., 1, 2, 3, 4, or 5) amino acid substitutions, a CDR-L2 comprising the amino acid sequence of SEQ ID NO: 27, or a variant thereof comprising up to 5 (e.g., 1, 2, 3, 4, or 5) amino acid substitutions, and a CDR-L3 comprising the amino acid sequence of SEQ ID NO: 43, or a variant thereof comprising up to 5 (e.g., 1, 2, 3, 4, or 5) amino acid substitutions, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In another aspect, an antibody that binds human CTLA4 is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO: 88; and a VL CDR1, a VL CDR2, and a VL CDR3, respectively comprising the amino acid sequences of a CDR1, a CDR2, and a CDR3 of a VL having the sequence set forth in SEQ ID NO: 89, and wherein the antibody comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, an anti-CTLA4 antibody of the present disclosure cross-competes for binding to human CTLA4 with an antibody comprising: a) a CDR-H1 comprising an amino acid sequence selected from SEQ ID NOS: 1, 7, 12, 18, 23, 29, 34, 38, 44, 49, and 60, or a variant thereof comprising up to 5 (e.g., 1, 2, 3, 4, or 5) amino acid substitutions; a CDR-H2 comprising an amino acid sequence selected from SEQ ID NOS: 2, 8, 13, 19, 24, 30, 39, 45, 56, and 61, or a variant thereof comprising up to 5 (e.g., 1, 2, 3, 4, or 5) amino acid substitutions; and a CDR-H3 comprising an amino acid sequence selected from SEQ ID NOS: 3, 9, 14, 20, 25, 31, 35, 40, 46, 50, 53, 57, and 62, or a variant thereof comprising up to 5 (e.g., 1, 2, 3, 4, or 5) amino acid substitutions; and/or b) a CDR-L1 comprising an amino acid sequence selected from SEQ ID NOS: 4, 10, 15, 21, 26, 32, 36, 41, 47, 51, 54, 58, and 63, or a variant thereof comprising up to 5 (e.g., 1, 2, 3, 4, or 5) amino acid substitutions; a CDR-L2 comprising an amino acid sequence selected from SEQ ID NOS: 5, 16, 27, and 42, or a variant thereof comprising up to 5 (e.g., 1, 2, 3, 4, or 5) amino acid substitutions; and a CDR-L3 comprising an amino acid sequence selected from SEQ ID NOS: 6, 11, 17, 22, 28, 33, 37, 43, 48, 52, and 59, or a variant thereof comprising up to 5 (e.g., 1, 2, 3, 4, or 5) amino acid substitutions. In some embodiments, an anti-CTLA4 antibody of the present disclosure cross-competes for binding to human CTLA4 with an antibody comprising one, two, three, four, five, or all six of the CDRs shown for any of the exemplary antibodies described in Table 2. In some embodiments, an anti-CTLA4 antibody of the present disclosure cross-competes for binding to human CTLA4 with an antibody comprising: a) a heavy chain variable region comprising an amino acid sequence selected from SEQ ID NOS: 64, 66, 68, 70, 72, 74, 76, 78, 80, 82, 84, 86, and 88; and/or b) a light chain variable region comprising an amino acid sequence selected from SEQ ID NOS: 65, 67, 69, 71, 73, 75, 77, 79, 81, 83, 85, 87, and 89. In some embodiments, the anti-CTLA4 antibody comprises a heavy chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 64, 66, 68, 70, 72, 74, 76, 78, 80, 82, 84, 86, and 88, and/or a light chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 65, 67, 69, 71, 73, 75, 77, 79, 81, 83, 85, 87, and 89. In some embodiments, an anti-CTLA4 antibody of the present disclosure cross-competes for binding to human CTLA4 with an antibody comprising a heavy chain variable region and a light chain variable region of any of the exemplary anti-CTLA4 antibodies described in Table 3. In some embodiments, an anti-CTLA4 antibody of the present disclosure cross-competes for binding to human CTLA4 with an antibody comprising one, two, or all three CDRs of the heavy chain variable region, and/or one, two, or all three CDRs of the light chain variable region shown for any of the exemplary anti-CTLA4 antibodies described in Table 3.

In some embodiments, the anti-CTLA4 antibodies described herein deplete Treg cells selectively in tumor microenvironment as compared to Treg depletions in PBMC or spleen. In some embodiments, the anti-CTLA4 antibodies described herein have higher Treg depletion activity in tumor microenvironment as compared to ipilimumab. Also provided herein are one or more anti-CTLA4 antibodies or antigen-binding fragments that cross-compete for binding to human CTLA4 with one or more of the antibodies or antigen-binding fragments described herein.

In some embodiments, the anti-CTLA4 antibodies or antigen-binding fragments bind to human, cynomolgus monkey, mouse, rat, and/or dog CTLA4 with a K_{D} of about 500 nM or less (e.g., about 500 nM or less, about 450 nM or less, about 400 nM or less, about 350 nM or less, about 300 nM or less, about 250 nM or less, about 200 nM or less, about 150 nM or less, about 100 nM or less, about 90 nM or less, about 80 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) In some embodiments, the anti-CTLA4 antibodies or antigen-binding fragments bind to human, cynomolgus monkey, mouse, rat, and/or dog CTLA4 with a K_{D} of about 350 nM or less. In some embodiments, the anti-CTLA4 antibodies or antigen-binding fragments bind to human CTLA4 with a K_{D} of about 100 nM or less. In some embodiments, the anti-CTLA4 antibodies or antigen-binding fragments bind to human CTLA4 with a K_{D} of about 50 nM or less. In some embodiments, the anti-CTLA4 antibodies or antigen-binding fragments bind to human CTLA4 with a K_{D} of about 10 nM or less. Methods of measuring the K_{D} of an anti-CTLA4 antibody or antigen-binding fragment may be carried out using any method known in the art, including for example, by surface plasmon resonance, an ELISA, isothermal titration calorimetry, a filter binding assay, an EMSA, etc. In some embodiments, the K_{D} is measured by surface plasmon resonance or an ELISA, for example, as described in U.S. Patent Application Publication No. US20210207126A1.

In some embodiments, the anti-CTLA4 antibodies or antigen-binding fragments described herein have antagonist activity on human CTLA4. In some embodiments, the anti-CTLA4 antibodies or antigen-binding fragments repress one or more activities of human CTLA4 when a cell (e.g., a human cell) expressing human CTLA4 is contacted by the anti-CTLA4 antibody or antigen binding fragment (e.g., CTLA4 blockade as measured by an increase in a reporter gene signal using a CLA4 blockage reporter gene assay).

In some embodiments, the anti-CTLA4 antibodies or antigen-binding fragments are cross-reactive with monkey (e.g., cynomolgus monkey), mouse, rat, and/or dog CTLA4. In some embodiments, the anti-CTLA4 antibodies or antigen-binding fragments are cross-reactive with monkey CTLA4. In some embodiments, the anti-CTLA4 antibodies or antigen-binding fragments are cross-reactive with mouse CTLA4. In some embodiments, the anti-CTLA4 antibodies or antigen-binding fragments are cross-reactive with rat CTLA4. In some embodiments, the anti-CTLA4 antibodies or antigen-binding fragments are cross-reactive with dog CTLA4. In some embodiments, the anti-CTLA4 antibodies or antigen binding fragments are cross reactive with monkey and mouse CTLA4; monkey and rat CTLA4; monkey and dog CTLA4; mouse and rat CTLA4; mouse and dog CTLA4; rat and dog CTLA4; monkey, mouse, and rat CTLA4; monkey, mouse, and dog CTLA4; monkey, rat, and dog CTLA4; mouse, rat, and dog CTLA4; or monkey, mouse, rat, and dog CTLA4. In some embodiments, the anti-CTLA4 antibodies or antigen binding fragments are cross-reactive if the anti-CTLA4 antibodies or antigen-binding fragments bind to a non-human CTLA4 molecule with a K_{D} less than about 500 nM (e.g., less than about 1 nM, less than about 10 nM, less than about 25 nM, less than about 50 nM, less than about 75 nM, less than about 100 nM, less than about 150 nM, less than about 200 nM, less than about 250 nM, less than about 300 nM, less than about 350 nM, etc.). Methods of measuring antibody cross-reactivity are known in the art, including, without limitation, surface plasmon resonance, an ELISA, isothermal titration calorimetry, a filter binding assay, an EMSA, etc. In some embodiments, the cross-reactivity is measured by ELISA, for example, as described in U.S. Patent Application Publication No. US20210207126A1

In some embodiments, the anti-CTLA4 antibodies induce antibody-dependent cellular cytotoxicity (ADCC) effects against a CTLA4 expressing cell (e.g., against CTLA4-expressing human cells such as Tregs) after the anti-CTLA4 antibody binds to the cell-expressed CTLA4. In some embodiments, the anti-CTLA4 antibodies induce ADCC effects by more than about 10% (e.g., induce ADCC by more than about 10%, more than about 15%, more than about 20%, more than about 25%, more than about 30%, more than about 35%, more than about 40%, etc.) relative to a control (e.g., an isotype control or ipilimumab). In certain embodiments wherein the anti-CTLA4 antibodies comprise an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibodies induce ADCC effects by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an Fc region that does not comprise the one or more substitutions. In certain embodiments wherein the anti-CTLA4 antibodies comprise an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibodies induce ADCC effects by at a rate of at least about 1.5 fold (e.g., at least about 1.5 fold, at least about 2 fold, at least about 2.5 fold, at least about 3 fold, at least about 4 fold, or at least about 5 fold) greater than an anti-CTLA4 antibody that comprises an Fc region that does not comprise the substitutions. In some embodiments, the anti-CTLA4 antibody has a half maximal effective concentration (EC50) of about 5 nM or less (e.g., about 5 nM or less, about 4 nM or less, about 3 nM or less, about 3 nM or less, about 2 nM or less, about 1 nM or less, about 0.5 nM or less, about 0.1 nM or less, or about 0.05 nM or less) for inducing ADCC of cells expressing CTLA4. Methods of measuring ADCC effects (e.g., in vitro methods) are known in the art, including, without limitation, via the methods described in Example 3 or for example, as described in U.S. Patent Application Publication No. US20210207126A1.

In some embodiments, the anti-CTLA4 antibodies induce antibody-dependent cellular phagocytosis (ADCP) effects against a CTLA4 expressing cell (e.g., against CTLA4-expressing human cells such as Tregs) after the anti-CTLA4 antibody binds to the cell-expressed CTLA4. In some embodiments, the anti-CTLA4 antibodies induce ADCP effects by more than about 10% (e.g., induce ADCP by more than about 10%, more than about 15%, more than about 20%, more than about 25%, more than about 30%, more than about 35%, more than about 40%, etc.) relative to a control (e.g., an isotype control or ipilimumab). In certain embodiments wherein the anti-CTLA4 antibodies comprise an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibodies induce ADCP effects by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an Fc region that does not comprise the substitutions. In certain embodiments wherein the anti-CTLA4 antibodies comprise an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibodies induce ADCP effects by at a rate of at least about 1.5 fold (e.g., at least about 1.5 fold, at least about 2 fold, at least about 2.5 fold, at least about 3 fold, at least about 4 fold, or at least about 5 fold) greater than an anti-CTLA4 antibody that comprises an Fc region that does not comprise the substitutions. In some embodiments, the anti-CTLA4 antibody has a half maximal effective concentration (EC50) of about 5 nM or less (e.g., about 5 nM or less, about 4 nM or less, about 3 nM or less, about 3 nM or less, about 2 nM or less, about 1 nM or less, about 0.5 nM or less, about 0.1 nM or less, or about 0.05 nM or less) for inducing ADCP of cells expressing CTLA4. Methods of measuring ADCP of antibodies and antigen binding fragments are also well known in the art. For example, to assess ADCP activity of a molecule of interest, an in vitro ADCP assay (see, e.g., Bracher et al., 2007, J. Immunol. Methods 323: 160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248: 167-82).

In some embodiments, the anti-CTLA4 antibodies or antigen-binding fragments are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the anti-CTLA4 antibodies or antigen-binding fragments relative to corresponding tumor cells not contacted with the anti-CTLA4 antibodies or antigen-binding fragments (or relative to corresponding tumor cells contacted with an isotype control antibody). In some embodiments, the anti-CTLA4 antibodies or antigen-binding fragments are capable of reducing tumor volume in a subject when the subject is administered the anti-CTLA4 antibodies or antigen-binding fragments. In some embodiments, the anti-CTLA4 antibodies or antigen-binding fragments are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the anti-CTLA4 antibodies or antigen-binding fragments; as compared to a corresponding tumor in a subject administered an isotype control antibody). Methods of monitoring tumor cell growth/proliferation, tumor volume, and/or tumor inhibition are known in the art, including, for example, via the methods described in Example 4 of for example, as described in U.S. Patent Application Publication No. US20210207126A1.

In some embodiments, the anti-CTLA4 antibodies or antigen-binding fragments have therapeutic effect on a cancer. In some embodiments, the anti-CTLA4 antibodies or antigen-binding fragments reduce one or more signs or symptoms of a cancer. In some embodiments, a subject suffering from a cancer goes into partial or complete remission when administered the anti-CTLA4 antibodies or antigen-binding fragments.

### Anti-CTLA4 Antibodies

In some aspects, the present disclosure provides an isolated antibody that specifically binds to an epitope similar to a ligand binding site of human CTLA4. In some embodiments, the antibody specifically binds to an epitope similar to CD80 binding site of human CTLA4. In some embodiments, the antibody specifically binds to an epitope similar to CD86 binding site of human CTLA4. In some embodiments, the antibody specifically binds to an epitope comprising one or more amino acid residues in a ligand binding site (e.g., CD80 and/or CD86 binding site) of human CTLA4. In some embodiments, the antibody specifically binds to an epitope on human CTLA4 that is different from the epitope of ipilimumab. In some embodiments, the epitope does not comprise amino acid residues in the CC′ loop motif of human CTLA4. In some embodiments, the epitope does not comprise amino acid residue L106 or 1108 of human CTLA4. In some embodiments, the antibody specifically binds to an epitope comprising amino acid residues Y105 and L106, but not 1108 of human CTLA4, wherein the numbering of the amino acid residues is according to SEQ ID NO: 134.

(SEQ ID NO: 134)
KAMHVAQPAVVLASSRGIASFVCEYASPGKATEVRVTVLRQADSQV
TEVCAATYMMGNELTFLDDSICTGTSSGNQVNLTIQGLRAMDTGLY
ICKVELMYPPPYYLGIGNGTQIYVIDPE

In some embodiments, an antibody of the present disclosure is a bispecific antibody that binds to a first and second target, where the first target is human CTLA4. In some embodiments, the bispecific antibody binds to a first and second target, where the first target is human CTLA4, and where the bispecific antibody comprises one, two, three, four, five, or all six of the CDRs shown for any of the exemplary antibodies described in Table 2. In some embodiments, the bispecific antibody binds to a first and second target, where the first target is human CTLA4, and where the bispecific antibody comprises a VH and/or VL shown for any of the exemplary antibodies described in Table 3. In some embodiments, the second target is PD-1, PD-L1, PD-L2, LAG3, TIM3, B7-H3, CD95, CD120a, OX40, CD40, BTLA, VISTA, ICOS, Her1, Her2, Her3, or B7-H4.

Anti-CTLA4 antibodies of the present disclosure may be produced by any techniques known in the art, including conventional monoclonal antibody methodology e.g., a standard somatic cell hybridization technique (see e.g., Kohler and Milstein, Nature 256: 495 (1975)), viral or oncogenic transformation of B lymphocytes, or recombinant antibody technologies as described in detail herein (see e.g., Examples 1 and 2). In some embodiments, anti-CTLA4 antibodies of the present disclosure are produced using any of the libraries and/or methods described in U.S. Patent Application Pub. No. US20200362019A1 (incorporated herein by reference in its entirety) and/or U.S. Patent Application Pub. No. US20200248336A1 (incorporated herein by reference in its entirety).

Hybridoma production is a very well-established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. One well-known method that may be used for making human CTLA4 antibodies provided by the present disclosure involves the use of a XenoMouse™ animal system. XenoMouse™ mice are engineered mouse strains that comprise large fragments of human immunoglobulin heavy chain and light chain loci and are deficient in mouse antibody production (see e.g., Green et al., (1994) Nature Genetics 7: 13-21; WO2003/040170). The animal is immunized with a CTLA4 antigen. The CTLA4 antigen is isolated and/or purified CTLA4. It may be a fragment of CTLA4, such as the extracellular domain of CTLA4. Immunization of animals can be carried out by any method known in the art (see e.g., Harlow and Lane, Antibodies: A Laboratory Manual, New York: Cold Spring Harbor Press, 1990). Methods for immunizing non-human animals such as mice, rats, sheep, goats, pigs, cattle and horses are well known in the art (see e.g., Harlow and Lane, supra, and U.S. Pat. No. 5,994,619). The CTLA4 antigen may be administered with an adjuvant to stimulate the immune response. Exemplary adjuvants include complete or incomplete Freund's adjuvant, RIBI (muramyl dipeptides) or ISCOM (immunostimulating complexes). After immunization of an animal with a CTLA4 antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene (see e.g., Harlow and Lane, supra). If fusion with myeloma cells is used, the myeloma cells preferably do not secrete immunoglobulin polypeptides (anon-secretory cell line). Immortalized cells are screened using CTLA4, a portion thereof, or a cell expressing CTLA4. CTLA4 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

Anti-CTLA4 antibodies of the present disclosure may also be prepared using phage display or yeast display methods. Such display methods for isolating human antibodies are established in the art (see e.g., Knappik, et al. (2000) J. Mol. Biol. 296, 57-86; Feldhaus et al. (2003) Nat Biotechnol 21: 163-170; see also the methods of Examples 1 and 2 below).

### Antigen Binding Fragments

In some other aspects, the present disclosure provides antigen-binding fragments of any of the CTLA4 antibodies described herein. In some embodiments, the antigen-binding fragment comprises a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

The antigen-binding fragment may comprise any sequences of any of the anti-CTLA4 antibodies described herein. In some embodiments, the antigen-binding fragment comprises the amino acid sequence of: (1) a light chain of a CTLA4 antibody; (2) a heavy chain of a CTLA4 antibody; (3) a variable region from the light chain of a CTLA4 antibody; (4) a variable region from the heavy chain of a CTLA4 antibody; (5) one or more CDRs (e.g., one, two, three, four, five, or six CDRs) of a CTLA4 antibody; or (6) three CDRs from the light chain and three CDRs from the heavy chain of a CTLA4 antibody.

In some embodiments, the present disclosure provides an antigen-binding fragment of an anti-CTLA4 antibody selected from those listed in Tables 2 and 3 and a human IgG1 Fc region comprising one or more substitutions selected from the group consisting of a L235V substitution, a S239D substitution, an F243L substitution, an R292P substitution, an S298A substitution, a Y300L substitution, a V305T substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering.

In some embodiments, the antigen-binding fragments of a CTLA4 antibody include: (i) a Fab fragment, which is a monovalent fragment consisting of the V_{L}, V_{H}, C_{L} and C_{H}1 domains; (ii) a F (ab′)₂ fragment, which is a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., (1989) Nature 341: 544-546), which consists of a V_{H} domain; (vi) an isolated CDR, and (vii) single chain antibody (scFv), which is a polypeptide comprising a V_{L} region of an antibody linked to a V_{H} region of an antibody (see e.g., Bird et al. (1988) Science 242: 423-426; Huston et al. (1988) Proc. Natl. Acad. Sci. USA 85: 5879-5883).

### Human IgG1 Fc Regions

In some embodiments, the anti-CTLA4 antibody comprises a human IgG1 Fc region. In some embodiments, the human IgG1 Fc region comprises one or more amino acid substitutions that confer enhanced effector function. In some embodiments, the human IgG1 Fc region comprises one or more amino acid substitutions that enhance ADCC. In some embodiments, the human IgG1 Fc region comprises one or more amino acid substitutions that decrease fucosylation. In some embodiments, the human IgG1 Fc region comprises one or more amino acid substitutions wherein the human IgG1 Fc region is afucosylated. In some embodiments, the human IgG1 Fc region is afucosylated. In some embodiments, the human IgG1 Fc region comprises one or more (e.g., 1, 2, 3, 4, 5, 6, or more) substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering.

In some embodiments, the human IgG1 Fc region comprises an S298A substitution, an E333A substitution, and a K334A substitution, wherein the residue numbering is according to EU numbering. In certain embodiments, the human IgG1 Fc region comprises an S298A substitution, an E333A substitution, and a K334A substitution, and does not comprise one or more (e.g., 1, 2, 3, 4, 5, 6, 7, 8, 9 or any) of a L235V substitution, a S239D substitution, an F243L substitution, an R292P substitution, a Y300L substitution, a V305I substitution, a K326A substitution, an A330L substitution, an I332E substitution, and a P396L substitution, wherein the residue numbering is according to EU numbering.

In some embodiments, the human IgG1 Fc region comprises an S298A substitution, an E333A substitution, a K334A substitution, and a K326A substitution, wherein the residue numbering is according to EU numbering. In some embodiments, the human IgG1 Fc region comprises an S298A substitution, an E333A substitution, a K334A substitution, and a K326A substitution, and does not comprise one or more (e.g., 1, 2, 3, 4, 5, 6, 7, 8, or any) of a L235V substitution, a S239D substitution, an F243L substitution, an R292P substitution, a Y300L substitution, a V305I substitution, an A330L substitution, an I332E substitution, and a P396L substitution, wherein the residue numbering is according to EU numbering.

In some embodiments, the human IgG1 Fc region comprises an F243L substitution, wherein the residue numbering is according to EU numbering. In some embodiments, the human IgG1 Fc region comprises an F243L substitution, and does not comprise one or more (e.g., 1, 2, 3, 4, 5, 6, 7, 8, 9, 10, 11, or any) of a L235V substitution, a S239D substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering.

In some embodiments, the human IgG1 Fc region comprises an F243L substitution, an R292P substitution, a Y300L substitution, and a P396L substitution, wherein the residue numbering is according to EU numbering. In some embodiments, the human IgG1 Fc region comprises an F243L substitution, an R292P substitution, a Y300L substitution, and a P396L substitution, and does not comprise one or more (e.g., 1, 2, 3, 4, 5, 6, 7, 8, or any) of a L235V substitution, a S239D substitution, an S298A substitution, a V305I substitution, a K326A substitution, an A330L substitution, an I332E substitution, an E333A substitution, and a K334A substitution, wherein the residue numbering is according to EU numbering.

In some embodiments, the human IgG1 Fc region comprises an F243L substitution, an R292P substitution, a Y300L substitution, a V305I substitution, and a P396L substitution, wherein the residue numbering is according to EU numbering. In some embodiments, the human IgG1 Fc region comprises an F243L substitution, an R292P substitution, a Y300L substitution, a V305I substitution, and a P396L substitution, and does not comprise one or more (e.g., 1, 2, 3, 4, 5, 6, 7, or any) of a L235V substitution, a S239D substitution, an S298A substitution, a K326A substitution, an A330L substitution, an I332E substitution, an E333A substitution, and a K334A substitution, wherein the residue numbering is according to EU numbering.

In some embodiments, the human IgG1 Fc region comprises an F243L substitution, an R292P substitution, a Y300L substitution, a L235V substitution, and a P396L substitution, wherein the residue numbering is according to EU numbering. In some embodiments, the human IgG1 Fc region comprises an F243L substitution, an R292P substitution, a Y300L substitution, a L235V substitution, and a P396L substitution, and does not comprise does not comprise one or more (e.g., 1, 2, 3, 4, 5, 6, 7, or any) of a S239D substitution, an S298A substitution, a V305I substitution, a K326A substitution, an A330L substitution, an I332E substitution, an E333A substitution, and a K334A substitution, wherein the residue numbering is according to EU numbering.

In some embodiments, the human IgG1 Fc region comprises a S239D substitution, an I332E substitution, and an A330L substitution, wherein the residue numbering is according to EU numbering. In some embodiments, the human IgG1 Fc region comprises a S239D substitution, an I332E substitution, and an A330L substitution, and does not comprise does not comprise one or more (e.g., 1, 2, 3, 4, 5, 6, 7, 8, 9, or any) of a L235V substitution, an F243L substitution, an R292P substitution, an S298A substitution, a Y300L substitution, a V305I substitution, a K326A substitution, an E333A substitution, a K334A substitution, and a P396L substitution, wherein the residue numbering is according to EU numbering.

In some embodiments, the human IgG1 Fc region comprises a S239D substitution and an I332E substitution, wherein the residue numbering is according to EU numbering. In some embodiments, the human IgG1 Fc region comprises a S239D substitution and an I332E substitution, and does not comprise one or more (e.g., 1, 2, 3, 4, 5, 6, 7, 8, 9, 10, or any) of a L235V substitution, an F243L substitution, an R292P substitution, an S298A substitution, a Y300L substitution, a V305I substitution, a K326A substitution, an A330L substitution, an E333A substitution, a K334A substitution, and a P396L substitution, wherein the residue numbering is according to EU numbering.

In some embodiments in which the anti-CTLA4 antibody comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibody induces antibody-dependent cell cytotoxicity (ADCC) against a CTLA4-expressing human cell or a human Treg cell. In some embodiments, the ADCC activity of the anti-CTLA4 antibody is higher than that of a control antibody comprising a human IgG1 Fc region that does not comprise the substitutions, and/or the ADCC activity of the anti-CTLA4 antibody is higher than the ADCC activity of ipilimumab. In some embodiments, the ADCC activity of the anti-CTLA4 antibody is higher than that of a control antibody comprising a human IgG1 Fc region that does not comprise the substitutions, and/or the ADCC activity of the anti-CTLA4 antibody is higher than the ADCC activity of an afucosylated variant of ipilimumab. In certain embodiments wherein the anti-CTLA4 antibody comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the ADCC activity of the anti-CTLA4 antibody exceeds the ADCC activity of the control antibody by at least about 5% (e.g., at least about 5%, at least about 10%, at least about 20%, at least about 30%, at least about 40%, at least about 50%, at least about 60%, at least about 70%, at least about 80%, or at least about 90%) greater than an anti-CTLA4 antibody that comprises an Fc region that does not comprise the substitutions (e.g., an isotype control or ipilimumab). In certain embodiments wherein the anti-CTLA4 antibody comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibody induces ADCP effects by more than about 10% (e.g., induce ADCP by more than about 10%, more than about 15%, more than about 20%, more than about 25%, more than about 30%, more than about 35%, more than about 40%, etc.) relative to a control (e.g., an isotype control or ipilimumab). In certain embodiments wherein the anti-CTLA4 antibody comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibody induces ADCC effects by at a rate of at least about 1.5 fold (e.g., at least about 1.5 fold, at least about 2 fold, at least about 2.5 fold, at least about 3 fold, at least about 4 fold, or at least about 5 fold) greater than an anti-CTLA4 antibody that comprises an Fc region that does not comprise the substitutions. In some embodiments, the anti-CTLA4 antibody has a half maximal effective concentration (EC50) of about 5 nM or less (e.g., about 5 nM or less, about 4 nM or less, about 3 nM or less, about 3 nM or less, about 2 nM or less, about 1 nM or less, about 0.5 nM or less, about 0.1 nM or less, or about 0.05 nM or less) for inducing ADCC of cells expressing CTLA4. Methods of measuring ADCP of antibodies and antigen binding fragments are also well known in the art. For example, to assess ADCP activity of a molecule of interest, an in vitro ADCP assay (see, e.g., Bracher et al., 2007, J. Immunol. Methods 323: 160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248: 167-82).

In some embodiments, the anti-CTLA4 antibody induces antibody-dependent cellular phagocytosis (ADCP) effects against a CTLA4 expressing cell (e.g., against CTLA4-expressing human cells such as Tregs) after the anti-CTLA4 antibody binds to the cell-expressed CTLA4. In some embodiments, the anti-CTLA4 antibody induces ADCP effects by more than about 10% (e.g., induce ADCP by more than about 10%, more than about 15%, more than about 20%, more than about 25%, more than about 30%, more than about 35%, more than about 40%, etc.) relative to a control (e.g., an isotype control or ipilimumab). In certain embodiments wherein the anti-CTLA4 antibody comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibody induces ADCP effects by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an Fc region that does not comprise the substitutions. In certain embodiments wherein the anti-CTLA4 antibody comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibody induces ADCP effects by at a rate of at least about 1.5 fold (e.g., at least about 1.5 fold, at least about 2 fold, at least about 2.5 fold, at least about 3 fold, at least about 4 fold, or at least about 5 fold) greater than an anti-CTLA4 antibody that comprises an Fc region that does not comprise the substitutions. In some embodiments, the anti-CTLA4 antibody has a half maximal effective concentration (EC50) of about 5 nM or less (e.g., about 5 nM or less, about 4 nM or less, about 3 nM or less, about 3 nM or less, about 2 nM or less, about 1 nM or less, about 0.5 nM or less, about 0.1 nM or less, or about 0.05 nM or less) for inducing ADCP of cells expressing CTLA4. Methods of measuring ADCP of antibodies and antigen binding fragments are also well known in the art. For example, to assess ADCP activity of a molecule of interest, an in vitro ADCP assay (see, e.g., Bracher et al., 2007, J. Immunol. Methods 323: 160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248: 167-82).

Anti-CTLA4 Antibodies with Human IgG1 Fc Regions

In some aspects, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering. In some embodiments, the antibody comprises two heavy chains and two light chains, wherein each human IgG1 Fc domain of the heavy chains comprises: i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution, wherein the residue numbering is according to EU numbering.

In some embodiments, the isolated antibody that binds to human CTLA4 comprises a heavy chain comprising a heavy chain constant region comprising a human IgG1 Fc domain, wherein the human IgG1 Fc domain comprises the substitutions of antibody TY24034 as shown in Table 14. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, a CDR-H3 comprising the amino acid sequence of SEQ ID NO: 3, or a variant thereof comprising up to 5 (e.g., 1, 2, 3, 4, or 5) amino acid substitutions; and the VL comprises a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions; wherein the antibody comprises two heavy chains and two light chains, wherein each human IgG1 Fc domain of the heavy chains comprises an S298A substitution, an E333A substitution, and a K334A substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74; and the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75; wherein the antibody comprises two heavy chains and two light chains, wherein each human IgG1 Fc domain of the heavy chains comprises an S298A substitution, an E333A substitution, and a K334A substitution, wherein the residue numbering is according to EU numbering. In certain embodiments, which may be combined with the preceding embodiments, each human IgG1 Fc domain of the heavy chain does not comprise a K326A substitution. In certain embodiments, which may be combined with the preceding embodiments, the isolated antibody induces antibody-dependent cell cytotoxicity (ADCC) against a CTLA4-expressing human cell or a human Treg cell, wherein the ADCC activity of the isolated antibody is higher (e.g., at least about 10%, at least about 20%, at least about 30%, at least about 40%, at least about 50%, at least about 60%, at least about 70%, at least about 80%, or at least about 90% higher) than that of a control antibody comprising a human IgG1 Fc region that does not comprise the S298A substitution, the E333A substitution, and/or the K334A substitution, and/or wherein the ADCC activity of the isolated antibody is higher than the ADCC activity of ipilimumab. In certain embodiments, which may be combined with the preceding embodiments, the anti-CTLA4 antibody induces antibody-dependent cellular phagocytosis (ADCP) effects against a CTLA4 expressing cell (e.g., against CTLA4-expressing human cells such as Tregs) by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an IgG1 Fc region that does not comprise the S298A substitution, the E333A substitution, and/or the K334A substitution, and/or wherein the ADCC activity of the isolated antibody is higher than the ADCC activity of ipilimumab.

In some embodiments, the isolated antibody that binds to human CTLA4 comprises a heavy chain comprising a heavy chain constant region comprising a human IgG1 Fc domain, wherein the human IgG1 Fc domain comprises the substitutions of antibody TY24035 as shown in Table 14. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, a CDR-H3 comprising the amino acid sequence of SEQ ID NO: 3, or a variant thereof comprising up to 5 (e.g., 1, 2, 3, 4, or 5) amino acid substitutions; and the VL comprises a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions; wherein the antibody comprises two heavy chains and two light chains, wherein each human IgG1 Fc domain of the heavy chains comprises an S298A substitution, an E333A substitution, a K334A substitution, and a K326A substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74; and the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75; wherein the antibody comprises two heavy chains and two light chains, wherein each human IgG1 Fc domain of the heavy chains comprises an S298A substitution, an E333A substitution, a K334A substitution, and a K326A substitution, wherein the residue numbering is according to EU numbering. In certain embodiments, which may be combined with the preceding embodiments, the isolated antibody induces antibody-dependent cell cytotoxicity (ADCC) against a CTLA4-expressing human cell or a human Treg cell, wherein the ADCC activity of the isolated antibody is higher (e.g., at least about 10%, at least about 20%, at least about 30%, at least about 40%, at least about 50%, at least about 60%, at least about 70%, at least about 80%, or at least about 90% higher) than that of a control antibody comprising a human IgG1 Fc region that does not comprise the S298A substitution, the E333A substitution, the K334A substitution, and/or the K326A substitution, and/or wherein the ADCC activity of the isolated antibody is higher than the ADCC activity of ipilimumab. In certain embodiments, which may be combined with the preceding embodiments, the anti-CTLA4 antibody induces antibody-dependent cellular phagocytosis (ADCP) effects against a CTLA4 expressing cell (e.g., against CTLA4-expressing human cells such as Tregs) by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an IgG1 Fc region that does not comprise the S298A substitution, the E333A substitution, the K334A substitution, and/or the K326A substitution, and/or wherein the ADCC activity of the isolated antibody is higher than the ADCC activity of ipilimumab.

In some embodiments, the isolated antibody that binds to human CTLA4 comprises a heavy chain comprising a heavy chain constant region comprising a human IgG1 Fc domain, wherein the human IgG1 Fc domain comprises the substitutions of antibody TY24036 as shown in Table 14. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, a CDR-H3 comprising the amino acid sequence of SEQ ID NO: 3, or a variant thereof comprising up to 5 (e.g., 1, 2, 3, 4, or 5) amino acid substitutions; and the VL comprises a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions; wherein the antibody comprises two heavy chains and two light chains, wherein each human IgG1 Fc domain of the heavy chains comprises an F243L substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74; and the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75; wherein the antibody comprises two heavy chains and two light chains, wherein each human IgG1 Fc domain of the heavy chains comprises an F243L substitution, wherein the residue numbering is according to EU numbering. In certain embodiments, which may be combined with the preceding embodiments, each human IgG1 Fc domain comprises an F243L substitution and does not comprise an F243L substitution, an R292P substitution, an Y300L substitution, a V305I substitution, a P396L substitution, and/or an L235V substitution. In certain embodiments, which may be combined with the preceding embodiments, the isolated antibody induces antibody-dependent cell cytotoxicity (ADCC) against a CTLA4-expressing human cell or a human Treg cell, wherein the ADCC activity of the isolated antibody is higher (e.g., at least about 10%, at least about 20%, at least about 30%, at least about 40%, at least about 50%, at least about 60%, at least about 70%, at least about 80%, or at least about 90% higher) than that of a control antibody comprising a human IgG1 Fc region that does not comprise the F243L substitution, and/or wherein the ADCC activity of the isolated antibody is higher than the ADCC activity of ipilimumab. In certain embodiments, which may be combined with the preceding embodiments, the anti-CTLA4 antibody induces antibody-dependent cellular phagocytosis (ADCP) effects against a CTLA4 expressing cell (e.g., against CTLA4-expressing human cells such as Tregs) by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an IgG1 Fc region that does not comprise the F243L substitution, and/or wherein the ADCC activity of the isolated antibody is higher than the ADCC activity of ipilimumab.

In some embodiments, the isolated antibody that binds to human CTLA4 comprises a heavy chain comprising a heavy chain constant region comprising a human IgG1 Fc domain, wherein the human IgG1 Fc domain comprises the substitutions of antibody TY24037 as shown in Table 14. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, a CDR-H3 comprising the amino acid sequence of SEQ ID NO: 3, or a variant thereof comprising up to 5 (e.g., 1, 2, 3, 4, or 5) amino acid substitutions; and the VL comprises a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions; wherein the antibody comprises two heavy chains and two light chains, wherein each human IgG1 Fc domain of the heavy chains comprises an F243L substitution, an R292P substitution, an Y300L substitution, an V305I substitution, and an P396L substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74; and the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75; wherein the antibody comprises two heavy chains and two light chains, wherein each human IgG1 Fc domain of the heavy chains comprises an F243L substitution, an R292P substitution, an Y300L substitution, an V305I substitution, and an P396L substitution, wherein the residue numbering is according to EU numbering. In certain embodiments, which may be combined with the preceding embodiments, each human IgG1 Fc domain does not comprise an L235V substitution. In certain embodiments, which may be combined with the preceding embodiments, the isolated antibody induces antibody-dependent cell cytotoxicity (ADCC) against a CTLA4-expressing human cell or a human Treg cell, wherein the ADCC activity of the isolated antibody is higher (e.g., at least about 10%, at least about 20%, at least about 30%, at least about 40%, at least about 50%, at least about 60%, at least about 70%, at least about 80%, or at least about 90% higher) than that of a control antibody comprising a human IgG1 Fc region that does not comprise the F243L substitution, the R292P substitution, the Y300L substitution, the V305I substitution, and/or the P396L substitution, and/or wherein the ADCC activity of the isolated antibody is higher than the ADCC activity of ipilimumab. In certain embodiments, which may be combined with the preceding embodiments, the anti-CTLA4 antibody induces antibody-dependent cellular phagocytosis (ADCP) effects against a CTLA4 expressing cell (e.g., against CTLA4-expressing human cells such as Tregs) by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an IgG1 Fc region that does not comprise the F243L substitution, the R292P substitution, the Y300L substitution, the V305I substitution, and/or the P396L substitution, and/or wherein the ADCC activity of the isolated antibody is higher than the ADCC activity of ipilimumab.

In some embodiments, the isolated antibody that binds to human CTLA4 comprises a heavy chain comprising a heavy chain constant region comprising a human IgG1 Fc domain, wherein the human IgG1 Fc domain comprises the substitutions of antibody TY24038 as shown in Table 14. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, a CDR-H3 comprising the amino acid sequence of SEQ ID NO: 3, or a variant thereof comprising up to 5 (e.g., 1, 2, 3, 4, or 5) amino acid substitutions; and the VL comprises a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions; wherein the antibody comprises two heavy chains and two light chains, wherein each human IgG1 Fc domain of the heavy chains comprises an F243L substitution, an R292P substitution, an Y300L substitution, an L235V substitution, and an P396L substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74; and the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75; wherein the antibody comprises two heavy chains and two light chains, wherein each human IgG1 Fc domain of the heavy chains comprises an F243L substitution, an R292P substitution, an Y300L substitution, an L235V substitution, and an P396L substitution, wherein the residue numbering is according to EU numbering. In certain embodiments, which may be combined with the preceding embodiments, each human IgG1 Fc domain does not comprise a V305I substitution. In certain embodiments, which may be combined with the preceding embodiments, the isolated antibody induces antibody-dependent cell cytotoxicity (ADCC) against a CTLA4-expressing human cell or a human Treg cell, wherein the ADCC activity of the isolated antibody is higher (e.g., at least about 10%, at least about 20%, at least about 30%, at least about 40%, at least about 50%, at least about 60%, at least about 70%, at least about 80%, or at least about 90% higher) than that of a control antibody comprising a human IgG1 Fc region that does not comprise the F243L substitution, the R292P substitution, the Y300L substitution, the L235V substitution, and/or the P396L substitution, and/or wherein the ADCC activity of the isolated antibody is higher than the ADCC activity of ipilimumab. In certain embodiments, which may be combined with the preceding embodiments, the anti-CTLA4 antibody induces antibody-dependent cellular phagocytosis (ADCP) effects against a CTLA4 expressing cell (e.g., against CTLA4-expressing human cells such as Tregs) by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an IgG1 Fc region that does not comprise the F243L substitution, the R292P substitution, the Y300L substitution, the L235V substitution, and/or the P396L substitution, and/or wherein the ADCC activity of the isolated antibody is higher than the ADCC activity of ipilimumab.

In some embodiments, the isolated antibody that binds to human CTLA4 comprises a heavy chain comprising a heavy chain constant region comprising a human IgG1 Fc domain, wherein the human IgG1 Fc domain comprises the substitutions of antibody TY24039 as shown in Table 14. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, a CDR-H3 comprising the amino acid sequence of SEQ ID NO: 3, or a variant thereof comprising up to 5 (e.g., 1, 2, 3, 4, or 5) amino acid substitutions; and the VL comprises a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions; wherein the antibody comprises two heavy chains and two light chains, wherein each human IgG1 Fc domain of the heavy chains comprises an S239D substitution, an I332E substitution, and an A330L substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74; and the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75; wherein the antibody comprises two heavy chains and two light chains, wherein each human IgG1 Fc domain of the heavy chains comprises an S239D substitution, an I332E substitution, and an A330L substitution, wherein the residue numbering is according to EU numbering. In certain embodiments, which may be combined with the preceding embodiments, the isolated antibody induces antibody-dependent cell cytotoxicity (ADCC) against a CTLA4-expressing human cell or a human Treg cell, wherein the ADCC activity of the isolated antibody is higher (e.g., at least about 10%, at least about 20%, at least about 30%, at least about 40%, at least about 50%, at least about 60%, at least about 70%, at least about 80%, or at least about 90% higher) than that of a control antibody comprising a human IgG1 Fc region that does not comprise the S239D substitution, the I332E substitution, and/or the A330L substitution, and/or wherein the ADCC activity of the isolated antibody is higher than the ADCC activity of ipilimumab. In certain embodiments, which may be combined with the preceding embodiments, the anti-CTLA4 antibody induces antibody-dependent cellular phagocytosis (ADCP) effects against a CTLA4 expressing cell (e.g., against CTLA4-expressing human cells such as Tregs) by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an IgG1 Fc region that does not comprise the S239D substitution, the I332E substitution, and/or the A330L substitution, and/or wherein the ADCC activity of the isolated antibody is higher than the ADCC activity of ipilimumab.

In some embodiments, the isolated antibody that binds to human CTLA4 comprises a heavy chain comprising a heavy chain constant region comprising a human IgG1 Fc domain, wherein the human IgG1 Fc domain comprises the substitutions of antibody TY24040 as shown in Table 14. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, a CDR-H3 comprising the amino acid sequence of SEQ ID NO: 3, or a variant thereof comprising up to 5 (e.g., 1, 2, 3, 4, or 5) amino acid substitutions; and the VL comprises a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions; wherein the antibody comprises two heavy chains and two light chains, wherein each human IgG1 Fc domain of the heavy chains comprises an S239D substitution and an I332E substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74; and the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75; wherein the antibody comprises two heavy chains and two light chains, wherein each human IgG1 Fc domain of the heavy chains comprises an S239D substitution and an I332E substitution, wherein the residue numbering is according to EU numbering. In some embodiments, provided herein is an isolated antibody that binds to human CTLA4, wherein the antibody comprises a heavy chain comprising a heavy chain variable region (VH) and a light chain comprising a light chain variable region (VL), wherein the heavy chain comprises the amino acid sequence of SEQ ID NO: 91, or an amino acid sequence having at least 80% (e.g., at least 85%, 90%, 95%, 98%, or 99%; or 100%) sequence identity with SEQ ID NO: 91; and the light chain comprises the amino acid sequence of SEQ ID NO: 90, or an amino acid sequence having at least 80% (e.g., at least 85%, 90%, 95%, 98%, or 99%; or 100%) sequence identity with SEQ ID NO: 91; wherein the antibody comprises two heavy chains and two light chains, wherein each human IgG1 Fc domain of the heavy chains comprises an S239D substitution and an I332E substitution, wherein the residue numbering is according to EU numbering. In certain embodiments, which may be combined with the preceding embodiments, each human IgG1 Fc domain does not comprise an A330L substitution. In certain embodiments, which may be combined with the preceding embodiments, the isolated antibody induces antibody-dependent cell cytotoxicity (ADCC) against a CTLA4-expressing human cell or a human Treg cell, wherein the ADCC activity of the isolated antibody is higher (e.g., at least about 10%, at least about 20%, at least about 30%, at least about 40%, at least about 50%, at least about 60%, at least about 70%, at least about 80%, or at least about 90% higher) than that of a control antibody comprising a human IgG1 Fc region that does not comprise the S239D substitution and/or the I332E substitution, and/or wherein the ADCC activity of the isolated antibody is higher than the ADCC activity of ipilimumab. In certain embodiments, which may be combined with the preceding embodiments, the anti-CTLA4 antibody induces antibody-dependent cellular phagocytosis (ADCP) effects against a CTLA4 expressing cell (e.g., against CTLA4-expressing human cells such as Tregs) by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an IgG1 Fc region that does not comprise the S239D substitution and/or the I332E substitution, and/or wherein the ADCC activity of the isolated antibody is higher than the ADCC activity of ipilimumab.

### Antibody Derivatives

In some further aspects, the present disclosure provides derivatives of any of the CTLA4 antibodies described herein.

In some embodiments, the anti-CTLA4 antibody derivative is derived from modifications of the amino acid sequences of an illustrative anti-CTLA4 antibody (e.g., a “parental antibody”) of the present disclosure while conserving the overall molecular structure of the parental antibody amino acid sequence. Amino acid sequences of any regions of the parental antibody chains may be modified, such as framework regions, CDR regions, or constant regions. Types of modifications include substitutions, insertions, deletions, or combinations thereof, of one or more amino acids of the parental antibody.

In some embodiments, the anti-CTLA4 antibody derivative comprises a VL or VH region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 64-89. In some embodiments, the anti-CTLA4 antibody derivative comprises an CDR-H1 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 1, 7, 12, 18, 23, 29, 34, 38, 44, 49, and 60. In some embodiments, the anti-CTLA4 antibody derivative comprises an CDR-H2 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 2, 8, 13, 19, 24, 30, 39, 45, 56, and 61. In some embodiments, the anti-CTLA4 antibody derivative comprises an CDR-H3 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 3, 9, 14, 20, 25, 31, 35, 40, 46, 50, 53, 57, and 62. In some embodiments, the anti-CTLA4 antibody derivative comprises an CDR-L1 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 4, 10, 15, 21, 26, 32, 36, 41, 47, 51, 54, 58, and 63. In some embodiments, the anti-CTLA4 antibody derivative comprises an CDR-L2 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 5, 16, 27, and 42. In some embodiments, the anti-CTLA4 antibody derivative comprises an CDR-L3 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 6, 11, 17, 22, 28, 33, 37, 43, 52, 55, and 59.

In some particular embodiments, the derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of SEQ ID NOS: 1-63.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of the anti-CTLA4 antibody, including the CDRs, framework regions, or constant regions. In one embodiment, the present disclosure provides an anti-CTLA4 antibody derivative that contains the V_{H} and V_{L} CDR sequences of an illustrative anti-CTLA4 antibody of this disclosure, yet contains framework sequences different from those of the illustrative anti-CTLA4 antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kaba et al., Sequences of Proteins of Immunological Interest, Fifth Edition, U.S. Department of Health and Human Services, NIH Publication No. 91-3242 (1991); Tomlinson et al., J. Mol. Biol. 227: 776-798 (1992); and Cox et al., Eur. J. Immunol. 24: 827-836 (1994)). Framework sequences that may be used in constructing an antibody derivative include those that are structurally similar to the framework sequences used by illustrative antibodies of the disclosure For example, the CDR-H1, CDR-H2, and CDR-H3 sequences, and the CDR-L1, CDR-L2, and CDR-L3 sequences of an illustrative antibody can be grafted onto framework regions that have the identical sequence as that found in the germline immunoglobulin gene from which the framework sequence derive, or the CDR sequences can be grafted onto framework regions that contain one or more mutations as compared to the germline sequences.

In some embodiments, the anti-CTLA4 antibody derivative is a chimeric anti-CTLA4 antibody which comprises an amino acid sequence of an illustrative antibody of the disclosure. In one example, one or more CDRs from one or more illustrative anti-CTLA4 antibodies are combined with CDRs from an anti-CTLA4 antibody from a non-human animal, such as mouse or rat. In another example, all of the CDRs of the chimeric anti-CTLA4 antibody are derived from one or more illustrative anti-CTLA4 antibodies. In some particular embodiments, the chimeric anti-CTLA4 antibody comprises one, two, or three CDRs from the heavy chain variable region and/or one, two, or three CDRs from the light chain variable region of an illustrative anti-CTLA4 antibody. Chimeric antibodies can be generated using conventional methods known in the art.

Another type of modification is to mutate amino acid residues within the CDR regions of the V_{H} and/or V_{L} chain. Site-directed mutagenesis or PCR-mediated mutagenesis can be performed to introduce the mutation (s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within a CDR region are altered. In some embodiments, the anti-CTLA4 antibody derivative comprises 1, 2, 3, or 4 amino acid substitutions in the heavy chain CDRs and/or in the light chain CDRs. In another embodiment, the amino acid substitution is to change one or more cysteines in an anti-CTLA4 antibody to another residue, such as, without limitation, alanine or serine. The cysteine may be a canonical or non-canonical cysteine. In one embodiment, the anti-CTLA4 antibody derivative has 1, 2, 3, or 4 conservative amino acid substitutions in the heavy chain HVR regions relative to the amino acid sequences of an illustrative anti-CTLA4 antibody.

Modifications may also be made to the framework residues within the V_{H} and/or V_{L} regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative anti-CTLA4 antibody, typically to alter one or more functional properties of the anti-CTLA4 antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

Furthermore, an anti-CTLA4 antibody of the present disclosure may be modified to alter its potential glycosylation site or pattern in accordance with routine experimentation known in the art. In another aspect, the present disclosure provides a derivative of a CTLA4 antibody that contains at least one mutation in a variable region of a light chain or heavy chain that changes the pattern of glycosylation in the variable region. Such an anti-CTLA4 antibody derivative may have an increased affinity and/or a modified specificity for binding an antigen. The mutations may add a novel glycosylation site in the V region, change the location of one or more V region glycosylation site (s), or remove a pre-existing V region glycosylation site. In one embodiment, the present disclosure provides a derivative of a CTLA4 antibody having a potential N-linked glycosylation site at asparagine in the heavy chain variable region, wherein the potential N-linked glycosylation site in one heavy chain variable region is removed. In another embodiment, the present disclosure provides a derivative of a CTLA4 antibody having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the disclosure of which incorporated herein by reference.

In another aspect, the present disclosure provides an antibody derivative that comprises a CTLA4 antibody, or antigen-binding fragment thereof, as described herein, linked to an additional molecular entity. Examples of additional molecular entities include pharmaceutical agents, peptides or proteins, detection agent or labels, and antibodies.

In some embodiments, the antibody derivative comprises an antibody of the disclosure linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Methods for linking an antibody to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies see e.g., Saito et al., Adv. Drug Deliv. Rev. 55: 199-215 (2003); Trail, et al., Cancer Immunol. Immunother. 52: 328-337 (2003); Payne, Cancer Cell 3: 207-212 (2003); Allen, Nat. Rev. Cancer 2: 750-763 (2002); Pastan and Kreitman, Curr. Opin. Investig. Drugs 3: 1089-1091 (2002); Senter and Springer (2001) Adv. Drug Deliv. Rev. 53: 247-264.

In some embodiments, the antibody derivative is a CTLA4 antibody multimer, which is a multimeric form of a CTLA4 antibody, such as antibody dimers, trimers, or higher-order oligomers of monomeric antibodies. Individual monomers within an antibody multimer may be identical or different. In addition, individual antibodies within a multimer may have the same or different binding specificities. Multimerization of antibodies may be accomplished through natural aggregation of antibodies. For example, some percentage of purified antibody preparations (e.g., purified IgG4 molecules) spontaneously form protein aggregates containing antibody homodimers, and other higher-order antibody multimers. Alternatively, antibody homodimers may be formed through chemical linkage techniques known in the art, such as through using crosslinking agents. Suitable crosslinkers include those that are heterobifunctional, having two distinctly reactive groups separated by an appropriate spacer (such as m-maleimidobenzoyl-N-hydroxysuccinimide ester, succinimidyl 4-(maleimidomethyl) cyclohexane-1-carboxylate, and N-succinimidyl S-acethylthio-acetate) or homobifunctional (such as disuccinimidyl suberate). Such linkers are commercially available from, for example, Pierce Chemical Company, Rockford, IL. Antibodies can also be made to multimerize through recombinant DNA techniques known in the art.

Examples of other anti-CTLA4 antibody derivatives provided by the present disclosure include single chain antibodies, diabodies, domain antibodies, nanobodies, and unibodies. A “single-chain antibody” (scFv) consists of a single polypeptide chain comprising a V_{L} domain linked to a V_{H} domain wherein V_{L} domain and V_{H} domain are paired to form a monovalent molecule. Single chain antibody can be prepared according to method known in the art (see e.g., Bird et al., (1988) Science 242: 423-426 and Huston et al., (1988) Proc. Natl. Acad. Sci. USA 85: 5879-5883). A “diabody” consists of two chains, each chain comprising a heavy chain variable region connected to a light chain variable region on the same polypeptide chain connected by a short peptide linker, wherein the two regions on the same chain do not pair with each other but with complementary domains on the other chain to form a bispecific molecule. Methods of preparing diabodies are known in the art (see e.g., Holliger P. et al., (1993) Proc. Natl. Acad. Sci. USA 90: 6444-6448, and Poljak R. J. et al., (1994) Structure 2: 1121-1123). Domain antibodies (dAbs) are small functional binding units of antibodies, corresponding to the variable regions of either the heavy or light chains of antibodies. Domain antibodies are well expressed in bacterial, yeast, and mammalian cell systems. Further details of domain antibodies and methods of production thereof are known in the art (see e.g., U.S. Pat. Nos. 6,291,158; 6,582,915; 6,593,081; 6,172,197; 6,696,245; European Patents 0368684 &0616640; WO05/035572, WO04/101790, WO04/081026, WO04/058821, WO04/003019 and WO03/002609). Nanobodies are derived from the heavy chains of an antibody. A nanobody typically comprises a single variable domain and two constant domains (CH2 and CH3) and retains antigen-binding capacity of the original antibody. Nanobodies can be prepared by methods known in the art (see e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### IV. Masked Binding Polypeptides Targeting CTLA4

The present disclosure also relates, in part, to masked binding polypeptides (i.e., masked antibodies) that bind to human CTLA4, including masked antibodies (e.g., activatable antibodies) comprising any of the anti-CTLA4 antibodies described herein (e.g., anti-CTLA4 antibodies, anti-CTLA4 antibody binding fragments, and/or anti-CTLA4 antibody derivatives), antigen binding fragments of the masked anti-CTLA4 antibodies (e.g., activatable anti-CTLA4 antibodies), and/or derivatives of the masked anti-CTLA4 antibodies (e.g., activatable anti-CTLA4 antibodies). In some embodiments, the masked anti-CTLA4 antibodies (e.g., activatable anti-CTLA4 antibodies) described herein may have improved safety profiles. For example, the masked anti-CTLA4 antibodies (e.g., activatable anti-CTLA4 antibodies) described herein may have better safety margin as assessed by spleen weight change. The change in spleen size with the increase in drug dose administered is used as a benchmark to assess the safety margin of the drug candidate used.

In some embodiments, an anti-CTLA4 masked antibody (e.g., an anti-CTLA4 activatable antibody) of the present disclosure comprises: (a) a masking peptide comprising, from N-terminus to C-terminus, a masking unit (MU) and a linkage unit (LU), and (b) a target binding moiety (TBM) comprising an antibody heavy chain variable region (VH) and an antibody light chain variable region (VL); wherein the masking peptide is linked to the N-terminus of the VH or the VL; wherein the TBM binds to human CTLA4; and wherein the MU competes with human CTLA4 to bind the TBM. In some embodiments, the LU comprises at least a first cleavage site. In some embodiments, the LU does not comprise a cleavage site.

In some embodiments, an anti-CTLA4 masked antibody (e.g., an anti-CTLA4 activatable antibody) of the present disclosure comprises: (a) a masking unit (MU); (b) a linkage unit (LU); and (c) a target binding moiety (TBM). In some embodiments, the MU is any of the masking units described herein. In some embodiments, the LU is any of the linkage units described herein. In some embodiments, the TBM is any of the target binding moieties described herein (e.g., a target binding moiety (TBM) comprising an antibody light chain variable region and/or an antibody heavy chain variable region, such as a VH and/or VL of any of the anti-CTLA4 antibodies described herein). In some embodiments, the MU interferes with and/or inhibits the binding of the anti-CTLA4 masked antibody (e.g., activatable antibody) to its target (e.g., human CTLA4). In some embodiments, the anti-CTLA4 masked antibody (e.g., activatable antibody) is capable of binding to its target (e.g., human CTLA4) at sites with a high local concentration of CTLA4 (e.g., cells or tissues that express CTLA4 at a high level). In some embodiments, the anti-CTLA4 masked antibody (e.g., activatable antibody) binds to its target (e.g., human CTLA4) at reduced binding affinity as compared to an antibody having the same TBM but lacking the MU. In some embodiments, the LU comprises at least a first cleavage site. In some embodiments, the LU does not comprise a cleavage site.

In some embodiments, the anti-CTLA4 masked antibody (e.g., activatable antibody) comprises: (a) a polypeptide comprising, from N-terminus to C-terminus, a masking unit (MU), a linkage unit (LU), and a target binding moiety (TBM), where the MU is any of the masking units described herein, the LU is any of the linkage units described herein, and where the TBM comprises an antibody light chain variable region (VL); and (b) an antibody heavy chain variable region (VH). In some embodiments, the LU comprises at least a first cleavage site. In some embodiments, the LU does not comprise a cleavage site.

In some embodiments, the anti-CTLA4 masked antibody (e.g., activatable antibody) comprises: (a) a polypeptide comprising, from N-terminus to C-terminus, a masking unit (MU), a linkage unit (LU), and a target binding moiety (TBM), where the MU is any of the masking units described herein, the LU is any of the linkage units described herein, and where the TBM comprises an antibody heavy chain variable region (VH); and (b) an antibody light chain variable region (VL). In some embodiments, the LU comprises at least a first cleavage site. In some embodiments, the LU does not comprise a cleavage site.

In some embodiments, the anti-CTLA4 masked antibody (e.g., activatable antibody) comprises: a polypeptide comprising, from N-terminus to C-terminus, a masking unit (MU), a linkage unit (LU), and a target binding moiety (TBM), where the MU is any of the masking units described herein, the LU is any of the linkage units described herein, and where the TBM comprises an antibody heavy chain variable region (VH) and an antibody light chain variable region (VL). In some embodiments, the LU comprises at least a first cleavage site. In some embodiments, the LU does not comprise a cleavage site.

The term “masked binding polypeptide” or “masked antibody” includes a polypeptide that comprises a target binding moiety (TBM), a linkage unit (LU), and a masking unit (MU). In some embodiments, the TBM comprises an amino acid sequence that binds to a target. In some embodiments, the TBM comprises an antigen binding domain (ABD) of an antibody or antibody fragment thereof (e.g., any of the anti-CTLA4 antibodies or antigen binding fragments described herein). In some embodiments, the antigen binding domain comprises a heavy chain variable region comprising one, two, or three of the heavy chain variable region CDRs described herein, and a light chain variable region comprising one, two, or three of the light chain variable region CDRs described herein (e.g., one, two, or three of the heavy chain variable region CDR sequences, and/or one, two, or three of the light chain variable region CDR sequences as shown in Table 2, including all six CDRs of any of the exemplary anti-CTLA4 antibodies as shown in Table 2). In some embodiments, the antigen binding domain comprises a heavy chain variable region comprising any of the heavy chain variable region sequences described herein, and a light chain variable region comprising any of the light chain variable region sequences described herein (e.g., a heavy chain variable region sequence and/or a light chain variable region sequence as shown in Table 3). In some embodiments, the TBM (e.g., comprising an ABD) comprises an antibody light chain variable region (VL) and an antibody heavy chain variable region (VH), wherein the VH and VL form a binding domain that binds to the target in the absence of the MU. In some embodiments, the VH and VL are covalently linked, e.g., in an scFv. In some embodiments, the VH and VL are not covalently linked. In some embodiments, the VH and VL form a Fab fragment. In some embodiments, the VH is linked to an antibody heavy chain constant region, and the VL is linked to an antibody light chain constant region.

In some embodiments, the anti-CTLA4 masked antibody (e.g., activatable antibody) comprises a polypeptide comprising the structure, from N-terminus to C-terminus, of: masking unit (MU)-linkage unit (LU)-VL, and the anti-CTLA4 masked antibody (e.g., activatable antibody) further comprises a second polypeptide comprising a VH (e.g., a Fab fragment). In some embodiments, the anti-CTLA4 masked antibody (e.g., activatable antibody) comprises a polypeptide comprising the structure, from N-terminus to C-terminus, of: masking unit (MU)-linkage unit (LU)-VL-VH (e.g., an scFv). In some embodiments, the anti-CTLA4 masked antibody (e.g., activatable antibody) comprises a polypeptide comprising the structure, from N-terminus to C-terminus, of: masking unit (MU)-linkage unit (LU)-VH, and the anti-CTLA4 masked antibody (e.g., activatable antibody) further comprises a second polypeptide comprising a VL (e.g., a Fab fragment). In some embodiments, the anti-CTLA4 masked antibody (e.g., activatable antibody) comprises a polypeptide comprising the structure, from N-terminus to C-terminus, of: masking unit (MU)-linkage unit (LU)-VH-VL (e.g., an scFv). In some embodiments, the LU comprises at least a first cleavage site. In some embodiments, the LU does not comprise a cleavage site.

In some embodiments, an anti-CTLA4 masked antibody (e.g., activatable antibody) of the present disclosure comprises a first polypeptide and a second polypeptide each comprising a VH; and a third polypeptide and a fourth polypeptide each comprising the structure, from N-terminus to C-terminus, of: masking unit (MU)-linkage unit (LU)-VL. In certain embodiments, the anti-CTLA4 masked antibody (e.g., activatable antibody) comprises a first polypeptide comprising a VH and a first Fc domain; a second polypeptide comprising a VH and a second Fc domain; and a third polypeptide and a fourth polypeptide each comprising the structure, from N-terminus to C-terminus, of: masking unit (MU)-linkage unit (LU)-VL. In certain embodiments, the first and second Fc domains are the same. In other embodiments, the first and second Fc domains are different. In some embodiments, the first and second Fc domains are both IgG1 Fc domains (e.g., human IgG1 Fc domains). The first and second IgG1 Fc domains may be the same IgG1 Fc domain or different IgG1 Fc domains. In some embodiments, the first and second IgG1 Fc domains each comprise one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In certain embodiments, the first and second IgG1 Fc domains are the same (i.e., comprise the same substitutions). In other embodiments, the first and second IgG1 Fc domains are different. In some embodiments, the LU comprises at least a first cleavage site. In some embodiments, the LU does not comprise a cleavage site. In some embodiments, the first polypeptide and second polypeptide are each heavy chains, and/or the third polypeptide and the fourth polypeptide are each light chains.

In some embodiments, an anti-CTLA4 masked antibody (e.g., activatable antibody) of the present disclosure comprises two heavy chains and two light chains, wherein the heavy chains each comprise the structure, from N-terminus to C-terminus, of: heavy chain variable region (VL)-IgG1 Fc domain (e.g., a human IgG1 Fc domain); and wherein the light chains each comprise, the structure, from N-terminus to C-terminus, of: masking unit (MU)-linkage unit (LU)-light chain variable region (VL). In certain embodiments, the first and second Fc domains are the same. In other embodiments, the first and second Fc domains are different. In some embodiments, the first and second Fc domains are both IgG1 Fc domains (e.g., human IgG1 Fc domains). The first and second IgG1 Fc domains may be the same IgG1 Fc domain or different IgG1 Fc domains. In some embodiments, the first and second IgG1 Fc domains each comprise one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In certain embodiments, the first and second IgG1 Fc domains are the same (i.e., comprise the same substitutions). In other embodiments, the first and second IgG1 Fc domains are different. In some embodiments, the LU comprises at least a first cleavage site. In some embodiments, the LU does not comprise a cleavage site.

In some embodiments, an anti-CTLA4 masked antibody (e.g., activatable antibody) of the present disclosure comprises a first polypeptide and a second polypeptide, wherein one or both of the first polypeptide and the second polypeptide comprise, from N- to C-terminus, an antibody heavy chain variable region (VH) and a heavy chain constant region (e.g., an Fc domain), wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 masked antibody (e.g., activatable antibody) comprises a first polypeptide and a second polypeptide, each comprising, from N- to C-terminus, an antibody heavy chain variable region (VH) and a heavy chain constant region (e.g., an Fc domain), wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the heavy chain constant region is an IgG1 Fc domain (e.g., a human IgG1 Fc domain). In certain embodiments, the IgG1 Fc domains (e.g., human IgG1 Fe domains) of the first and second polypeptide dimerize to form an IgG1 Fc region (e.g., a human IgG1 Fc region). In certain embodiments, the first and second heavy chain constant regions (e.g., human IgG1 Fe domains) are the same. In other embodiments, the first and second heavy chain constant regions (e.g., human IgG1 Fe domains) are different. In some embodiments, the first and second heavy chain constant regions (e.g., human IgG1 Fe domains) each comprise a S239D substitution and/or an I332E substitution. In some embodiments, the anti-CTLA4 masked antibody (e.g., activatable antibody) further comprises a third polypeptide and a fourth polypeptide, each comprising a VL. In some embodiments, the anti-CTLA4 antibody is a masked antibody (e.g., activatable antibody) comprising a third polypeptide and a fourth polypeptide, each comprising the structure, from N-terminus to C-terminus, of: masking unit (MU)-linkage unit (LU)-VL. In some embodiments, the first polypeptide and second polypeptide are each heavy chains, and/or the third polypeptide and the fourth polypeptide are each light chains.

The MU refers to an amino acid sequence that interferes with or inhibits binding of the TBM to its target. In some embodiments, the MU interferes with or inhibits binding of the TBM to its target so efficiently that binding of the TBM to its target is extremely low and/or below the limit of detection (e.g., binding cannot be detected in an ELISA or flow cytometry assay). In some embodiments, the masking unit (MU) interferes with, obstructs, reduces the ability of, prevents, inhibits, or competes with the target binding moiety for binding to its target (e.g., human CTLA4). In some embodiments, the masking unit (MU) has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.). In some embodiments, masking efficiency is measured as the difference in affinity of an anti-CTLA4 masked antibody (e.g., activatable antibody) comprising a target-binding moiety (TBM) and the masking unit (MU) for binding its target relative to the affinity of a polypeptide having the same TBM but lacking the MU for binding its target (e.g., the difference in affinity for a target antigen (such as CTLA4) of an anti-CTLA4 masked antibody (e.g., activatable antibody) comprising a masking unit (MU) relative to a parental antibody lacking the masking unit (MU). In some embodiments, the masking efficiency is measured by dividing the EC₅₀ for binding of an anti-CTLA4 masked antibody (e.g., activatable antibody) comprising a target-binding moiety (TBM) and a masking unit (MU) by the EC₅₀ of the parental antibody having the same TBM but lacking the MU (e.g., by measuring EC₅₀ by ELISA). In some embodiments, the masking unit (MU) binds to the target binding moiety (TBM), and prevents the anti-CTLA4 masked antibody (e.g., activatable antibody) from binding to its target. In some embodiments, the masking unit (MU) has a dissociation constant for binding to the target binding moiety (TBM) that is greater than the dissociation constant of the target binding moiety (TBM) for its target.

In some embodiments, an anti-CTLA4 masked antibody (e.g., activatable antibody) of the present disclosure contains a masking unit (MU) comprising a pair of cysteine residues at fixed positions to ensure that the anti-CTLA4 masked antibodies (e.g., activatable antibodies) have constrained conformations, and/or harbor few or no chemically labile residues (such as methionine or tryptophan). Advantageously, the inclusion of a pair of cysteine residues at fixed positions ensured that the anti-CTLA4 masked antibodies (e.g., activatable antibodies) had constrained conformations, tending to exhibit increased binding affinity and/or specificity. Furthermore, anti-CTLA4 masked antibodies (e.g., activatable antibodies) of the present disclosure included masking units with few to no unfavorable residues for manufacturing processes, such as methionine or tryptophan.

In some embodiments, anti-CTLA4 masked antibodies (e.g., activatable antibodies) of the present disclosure are context-dependent (e.g., selectively bind their target (e.g., human CTLA4) in certain contexts (such as in cells or tissues expressing the target (e.g., human CTLA4) at high levels)). In some embodiments, the anti-CTLA4 masked antibodies (e.g., activatable antibodies) of the present disclosure provide improved safety over more traditional, non-masked antibodies (e.g., show reduced toxicity, do not induce significant alterations to the weights of many organs, do not alter liver histopathology, hematology, and/or blood biochemistry, etc.). In some embodiments, anti-CTLA4 masked antibodies (e.g., activatable antibodies) of the present disclosure have improved pharmacokinetic properties as compared to more traditional, non-masked antibodies (e.g., have longer in vivo half-lives).

In some embodiments, the anti-CTLA4 masked antibodies disclosed herein selectively bind to tissues and cells with high CTLA4 expression levels. Without wishing to be bound by theory, because the MU competes with human CTLA4 to bind the TBM, the masked antibody selectively binds to CTLA4 in cells and tissues with adequately high CTLA4 expression levels and, thus, adequately high local concentrations of CTLA4 on the cell surface. This phenomenon of binding to the target (e.g., CTLA4) selectively in tissues and on cells having high expression levels of the target (e.g., CTLA4) may be referred to as selective target engagement. In certain embodiments, selective target engagement of an anti-CTLA4 masked antibody described herein results in a reduction of on-target, off-tumor effects (e.g., antigen sink effects, cytokine storm) as compared to an anti-CTLA4 antibody having the same TBM but lacking the masking peptide comprising the masking unit. In some embodiments, the masked anti-CTLA4 antibody is not an activatable antibody.

### Anti-CTLA4 Masked Antibody Activities

In some embodiments, the present disclosure relates to masked antibodies (e.g., activatable antibodies) that bind to human CTLA4. In some embodiments, the anti-CTLA4 masked antibodies (e.g., activatable antibodies) have at least one (e.g., at least one, at least two, at least three, at least four, at least five, at least six, at least seven, or all eight) of the following functional properties: (a) bind to human, cynomolgus monkey, mouse, rat, and/or dog CTLA4 with a K_{D} of 100 nM or more in vitro; (b) have antagonist activity on human CTLA4 on cells or in tissues with high CTLA4 expression levels; (c) do not bind to human PD-1, PD-L1, PD-L2, LAG3, TIM3, B7-H3, CD95, CD120a, OX40, CD40, BTLA, VISTA, ICOS, and/or B7-H4 at concentration up to 100 nM; (d) are cross-reactive with monkey, mouse, rat, and/or dog CTLA4 on cells or in tissues with high CTLA4 expression levels; (e) induces ADCC effects (e.g., on Tregs) on cells or in tissues with high CTLA4 expression levels; (f) are capable of inhibiting tumor cell growth in tumors with high CTLA4 expression levels; (g) have therapeutic effect on a cancer with high CTLA4 expression levels; and (h) inhibit binding of human CTLA4 to human CD80 and/or human CD86 on cells or in tissues with high CTLA4 expression levels. Also provided herein are one or more masked antibodies (e.g., activatable antibodies) that compete or cross-compete for binding to human CTLA4 with one or more of the CTLA4-targeting masked antibodies (e.g., activatable antibodies) and/or anti-CTLA4 antibodies described herein.

In some embodiments, the anti-CTLA4 masked antibodies (e.g., activatable antibodies) bind to human, cynomolgus monkey, mouse, rat, and/or dog CTLA4 with a K_{D} of about 100 nM or more (e.g., about 100 nM or more, about 200 nM or more, about 300 nM or more, about 400 nM or more, or about 100 nM or more) in vitro. Methods of measuring the K_{D} of a masked antibody (e.g., an activatable antibody) in vitro may be carried out using any method known in the art, including for example, by surface plasmon resonance, an ELISA, isothermal titration calorimetry, a filter binding assay, an EMSA, etc. In some embodiments, the K_{D} is measured by an ELISA (see e.g., the Examples below).

In some embodiments, the anti-CTLA4 masked antibodies (e.g., activatable antibodies) have antagonist activity on human CTLA4 on cells or in tissues with high CTLA4 expression levels (e.g., induces ADCC effects (such as against Tregs), activates PBMCs (such as by activating, inducing, and/or stimulating IL-2 and/or IFNγ secretion), bocks binding of human CTLA4 to human CD80 and/or human CD86, etc.). In some embodiments, the anti-CTLA4 masked antibodies (e.g., activatable antibodies) repress one or more activities of human CTLA4 on cells or in tissues with high CTLA4 expression levels (e.g., repress one or more activities of human CTLA4 when a cell (such as a human cell) expressing human CTLA4 is contacted by an anti-CTLA4 masked antibody).

In some embodiments, the anti-CTLA4 masked antibodies (e.g., activatable antibodies) are cross-reactive with monkey (e.g., cynomolgus monkey), mouse, rat, and/or dog CTLA4. In some embodiments, the anti-CTLA4 masked antibodies (e.g., activatable antibodies) are cross-reactive with monkey CTLA4. In some embodiments, the anti-CTLA4 masked antibodies (e.g., activatable antibodies) are cross-reactive with mouse CTLA4. In some embodiments, the anti-CTLA4 masked antibodies (e.g., activatable antibodies) are cross-reactive with rat CTLA4. In some embodiments, the anti-CTLA4 masked antibodies (e.g., activatable antibodies) are cross-reactive with dog CTLA4. In some embodiments, the anti-CTLA4 masked antibodies (e.g., activatable antibodies) are cross reactive with monkey and mouse CTLA4; monkey and rat CTLA4; monkey and dog CTLA4; mouse and rat CTLA4; mouse and dog CTLA4; rat and dog CTLA4; monkey, mouse, and rat CTLA4; monkey, mouse, and dog CTLA4; monkey, rat, and dog CTLA4; mouse, rat, and dog CTLA4; or monkey, mouse, rat, and dog CTLA4. In some embodiments, the anti-CTLA4 masked antibodies (e.g., activatable antibodies) are cross-reactive at about 350 nM or less (e.g., at about 1 nM or less, at about 10 nM or less, at about 25 nM or less, at about 50 nM or less, at about 75 nM or less, at about 100 nM or less, at about 150 nM or less, at about 200 nM or less, at about 250 nM or less, at about 300 nM or less, or at about 350 nM). Methods of measuring cross-reactivity are known in the art, including, without limitation, surface plasmon resonance, an ELISA, isothermal titration calorimetry, a filter binding assay, an EMSA, etc.

In some embodiments, the anti-CTLA4 masked antibodies (e.g., activatable antibodies) have reduced ADCC effects (e.g., against CTLA4-expressing human cells such as Tregs) in vitro, or on cells or in tissues with low CTLA4 expression levels, as compared to a control binding polypeptide (e.g., a parental antibody). In some embodiments, the anti-CTLA4 masked antibodies (e.g., activatable antibodies) induce ADCC effects (e.g., against CTLA4-expressing such as Tregs) on cells or in tissues with high CTLA4 expression levels. Methods of measuring ADCC effects (e.g., in vitro methods) are known in the art, including, without limitation, via the methods described in the Examples below. In some embodiments, the anti-CTLA4 masked antibodies (e.g., activatable antibodies) induce ADCC effects by less than about 10% (e.g., induce ADCC by less than about 10%, less than about 5%, less than about 1%, etc.) relative to a control (e.g., a parental antibody) in vitro, or on cells or in tissues with low CTLA4 expression levels. In some embodiments, the anti-CTLA4 masked antibodies (e.g., activatable antibodies) induce ADCC effects by more than about 10% (e.g., induce ADCC by more than about 10%, more than about 15%, more than about 20%, more than about 25%, more than about 30%, more than about 35%, more than about 40%, etc.) relative to a control (e.g., an isotype control) on cells or in tissues with high CTLA4 expression levels.

In some embodiments, the anti-CTLA4 masked antibodies (e.g., activatable antibodies) are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the anti-CTLA4 masked antibodies (e.g., activatable antibodies) relative to corresponding tumor cells not contacted with the anti-CTLA4 masked antibodies (e.g., activatable antibodies) (or relative to corresponding tumor cells contacted with an isotype control antibody). In some embodiments, the anti-CTLA4 masked antibodies (e.g., activatable antibodies) are capable of reducing tumor volume in a subject when the subject is administered the anti-CTLA4 masked antibodies (e.g., activatable antibodies). In some embodiments, the anti-CTLA4 masked antibodies (e.g., activatable antibodies) are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the anti-CTLA4 masked antibodies (e.g., activatable antibodies); as compared to a corresponding tumor in a subject administered an isotype control antibody). Methods of monitoring tumor cell growth/proliferation, tumor volume, and/or tumor inhibition are known in the art, including, for example, via the methods described in the Examples below.

In some embodiments, the anti-CTLA4 masked antibodies (e.g., activatable antibodies) have therapeutic effect on a cancer. In some embodiments, the anti-CTLA4 masked antibodies (e.g., activatable antibodies) reduce one or more signs or symptoms of a cancer. In some embodiments, a subject suffering from a cancer goes into partial or complete remission when administered the anti-CTLA4 masked antibodies (e.g., activatable antibodies).

In some embodiments, the present disclosure provides isolated anti-CTLA4 masked antibodies (e.g., activatable antibodies) that compete or cross-compete for binding to human CTLA4 with an antibody comprising: a) a CDR-H1 comprising the amino acid sequence of SEQ ID NO: 1, or a variant thereof comprising up to 5 (e.g., 1, 2, 3, 4, or 5) amino acid substitutions; a CDR-H2 comprising the amino acid sequence of SEQ ID NO: 2, or a variant thereof comprising up to 5 (e.g., 1, 2, 3, 4, or 5) amino acid substitutions; and a CDR-H3 comprising the amino acid sequence of SEQ ID NO: 3, or a variant thereof comprising up to 5 (e.g., 1, 2, 3, 4, or 5) amino acid substitutions; and/or b) a CDR-L1 comprising the amino acid sequence of SEQ ID NO: 4; a CDR-L2 comprising the amino acid sequence of SEQ ID NO: 5, or a variant thereof comprising up to 5 (e.g., 1, 2, 3, 4, or 5) amino acid substitutions; and a CDR-L3 comprising the amino acid sequence of SEQ ID NO: 6, or a variant thereof comprising up to 5 (e.g., 1, 2, 3, 4, or 5) amino acid substitutions. In some embodiments, the present disclosure provides isolated anti-CTLA4 masked antibodies (e.g., activatable antibodies) that compete or cross-compete for binding to human CTLA4 with an antibody comprising: a) a heavy chain variable region comprising the amino acid sequence of SEQ ID NO: 74, or an amino acid sequence having at least 80% (e.g., at least 85%, 90%, 95%, 98%, or 99%; or 100%) sequence identity with SEQ ID NO: 74; and/or b) a light chain variable region comprising the amino acid sequence of SEQ ID NO: 75, or an amino acid sequence having at least 80% (e.g., at least 85%, 90%, 95%, 98%, or 99%; or 100%) sequence identity with SEQ ID NO: 75. The ability of a masked antibody (e.g., an activatable antibody) to compete or cross-compete for binding with an antibody can be determined using standard binding assays known in the art, such as BIAcore analysis, ELISA assays, or flow cytometry. For example, one can allow an antibody (e.g., as described above) to bind to human CTLA4 under saturating conditions and then measure the ability of the test masked antibody (e.g., activatable antibody) to bind to the CTLA4. If the masked antibody (e.g., activatable antibody) is able to bind to the CTLA4 at the same time as the antibody, then the test masked antibody (e.g., activatable antibody) binds to a different epitope then the antibody. However, if the test masked antibody (e.g., activatable antibody) is not able to bind to the CTLA4 at the same time, then the test masked antibody (e.g., activatable antibody) binds to the same epitope, an overlapping epitope, or an epitope that is in close proximity to the epitope bound by the antibody. This experiment can be performed using various methods, such as ELISA, RIA, FACS or surface plasmon resonance.

In some embodiments, the anti-CTLA4 masked antibodies (e.g., activatable antibodies) do not inhibit the binding between CTLA4 and one or more of its binding partners (e.g., human CTLA4 and human CD80, human CTLA4 and human CD86) in vitro, or on cells or in tissues with low CTLA4 expression levels. In some embodiments, the anti-CTLA4 masked antibodies (e.g., activatable antibodies) inhibit the binding between CTLA4 and one or more of its binding partners (e.g., human CTLA4 and human CD80, human CTLA4 and human CD86) on cells or in tissues with high CTLA4 expression levels. In some embodiments, the anti-CTLA4 masked antibodies (e.g., activatable antibodies) inhibit the binding between CTLA4 and its ligand in vitro. In some embodiments, the anti-CTLA4 masked antibodies (e.g., activatable antibodies) have a half maximal inhibitory concentration (IC₅₀) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for inhibiting binding of CTLA4 to CD80 and/or CD86. In some embodiments, the anti-CTLA4 masked antibodies (e.g., activatable antibodies) have a half maximal inhibitory concentration (IC₅₀) of about 100 nM or less for inhibiting binding of CTLA4 to CD80 and/or CD86. In some embodiments, the anti-CTLA4 masked antibodies (e.g., activatable antibodies) completely inhibit binding of human CTLA4 to CD80 and/or CD86 when provided at a concentration of about 100 nM or greater (e.g., about 100 nM or greater, about 500 nM or greater, about 1 μM or greater, about 10 μM or greater, etc.). As used herein, the term “complete inhibiting” or “completely inhibits” refers to the ability of the anti-CTLA4 masked antibody (e.g., activatable antibody) to reduce binding between a first protein and a second protein by at least about 80% (e.g., at least about 80%, at least about 85%, at least about 90%, at least about 95%, at least about 99%, etc.). Methods of measuring the ability of an a polypeptide to inhibit binding of a first protein (e.g., human CTLA4) and a second protein (e.g., human CD80 or human CD86) are known in the art, including, without limitation, via BIAcore analysis, ELISA assays, and flow cytometry.

### Target Binding Moieties (TBMs)

In some embodiments, the present disclosure relates to anti-CTLA4 masked antibodies (e.g., activatable antibodies) comprising a target binding moiety (TBM). In some embodiments, the target binding moiety (TBM) comprises an antibody light chain variable region and/or an antibody heavy chain variable region. In some embodiments, the target binding moiety (TBM) comprises an antibody light chain variable region. In some embodiments, the target binding moiety (TBM) comprises an antibody heavy chain variable region. In some embodiments, the target binding moiety (TBM) comprises an antibody light chain variable region and an antibody heavy chain variable region.

In some embodiments, the target binding moiety (TBM) comprises a full length antibody light chain and/or a full length antibody heavy chain. The antibody light chain may be a kappa or lambda light chain. The antibody heavy chain may be in any class, such as IgG, IgM, IgE, IgA, or IgD. In some embodiments, the antibody heavy chain is in the IgG class, such as IgG1, IgG2, IgG3, or IgG4 subclass. In some embodiments, the antibody heavy chain is in the IgG1 subclass. In some embodiments, the antibody heavy chain comprises a human IgG1 Fc region, such as a human IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering. In certain embodiments, the first and second IgG1 Fc domains are the same (i.e., comprise the same substitutions). An antibody heavy chain described herein may be converted from one class or subclass to another class or subclass using methods known in the art.

Any one or more of the target binding moieties (TBMs) described herein may incorporate: any of the CDR sequences described herein (e.g., one, two, or three of the heavy chain variable region CDR sequences, and/or one, two, or three of the light chain variable region CDR sequences as shown in Table 2 above); any of the heavy chain variable region sequences and/or light chain variable region sequences described herein (e.g., a heavy chain variable region sequence and/or a light chain variable region sequence as shown in Table 3 above); and/or any of any of the anti-CTLA4 antibodies described herein or in U.S. Patent Application Publication No. US20210207126A1

In some embodiments, the TBM comprises one, two, three, four, five, or six CDRs of antibody TY21580 as shown in Table 2. In some embodiments, the TBM comprises the VH and/or the VL of antibody TY21580 as shown in Table 3.

In some embodiments, the TBM comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 74. In certain embodiments, a VH sequence contains substitutions (e.g., conservative substitutions), insertions, or deletions relative to the amino acid sequence of SEQ ID NO: 74, but retains the ability to bind human CTLA4 as the antibody comprising SEQ ID NO: 74. In certain embodiments, a total of 1 to 13 (e.g., 1, 2, 3, 4, 5, 6, 7, 8, 9, 10, 11, 12, or 13) amino acids have been substituted, inserted and/or deleted in SEQ ID NO: 74. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO: 1, or a variant thereof comprising up to 5 (e.g., 1, 2, 3, 4, or 5) amino acid substitutions, (b) a CDR-H2 comprising the amino acid sequence of SEQ ID NO: 2, or a variant thereof comprising up to 5 (e.g., 1, 2, 3, 4, or 5) amino acid substitutions, and (c) a CDR-H3 comprising the amino acid sequence of SEQ ID NO: 3, or a variant thereof comprising up to 5 (e.g., 1, 2, 3, 4, or 5) amino acid substitutions.

In another aspect, the TBM comprises a light chain variable domain (VL) having at least 90%, 91%, 92%, 93%, 94%, 95%, 96%, 97%, 98%, 99%, or 100% sequence identity to the amino acid sequence of SEQ ID NO: 75. In certain embodiments, a VL sequence contains substitutions (e.g., conservative substitutions), insertions, or deletions relative to the amino acid sequence of SEQ ID NO: 75, but retains the ability to bind human CTLA4 as the antibody comprising SEQ ID NO: 75. In certain embodiments, a total of 1 to 11 (e.g., 1, 2, 3, 4, 5, 6, 7, 8, 9, 10, or 11) amino acids have been substituted, inserted and/or deleted in SEQ ID NO: 75. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO: 4, or a variant thereof comprising up to 5 (e.g., 1, 2, 3, 4, or 5) amino acid substitutions; (b) a CDR-L2 comprising the amino acid sequence of SEQ ID NO: 5, or a variant thereof comprising up to 5 (e.g., 1, 2, 3, 4, or 5) amino acid substitutions; and (c) a CDR-L3 comprising the amino acid sequence of SEQ ID NO: 6, or a variant thereof comprising up to 5 (e.g., 1, 2, 3, 4, or 5) amino acid substitutions.

In one embodiment, the TBM comprises a VH comprising the amino acid sequence of SEQ ID NO: 74, or an amino acid sequence having at least 80% (e.g., at least 85%, 90%, 95%, 98%, or 99%; or 100%) sequence identity with SEQ ID NO: 74, and a VL comprising the amino acid sequence of SEQ ID NO: 75, or an amino acid sequence having at least 80% (e.g., at least 85%, 90%, 95%, 98%, or 99%; or 100%) sequence identity with SEQ ID NO: 75.

In some embodiments, the TBM comprises a VH comprising a CDR-H1 comprising the amino acid sequence of SEQ ID NO: 1, or a variant thereof comprising up to 5 (e.g., 1, 2, 3, 4, or 5) amino acid substitutions, a CDR-H2 comprising the amino acid sequence of SEQ ID NO: 2, or a variant thereof comprising up to 5 (e.g., 1, 2, 3, 4, or 5) amino acid substitutions, and a CDR-H3 comprising the amino acid sequence of SEQ ID NO: 3, or a variant thereof comprising up to 5 (e.g., 1, 2, 3, 4, or 5) amino acid substitutions; and a VL comprising a CDR-L1 comprising the amino acid sequence of SEQ ID NO: 4, or a variant thereof comprising up to 5 (e.g., 1, 2, 3, 4, or 5) amino acid substitutions, a CDR-L2 comprising the amino acid sequence of SEQ ID NO: 5, or a variant thereof comprising up to 5 (e.g., 1, 2, 3, 4, or 5) amino acid substitutions, and a CDR-L3 comprising the amino acid sequence of SEQ ID NO: 6, or a variant thereof comprising up to 5 (e.g., 1, 2, 3, 4, or 5) amino acid substitutions.

In some embodiments, the TBM comprises a VH CDR1, a VH CDR2, and a VH CDR3, respectively comprising the amino acid sequences of a CDR1, a CDR2, and a CDR3 of a VH having the sequence set forth in SEQ ID NO: 74; and a VL CDR1, a VL CDR2, and a VL CDR3, respectively comprising the amino acid sequences of a CDR1, a CDR2, and a CDR3 of a VL having the sequence set forth in SEQ ID NO: 75.

### Masking Units (MUs)

In some embodiments, the present disclosure relates to anti-CTLA4 masked antibodies (e.g., activatable antibodies) comprising a masking unit (MU).

Exemplary masking units are provided in the Examples (see, e.g., Example 2, Tables 5A-5E). In some embodiments, the MU comprises the amino acid sequence of SEQ ID NO: 146, or a variant thereof comprising up to 5 (e.g., 1, 2, 3, 4, or 5) amino acid substitutions. In some embodiments, the MU comprises the amino acid sequence of SEQ ID NO: 147, or a variant thereof comprising up to 5 (e.g., 1, 2, 3, 4, or 5) amino acid substitutions. In some embodiments, the MU comprises the amino acid sequence of SEQ ID NO: 148, or a variant thereof comprising up to 5 (e.g., 1, 2, 3, 4, or 5) amino acid substitutions. In some embodiments, the MU comprises the amino acid sequence of SEQ ID NO: 149, or a variant thereof comprising up to 5 (e.g., 1, 2, 3, 4, or 5) amino acid substitutions. In some embodiments, the MU comprises the amino acid sequence of SEQ ID NO: 150, or a variant thereof comprising up to 5 (e.g., 1, 2, 3, 4, or 5) amino acid substitutions. In some embodiments, the MU comprises the amino acid sequence of SEQ ID NO: 151, or a variant thereof comprising up to 5 (e.g., 1, 2, 3, 4, or 5) amino acid substitutions. In some embodiments, the MU comprises an amino acid sequence selected from the group consisting of SEQ ID NO: 146-151, or a variant thereof comprising up to 5 (e.g., 1, 2, 3, 4, or 5) amino acid substitutions.

In some embodiments, any of the masking units (MUs) described herein may further comprise one or more additional amino acid sequences (e.g., one or more polypeptide tags). Examples of suitable additional amino acid sequence may include, without limitation, purification tags (such as his-tags, flag-tags, maltose binding protein and glutathione-S-transferase tags), detection tags (such as tags that may be detected photometrically (e.g., red or green fluorescent protein, etc.)), tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the one or more additional amino acid sequences are at the N-terminus of the masking unit (MU). In some embodiments, the additional amino acid sequence comprises or consists of the sequence EVGSY (SEQ ID NO: 122).

### Linkage Units (LUs) of Masked Antibodies

In some embodiments, the present disclosure relates to anti-CTLA4 masked antibodies (e.g., activatable antibodies) comprising a linkage unit (LU). In some embodiments, the linkage unit (LU) does not comprise a cleavage site. In other embodiments, the linkage unit (LU) comprises at least a first cleavage site. The cleavage site may be any cleavage site known in the art or described herein.

### Anti-CTLA4 Activatable Antibodies

In some embodiments, an anti-CTLA4 masked antibody of the present disclosure is an anti-CTLA4 activatable antibody. Thus, the present disclosure also relates, in part, to activatable binding polypeptides (i.e., activatable antibodies) that bind to human CTLA4, including activatable antibodies comprising any of the anti-CTLA4 antibodies described herein (e.g., anti-CTLA4 antibodies, anti-CTLA4 antibody binding fragments, and/or anti-CTLA4 antibody derivatives), antigen binding fragments of the activatable anti-CTLA4 antibodies, and/or derivatives of the activatable anti-CTLA4 antibodies. In some embodiments, the activatable anti-CTLA4 antibodies described herein may have improved safety profiles. For example, the activatable anti-CTLA4 antibodies described herein may have better safety margin as assessed by spleen weight change. The change in spleen size with the increase in drug dose administered is used as a benchmark to assess the safety margin of the drug candidate used.

In some embodiments, an anti-CTLA4 activatable antibody of the present disclosure comprises: (a) a masking unit (MU); (b) a linkage unit comprising at least a first cleavage site; and (c) a target binding moiety (TBM). In some embodiments, the MU is any of the masking units described herein. In some embodiments, the masking unit (MU) binds to the target binding moiety (TBM) and inhibits the activatable antibody from binding to its target before activation (e.g., before treatment with one or more proteases that cleave within the cleavage site, before undergoing a (local) change in pH (increased or decreased), before a temperature shift (increased or decreased), before being contacted with a second molecule (such as a small molecule or a protein ligand), etc.), but does not bind to the TBM and/or inhibit the anti-CTLA4 activatable antibody from binding to its target after activation (e.g., after treatment with one or more proteases that cleave within the cleavage site, after undergoing a (local) change in pH (increased or decreased), after a temperature shift (increased or decreased), after being contacted with a second molecule (such as a small molecule or a protein ligand), etc.). In some embodiments, the masking unit (MU) has a dissociation constant for binding to the TBM that is greater (e.g., at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 10-fold greater, at least about 100-fold greater, at least about 500-fold greater, etc.) than the dissociation constant of the anti-CTLA4 activatable antibody for its target (when in active form, e.g., when the cleavage site is cleaved). The amino acid sequence of the cleavage site may overlap with or be included within the MU.

In some embodiments, an anti-CTLA4 activatable antibody of the present disclosure comprises: (a) a masking peptide comprising, from N-terminus to C-terminus, a masking unit (MU) and a linkage unit (LU) comprising at least a first cleavage site, and (b) a target binding moiety (TBM) comprising an antibody heavy chain variable region (VH) and an antibody light chain variable region (VL); wherein the masking peptide is linked to the N-terminus of the VH or the VL; and wherein the anti-CTLA4 activatable antibody has a higher binding affinity to human CTLA4 in vitro upon cleavage of the cleavage site than before the cleavage of the cleavage site.

In some embodiments, an anti-CTLA4 activatable antibody of the present disclosure comprises: (a) a masking unit (MU); (b) a linkage unit (LU) comprising at least a first cleavage site; and (c) a target binding moiety (TBM). In some embodiments, the MU is any of the masking units described herein. In some embodiments, the cleavage site is any of the cleavage sites described herein. In some embodiments, the TBM is any of the target binding moieties described herein (e.g., a target binding moiety (TBM) comprising an antibody light chain variable region and/or an antibody heavy chain variable region, such as a VH and/or VL of any of the anti-CTLA4 antibodies described herein). In some embodiments, the MU interferes with and/or inhibits the binding of the anti-CTLA4 activatable antibody to its target (e.g., human CTLA4) when the cleavage site is not cleaved. In some embodiments, the anti-CTLA4 activatable antibody is capable of binding to its target (e.g., human CTLA4) when the cleavage site is cleaved. In some embodiments, the anti-CTLA4 activatable antibody binds to its target (e.g., human CTLA4) at reduced binding affinity when the cleavage site is not cleaved, as compared the anti-CTLA4 activatable antibody when the cleavage site is cleaved. In some embodiments, the anti-CTLA4 activatable antibody has a higher binding affinity to its target (e.g., human CTLA4) in vitro upon cleavage of the cleavage site than before cleavage of the cleavage site.

In some embodiments, the anti-CTLA4 activatable antibody comprises: (a) a polypeptide comprising, from N-terminus to C-terminus, a masking unit (MU), a linkage unit (LU) comprising at least a first cleavage site, and a target binding moiety (TBM), where the MU is any of the masking units described herein, the cleavage site is any of the cleavage sites described herein, and where the TBM comprises an antibody light chain variable region (VL); and (b) an antibody heavy chain variable region (VH).

In some embodiments, the anti-CTLA4 activatable antibody comprises: (a) a polypeptide comprising, from N-terminus to C-terminus, a masking unit (MU), linkage unit (LU) comprising at least a first cleavage site, and a target binding moiety (TBM), where the MU is any of the masking units described herein, the cleavage site is any of the cleavage sites described herein, and where the TBM comprises an antibody heavy chain variable region (VH); and (b) an antibody light chain variable region (VL).

In some embodiments, the anti-CTLA4 activatable antibody comprises: a polypeptide comprising, from N-terminus to C-terminus, a masking unit (MU), a linkage unit (LU) comprising at least a first cleavage site, and a target binding moiety (TBM), where the MU is any of the masking units described herein, the cleavage site is any of the cleavage sites described herein, and where the TBM comprises an antibody heavy chain variable region (VH) and an antibody light chain variable region (VL).

The term “activatable binding polypeptide” or “activatable antibody” includes a polypeptide that comprises a target binding moiety (TBM), linkage unit (LU) comprising at least a first cleavage site, and a masking unit (MU). In some embodiments, the TBM comprises an amino acid sequence that binds to a target. In some embodiments, the TBM comprises an antigen binding domain (ABD) of an anti-CTLA4 antibody or antibody fragment thereof (e.g., any of the anti-CTLA4 antibodies or antigen binding fragments described herein). In some embodiments, the antigen binding domain comprises a heavy chain variable region comprising one, two, or three of the heavy chain variable region CDRs described herein, and a light chain variable region comprising one, two, or three of the light chain variable region CDRs described herein (e.g., one, two, or three of the heavy chain variable region CDR sequences, and/or one, two, or three of the light chain variable region CDR sequences as shown in Table 2, including all six CDRs of any of the exemplary anti-CTLA4 antibodies as shown in Table 2). In some embodiments, the antigen binding domain comprises a heavy chain variable region comprising any of the heavy chain variable region sequences described herein, and a light chain variable region comprising any of the light chain variable region sequences described herein (e.g., a heavy chain variable region sequence and/or a light chain variable region sequence as shown in Table 3). In some embodiments, the TBM (e.g., comprising an ABD) comprises an antibody light chain variable region (VL) and an antibody heavy chain variable region (VH), wherein the VH and VL forms a binding domain that binds to the target in the absence of the MU. In some embodiments, the VH and VL are covalently linked, e.g., in an scFv. In some embodiments, the VH and VL are not covalently linked. In some embodiments, the VH and VL form a Fab fragment. In some embodiments, the VH is linked to an antibody heavy chain constant region, and the VL is linked to an antibody light chain constant region.

In some embodiments, the anti-CTLA4 activatable antibody comprises a polypeptide comprising the structure, from N-terminus to C-terminus, of: masking unit (MU)-linkage unit (LU)-VL, and the anti-CTLA4 activatable antibody further comprises a second polypeptide comprising a VH (e.g., a Fab fragment). In some embodiments, the anti-CTLA4 activatable antibody comprises a polypeptide comprising the structure, from N-terminus to C-terminus, of: masking unit (MU)-linkage unit (LU)-VL-VH (e.g., an scFv). In some embodiments, the anti-CTLA4 activatable antibody comprises a polypeptide comprising the structure, from N-terminus to C-terminus, of: masking unit (MU)-linkage unit LU)-VH, and the activatable antibody further comprises a second polypeptide comprising a VL (e.g., a Fab fragment). In some embodiments, the anti-CTLA4 activatable antibody comprises a polypeptide comprising the structure, from N-terminus to C-terminus, of: masking unit (MU)-linkage unit (LU)-VH-VL (e.g., an scFv). In some embodiments which may be combined with the preceding embodiments, the LU comprises at least a first cleavage site (e.g., a first cleavage site, or a first cleavage site and a second cleavage site).

In some embodiments, an anti-CTLA4 activatable antibody of the present disclosure comprises a first polypeptide and a second polypeptide each comprising a VH; and a third polypeptide and a fourth polypeptide each comprising the structure, from N-terminus to C-terminus, of: masking unit (MU)-linkage unit (LU)-VL. In certain embodiments, the anti-CTLA4 activatable antibody comprises a first polypeptide comprising a VH and a first Fc domain; a second polypeptide comprising a VH and a second Fc domain; and a third polypeptide and a fourth polypeptide each comprising the structure, from N-terminus to C-terminus, of: masking unit (MU)-linkage unit (LU)-VL. In certain embodiments, the first and second Fc domains are the same. In other embodiments, the first and second Fc domains are different. In some embodiments, the first and second Fc domains are both IgG1 Fc domains (e.g., human IgG1 Fc domains). The first and second IgG1 Fc domains may be the same IgG1 Fc domain or different IgG1 Fc domains. In some embodiments, the first and second IgG1 Fc domains each comprise one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the LU comprises at least a first cleavage site. In some embodiments, the LU does not comprise a cleavage site. In some embodiments which may be combined with the preceding embodiments, the LU comprises at least a first cleavage site (e.g., a first cleavage site, or a first cleavage site and a second cleavage site). In some embodiments, the first polypeptide and second polypeptide are each heavy chains, and/or the third polypeptide and the fourth polypeptide are each light chains.

In some embodiments, an anti-CTLA4 activatable antibody of the present disclosure comprises a first polypeptide and a second polypeptide, wherein one or both of the first polypeptide and the second polypeptide comprise, from N- to C-terminus, an antibody heavy chain variable region (VH) and a heavy chain constant region (e.g., an Fc domain), wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 activatable antibody comprises a first polypeptide and a second polypeptide, each comprising, from N- to C-terminus, an antibody heavy chain variable region (VH) and a heavy chain constant region (e.g., an Fc domain), wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the heavy chain constant region is an IgG1 Fc domain (e.g., a human IgG1 Fc domain). In certain embodiments, the IgG1 Fc domains (e.g., human IgG1 Fc domains) of the first and second polypeptide dimerize to form an IgG1 Fc region (e.g., a human IgG1 Fc region). In certain embodiments, the first and second heavy chain constant regions (e.g., human IgG1 Fc domains) are the same. In other embodiments, the first and second heavy chain constant regions (e.g., human IgG1 Fc domains) are different. In some embodiments, the first and second heavy chain constant regions (e.g., human IgG1 Fc domains) each comprise a S239D substitution and/or an I332E substitution.

In some embodiments, the anti-CTLA4 activatable antibody further comprises a third polypeptide and a fourth polypeptide, each comprising a VL. In some embodiments, the anti-CTLA4 antibody is a masked antibody (e.g., activatable antibody) comprising a third polypeptide and a fourth polypeptide, each comprising the structure, from N-terminus to C-terminus, of: masking unit (MU)-linkage unit (LU)-VL. In some embodiments, the first polypeptide and second polypeptide are each heavy chains, and/or the third polypeptide and the fourth polypeptide are each light chains. In some embodiments, the anti-CTLA4 activatable antibody further comprises a third polypeptide and a fourth polypeptide, each comprising the structure, from N-terminus to C-terminus, of: masking unit (MU)-linkage unit (LU)-VL. In some embodiments, the LU comprises at least a first cleavage site (e.g., a first cleavage site, or a first cleavage site and a second cleavage site). In some embodiments, the first polypeptide and second polypeptide are each heavy chains, and/or the third polypeptide and the fourth polypeptide are each light chains.

In embodiments wherein the linkage unit (LU) comprises at least a first cleavage site, the cleavage site generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a cleavage site, the terms encompass enzymatic cleavage, e.g., by a protease, as well as disruption of a disulfide bond between a cysteine-cysteine pair via reduction of the disulfide bond that can result from exposure to a reducing agent.

The MU refers to an amino acid sequence that interferes with or inhibits binding of the TBM to its target. In some embodiments, the MU interferes with or inhibits binding of the TBM to its target so efficiently that binding of the TBM to its target is extremely low and/or below the limit of detection (e.g., binding cannot be detected in an ELISA or flow cytometry assay). The amino acid sequence of the linkage unit (LU) may overlap with or be included within the MU. It should be noted that for sake of convenience “activatable antibody” is used herein to refer to an activatable antibody in both their unactivated (e.g., uncleaved or “native”) state, as well as in their activated (e.g., cleaved) state. It will be apparent to the ordinarily skilled artisan that in some embodiments a activated (e.g., cleaved) activatable antibody may lack an MU due to cleavage of one or more cleavage sites in the LU, e.g., by a protease, resulting in release of at least the MU (e.g., where the MU is not joined to the activatable antibody by a covalent bond (e.g., a disulfide bond between cysteine residues)). Exemplary activatable antibodies are described in more detail below. As used herein, the terms “masking unit” and “masking moiety” are used interchangeably.

In some embodiments, the masking unit (MU) interferes with, obstructs, reduces the ability of, prevents, inhibits, or competes with the target binding moiety for binding to its target (e.g., an “inactive activatable antibody”). In some embodiments, the masking unit (MU) interferes with, obstructs, reduces, prevents, inhibits, or competes with the target binding moiety for binding to its target only when the polypeptide has not been activated (e.g., activated by a change in pH (increased or decreased), activated by a temperature shift (increased or decreased), activated after being contacted with a second molecule (such as a small molecule or a protein ligand), etc.). In some embodiments wherein the LU comprises at least a first cleavage site, activation induces cleavage of the polypeptide within the cleavage site. In some embodiments, activation induces conformation changes in the polypeptide (e.g., displacement of the masking unit (MU), leading to the masking unit no longer preventing the activatable antibody from binding to its target. In some embodiments wherein the LU comprises at least a first cleavage site, the masking unit (MU) interferes with, obstructs, reduces the ability of, prevents, inhibits, or competes with the target binding moiety for binding to its target only when the linkage unit (LU) has not been cleaved by one or more proteases that cleave within the cleavage site. In some embodiments wherein the LU comprises at least a first cleavage site, the masking unit (MU) inhibits binding of an anti-CTLA4 activatable antibody to its target when the cleavage site is not cleaved, but does not inhibit binding of the anti-CTLA4 activatable antibody to its target when the cleavage site is cleaved.

In some embodiments, the masking unit (MU) has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.) prior to activation (e.g., before cleavage of the cleavage site). In some embodiments, masking efficiency is measured as the difference in affinity of an anti-CTLA4 activatable antibody comprising the masking unit (MU) for binding its target (before activation, e.g., before cleavage of the cleavage site) relative to the affinity of a polypeptide lacking the masking unit for binding its target (e.g., the difference in affinity for a target antigen of an anti-CTLA4 activatable antibody comprising a target-binding moiety (TBM) and a masking unit (MU) (before activation, e.g., before cleavage of the cleavage site) relative to a parental antibody having the same target-binding moiety (TBM) but lacking the masking unit (MU), or the difference in affinity for a target antigen (such as CTLA4) of an anti-CTLA4 activatable antibody comprising a masking unit (MU) (before activation, e.g., before cleavage of the cleavage site) relative to the affinity for the target antigen of the anti-CTLA4 activatable antibody after activation (e.g., after cleavage of the cleavage site)). In some embodiments, the masking efficiency is measured by dividing the EC₅₀ for binding of an anti-CTLA4 activatable antibody comprising a target-binding moiety (TBM) and a masking unit (MU) (before activation, e.g., before cleavage of the cleavage site) by the EC₅₀ of the parental antibody (e.g., by measuring EC₅₀ by ELISA). In some embodiments, masking efficiency is measured as the difference in affinity of an anti-CTLA4 activatable antibody comprising the masking unit (MU) for binding its target before relative to the affinity of the anti-CTLA4 activatable antibody comprising the masking unit (MU) for binding its target after activation (e.g., the difference in affinity for a target antigen (such as CTLA4) of an anti-CTLA4 activatable antibody before activation (e.g., before cleavage of the cleavage site) relative to the anti-CTLA4 activatable antibody after activation (e.g., after cleavage of the cleavage site)). In some embodiments, the masking unit (MU) binds to the target binding moiety (TBM), and prevents the anti-CTLA4 activatable antibody from binding to its target (e.g., an “inactive” activatable antibody). In some embodiments, the masking unit (MU) has a dissociation constant for binding to the target binding moiety (TBM) that is greater than the dissociation constant of the target binding moiety (TBM) for its target.

In some embodiments, the masking unit (MU) does not interfere with, obstruct, reduce the ability of, prevent, inhibit, or compete with the target binding moiety (TBM) for binding to its target after the anti-CTLA4 activatable antibody has been activated (e.g., activated by treatment with one or more proteases that cleave within the cleavage site, activated by a change in pH (increased or decreased), activated by a temperature shift (increased or decreased), activated after being contacted with a second molecule (such as an enzyme or a protein ligand), etc.). In some embodiments wherein the linkage unit (LU) comprises at least a first cleavage site, the masking unit (MU) does not interfere with, obstruct, reduce the ability of, prevent, inhibit, or compete with the target binding moiety (TBM) for binding its target after the linkage unit (LU) has been cleaved by one or more proteases that cleave within the cleavage site. In some embodiments, the masking unit (MU) has a masking efficiency of at most about 1.75 (e.g., at most about 1.75, at most about 1.5, at most about 1.4, at most about 1.3, at most about 1.2, at most about 1.1, at most about 1.0, at most about 0.9, at most about 0.8, at most about 0.7, at most about 0.6, or at most about 0.5, etc.) after activation (e.g., the relative affinity of the anti-CTLA4 activatable antibody after activation as compared to the affinity of a parental antibody).

In some embodiments, an anti-CTLA4 activatable antibody of the present disclosure contains a masking unit (MU) comprising a pair of cysteine residues at fixed positions to ensure that the anti-CTLA4 activatable antibodies have constrained conformations, and/or harbor few or no chemically labile residues (such as methionine or tryptophan). Advantageously, the inclusion of a pair of cysteine residues at fixed positions ensured that the anti-CTLA4 activatable antibodies had constrained conformations, tending to exhibit increased binding affinity and/or specificity. Furthermore, anti-CTLA4 activatable antibodies of the present disclosure included masking units with few to no unfavorable residues for manufacturing processes, such as methionine or tryptophan.

In some embodiments, anti-CTLA4 activatable antibodies of the present disclosure are context-dependent (e.g., are activated (are only capable of binding their targets) in certain contexts (such as in the protease-rich tumor microenvironment)). In some embodiments, the anti-CTLA4 activatable antibodies of the present disclosure provide improved safety over more traditional, non-activatable anti-CTLA4 antibodies (e.g., show reduced toxicity, do not induce significant alterations to the weights of many organs, do not alter liver histopathology, hematology, and/or blood biochemistry, etc.). In some embodiments, anti-CTLA4 activatable antibodies of the present disclosure have improved pharmacokinetic properties as compared to more traditional, non-activatable anti-CTLA4 antibodies (e.g., have longer in vivo half-lives).

### Anti-CTLA4 Activatable Antibody Activities

In some embodiments, the present disclosure relates to activatable antibodies that bind to human CTLA4 when in active form (e.g., the activatable antibodies are active after cleavage of the cleavage site (e.g., with one or more proteases)), but do not bind to CTLA4 prior to cleavage of the cleavage site (e.g., with one or more proteases). In some embodiments, the anti-CTLA4 activatable antibodies when in active form (e.g., after cleavage of the cleavage site) have at least one (e.g., at least one, at least two, at least three, at least four, at least five, at least six, at least seven, at least eight, or all nine) of the following functional properties: (a) bind to human, cynomolgus monkey, mouse, rat, and/or dog CTLA4 with a K_{D} of 500 nM or less, e.g., about 10 nM or less; (b) have antagonist activity on human CTLA4; (c) do not bind to human PD-1, PD-L1, PD-L2, LAG3, TIM3, B7-H3, CD95, CD120a, OX40, CD40, BTLA, VISTA, ICOS, and/or B7-H4 at concentration up to 100 nM; (d) are cross-reactive with monkey, mouse, rat, and/or dog CTLA4; (e) induces ADCC effects (e.g., on Tregs); (f) activates human PBMCs (e.g., stimulates secretion of IL-2 and/or IFNγ); (g) are capable of inhibiting tumor cell growth; (h) have therapeutic effect on a cancer; and (i) inhibit binding of human CTLA4 to human CD80 and/or human CD86. In certain embodiments wherein the linkage unit (LU) comprises at least a first cleavage site, the anti-CTLA4 activatable antibodies have at least one (e.g., at least one, at least two, at least three, at least four, or all five) of the following functional properties: (a) have a higher binding affinity to human, cynomolgus monkey, mouse, rat, and/or dog CTLA4, or any combination thereof, when the cleavage site is cleaved than when the cleavage site is not cleaved; (b) have increased antagonist activity on human CTLA4 when the cleavage site is cleaved than when the cleavage site is not cleaved; (c) induce ADCC effects (e.g., on Tregs) to a greater extent when the cleavage site is cleaved than when the cleavage site is not cleaved; (d) activates human PBMCs (e.g., stimulates secretion of IL-2 and/or IFNγ) to a greater extent when the cleavage site is cleaved than when the cleavage site is not cleaved; and (e) have a lower IC50 for inhibiting binding of human CTLA4 to human CD80 and/or human CD86 when the cleavage site is cleaved than when the cleavage site is not cleaved. Also provided herein are one or more anti-CTLA4 activatable antibodies that compete or cross-compete for binding to human CTLA4 with one or more of the CTLA4-targeting activatable antibodies and/or anti-CTLA4 antibodies described herein.

In some embodiments, the anti-CTLA4 activatable antibodies bind to human, cynomolgus monkey, mouse, rat, and/or dog CTLA4 with a K_{D} of about 500 nM or more when in inactive form (e.g., when the cleavage site is not cleaved). In some embodiments, the anti-CTLA4 activatable antibodies bind to human, cynomolgus monkey, mouse, rat, and/or dog CTLA4 with a K_{D} of about 500 nM or less (e.g., about 500 nM or less, about 450 nM or less, about 400 nM or less, about 350 nM or less, about 300 nM or less, about 250 nM or less, about 200 nM or less, about 150 nM or less, about 100 nM or less, about 90 nM or less, about 80 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) when in active form (e.g., when the cleavage site is cleaved). In some embodiments, the anti-CTLA4 activatable antibodies bind to human, cynomolgus monkey, mouse, rat, and/or dog CTLA4 with a K_{D} of about 350 nM or less when in active form (e.g., when the cleavage site is cleaved). In some embodiments, the anti-CTLA4 activatable antibodies bind to human CTLA4 with a K_{D} of about 100 nM or less when in active form (e.g., when the cleavage site is cleaved). In some embodiments, the anti-CTLA4 activatable antibodies bind to human CTLA4 with a K_{D} of about 50 nM or less when in active form (e.g., when the cleavage site is cleaved). In some embodiments, the anti-CTLA4 activatable antibodies bind to human CTLA4 with a K_{D} of about 10 nM or less when in active form (e.g., when the cleavage site is cleaved). Methods of measuring the K_{D} of an anti-CTLA4 activatable antibody may be carried out using any method known in the art, including for example, by surface plasmon resonance, an ELISA, isothermal titration calorimetry, a filter binding assay, an EMSA, etc. In some embodiments, the K_{D} is measured by an ELISA (see e.g., the Examples below).

In some embodiments, the anti-CTLA4 activatable antibodies do not have antagonist activity on human CTLA4 when in inactive form (e.g., when the cleavage site is not cleaved). In some embodiments, the anti-CTLA4 activatable antibodies have antagonist activity on human CTLA4 (e.g., induces ADCC effects (such as against Tregs), activates PBMCs (such as by activating, inducing, and/or stimulating IL-2 and/or IFNγ secretion), bocks binding of human CTLA4 to human CD80 and/or human CD86, etc.) when in active form (e.g., when the cleavage site is cleaved). In some embodiments, the anti-CTLA4 activatable antibodies repress one or more activities of human CTLA4 when in active form (e.g., repress one or more activities of human CTLA4 when a cell (such as a human cell) expressing human CTLA4 is contacted by an anti-CTLA4 activatable antibody when in active form (e.g., when the cleavage site is cleaved)).

In some embodiments, when in inactive form (e.g., when the cleavage site is not cleaved), the anti-CTLA4 activatable antibodies are not cross-reactive with monkey (e.g., cynomolgus monkey), mouse, rat, and/or dog CTLA4. In some embodiments, when in active form (e.g., when the cleavage site is cleaved), the anti-CTLA4 activatable antibodies are cross-reactive with monkey (e.g., cynomolgus monkey), mouse, rat, and/or dog CTLA4. In some embodiments, when in active form (e.g., when the cleavage site is cleaved), the anti-CTLA4 activatable antibodies are cross-reactive with monkey CTLA4. In some embodiments, when in active form (e.g., when the cleavage site is cleaved), the anti-CTLA4 activatable antibodies are cross-reactive with mouse CTLA4. In some embodiments, when in active form (e.g., when the cleavage site is cleaved), the anti-CTLA4 activatable antibodies are cross-reactive with rat CTLA4. In some embodiments, when in active form (e.g., when the cleavage site is cleaved), the anti-CTLA4 activatable antibodies are cross-reactive with dog CTLA4. In some embodiments, when in active form (e.g., when the cleavage site is cleaved), the anti-CTLA4 activatable antibodies are cross reactive with monkey and mouse CTLA4; monkey and rat CTLA4; monkey and dog CTLA4; mouse and rat CTLA4; mouse and dog CTLA4; rat and dog CTLA4; monkey, mouse, and rat CTLA4; monkey, mouse, and dog CTLA4; monkey, rat, and dog CTLA4; mouse, rat, and dog CTLA4; or monkey, mouse, rat, and dog CTLA4. In some embodiments, when in active form (e.g., when the cleavage site is cleaved), the anti-CTLA4 activatable binding polypeptides are cross-reactive at about 350 nM (e.g., at about 1 nM, at about 10 nM, at about 25 nM, at about 50 nM, at about 75 nM, at about 100 nM, at about 150 nM, at about 200 nM, at about 250 nM, at about 300 nM, at about 350 nM). Methods of measuring cross-reactivity are known in the art, including, without limitation, surface plasmon resonance, an ELISA, isothermal titration calorimetry, a filter binding assay, an EMSA, etc.

In some embodiments, the anti-CTLA4 activatable antibodies do not induce ADCC effects (e.g., against CTLA4-expressing human cells such as Tregs) when in inactive form (e.g., when the cleavage site is not cleaved). In some embodiments, the anti-CTLA4 activatable antibodies have reduced ADCC effects (e.g., against CTLA4-expressing human cells such as Tregs) when in inactive form (e.g., when the cleavage site is not cleaved) as compared to a control binding polypeptide (e.g., a parental antibody). In some embodiments, the anti-CTLA4 activatable antibodies induce ADCC effects (e.g., against CTLA4-expressing such as Tregs) when in active form (e.g., when the cleavage site is cleaved). Methods of measuring ADCC effects (e.g., in vitro methods) are known in the art, including, without limitation, via the methods described in the Examples below. In some embodiments, when in inactive form (e.g., when the cleavage site is not cleaved), the anti-CTLA4 activatable antibodies induce ADCC effects by less than about 10% (e.g., induce ADCC by less than about 10%, less than about 5%, less than about 1%, etc.) relative to a control (e.g., a parental antibody). In some embodiments, when in active form (e.g., when the cleavage site is cleaved), the anti-CTLA4 activatable antibodies induce ADCC effects by more than about 10% (e.g., induce ADCC by more than about 10%, more than about 15%, more than about 20%, more than about 25%, more than about 30%, more than about 35%, more than about 40%, etc.) relative to a control (e.g., an isotype control).

In some embodiments, the anti-CTLA4 activatable antibodies are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the anti-CTLA4 activatable antibodies relative to corresponding tumor cells not contacted with the anti-CTLA4 activatable antibodies (or relative to corresponding tumor cells contacted with an isotype control antibody). In some embodiments, the anti-CTLA4 activatable antibodies are capable of reducing tumor volume in a subject when the subject is administered the anti-CTLA4 activatable antibodies. In some embodiments, the anti-CTLA4 activatable antibodies are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the anti-CTLA4 activatable antibodies; as compared to a corresponding tumor in a subject administered an isotype control antibody). Methods of monitoring tumor cell growth/proliferation, tumor volume, and/or tumor inhibition are known in the art, including, for example, via the methods described in the Examples below.

In some embodiments, the anti-CTLA4 activatable antibodies have therapeutic effect on a cancer. In some embodiments, the anti-CTLA4 activatable antibodies reduce one or more signs or symptoms of a cancer. In some embodiments, a subject suffering from a cancer goes into partial or complete remission when administered the anti-CTLA4 activatable antibodies.

In some embodiments, the present disclosure provides isolated anti-CTLA4 activatable antibodies that, when in active form (e.g., when the cleavage site is cleaved), compete or cross-compete for binding to human CTLA4 with an antibody comprising: a) a CDR-H1 comprising the amino acid sequence of SEQ ID NO: 1, or a variant thereof comprising up to 5 (e.g., 1, 2, 3, 4, or 5) amino acid substitutions; a CDR-H2 comprising the amino acid sequence of SEQ ID NO: 2, or a variant thereof comprising up to 5 (e.g., 1, 2, 3, 4, or 5) amino acid substitutions; and a CDR-H3 comprising the amino acid sequence of SEQ ID NO: 3, or a variant thereof comprising up to 5 (e.g., 1, 2, 3, 4, or 5) amino acid substitutions; and/or b) a CDR-L1 comprising the amino acid sequence of SEQ ID NO: 4, or a variant thereof comprising up to 5 (e.g., 1, 2, 3, 4, or 5) amino acid substitutions; a CDR-L2 comprising the amino acid sequence of SEQ ID NO: 5, or a variant thereof comprising up to 5 (e.g., 1, 2, 3, 4, or 5) amino acid substitutions; and a CDR-L3 comprising the amino acid sequence of SEQ ID NO: 6, or a variant thereof comprising up to 5 (e.g., 1, 2, 3, 4, or 5) amino acid substitutions. In some embodiments, the present disclosure provides isolated anti-CTLA4 activatable antibodies that, when in active form (e.g., when the cleavage site is cleaved), compete or cross-compete for binding to human CTLA4 with an antibody comprising: a) a heavy chain variable region comprising the amino acid sequence of SEQ ID NO: 74, or an amino acid sequence having at least 80% (e.g., at least 85%, 90%, 95%, 98%, or 99%; or 100%) sequence identity with SEQ ID NO: 74; and/or b) a light chain variable region comprising the amino acid sequence of SEQ ID NO: 75, or an amino acid sequence having at least 80% (e.g., at least 85%, 90%, 95%, 98%, or 99%; or 100%) sequence identity with SEQ ID NO: 75.

In some embodiments, the present disclosure provides isolated anti-CTLA4 activatable antibodies that, when in active form (e.g., when the cleavage site is cleaved), compete or cross-compete for binding to human CTLA4 with an antibody comprising: a) a CDR-H1 comprising the amino acid sequence of SEQ ID NO: 12, or a variant thereof comprising up to 5 (e.g., 1, 2, 3, 4, or 5) amino acid substitutions; a CDR-H2 comprising the amino acid sequence of SEQ ID NO: 13, or a variant thereof comprising up to 5 (e.g., 1, 2, 3, 4, or 5) amino acid substitutions; and a CDR-H3 comprising the amino acid sequence of SEQ ID NO: 14, or a variant thereof comprising up to 5 (e.g., 1, 2, 3, 4, or 5) amino acid substitutions; and/or b) a CDR-L1 comprising the amino acid sequence of SEQ ID NO: 15, or a variant thereof comprising up to 5 (e.g., 1, 2, 3, 4, or 5) amino acid substitutions; a CDR-L2 comprising the amino acid sequence of SEQ ID NO: 16, or a variant thereof comprising up to 5 (e.g., 1, 2, 3, 4, or 5) amino acid substitutions; and a CDR-L3 comprising the amino acid sequence of SEQ ID NO: 17, or a variant thereof comprising up to 5 (e.g., 1, 2, 3, 4, or 5) amino acid substitutions. In some embodiments, the present disclosure provides isolated anti-CTLA4 activatable antibodies that, when in active form (e.g., when the cleavage site is cleaved), compete or cross-compete for binding to human CTLA4 with an antibody comprising: a) a heavy chain variable region comprising the amino acid sequence of SEQ ID NO: 66, or an amino acid sequence having at least 80% (e.g., at least 85%, 90%, 95%, 98%, or 99%; or 100%) sequence identity with SEQ ID NO: 66; and/or b) a light chain variable region comprising the amino acid sequence of SEQ ID NO: 67, or an amino acid sequence having at least 80% (e.g., at least 85%, 90%, 95%, 98%, or 99%; or 100%) sequence identity with SEQ ID NO: 67.

In some embodiments, the present disclosure provides isolated anti-CTLA4 activatable antibodies that, when in active form (e.g., when the cleavage site is cleaved), compete or cross-compete for binding to human CTLA4 with an antibody comprising: a) a CDR-H1 comprising the amino acid sequence of SEQ ID NO: 44, or a variant thereof comprising up to 5 (e.g., 1, 2, 3, 4, or 5) amino acid substitutions; a CDR-H2 comprising the amino acid sequence of SEQ ID NO: 45, or a variant thereof comprising up to 5 (e.g., 1, 2, 3, 4, or 5) amino acid substitutions; and a CDR-H3 comprising the amino acid sequence of SEQ ID NO: 53, or a variant thereof comprising up to 5 (e.g., 1, 2, 3, 4, or 5) amino acid substitutions; and/or b) a CDR-L1 comprising the amino acid sequence of SEQ ID NO: 54, or a variant thereof comprising up to 5 (e.g., 1, 2, 3, 4, or 5) amino acid substitutions; a CDR-L2 comprising the amino acid sequence of SEQ ID NO: 16, or a variant thereof comprising up to 5 (e.g., 1, 2, 3, 4, or 5) amino acid substitutions; and a CDR-L3 comprising the amino acid sequence of SEQ ID NO: 55, or a variant thereof comprising up to 5 (e.g., 1, 2, 3, 4, or 5) amino acid substitutions. In some embodiments, the present disclosure provides isolated anti-CTLA4 activatable antibodies that, when in active form (e.g., when the cleavage site is cleaved), compete or cross-compete for binding to human CTLA4 with an antibody comprising: a) a heavy chain variable region comprising the amino acid sequence of SEQ ID NO: 84, or an amino acid sequence having at least 80% (e.g., at least 85%, 90%, 95%, 98%, or 99%; or 100%) sequence identity with SEQ ID NO: 84; and/or b) a light chain variable region comprising the amino acid sequence of SEQ ID NO: 85, or an amino acid sequence having at least 80% (e.g., at least 85%, 90%, 95%, 98%, or 99%; or 100%) sequence identity with SEQ ID NO: 85.

In some embodiments, the present disclosure provides isolated anti-CTLA4 activatable antibodies that, when in active form (e.g., when the cleavage site is cleaved), compete or cross-compete for binding to human CTLA4 with an antibody comprising: a) a CDR-H1 comprising the amino acid sequence of SEQ ID NO: 44, or a variant thereof comprising up to 5 (e.g., 1, 2, 3, 4, or 5) amino acid substitutions; a CDR-H2 comprising the amino acid sequence of SEQ ID NO: 45, or a variant thereof comprising up to 5 (e.g., 1, 2, 3, 4, or 5) amino acid substitutions; and a CDR-H3 comprising the amino acid sequence of SEQ ID NO: 46, or a variant thereof comprising up to 5 (e.g., 1, 2, 3, 4, or 5) amino acid substitutions; and/or b) a CDR-L1 comprising the amino acid sequence of SEQ ID NO: 47, or a variant thereof comprising up to 5 (e.g., 1, 2, 3, 4, or 5) amino acid substitutions; a CDR-L2 comprising the amino acid sequence of SEQ ID NO: 5, or a variant thereof comprising up to 5 (e.g., 1, 2, 3, 4, or 5) amino acid substitutions; and a CDR-L3 comprising the amino acid sequence of SEQ ID NO: 48, or a variant thereof comprising up to 5 (e.g., 1, 2, 3, 4, or 5) amino acid substitutions. In some embodiments, the present disclosure provides isolated anti-CTLA4 activatable antibodies that, when in active form (e.g., when the cleavage site is cleaved), compete or cross-compete for binding to human CTLA4 with an antibody comprising: a) a heavy chain variable region comprising the amino acid sequence of SEQ ID NO: 80, or an amino acid sequence having at least 80% (e.g., at least 85%, 90%, 95%, 98%, or 99%; or 100%) sequence identity with SEQ ID NO: 80; and/or b) a light chain variable region comprising the amino acid sequence of SEQ ID NO: 81, or an amino acid sequence having at least 80% (e.g., at least 85%, 90%, 95%, 98%, or 99%; or 100%) sequence identity with SEQ ID NO: 81.

The ability of an activatable antibody to compete or cross-compete for binding with an antibody can be determined using standard binding assays known in the art, such as BIAcore analysis, ELISA assays, or flow cytometry. For example, one can allow an antibody (e.g., as described above) to bind to human CTLA4 under saturating conditions and then measure the ability of the test activatable antibody (when in active form, e.g., when the cleavage site is cleaved) to bind to the CTLA4. If the test activatable antibody is able to bind to the CTLA4 at the same time as the antibody, then the test activatable antibody binds to a different epitope then the antibody. However, if the test activatable antibody is not able to bind to the CTLA4 at the same time, then the test activatable antibody binds to the same epitope, an overlapping epitope, or an epitope that is in close proximity to the epitope bound by the antibody. This experiment can be performed using various methods, such as ELISA, RIA, FACS or surface plasmon resonance.

In some embodiments, the anti-CTLA4 activatable antibodies (when in inactive form, e.g., when the cleavage site is not cleaved) do not inhibit the binding between CTLA4 and one or more of its binding partners (e.g., human CTLA4 and human CD80, human CTLA4 and human CD86). In some embodiments, the anti-CTLA4 activatable antibodies (when in active form e.g., when the cleavage site is cleaved) inhibit the binding between CTLA4 and one or more of its binding partners (e.g., human CTLA4 and human CD80, human CTLA4 and human CD86). In some embodiments, the anti-CTLA4 activatable antibodies inhibit the binding between CTLA4 and its ligand in vitro. In some embodiments wherein the linkage unit (LU) comprises at least a first cleavage site, the anti-CTLA4 activatable antibodies inhibit the binding between CTLA4 and its ligand in vitro when the cleavage site is cleaved. In some embodiments wherein the linkage unit (LU) comprises at least a first cleavage site, the anti-CTLA4 activatable antibodies have a half maximal inhibitory concentration (IC₅₀) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for inhibiting binding of CTLA4 to CD80 and/or CD86 when the cleavage site is cleaved. In some embodiments wherein the linkage unit (LU) comprises at least a first cleavage site, the anti-CTLA4 activatable antibodies have a half maximal inhibitory concentration (IC₅₀) of about 100 nM or less for inhibiting binding of CTLA4 to CD80 and/or CD86 when the cleavage site is cleaved. In some embodiments, the anti-CTLA4 activatable antibodies completely inhibit binding of human CTLA4 to CD80 and/or CD86 when provided at a concentration of about 100 nM or greater (e.g., about 100 nM or greater, about 500 nM or greater, about 1 μM or greater, about 10 μM or greater, etc.). As used herein, the term “complete inhibiting” or “completely inhibits” refers to the anti-CTLA4 activatable antibody's ability to reduce binding between a first protein and a second protein by at least about 80% (e.g., at least about 80%, at least about 85%, at least about 90%, at least about 95%, at least about 99%, etc.). Methods of measuring the ability of an a polypeptide to inhibit binding of a first protein (e.g., human CTLA4) and a second protein (e.g., human CD80 or human CD86) are known in the art, including, without limitation, via BIAcore analysis, ELISA assays, and flow cytometry.

### Linkage Units (LUs) of Activatable Antibodies

In some embodiments, the present disclosure relates to anti-CTLA4 masked antibodies (e.g., activatable antibodies) comprising a linkage unit (LU). In some embodiments, the linkage unit (LU) is cleaved and/or disrupted by treatment with one or more proteases that cleave within the a cleavage site of the LU, by a change in pH (increased or decreased), by a temperature shift (increased or decreased), and/or by contact with a second molecule (such as a small molecule or a protein ligand), etc.).

In some embodiments, the linkage unit (LU) comprises at least a first cleavage site (C₁) (e.g., a first protease cleavage site). In some embodiments, the first cleavage site is a first protease cleavage site. Any suitable protease cleavage site recognized and/or cleaved by any protease (e.g., a protease that is known to be co-localized with a target of an anti-CTLA4 masked antibody (e.g., an activatable antibody) comprising the LU) known in the art may be used, including, for example, a protease cleavage site recognized and/or cleaved by urokinase-type plasminogen activator (uPA); matrix metalloproteinases (e.g., MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-23, MMP-24, MMP-26, and/or MMP-27); Tobacco Etch Virus (TEV) protease; plasmin; Thrombin; PSA; PSMA; ADAMS/ADAMTS (e.g., ADAM 8, ADAM 9, ADAM10, ADAM12, ADAM15, ADAM17/TACE, ADAMDEC1, ADAMTS1, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK11, KLK13, and/or KLK14); metallo proteainases (e.g., Meprin, Neprilysin, PSMA, and/or BMP-1); serine proteases (e.g., activated protein C, Cathepsin A, Cathepsin G, Chymase, and/or coagulation factor proteases (such as FVIIa, FIXa, FXa, FXIa, FXIIa)); elastase; granzyme B; guanidinobenzoatase; HtrA1; human neutrophil elastase; lactoferrin; marapsin; NS3/4A; PACE4; tPA; tryptase; type II transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc. In some embodiments, the first protease cleavage site is a cleavage site for a protease selected from uPA, MMP-1, MMP-2, MMP-3, MMP-8, MMP-9, MMP-14, TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the first protease cleavage site is a cleavage site for a protease selected from uPA, MMP-2, MMP-9, and/or TEV protease. In some embodiments, the cleavage site comprises an amino acid sequence selected from SGRSA (SEQ ID NO: 123), PLGLAG (SEQ ID NO: 124), and ENLYFQG (SEQ ID NO: 125). In some embodiments, the cleavage site comprises an amino acid sequence selected from the group consisting of SEQ ID NOs: 152-154. In some embodiments, the cleavage site comprises an amino acid sequence selected from the group consisting of SEQ ID NOs: 123-125 and 152-154. In some embodiments, the cleavage site is a cleavage site listed in Table 5D.

Exemplary linkage units (LUs) are provided in the Examples (see, e.g., Example 2, Table 5E). In some embodiments, the LU comprises the amino acid sequence of SEQ ID NO: 99. In some embodiments, the LU comprises the amino acid sequence of SEQ ID NO: 100. In some embodiments, the LU comprises the amino acid sequence of SEQ ID NO: 101. In some embodiments, the LU comprises the amino acid sequence of SEQ ID NO: 102. In some embodiments, the LU comprises the amino acid sequence of SEQ ID NO: 103. In some embodiments, the LU comprises an amino acid sequence selected from the group consisting of SEQ ID NO: 99-103.

In some embodiments, the linkage unit (LU) further comprises a first linker (L₁). In some embodiments, the first linker (L₁) is C-terminal to the first cleavage site (C₁) (e.g., a first protease cleavage site). In some embodiments, the linkage unit (LU) comprises a structure, from N-terminus to C-terminus, of: (C₁)-L₁.

Any suitable linker (e.g., a flexible linker) known in the art may be used, including, for example: glycine polymers (G) n, where n is an integer of at least 1 (e.g., at least one, at least 2, at least 3, at least 4, at least 5, at least 6, at least 7, at least 8, at least 9, at least 10, etc.); glycine-serine polymers (GS) n, where n is an integer of at least 1 (e.g., at least one, at least 2, at least 3, at least 4, at least 5, at least 6, at least 7, at least 8, at least 9, at least 10, etc.) such as GGGGS (SEQ ID NO: 126), SGGS (SEQ ID NO: 127), GGSG (SEQ ID NO: 128), GGSGG (SEQ ID NO: 129), GSGSG (SEQ ID NO: 130), GSGGG (SEQ ID NO: 131), GGGSG (SEQ ID NO: 132), and/or GSSSG (SEQ ID NO: 133)); glycine-alanine polymers; alanine-serine polymers; and the like. Linker sequences may be of any length, such as from about 1 amino acid (e.g., glycine or serine) to about 20 amino acids (e.g., 20 amino acid glycine polymers or glycine-serine polymers), about 1 amino acid to about 15 amino acids, about 3 amino acids to about 12 amino acids, about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length. In some embodiments, the linker comprises an amino acid sequence selected from SEQ ID NOS: 129-133. In some embodiments, the linker comprises an amino acid sequence of SEQ ID NO: 126 or 127. In some embodiments, the linker comprises an amino acid sequence selected from SEQ ID NOs: 155-161. In certain embodiments, the linker is a linker described in Table 5D.

In some embodiments, linkage unit (LU) further comprises at least a second cleavage site (e.g., at least a second, at least a third, at least a fourth, at least a fifth, etc.). In some embodiments, the linkage unit (LU) further comprises a second cleavage site (C2). In some embodiments, the second cleavage site is a second protease cleavage site. The second protease cleavage site may be any suitable protease cleavage site recognized and/or cleaved by any of the proteases described above. In some embodiments, the first (C₁) and second (C₂) cleavage sites are protease cleavage sites recognized and/or cleaved by the same protease. In some embodiments, the first (C₁) and second (C₂) cleavage sites are protease cleavage sites recognized and/or cleaved by different proteases (e.g., the first protease cleavage site is recognized and/or cleaved by uPA, and the second protease cleavage site is recognized and/or cleaved by MMP-2; the first protease cleavage site is recognized and/or cleaved by uPA, and the second protease cleavage site is recognized and/or cleaved by MMP-9; the first protease cleavage site is recognized and/or cleaved by uPA, and the second protease cleavage site is recognized and/or cleaved by TEV protease; etc.). In some embodiments, the at least second cleavage site (C₂) is C-terminal to the first linker (L₁). In some embodiments, the linkage unit (LU) comprises a structure, from N-terminus to C-terminus, of: (C₁)-L₁-(C₂).

In some embodiments, the linkage unit (LU) further comprises at least a second linker (e.g., at least a second, at least a third, at least a fourth, at least a fifth, etc.). In some embodiments, the linkage unit (LU) further comprises a second linker (L₂). The second linker (L₂) may be any suitable linker described above. In some embodiments, the second linker comprises an amino acid sequence selected from SEQ ID NO: 126-133. In some embodiments, the first (L₁) and second (L₂) linkers are the same (e.g., both linkers comprise the sequence of SEQ ID NO: 126 or 127). In some embodiments, the first (L₁) and second (L₂) linkers are different (e.g., the first linker (L₁) comprises the amino acid sequence of SEQ ID NO: 156, and the second linker (L₂) comprises the amino acid sequence of SEQ ID NO: 157, etc.). In some embodiments, the at least second linker (L₂) is C-terminal to the second cleavage site (C₂). In some embodiments, the linkage unit (LU) comprises a structure, from N-terminus to C-terminus, of: C₁-L₁-C₂-L₂. In some embodiments, the first linker (L₁) is N-terminal to the first cleavage site (C₁), and the second linker (L₂) is C-terminal to the first cleavage site (C₁). In some embodiments, the linkage unit (LU) comprises a structure, from N-terminus to C-terminus, of: L₁-C₁-L₂.

### Exemplary MU-LU Sequences of Masked Anti-CTLA4 Antibodies

Exemplary combinations of masking unit (MU) and linkage unit (LU) sequences of masked anti-CTLA4 antibodies are described in Example 2 (see, e.g., Tables 5A-5E).

In some embodiments, an activatable antibody of the present disclosure comprises a MU having an amino acid sequence selected from the group consisting of SEQ ID NOs: 146-151, or a variant thereof comprising one or more (e.g., 1, 2, 3, 4, 5, or 6) amino acid substitutions. In certain embodiments, an activatable antibody of the present disclosure comprises an MU having an amino acid sequence of any of the exemplary antibodies shown in Tables 5A-5C, or a variant thereof comprising one or more (e.g., 1, 2, 3, 4, 5, or 6) amino acid substitutions. In some embodiments, the activatable antibody further comprise an N-terminal unit linked to the N-terminus of the MU. In some embodiments, the N-terminal unit comprises an amino acid sequence selected from the group consisting of SEQ ID NOs: 143-146. In certain embodiments, the activatable antibody comprises an N-terminal unit of any of the exemplary antibodies shown in Tables 5A-5C. In some embodiments, the activatable antibody comprises a masking peptide comprising, from N-terminus to C-terminus, an N-terminal unit and a masking unit (MU), wherein the N-terminal unit and the MU together comprise an amino acid sequence selected from the group consisting of SEQ ID NOs: 92-98. In certain embodiments, the N-terminal unit and the MU together comprise an amino acid sequence of any of the exemplary antibodies described in Table 5E.

In some embodiments, an activatable antibody of the present disclosure comprises a LU having an amino acid sequence selected from the group consisting of SEQ ID NOs: 99-103 and 161, or a variant thereof comprising one or more (e.g., 1, 2, 3, 4, 5, or 6) amino acid substitutions. In certain embodiments, an activatable antibody of the present disclosure comprises an LU sequence of any of the Exemplary antibodies shown in Table 5E or a variant thereof comprising one or more (e.g., 1, 2, 3, 4, 5, or 6) amino acid substitutions. In some embodiments, the activatable antibody comprises an LU comprising at least a first cleavage site (e.g., a first cleavage site, or a first cleavage site and a second cleavage site). In some embodiments, the first cleavage site and/or the second cleavage site has an amino acid sequence selected from the group consisting of SEQ ID NOs: 152-154. In certain embodiments, the first cleavage site and/or the second cleavage is a cleavage site of any of the exemplary antibodies shown in Tables 5A-5C and 5E. In some embodiments, the LU further comprises one or more linkers (e.g., a first linker, or a first linker and second linker). In some embodiments, the first linker and/or the second linker comprise an amino acid sequence selected from the group consisting of SEQ ID NOs: 155-161. In certain embodiments, the first linker and/or the second linker is a linker of any one of the exemplary antibodies shown in Tables 5A-5C and 5E. In some embodiments, the LU comprises, from N-terminus to C-terminus the first cleavage site (C₁), the first linker (L₁), the second cleavage site (C₂), and the second linker (L₂) (i.e., a C₁-L₁-C₂-L₂ configuration). In some embodiments, the linkage unit (LU) comprises, from N-terminus to C-terminus the first linker (L₁), the first cleavage site (C₁), and the second linker (L₂) (i.e., a L₁-C₁-L₂ configuration).

In some embodiments, an activatable antibody of the present disclosure comprises a MU comprising the amino acid sequence of SEQ ID NO: 146, and a LU comprising the amino acid sequence of SEQ ID NO: 99. In some embodiments, an activatable antibody of the present disclosure comprises a MU comprising the amino acid sequence of SEQ ID NO: 147, and a LU comprising the amino acid sequence of SEQ ID NO: 99. In some embodiments, an activatable antibody of the present disclosure comprises a MU comprising the amino acid sequence of SEQ ID NO: 148, and a LU comprising the amino acid sequence of SEQ ID NO: 99. In some embodiments, an activatable antibody of the present disclosure comprises a MU comprising the amino acid sequence of SEQ ID NO: 149, and a LU comprising the amino acid sequence of SEQ ID NO: 99. In some embodiments, an activatable antibody of the present disclosure comprises a MU comprising the amino acid sequence of SEQ ID NO: 150, and a LU comprising the amino acid sequence of SEQ ID NO: 99. In some embodiments, an activatable antibody of the present disclosure comprises a MU comprising the amino acid sequence of SEQ ID NO: 151, and a LU comprising the amino acid sequence of SEQ ID NO: 99. In some embodiments, an activatable antibody of the present disclosure comprises a MU comprising the amino acid sequence of SEQ ID NO: 148, and a LU comprising the amino acid sequence of SEQ ID NO: 100. In some embodiments, an activatable antibody of the present disclosure comprises a MU comprising the amino acid sequence of SEQ ID NO: 148, and a LU comprising the amino acid sequence of SEQ ID NO: 101. In some embodiments, an activatable antibody of the present disclosure comprises a MU comprising the amino acid sequence of SEQ ID NO: 148, and a LU comprising the amino acid sequence of SEQ ID NO: 102. In some embodiments, an activatable antibody of the present disclosure comprises a MU comprising the amino acid sequence of SEQ ID NO: 148, and a LU comprising the amino acid sequence of SEQ ID NO: 101. In some embodiments, an activatable antibody of the present disclosure comprises a MU comprising the amino acid sequence of SEQ ID NO: 150, and a LU comprising the amino acid sequence of SEQ ID NO: 100. In some embodiments, an activatable antibody of the present disclosure comprises a MU comprising the amino acid sequence of SEQ ID NO: 151, and a LU comprising the amino acid sequence of SEQ ID NO: 100. In some embodiments, an activatable antibody of the present disclosure comprises a MU comprising the amino acid sequence of SEQ ID NO: 150, and a LU comprising the amino acid sequence of SEQ ID NO: 101. In some embodiments, an activatable antibody of the present disclosure comprises a MU comprising the amino acid sequence of SEQ ID NO: 151, and a LU comprising the amino acid sequence of SEQ ID NO: 101. In some embodiments, an activatable antibody of the present disclosure comprises a MU comprising the amino acid sequence of SEQ ID NO: 150, and a LU comprising the amino acid sequence of SEQ ID NO: 102. In some embodiments, an activatable antibody of the present disclosure comprises a MU comprising the amino acid sequence of SEQ ID NO: 151, and a LU comprising the amino acid sequence of SEQ ID NO: 102. In some embodiments, an activatable antibody of the present disclosure comprises a MU comprising the amino acid sequence of SEQ ID NO: 150, and a LU comprising the amino acid sequence of SEQ ID NO: 103. In some embodiments, an activatable antibody of the present disclosure comprises a MU comprising the amino acid sequence of SEQ ID NO: 151, and a LU comprising the amino acid sequence of SEQ ID NO: 103.

In some embodiments, an activatable antibody of the present disclosure comprises a masking peptide having an amino acid sequence of any of the exemplary antibodies shown in Table 5F. In some embodiments, the activatable antibody comprises a masking peptide comprising an amino acid sequence selected from the group consisting of SEQ ID NOs: 162-177, or a variant thereof comprising one or more (e.g., 1, 2, 3, 4, 5, 6, or more) amino acid substitutions.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24148, as described in Tables 5A, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 146, a first cleavage site (C₁) comprising the amino acid sequence of SEQ ID NO: 152, a first linker (L₁) comprising the amino acid sequence of SEQ ID NO: 155, a second cleavage site (C₂) comprising the amino acid sequence of SEQ ID NO: 154, and a second linker (L₂) comprising the amino acid sequence of SEQ ID NO: 160. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 92, and an LU comprising the amino acid sequence of SEQ ID NO: 99. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 162.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24149, as described in Tables 5A, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 147, a first cleavage site (C₁) comprising the amino acid sequence of SEQ ID NO: 152, a first linker (L₁) comprising the amino acid sequence of SEQ ID NO: 155, a second cleavage site (C₂) comprising the amino acid sequence of SEQ ID NO: 154, and a second linker (L₂) comprising the amino acid sequence of SEQ ID NO: 160. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 93, and an LU comprising the amino acid sequence of SEQ ID NO: 99. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 163.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24155, as described in Tables 5A, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 148, a first cleavage site (C₁) comprising the amino acid sequence of SEQ ID NO: 152, a first linker (L₁) comprising the amino acid sequence of SEQ ID NO: 155, a second cleavage site (C₂) comprising the amino acid sequence of SEQ ID NO: 154, and a second linker (L₂) comprising the amino acid sequence of SEQ ID NO: 160. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 94, and an LU comprising the amino acid sequence of SEQ ID NO: 99. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 164.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24157, as described in Tables 5A, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 149, a first cleavage site (C₁) comprising the amino acid sequence of SEQ ID NO: 152, a first linker (L₁) comprising the amino acid sequence of SEQ ID NO: 155, a second cleavage site (C₂) comprising the amino acid sequence of SEQ ID NO: 154, and a second linker (L₂) comprising the amino acid sequence of SEQ ID NO: 160. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 95, and an LU comprising the amino acid sequence of SEQ ID NO: 99. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 165.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24158, as described in Tables 5A, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 150, a first cleavage site (C₁) comprising the amino acid sequence of SEQ ID NO: 152, a first linker (L₁) comprising the amino acid sequence of SEQ ID NO: 155, a second cleavage site (C₂) comprising the amino acid sequence of SEQ ID NO: 154, and a second linker (L₂) comprising the amino acid sequence of SEQ ID NO: 160. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 96, and an LU comprising the amino acid sequence of SEQ ID NO: 99. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 166.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24459, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 148, a first linker (L1) comprising the amino acid sequence of SEQ ID NO: 153, a first cleavage site (C1) comprising the amino acid sequence of SEQ ID NO: 154, and a second linker (L2) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 94, and an LU comprising the amino acid sequence of SEQ ID NO: 100. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 167.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24463, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 151, a first linker (L1) comprising the amino acid sequence of SEQ ID NO: 153, a first cleavage site (C1) comprising the amino acid sequence of SEQ ID NO: 154, and a second linker (L2) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 96, and an LU comprising the amino acid sequence of SEQ ID NO: 100. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 171.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24460, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 148, a first linker (L₁) comprising the amino acid sequence of SEQ ID NO: 157, a first cleavage site (C₁) comprising the amino acid sequence of SEQ ID NO: 154, and a second linker (L₂) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 94, and an LU comprising the amino acid sequence of SEQ ID NO: 101. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 168.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24461, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 148, a first linker (L₁) comprising the amino acid sequence of GGG, a first cleavage site (C₁) comprising the amino acid sequence of SEQ ID NO: 154, and a second linker (L₂) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 94, and an LU comprising the amino acid sequence of SEQ ID NO: 102. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 169.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24462, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of E, an MU comprising the amino acid sequence of SEQ ID NO: 148, a first linker (L₁) comprising the amino acid sequence of SEQ ID NO: 157, a first cleavage site (C₁) comprising the amino acid sequence of SEQ ID NO: 154, and a second linker (L₂) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 97, and an LU comprising the amino acid sequence of SEQ ID NO: 101. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 170.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24464, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 151, a first linker (L₁) comprising the amino acid sequence of SEQ ID NO: 157, a first cleavage site (C₁) comprising the amino acid sequence of SEQ ID NO: 154, and a second linker (L₂) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 96, and an LU comprising the amino acid sequence of SEQ ID NO: 101. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 172.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24466, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of E, an MU comprising the amino acid sequence of SEQ ID NO: 150, a first linker (L₁) comprising the amino acid sequence of SEQ ID NO: 157, a first cleavage site (C₁) comprising the amino acid sequence of SEQ ID NO: 154, and a second linker (L₂) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 98, and an LU comprising the amino acid sequence of SEQ ID NO: 101. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 173.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24465, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 143, an MU comprising the amino acid sequence of SEQ ID NO: 150, a first linker (L₁) comprising the amino acid sequence of GGG, a first cleavage site (C₁) comprising the amino acid sequence of SEQ ID NO: 154, and a second linker (L₂) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 96, and an LU comprising the amino acid sequence of SEQ ID NO: 102. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 174.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24800, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of E, an MU comprising the amino acid sequence of SEQ ID NO: 150, a first linker (L₁) comprising the amino acid sequence of GGG, a first cleavage site (C₁) comprising the amino acid sequence of SEQ ID NO: 154, and a second linker (L₂) comprising the amino acid sequence of GS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 98, and an LU comprising the amino acid sequence of SEQ ID NO: 102. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 175.

In some embodiments, provided herein a activatable antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24649 or TY24652, as described in Tables 5B, 5E, and 5F. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of E, an MU comprising the amino acid sequence of SEQ ID NO: 150, a first linker (L₁) comprising the amino acid sequence of GGG, a first cleavage site (C₁) comprising the amino acid sequence of SEQ ID NO: 154, and a second linker (L₂) comprising the amino acid sequence of GGS. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 98, and an LU comprising the amino acid sequence of SEQ ID NO: 103. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 176.

In some embodiments, provided herein a masked antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises an amino acid sequence of antibody TY24851 or TY24841, as described in Tables 5C, 5E, and 5F. In some embodiments, provided herein a masked antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit comprising the amino acid sequence of SEQ ID NO: 144, an MU comprising the amino acid sequence of SEQ ID NO: 150, and a first linker (L₁) comprising the amino acid sequence of SEQ ID NO: 159. In some embodiments, provided herein a masked antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises, from N-terminus to C-terminus, an N-terminal unit and an MU together comprising the amino acid sequence of SEQ ID NO: 98, and an LU comprising the amino acid sequence of SEQ ID NO: 159. In some embodiments, provided herein a activatable antibody that binds to human CTLA4, comprising an MP and an antibody, wherein the antibody comprises a heavy chain comprising a VH and a light chain comprising a VL, wherein the MP is linked to the N-terminal of the VH or VL, wherein the MP comprises the amino acid sequence of SEQ ID NO: 177.

### Human IgG1 Fc Regions of Anti-CTLA4 Masked Antibodies

In some embodiments, the anti-CTLA4 masked antibody (e.g., activatable antibody) comprises a human IgG1 Fc region. In some embodiments, the human IgG1 Fc region comprises one or more amino acid substitutions that confer enhanced effector function. In some embodiments, the human IgG1 Fc region comprises one or more amino acid substitutions that enhance ADCC. In some embodiments, the human IgG1 Fc region comprises one or more (e.g., 1, 2, 3, 4, 5, 6, or more) substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering.

In some embodiments, the human IgG1 Fc region comprises an S298A substitution, an E333A substitution, and a K334A substitution, wherein the residue numbering is according to EU numbering. In certain embodiments, the human IgG1 Fc region comprises an S298A substitution, an E333A substitution, and a K334A substitution, and does not comprise one or more (e.g., 1, 2, 3, 4, 5, 6, 7, 8, 9 or any) of a L235V substitution, a S239D substitution, an F243L substitution, an R292P substitution, a Y300L substitution, a V305I substitution, a K326A substitution, an A330L substitution, an I332E substitution, and a P396L substitution, wherein the residue numbering is according to EU numbering.

In some embodiments, the human IgG1 Fc region comprises an S298A substitution, an E333A substitution, a K334A substitution, and a K326A substitution, wherein the residue numbering is according to EU numbering. In some embodiments, the human IgG1 Fc region comprises an S298A substitution, an E333A substitution, a K334A substitution, and a K326A substitution, and does not comprise one or more (e.g., 1, 2, 3, 4, 5, 6, 7, 8, or any) of a L235V substitution, a S239D substitution, an F243L substitution, an R292P substitution, a Y300L substitution, a V305I substitution, an A330L substitution, an I332E substitution, and a P396L substitution, wherein the residue numbering is according to EU numbering.

In some embodiments, the human IgG1 Fc region comprises an F243L substitution, wherein the residue numbering is according to EU numbering. In some embodiments, the human IgG1 Fc region comprises an F243L substitution, and does not comprise one or more (e.g., 1, 2, 3, 4, 5, 6, 7, 8, 9, 10, 11, or any) of a L235V substitution, a S239D substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering.

In some embodiments, the human IgG1 Fc region comprises an F243L substitution, an R292P substitution, a Y300L substitution, and a P396L substitution, wherein the residue numbering is according to EU numbering. In some embodiments, the human IgG1 Fc region comprises an F243L substitution, an R292P substitution, a Y300L substitution, and a P396L substitution, and does not comprise one or more (e.g., 1, 2, 3, 4, 5, 6, 7, 8, or any) of a L235V substitution, a S239D substitution, an S298A substitution, a V305I substitution, a K326A substitution, an A330L substitution, an I332E substitution, an E333A substitution, and a K334A substitution, wherein the residue numbering is according to EU numbering.

In some embodiments, the human IgG1 Fc region comprises an F243L substitution, an R292P substitution, a Y300L substitution, a V305I substitution, and a P396L substitution, wherein the residue numbering is according to EU numbering. In some embodiments, the human IgG1 Fc region comprises an F243L substitution, an R292P substitution, a Y300L substitution, a V305I substitution, and a P396L substitution, and does not comprise one or more (e.g., 1, 2, 3, 4, 5, 6, 7, or any) of a L235V substitution, a S239D substitution, an S298A substitution, a K326A substitution, an A330L substitution, an I332E substitution, an E333A substitution, and a K334A substitution, wherein the residue numbering is according to EU numbering.

In some embodiments, the human IgG1 Fc region comprises an F243L substitution, an R292P substitution, a Y300L substitution, a L235V substitution, and a P396L substitution, wherein the residue numbering is according to EU numbering. In some embodiments, the human IgG1 Fc region comprises an F243L substitution, an R292P substitution, a Y300L substitution, a L235V substitution, and a P396L substitution, and does not comprise does not comprise one or more (e.g., 1, 2, 3, 4, 5, 6, 7, or any) of a S239D substitution, an S298A substitution, a V305I substitution, a K326A substitution, an A330L substitution, an I332E substitution, an E333A substitution, and a K334A substitution, wherein the residue numbering is according to EU numbering.

In some embodiments, the human IgG1 Fc region comprises a S239D substitution, an I332E substitution, and an A330L substitution, wherein the residue numbering is according to EU numbering. In some embodiments, the human IgG1 Fc region comprises a S239D substitution, an I332E substitution, and an A330L substitution, and does not comprise does not comprise one or more (e.g., 1, 2, 3, 4, 5, 6, 7, 8, 9, or any) of a L235V substitution, an F243L substitution, an R292P substitution, an S298A substitution, a Y300L substitution, a V305I substitution, a K326A substitution, an E333A substitution, a K334A substitution, and a P396L substitution, wherein the residue numbering is according to EU numbering.

In some embodiments, the human IgG1 Fc region comprises a S239D substitution and an I332E substitution, wherein the residue numbering is according to EU numbering. In some embodiments, the human IgG1 Fc region comprises a S239D substitution and an I332E substitution, and does not comprise one or more (e.g., 1, 2, 3, 4, 5, 6, 7, 8, 9, 10, or any) of a L235V substitution, an F243L substitution, an R292P substitution, an S298A substitution, a Y300L substitution, a V305I substitution, a K326A substitution, an A330L substitution, an E333A substitution, a K334A substitution, and a P396L substitution, wherein the residue numbering is according to EU numbering.

In some embodiments in which the anti-CTLA4 masked antibody (e.g., activatable antibody) comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 masked antibody (e.g., activatable antibody) induces antibody-dependent cell cytotoxicity (ADCC) against a CTLA4-expressing human cell or a human Treg cell. In some embodiments, the ADCC activity of the anti-CTLA4 masked antibody (e.g., activatable antibody) is higher than that of a control antibody comprising a human IgG1 Fc region that does not comprise the substitutions, and/or the ADCC activity of the anti-CTLA4 masked antibody (e.g., activatable antibody) is higher than the ADCC activity of ipilimumab. In certain embodiments wherein the anti-CTLA4 masked antibody (e.g., activatable antibody) comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the ADCC activity of the anti-CTLA4 masked antibody (e.g., activatable antibody) exceeds the ADCC activity of the control antibody by at least about 5% (e.g., at least about 5%, at least about 10%, at least about 20%, at least about 30%, at least about 40%, at least about 50%, at least about 60%, at least about 70%, at least about 80%, or at least about 90%) greater than an anti-CTLA4 antibody that comprises an Fc region that does not comprise the substitutions (e.g., an isotype control or ipilimumab). In certain embodiments wherein the anti-CTLA4 masked antibody (e.g., activatable antibody) comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 masked antibody (e.g., activatable antibody) induces ADCP effects by more than about 10% (e.g., induce ADCP by more than about 10%, more than about 15%, more than about 20%, more than about 25%, more than about 30%, more than about 35%, more than about 40%, etc.) relative to a control (e.g., an isotype control or ipilimumab). In certain embodiments wherein the anti-CTLA4 masked antibodies (e.g., activatable antibodies) comprise an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibodies induce ADCC effects by at a rate of at least about 1.5 fold (e.g., at least about 1.5 fold, at least about 2 fold, at least about 2.5 fold, at least about 3 fold, at least about 4 fold, or at least about 5 fold) greater than an anti-CTLA4 antibody that comprises an Fc region that does not comprise the substitutions. In some embodiments, the anti-CTLA4 masked antibody (e.g., activatable antibody) has a half maximal effective concentration (EC50) of about 5 nM or less (e.g., about 5 nM or less, about 4 nM or less, about 3 nM or less, about 3 nM or less, about 2 nM or less, about 1 nM or less, about 0.5 nM or less, about 0.1 nM or less, or about 0.05 nM or less) for inducing ADCC of cells expressing CTLA4. Methods of measuring ADCP of antibodies and antigen binding fragments are also well known in the art. For example, to assess ADCP activity of a molecule of interest, an in vitro ADCP assay (see, e.g., Bracher et al., 2007, J. Immunol. Methods 323: 160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248: 167-82).

In some embodiments, the anti-CTLA4 masked antibodies (e.g. activatable antibodies) induce antibody-dependent cellular phagocytosis (ADCP) effects against a CTLA4 expressing cell (e.g., against CTLA4-expressing human cells such as Tregs) after the anti-CTLA4 antibody binds to the cell-expressed CTLA4. In some embodiments, the anti-CTLA4 masked antibodies (e.g. activatable antibodies) induce ADCP effects by more than about 10% (e.g., induce ADCP by more than about 10%, more than about 15%, more than about 20%, more than about 25%, more than about 30%, more than about 35%, more than about 40%, etc.) relative to a control (e.g., an isotype control or ipilimumab). In certain embodiments wherein the anti-CTLA4 masked antibodies (e.g. activatable antibodies) comprise an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 masked antibodies (e.g. activatable antibodies) induce ADCP effects by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an Fc region that does not comprise the substitutions. In certain embodiments wherein the anti-CTLA4 masked antibodies (e.g. activatable antibodies) comprise an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 masked antibodies (e.g. activatable antibodies) induce ADCP effects by at a rate of at least about 1.5 fold (e.g., at least about 1.5 fold, at least about 2 fold, at least about 2.5 fold, at least about 3 fold, at least about 4 fold, or at least about 5 fold) greater than an anti-CTLA4 antibody that comprises an Fc region that does not comprise the substitutions. In some embodiments, the anti-CTLA4 masked antibody (e.g. activatable antibody) has a half maximal effective concentration (EC50) of about 5 nM or less (e.g., about 5 nM or less, about 4 nM or less, about 3 nM or less, about 3 nM or less, about 2 nM or less, about 1 nM or less, about 0.5 nM or less, about 0.1 nM or less, or about 0.05 nM or less) for inducing ADCP of cells expressing CTLA4. Methods of measuring ADCP of antibodies and antigen binding fragments are also well known in the art. For example, to assess ADCP activity of a molecule of interest, an in vitro ADCP assay (see, e.g., Bracher et al., 2007, J. Immunol. Methods 323: 160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248: 167-82).

### Full-Length Anti-CTLA4 Masked Antibodies

In some embodiments, an anti-CTLA4 masked antibody (e.g., an activatable antibody) of the present disclosure comprises a first polypeptide comprising, from N-terminus to C-terminus, the masking peptide and the VL; and a second polypeptide comprising, from N-terminus to C-terminus, the VH and the human IgG1 Fc region.

In some embodiments, provided herein is an anti-CTLA4 masked antibody comprising: (1) a first polypeptide and a second polypeptide each comprising, from N- to C-terminus, a VH and a human IgG1 Fc domain, wherein the human IgG1 Fc domain of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising, from N- to C-terminus, (a) a masking peptide comprising a masking unit (MU) and a linkage unit (LU), wherein the MU comprises an amino acid sequence selected from the group consisting of SEQ ID NOS: 146-151; and (b) a VL comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and/or the second polypeptide comprise an IgG1 Fc domain. In some embodiments, the first polypeptide and/or the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 masked antibody is an activatable antibody. In some embodiments the linkage unit (LU) comprises at least a first cleavage site. In certain embodiments, the linkage unit (LU) comprises an amino acid sequence selected from the group consisting of SEQ ID NOS: 99-103.

In some embodiments, provided herein is an anti-CTLA4 masked antibody comprising: (1) a first polypeptide and a second polypeptide each comprising, from N- to C-terminus, a VH and a human IgG1 Fc domain, wherein the human IgG1 Fc domain of the first and second polypeptide dimerize to form a human IgG1 Fc region, and wherein the VH comprises a CDR-H1 comprising the amino acid sequence of SEQ ID NO: 12; a CDR-H2 comprising the amino acid sequence of SEQ ID NO: 13; and a CDR-H3 comprising the amino acid sequence of SEQ ID NO: 14; and (2) and a third and a fourth polypeptide each comprising, from N- to C-terminus, (a) a masking peptide comprising a masking unit (MU) and a linkage unit (LU), wherein the MU comprises an amino acid sequence selected from the group consisting of SEQ ID NOS: 146-151; and (b) a VL comprising a CDR-L1 comprising the amino acid sequence of SEQ ID NO: 15; a CDR-L2 comprising the amino acid sequence of SEQ ID NO: 16; and a CDR-L3 comprising the amino acid sequence of SEQ ID NO: 17. In some embodiments, the VH comprises the amino acid sequence of SEQ ID NO: 66, or an amino acid sequence having at least 80% (e.g., at least 85%, 90%, 95%, 98%, or 99%; or 100%) sequence identity with SEQ ID NO: 66, and/or the VL comprises the amino acid sequence of SEQ ID NO: 67, or an amino acid sequence having at least 80% (e.g., at least 85%, 90%, 95%, 98%, or 99%; or 100%) sequence identity with SEQ ID NO: 67. In some embodiments, the first polypeptide and second polypeptide are each heavy chains, and/or the third polypeptide and the fourth polypeptide are each light chains. In some embodiments, the first polypeptide and/or the second polypeptide comprise an IgG1 Fc domain. In some embodiments, the first polypeptide and/or the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 masked antibody is an activatable antibody. In some embodiments the linkage unit (LU) comprises at least a first cleavage site. In certain embodiments, the linkage unit (LU) comprises an amino acid sequence selected from the group consisting of SEQ ID NOS: 99-103.

In some embodiments, provided herein is an anti-CTLA4 masked antibody comprising: (1) a first polypeptide and a second polypeptide each comprising, from N- to C-terminus, a VH and a human IgG1 Fc domain, wherein the human IgG1 Fc domain of the first and second polypeptide dimerize to form a human IgG1 Fc region, and wherein the VH comprises a CDR-H1 comprising the amino acid sequence of SEQ ID NO: 44; a CDR-H2 comprising the amino acid sequence of SEQ ID NO: 45; and a CDR-H3 comprising the amino acid sequence of SEQ ID NO: 53; and (2) and a third and a fourth polypeptide each comprising, from N- to C-terminus, (a) a masking peptide comprising a masking unit (MU) and a linkage unit (LU), wherein the MU comprises an amino acid sequence selected from the group consisting of SEQ ID NOS: 146-151; and (b) a VL comprising a CDR-L1 comprising the amino acid sequence of SEQ ID NO: 54; a CDR-L2 comprising the amino acid sequence of SEQ ID NO: 16; and a CDR-L3 comprising the amino acid sequence of SEQ ID NO: 55. In some embodiments, the VH comprises the amino acid sequence of SEQ ID NO: 84, or an amino acid sequence having at least 80% (e.g., at least 85%, 90%, 95%, 98%, or 99%; or 100%) sequence identity with SEQ ID NO: 84, and/or the VL comprises the amino acid sequence of SEQ ID NO: 85, or an amino acid sequence having at least 80% (e.g., at least 85%, 90%, 95%, 98%, or 99%; or 100%) sequence identity with SEQ ID NO: 85. In some embodiments, the first polypeptide and second polypeptide are each heavy chains, and/or the third polypeptide and the fourth polypeptide are each light chains. In some embodiments, the first polypeptide and/or the second polypeptide comprise an IgG1 Fc domain. In some embodiments, the first polypeptide and/or the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 masked antibody is an activatable antibody. In some embodiments the linkage unit (LU) comprises at least a first cleavage site. In certain embodiments, the linkage unit (LU) comprises an amino acid sequence selected from the group consisting of SEQ ID NOS: 99-103.

In some embodiments, provided herein is an anti-CTLA4 masked antibody comprising: (1) a first polypeptide and a second polypeptide each comprising, from N- to C-terminus, a VH and a human IgG1 Fc domain, wherein the human IgG1 Fc domain of the first and second polypeptide dimerize to form a human IgG1 Fc region, and wherein the VH comprises a CDR-H1 comprising the amino acid sequence of SEQ ID NO: 44; a CDR-H2 comprising the amino acid sequence of SEQ ID NO: 45; and a CDR-H3 comprising the amino acid sequence of SEQ ID NO: 46; and (2) and a third and a fourth polypeptide each comprising, from N- to C-terminus, (a) a masking peptide comprising a masking unit (MU) and a linkage unit (LU), wherein the MU comprises an amino acid sequence selected from the group consisting of SEQ ID NOS: 146-151; and (b) a VL comprising a CDR-L1 comprising the amino acid sequence of SEQ ID NO: 47; a CDR-L2 comprising the amino acid sequence of SEQ ID NO: 5; and a CDR-L3 comprising the amino acid sequence of SEQ ID NO: 48. In some embodiments, the VH comprises the amino acid sequence of SEQ ID NO: 80, or an amino acid sequence having at least 80% (e.g., at least 85%, 90%, 95%, 98%, or 99%; or 100%) sequence identity with SEQ ID NO: 80, and/or the VL comprises the amino acid sequence of SEQ ID NO: 81, or an amino acid sequence having at least 80% (e.g., at least 85%, 90%, 95%, 98%, or 99%; or 100%) sequence identity with SEQ ID NO: 81. In some embodiments, the first polypeptide and second polypeptide are each heavy chains, and/or the third polypeptide and the fourth polypeptide are each light chains. In some embodiments, the first polypeptide and/or the second polypeptide comprise an IgG1 Fc domain. In some embodiments, the first polypeptide and/or the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 masked antibody is an activatable antibody. In some embodiments the linkage unit (LU) comprises at least a first cleavage site. In certain embodiments, the linkage unit (LU) comprises an amino acid sequence selected from the group consisting of SEQ ID NOS: 99-103.

In some embodiments, provided herein is an activatable antibody comprising: (1) a first polypeptide and a second polypeptide each comprising, from N- to C-terminus, a VH and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising, from N- to C-terminus, (a) a masking peptide comprising a masking unit (MU) and a linkage unit (LU) comprising at least a first cleavage site, wherein the MU comprises an amino acid sequence selected from the group consisting of SEQ ID NOS: 146-151, and (b) a VL comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In certain embodiments, the linkage unit (LU) comprises an amino acid sequence selected from the group consisting of SEQ ID NOS: 99-103.

In some embodiments, provided herein is an activatable antibody comprising: (1) a first polypeptide and a second polypeptide each comprising, from N- to C-terminus, a VH and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising, from N- to C-terminus, (a) a masking peptide comprising a masking unit (MU) and a linkage unit (LU) comprising at least a first cleavage site, wherein the MU comprises the amino acid sequence of SEQ ID NO: 150, and the LM comprises the amino acid sequence of SEQ ID NO: 103; and (b) a VL comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution n, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305T substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In some embodiments, the first and second polypeptide comprise the amino acid sequence of SEQ ID NO: 105, or an amino acid sequence having at least 80% (e.g., at least 85%, 90%, 95%, 98%, or 99%; or 100%) sequence identity with SEQ ID NO: 105, and the third and fourth polypeptide comprise the amino acid sequence of SEQ ID NO: 121 or an amino acid sequence having at least 80% (e.g., at least 85%, 90%, 95%, 98%, or 99%; or 100%) sequence identity with SEQ ID NO: 121.

In some embodiments, the activatable antibody comprises a first polypeptide comprising a sequence of any one of the light chains provided in Table 4, below. In some embodiments, the activatable antibody comprises a second polypeptide comprising a sequence of any one of the heavy chains provided in Table 4, below.

Exemplary sequences of activatable antibodies are provided in Table 4, below.

TABLE 4
Anti-CTLA4 activatable antibody sequences

| Ab name | Heavy Chain | Light Chain |
|---|---|---|
| TY24148 | EVQLVESGGGLVQPGGSLRLSCAASGYSISSGYHWS | EVGSYVLCAKFPFCITSGRS |
| | WIRQAPGKGLEWLARIDWDDDKYYSTSLKSRLTIS | AGGGGTPLGLAGSGGSDIQ |
| | RDNSKNTLYLQLNSLRAEDTAVYYCARSYVYFDY | LTQSPSSLSASVGDRVTITC |
| | WGQGTLVTVSSASTKGPSVFPLAPSSKSTSGGTAAL | RASQSVRGRFLAWYQQKP |
| | GCLVKDYFPEPVTVSWNSGALTSGVHTFPAVLQSSG | GKAPKLLIYDASNRATGIPS |
| | LYSLSSVVTVPSSSLGTQTYICNVNHKPSNTKVDKK | RFSGSGSGTDFTLTISSLQPE |
| | VEPKSCDKTHTCPPCPAPELLGGPSVFLFPPKPKDTL | DFATYYCQQSSSWPPTFGQ |
| | MISRTPEVTCVVVDVSHEDPEVKFNWYVDGVEVH | GTKVEIKRTVAAPSVFIFPPS |
| | NAKTKPREEQYNSTYRVVSVLTVLHQDWLNGKEY | DEQLKSGTASVVCLLNNFY |
| | KCKVSNKALPAPIEKTISKAKGQPREPQVYTLPPSR | PREAKVQWKVDNALQSGN |
| | DELTKNQVSLTCLVKGFYPSDIAVEWESNGQPENNY | SQESVTEQDSKDSTYSLSST |
| | KTTPPVLDSDGSFFLYSKLTVDKSRWQQGNVFSCSV | LTLSKADYEKHKVYACEVT |
| | MHEALHNHYTQKSLSLSPGK | HQGLSSPVTKSFNRGEC |
| | (SEQ ID NO: 104) | (SEQ ID NO: 106) |
| TY24149 | EVQLVESGGGLVQPGGSLRLSCAASGYSISSGYHW | EVGSYPPCVGFYPYCKTSG |
| | SWIRQAPGKGLEWLARIDWDDDKYYSTSLKSRLTI | RSAGGGGTPLGLAGSGGSD |
| | SRDNSKNTLYLQLNSLRAEDTAVYYCARSYVYFDY | IQLTQSPSSLSASVGDRVTIT |
| | WGQGTLVTVSSASTKGPSVFPLAPSSKSTSGGTAAL | CRASQSVRGRFLAWYQQK |
| | GCLVKDYFPEPVTVSWNSGALTSGVHTFPAVLQSS | PGKAPKLLIYDASNRATGIP |
| | GLYSLSSVVTVPSSSLGTQTYICNVNHKPSNTKVDK | SRFSGSGSGTDFTLTISSLQP |
| | KVEPKSCDKTHTCPPCPAPELLGGPSVFLFPPKPKD | EDFATYYCQQSSSWPPTFG |
| | TLMISRTPEVTCVVVDVSHEDPEVKFNWYVDGVEV | QGTKVEIKRTVAAPSVFIFPP |
| | HNAKTKPREEQYNSTYRVVSVLTVLHQDWLNGKE | SDEQLKSGTASVVCLLNNF |
| | YKCKVSNKALPAPIEKTISKAKGQPREPQVYTLPPS | YPREAKVQWKVDNALQSG |
| | RDELTKNQVSLTCLVKGFYPSDIAVEWESNGQPEN | NSQESVTEQDSKDSTYSLSS |
| | NYKTTPPVLDSDGSFFLYSKLTVDKSRWQQGNVFS | TLTLSKADYEKHKVYACEV |
| | CSVMHEALHNHYTQKSLSLSPGK | THQGLSSPVTKSFNRGEC |
| | (SEQ ID NO: 104) | (SEQ ID NO: 107) |
| TY24155 | EVQLVESGGGLVQPGGSLRLSCAASGYSISSGYHWS | EVQSYSSCKAPYPYCKGSG |
| | WIRQAPGKGLEWLARIDWDDDKYYSTSLKSRLTIS | RSAGGGGTPLGLAGSGGSD |
| | RDNSKNTLYLQLNSLRAEDTAVYYCARSYVYFDY | IQLTQSPSSLSASVGDRVTIT |
| | WGQGTLVTVSSASTKGPSVFPLAPSSKSTSGGTAAL | CRASQSVRGRFLAWYQQK |
| | GCLVKDYFPEPVTVSWNSGALTSGVHTFPAVLQSSG | PGKAPKLLIYDASNRATGIP |
| | LYSLSSVVTVPSSSLGTQTYICNVNHKPSNTKVDKK | SRFSGSGSGTDFTLTISSLQP |
| | VEPKSCDKTHTCPPCPAPELLGGPSVFLFPPKPKDTL | EDFATYYCQQSSSWPPTFG |
| | MISRTPEVTCVVVDVSHEDPEVKFNWYVDGVEVH | QGTKVEIKRTVAAPSVFIFPP |
| | NAKTKPREEQYNSTYRVVSVLTVLHQDWLNGKEY | SDEQLKSGTASVVCLLNNF |
| | KCKVSNKALPAPIEKTISKAKGQPREPQVYTLPPSR | YPREAKVQWKVDNALQSG |
| | DELTKNQVSLTCLVKGFYPSDIAVEWESNGQPENNY | NSQESVTEQDSKDSTYSLSS |
| | KTTPPVLDSDGSFFLYSKLTVDKSRWQQGNVFSCSV | TLTLSKADYEKHKVYACEV |
| | MHEALHNHYTQKSLSLSPGK | THQGLSSPVTKSFNRGEC |
| | (SEQ ID NO: 104) | (SEQ ID NO: 108) |
| TY24157 | EVQLVESGGGLVQPGGSLRLSCAASGYSISSGYHWS | EVGSYALCEPFATSVCRPSG |
| | WIRQAPGKGLEWLARIDWDDDKYYSTSLKSRLTIS | RSAGGGGTPLGLAGSGGSD |
| | RDNSKNTLYLQLNSLRAEDTAVYYCARSYVYFDY | IQLTQSPSSLSASVGDRVTIT |
| | WGQGTLVTVSSASTKGPSVFPLAPSSKSTSGGTAAL | CRASQVRGRFLAWYQQKP |
| | GCLVKDYFPEPVTVSWNSGALTSGVHTFPAVLQSSG | GKAPKLLIYDASNRATGIPS |
| | LYSLSSVVTVPSSSLGTQTYICNVNHKPSNTKVDKK | RFSGSGSGTDFTLTISSLQPE |
| | VEPKSCDKTHTCPPCPAPELLGGPSVFLFPPKPKDTL | DFATYYCQQSSSWPPTFGQ |
| | MISRTPEVTCVVVDVSHEDPEVKFNWYVDGVEVH | GTKVEIKRTVAAPSVFIFPPS |
| | NAKTKPREEQYNSTYRVVSVLTVLHQDWLNGKEY | DEQLKSGTASVVCLLNNFY |
| | KCKVSNKALPAPIEKTISKAKGQPREPQVYTLPPSR | PREAKVQWKVDNALQSGN |
| | DELTKNQVSLTCLVKGFYPSDIAVEWESNGQPENNY | SQESVTEQDSKDSTYSLSST |
| | KTTPPVLDSDGSFFLYSKLTVDKSRWQQGNVFSCSV | LTLSKADYEKHKVYACEVT |
| | MHEALHNHYTQKSLSLSPGK | HQGLSSPVTKSFNRGEC |
| | (SEQ ID NO: 104) | (SEQ ID NO: 109) |
| TY24158 | EVQLVESGGGLVQPGGSLRLSCAASGYSISSGYHWS | EVGSYTLCEPFATSVCRPSG |
| | WIRQAPGKGLEWLARIDWDDDKYYSTSLKSRLTIS | RSAGGGGTPLGLAGSGGSD |
| | RDNSKNTLYLQLNSLRAEDTAVYYCARSYVYFDY | IQLTQSPSSLSASVGDRVTIT |
| | WGQGTLVTVSSASTKGPSVFPLAPSSKSTSGGTAAL | CRASQSVRGRFLAWYQQK |
| | GCLVKDYFPEPVTVSWNSGALTSGVHTFPAVLQSSG | PGKAPKLLIYDASNRATGIP |
| | LYSLSSVVTVPSSSLGTQTYICNVNHKPSNTKVDKK | SRFSGSGSGTDFTLTISSLQP |
| | VEPKSCDKTHTCPPCPAPELLGGPSVFLFPPKPKDTL | EDFATYYCQQSSSWPPTFG |
| | MISRTPEVTCVVVDVSHEDPEVKFNWYVDGVEVH | QGTKVEIKRTVAAPSVFIFPP |
| | NAKTKPREEQYNSTYRVVSVLTVLHQDWLNGKEY | SDEQLKSGTASVVCLLNNF |
| | KCKVSNKALPAPIEKTISKAKGQPREPQVYTLPPSR | YPREAKVQWKVDNALQSG |
| | DELTKNQVSLTCLVKGFYPSDIAVEWESNGQPENNY | NSQESVTEQDSKDSTYSLSS |
| | KTTPPVLDSDGSFFLYSKLTVDKSRWQQGNVFSCSV | TLTLSKADYEKHKVYACEV |
| | MHEALHNHYTQKSLSLSPGK | THQGLSSPVTKSFNRGEC |
| | (SEQ ID NO: 104) | (SEQ ID NO: 110) |
| TY24459 | EVQLVESGGGLVQPGGSLRLSCAASGYSISSGYHWS | EVGSYSSCKAPYPYCKGSG |
| | WIRQAPGKGLEWLARIDWDDDKYYSTSLKSRLTIS | RGPLGLAGGSDIQLTQSPSS |
| | RDNSKNTLYLQLNSLRAEDTAVYYCARSYVYFDY | LSASVGDRVTITCRASQSVR |
| | WGQGTLVTVSSASTKGPSVFPLAPSSKSTSGGTAAL | GRFLAWYQQKPGKAPKLLI |
| | GCLVKDYFPEPVTVSWNSGALTSGVHTFPAVLQSSG | YDASNRATGIPSRFSGSGSG |
| | LYSLSSVVTVPSSSLGTQTYICNVNHKPSNTKVDKK | TDFTLTISSLQPEDFATYYC |
| | VEPKSCDKTHTCPPCPAPELLGGPSVFLFPPKPKDTL | QQSSSWPPTFGQGTKVEIK |
| | MISRTPEVTCVVVDVSHEDPEVKFNWYVDGVEVH | RTVAAPSVFIFPPSDEQLKS |
| | NAKTKPREEQYNSTYRVVSVLTVLHQDWLNGKEY | GTASVVCLLNNFYPREAKV |
| | KCKVSNKALPAPIEKTISKAKGQPREPQVYTLPPSR | QWKVDNALQSGNSQESVT |
| | DELTKNQVSLTCLVKGFYPSDIAVEWESNGQPENNY | EQDSKDSTYSLSSTLTLSKA |
| | KTTPPVLDSDGSFFLYSKLTVDKSRWQQGNVFSCSV | DYEKHKVYACEVTHQGLSS |
| | MHEALHNHYTQKSLSLSPGK | PVTKSFNRGEC |
| | (SEQ ID NO: 104) | (SEQ ID NO: 111) |
| TY24460 | EVQLVESGGGLVQPGGSLRLSCAASGYSISSGYHWS | EVGSYSSCKAPYPYCKGSG |
| | WIRQAPGKGLEWLARIDWDDDKYYSTSLKSRLTIS | GGPLGLAGGSDIQLTQSPSS |
| | RDNSKNTLYLQLNSLRAEDTAVYYCARSYVYFDY | LSASVGDRVTITCRASQSVR |
| | WGQGTLVTVSSASTKGPSVFPLAPSSKSTSGGTAAL | GRFLAWYQQKPGKAPKLLI |
| | GCLVKDYFPEPVTVSWNSGALTSGVHTFPAVLQSSG | YDASNRATGIPSRFSGSGSG |
| | LYSLSSVVTVPSSSLGTQTYICNVNHKPSNTKVDKK | TDFTLTISSLQPEDFATYYC |
| | VEPKSCDKTHTCPPCPAPELLGGPSVFLFPPKPKDTL | QQSSSWPPTFGQGTKVEIK |
| | MISRTPEVTCVVVDVSHEDPEVKFNWYVDGVEVH | RTVAAPSVFIFPPSDEQLKS |
| | NAKTKPREEQYNSTYRVVSVLTVLHQDWLNGKEY | GTASVVCLLNNFYPREAKV |
| | KCKVSNKALPAPIEKTISKAKGQPREPQVYTLPPSR | QWKVDNALQSGNSQESVT |
| | DELTKNQVSLTCLVKGFYPSDIAVEWESNGQPENNY | EQDSKDSTYSLSSTLTLSKA |
| | KTTPPVLDSDGSFFLYSKLTVDKSRWQQGNVFSCSV | DYEKHKVYACEVTHQGLSS |
| | MHEALHNHYTQKSLSLSPGK | PVTKSFNRGEC |
| | (SEQ ID NO: 104) | (SEQ ID NO: 112) |
| TY24461 | EVQLVESGGGLVQPGGSLRLSCAASGYSISSGYHWS | EVGSYSSCKAPYPYCKGGG |
| | WIRQAPGKGLEWLARIDWDDDKYYSTSLKSRLTIS | GPLGLAGGSDIQLTQSPSSL |
| | RDNSKNTLYLQLNSLRAEDTAVYYCARSYVYFDY | SASVGDRVTITCRASQSVR |
| | WGQGTLVTVSSASTKGPSVFPLAPSSKSTSGGTAAL | GRFLAWYQQKPGKAPKLLI |
| | GCLVKDYFPEPVTVSWNSGALTSGVHTFPAVLQSSG | YDASNRATGIPSRFSGSGSG |
| | LYSLSSVVTVPSSSLGTQTYICNVNHKPSNTKVDKK | TDFTLTISSLQPEDFATYYC |
| | VEPKSCDKTHTCPPCPAPELLGGPSVFLFPPKPKDTL | QQSSSWPPTFGQGTKVEIK |
| | MISRTPEVTCVVVDVSHEDPEVKFNWYVDGVEVH | RTVAAPSVFIFPPSDEQLKS |
| | NAKTKPREEQYNSTYRVVSVLTVLHQDWLNGKEY | GTASVVCLLNNFYPREAKV |
| | KCKVSNKALPAPIEKTISKAKGQPREPQVYTLPPSR | QWKVDNALQSGNSQESVT |
| | DELTKNQVSLTCLVKGFYPSDIAVEWESNGQPENNY | EQDSKDSTYSLSSTLTLSKA |
| | KTTPPVLDSDGSFFLYSKLTVDKSRWQQGNVFSCSV | DYEKHKVYACEVTHQGLSS |
| | MHEALHNHYTQKSLSLSPGK | PVTKSFNRGEC |
| | (SEQ ID NO: 104) | (SEQ ID NO: 113) |
| TY24462 | EVQLVESGGGLVQPGGSLRLSCAASGYSISSGYHWS | ESSCKAPYPYCKGSGGGPL |
| | WIRQAPGKGLEWLARIDWDDDKYYSTSLKSRLTIS | GLAGGSDIQLTQSPSSLSAS |
| | RDNSKNTLYLQLNSLRAEDTAVYYCARSYVYFDY | VGDRVTITCRASQSVRGRF |
| | WGQGTLVTVSSASTKGPSVFPLAPSSKSTSGGTAAL | LAWYQQKPGKAPKLLIYDA |
| | GCLVKDYFPEPVTVSWNSGALTSGVHTFPAVLQSSG | SNRATGIPSRFSGSGSGTDF |
| | LYSLSSVVTVPSSSLGTQTYICNVNHKPSNTKVDKK | TLTISSLQPEDFATYYCQQSS |
| | VEPKSCDKTHTCPPCPAPELLGGPSVFLFPPKPKDTL | SWPPTFGQGTKVEIKRTVA |
| | MISRTPEVTCVVVDVSHEDPEVKFNWYVDGVEVH | APSVFIFPPSDEQLKSGTAS |
| | NAKTKPREEQYNSTYRVVSVLTVLHQDWLNGKEY | VVCLLNNFYPREAKVQWK |
| | KCKVSNKALPAPIEKTISKAKGQPREPQVYTLPPSR | VDNALQSGNSQESVTEQDS |
| | DELTKNQVSLTCLVKGFYPSDIAVEWESNGQPENNY | KDSTYSLSSTLTLSKADYEK |
| | KTTPPVLDSDGSFFLYSKLTVDKSRWQQGNVFSCSV | HKVYACEVTHQGLSSPVTK |
| | MHEALHNHYTQKSLSLSPGK | SFNRGEC |
| | (SEQ ID NO: 104) | (SEQ ID NO: 114) |
| TY24463 | EVQLVESGGGLVQPGGSLRLSCAASGYSISSGYHWS | EVGSYTLCEPFATSVCRPSG |
| | WIRQAPGKGLEWLARIDWDDDKYYSTSLKSRLTIS | RGPLGLAGGSDIQLTQSPSS |
| | RDNSKNTLYLQLNSLRAEDTAVYYCARSYVYFDY | LSASVGDRVTITCRASQSVR |
| | WGQGTLVTVSSASTKGPSVFPLAPSSKSTSGGTAAL | GRFLAWYQQKPGKAPKLLI |
| | GCLVKDYFPEPVTVSWNSGALTSGVHTFPAVLQSSG | YDASNRATGIPSRFSGSGSG |
| | LYSLSSVVTVPSSSLGTQTYICNVNHKPSNTKVDKK | TDFTLTISSLQPEDFATYYC |
| | VEPKSCDKTHTCPPCPAPELLGGPSVFLFPPKPKDTL | QQSSSWPPTFGQGTKVEIK |
| | MISRTPEVTCVVVDVSHEDPEVKFNWYVDGVEVH | RTVAAPSVFIFPPSDEQLKS |
| | NAKTKPREEQYNSTYRVVSVLTVLHQDWLNGKEY | GTASVVCLLNNFYPREAKV |
| | KCKVSNKALPAPIEKTISKAKGQPREPQVYTLPPSR | QWKVDNALQSGNSQESVT |
| | DELTKNQVSLTCLVKGFYPSDIAVEWESNGQPENNY | EQDSKDSTYSLSSTLTLSKA |
| | KTTPPVLDSDGSFFLYSKLTVDKSRWQQGNVFSCSV | DYEKHKVYACEVTHQGLSS |
| | MHEALHNHYTQKSLSLSPGK | PVTKSFNRGEC |
| | (SEQ ID NO: 104) | (SEQ ID NO: 115) |
| TY24464 | EVQLVESGGGLVQPGGSLRLSCAASGYSISSGYHWS | EVGSYTLCEPFATSVCRPSG |
| | WIRQAPGKGLEWLARIDWDDDKYYSTSLKSRLTIS | GGPLGLAGGSDIQLTQSPSS |
| | RDNSKNTLYLQLNSLRAEDTAVYYCARSYVYFDY | LSASVGDRVTITGRASQSVR |
| | WGQGTLVTVSSASTKGPSVFPLAPSSKSTSGGTAAL | GRFLAWYQQKPGKAPKLLI |
| | GCLVKDYFPEPVTVSWNSGALTSGVHTFPAVLQSSG | YDASNRATGIPSRFSQSGSG |
| | LYSLSSVVTVPSSSLGTQTYICNVNHKPSNTKVDKK | TDFTLTISSLQPEDFATYYC |
| | VEPKSCDKTHTCPPCPAPELLGGPSVFLFPPKPKDTL | QQSSSWPPTFGQGTKVEIK |
| | MISRTPEVTCVVVDVSHEDPEVKFNWYVDGVEVH | RTVAAPSVFIFPPSDEQLKS |
| | NAKTKPREEQYNSTYRVVSVLTVLHQDWLNGKEY | GTASVVCLLNNFYPREAKV |
| | KCKVSNKALPAPIEKTISKAKGQPREPQVYTLPPSR | QWKVDNALQSGNSQESVT |
| | DELTKNQVSLTCLVKGFYPSDIAVEWESNGQPENNY | EQDSKDSTYSLSSTLTLSKA |
| | KTTPPVLDSDGSFFLYSKLTVDKSRWQQGNVFSCSV | DYEKHKVYACEVTHQGLSS |
| | MHEALHNHYTQKSLSLSPGK | PVTKSFNRGEC |
| | (SEQ ID NO: 104) | (SEQ ID NO: 116) |
| TY24466 | EVQLVESGGGLVQPGGSLRLSCAASGYSISSGYHWS | ETLCEPFATSVCRPSGGGPL |
| | WIRQAPGKGLEWLARIDWDDDKYYSTSLKSRLTIS | GLAGGSDIQLTQSPSSLSAS |
| | RDNSKNTLYLQLNSLRAEDTAVYYCARSYVYFDY | VGDRVTITCRASQSVRGRF |
| | WGQGTLVTVSSASTKGPSVFPLAPSSKSTSGGTAAL | LAWYQQKPGKAPKLLIYDA |
| | GCLVKDYFPEPVTVSWNSGALTSGVHTFPAVLQSSG | SNRATGIPSRFSGSGSGTDF |
| | LYSLSSVVTVPSSSLGTQTYICNVNHKPSNTKVDKK | TLTISSLQPEDFATYYCQQSS |
| | VEPKSCDKTHTCPPCPAPELLGGPSVFLFPPKPKDTL | SWPPTFGQGTKVEIKRTVA |
| | MISRTPEVTCVVVDVSHEDPEVKFNWYVDGVEVH | APSVFIFPPSDEQLKSGTAS |
| | NAKTKPREEQYNSTYRVVSVLTVLHQDWLNGKEY | VVCLLNNFYPREAKVQWK |
| | KCKVSNKALPAPIEKTISKAKGQPREPQVYTLPPSR | VDNALQSGNSQESVTEQDS |
| | DELTKNQVSLTCLVKGFYPSDIAVEWESNGQPENNY | KDSTYSLSSTLTLSKADYEK |
| | KTTPPVLDSDGSFFLYSKLTVDKSRWQQGNVFSCSV | HKVYACEVTHQGLSSPVTK |
| | MHEALHNHYTQKSLSLSPGK | SFNRGEC |
| | (SEQ ID NO: 104) | (SEQ ID NO: 117) |
| TY24465 | EVQLVESGGGLVQPGGSLRLSCAASGYSISSGYHWS | EVGSYTLCEPFATSVCRPGG |
| | WIRQAPGKGLEWLARIDWDDDKYYSTSLKSRLTIS | GPLGLAGGSDIQLTQSPSSL |
| | RDNSKNTLYLQLNSLRAEDTAVYYCARSYVYFDY | SASVGDRVTITCRASQSVR |
| | WGQGTLVTVSSASTKGPSVFPLAPSSKSTSGGTAAL | GRFLAWYQQKPGKAPKLLI |
| | GCLVKDYFPEPVTVSWNSGALTSGVHTFPAVLQSSG | YDASNRATGIPSRFSGSGSG |
| | LYSLSSVVTVPSSSLGTQTYICNVNHKPSNTKVDKK | TDFTLTISSLQPEDFATYYC |
| | VEPKSCDKTHTCPPCPAPELLGGPSVFLFPPKPKDTL | QQSSSWPPTFGQGTKVEIK |
| | MISRTPEVTCVVVDVSHEDPEVKFNWYVDGVEVH | RTVAAPSVFIFPPSDEQLKS |
| | NAKTKPREEQYNSTYRVVSVLTVLHQDWLNGKEY | GTASVVCLLNNFYPREAKV |
| | KCKVSNKALPAPIEKTISKAKGQPREPQVYTLPPSR | QWKVDNALQSGNSQESVT |
| | DELTKNQVSLTCLVKGFYPSDIAVEWESNGQPENNY | EQDSKDSTYSLSSTLTLSKA |
| | KTTPPVLDSDGSFFLYSKLTVDKSRWQQGNVFSCSV | DYEKHKVYACEVTHQGLSS |
| | MHEALHNHYTQKSLSLSPGK | PVTKSFNRGEC |
| | (SEQ ID NO: 104) | (SEQ ID NO: 118) |
| TY24800 | EVQLVESGGGLVQPGGSLRLSCAASGYSISSGYHWS | ETLCEPFATSVCRPGGGPLG |
| | WIRQAPGKGLEWLARIDWDDDKYYSTSLKSRLTIS | LAGGSDIQLTQSPSSLSASV |
| | RDNSKNTLYLQLNSLRAEDTAVYYCARSYVYFDY | GDRVTITCRASQSVRGRFL |
| | WGQGTLVTVSSASTKGPSVFPLAPSSKSTSGGTAAL | AWYQQKPGKAPKLLIYDAS |
| | GCLVKDYFPEPVTVSWNSGALTSGVHTFPAVLQSSG | NRATGIPSRFSGSGSGTDFT |
| | LYSLSSVVTVPSSSLGTQTYICNVNHKPSNTKVDKK | LTISSLQPEDFATYYCQQSSS |
| | VEPKSCDKTHTCPPCPAPELLGGPDVFLFPPKPKDT | WPPTFGQGTKVEIKRTVAA |
| | LMISRTPEVTCVVVDVSHEDPEVKFNWYVDGVEV | PSVFIFPPSDEQLKSGTASV |
| | HNAKTKPREEQYNSTYRVVSVLTVLHQDWLNGKE | VCLLNNFYPREAKVQWKV |
| | YKCKVSNKALPAPEEKTISKAKGQPREPQVYTLPPS | DNALQSGNSQESVTEQDSK |
| | RDELTKNQVSLTCLVKGFYPSDIAVEWESNGQPENN | DSTYSLSSTLTLSKADYEKH |
| | YKTTPPVLDSDGSFFLYSKLTVDKSRWQQGNVFSCS | KVYACEVTHQGLSSPVTKS |
| | VMHEALHNHYTQKSLSLSPGK | FNRGEC |
| | (SEQ ID NO: 105) | (SEQ ID NO: 119) |
| TY24649 | EVQLVESGGGLVQPGGSLRLSCAASGYSISSGYHWS | ETLCEPFATSVCRPGGGPLG |
| | WIRQAPGKGLEWLARIDWDDDKYYSTSLKSRLTIS | LAGGGSDIQLTQSPSSLSAS |
| | RDNSKNTLYLQLNSLRAEDTAVYYCARSYVYFDY | VGDRVTITCRASQSVRGRF |
| | WGQGTLVTVSSASTKGPSVFPLAPSSKSTSGGTAAL | LAWYQQKPGKAPKLLIYDA |
| | GCLVKDYFPEPVTVSWNSGALTSGVHTFPAVLQSSG | SNRATGIPSRFSGSGSGTDF |
| | LYSLSSVVTVPSSSLGTQTYICNVNHKPSNTKVDKK | TLTISSLQPEDFATYYCQQSS |
| | VEPKSCDKTHTCPPCPAPELLGGPSVFLFPPKPKDTL | SWPPTFGQGTKVEIKRTVA |
| | MISRTPEVTCVVVDVSHEDPEVKFNWYVDGVEVH | APSVFIFPPSDEQLKSGTAS |
| | NAKTKPREEQYNSTYRVVSVLTVLHQDWLNGKEY | VVCLLNNFYPREAKVQWK |
| | KCKVSNKALPAPIEKTISKAKGQPREPQVYTLPPSR | VDNALQSGNSQESVTEQDS |
| | DELTKNQVSLTCLVKGFYPSDIAVEWESNGQPENNY | KDSTYSLSSTLTLSKADYEK |
| | KTTPPVLDSDGSFFLYSKLTVDKSRWQQGNVFSCSV | HKVYACEVTHQGLSSPVTK |
| | MHEALHNHYTQKSLSLSPGK | SFNRGEC |
| | (SEQ ID NO: 104) | (SEQ ID NO: 120) |
| TY24652 | EVQLVESGGGLVQPGGSLRLSCAASGYSISSGYHWS | ETLCEPFATSVCRPGGGPLG |
| | WIRQAPGKGLEWLARIDWDDDKYYSTSLKSRLTIS | LAGGGSDIQLTQSPSSLSAS |
| | RDNSKNTLYLQLNSLRAEDTAVYYCARSYVYFDY | VGDRVTITCRASQSVRGRF |
| | WGQGTLVTVSSASTKGPSVFPLAPSSKSTSGGTAAL | LAWYQQKPGKAPKLLIYDA |
| | GCLVKDYFPEPVTVSWNSGALTSGVHTFPAVLQSSG | SNRATGIPSRFSGSGSGTDF |
| | LYSLSSVVTVPSSSLGTQTYICNVNHKPSNTKVDKK | TLTISSLQPEDFATYYCQQSS |
| | VEPKSCDKTHTCPPCPAPELLGGPDVFLFPPKPKDT | SWPPTFGQGTKVEIKRTVA |
| | LMISRTPEVTCVVVDVSHEDPEVKFNWYVDGVEV | APSVFIFPPSDEQLKSGTAS |
| | HNAKTKPREEQYNSTYRVVSVLTVLHQDWLNGKE | VVCLLNNFYPREAKVQWK |
| | YKCKVSNKALPAPEEKTISKAKGQPREPQVYTLPPS | VDNALQSGNSQESVTEQDS |
| | RDELTKNQVSLTCLVKGFYPSDIAVEWESNGQPENN | KDSTYSLSSTLTLSKADYEK |
| | YKTTPPVLDSDGSFFLYSKLTVDKSRWQQGNVFSCS | HKVYACEVTHQGLSSPVTK |
| | VMHEALHNHYTQKSLSLSPGK | SFNRGEC |
| | (SEQ ID NO: 105) | (SEQ ID NO: 121) |

In some embodiments, provided herein is an anti-CTLA4 masked antibody comprising: (1) a first polypeptide and a second polypeptide each comprising, from N-terminus to C-terminus, an antibody heavy chain variable region (VH) and a human IgG1 Fc domain, wherein the human IgG1 Fc domains of the first and second polypeptide dimerize to form a human IgG1 Fc region; and (2) and a third and a fourth polypeptide each comprising, from N- to C-terminus, a masking peptide comprising a masking unit (MU) and a linkage unit (LU) and an antibody light chain variable region (VL). In some embodiments, the first polypeptide and second polypeptide are each heavy chains, and/or the third polypeptide and the fourth polypeptide are each light chains. In some embodiments, the third polypeptide and/or fourth polypeptide comprises an amino acid sequence selected from the group consisting of SEQ ID NOS: 106-121; and wherein the first polypeptide and/or second polypeptide comprises an amino acid sequence selected from the group consisting of SEQ ID NOS: 104-105. In some embodiments, the third polypeptide and/or fourth polypeptide comprises the amino acid sequence of SEQ ID NO: 106, or an amino acid sequence having at least 80% (e.g., at least 85%, 90%, 95%, 98%, or 99%; or 100%) sequence identity with SEQ ID NO: 106, and the second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 107, or an amino acid sequence having at least 80% (e.g., at least 85%, 90%, 95%, 98%, or 99%; or 100%) sequence identity with SEQ ID NO: 107, and the second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 108, or an amino acid sequence having at least 80% (e.g., at least 85%, 90%, 95%, 98%, or 99%; or 100%) sequence identity with SEQ ID NO: 108, and the second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 109, or an amino acid sequence having at least 80% (e.g., at least 85%, 90%, 95%, 98%, or 99%; or 100%) sequence identity with SEQ ID NO: 109, and the second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 110, or an amino acid sequence having at least 80% (e.g., at least 85%, 90%, 95%, 98%, or 99%; or 100%) sequence identity with SEQ ID NO: 110, and the second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 111, or an amino acid sequence having at least 80% (e.g., at least 85%, 90%, 95%, 98%, or 99%; or 100%) sequence identity with SEQ ID NO: 111, and the second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 112, or an amino acid sequence having at least 80% (e.g., at least 85%, 90%, 95%, 98%, or 99%; or 100%) sequence identity with SEQ ID NO: 112, and the first polypeptide and/or second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 113, or an amino acid sequence having at least 80% (e.g., at least 85%, 90%, 95%, 98%, or 99%; or 100%) sequence identity with SEQ ID NO: 113, and the first polypeptide and/or second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 115, or an amino acid sequence having at least 80% (e.g., at least 85%, 90%, 95%, 98%, or 99%; or 100%) sequence identity with SEQ ID NO: 115, and the first polypeptide and/or second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 116, or an amino acid sequence having at least 80% (e.g., at least 85%, 90%, 95%, 98%, or 99%; or 100%) sequence identity with SEQ ID NO: 116, and the first polypeptide and/or second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 117, or an amino acid sequence having at least 80% (e.g., at least 85%, 90%, 95%, 98%, or 99%; or 100%) sequence identity with SEQ ID NO: 117, and the first polypeptide and/or second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 118, or an amino acid sequence having at least 80% (e.g., at least 85%, 90%, 95%, 98%, or 99%; or 100%) sequence identity with SEQ ID NO: 118, and the first polypeptide and/or second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 119, or an amino acid sequence having at least 80% (e.g., at least 85%, 90%, 95%, 98%, or 99%; or 100%) sequence identity with SEQ ID NO: 119, and the first polypeptide and/or second polypeptide comprises the amino acid sequence of SEQ ID NO: 105 or an amino acid sequence having at least 80% (e.g., at least 85%, 90%, 95%, 98%, or 99%; or 100%) sequence identity with SEQ ID NO: 105. In some embodiments, the third polypeptide and/or fourth polypeptide comprises the amino acid sequence of SEQ ID NO: 120, or an amino acid sequence having at least 80% (e.g., at least 85%, 90%, 95%, 98%, or 99%; or 100%) sequence identity with SEQ ID NO: 120, and the first polypeptide and/or second polypeptide comprises the amino acid sequence of SEQ ID NO: 104, or an amino acid sequence having at least 80% (e.g., at least 85%, 90%, 95%, 98%, or 99%; or 100%) sequence identity with SEQ ID NO: 104. In some embodiments, the third polypeptide and/or fourth polypeptide comprises the amino acid sequence of SEQ ID NO: 121, or an amino acid sequence having at least 80% (e.g., at least 85%, 90%, 95%, 98%, or 99%; or 100%) sequence identity with SEQ ID NO: 121, and the first polypeptide and/or second polypeptide comprises the amino acid sequence of SEQ ID NO: 105, or an amino acid sequence having at least 80% (e.g., at least 85%, 90%, 95%, 98%, or 99%; or 100%) sequence identity with SEQ ID NO: 105.

### Masked Binding Polypeptide Properties

In some embodiments, an anti-CTLA4 masked binding polypeptide (i.e., a masked antibody) of the present disclosure comprises: (a) a masking unit (MU), (b) a linkage unit (LU), and (c) a target binding moiety (TBM). In some embodiments, the masking unit (MU) binds to the target binding moiety (TBM) of the activatable antibody and reduces or inhibits binding of the activatable binding moiety to CTLA4 (e.g., human CTLA4), as compared to the binding of a corresponding binding polypeptide lacking the masking unit to CTLA4 (e.g., human CTLA4) and/or as compared to the binding of a parental antibody (e.g., an antibody having the same TBM but lacking the MU) to CTLA4 (e.g., human CTLA4).

In some embodiments, a masked antibody (e.g., activatable antibody) exhibits a first level of binding to CTLA4 when in an inhibited, masked, and/or uncleaved state, and exhibits a second level of binding to CTLA4 in an uninhibited, unmasked, and/or cleaved state, where the second level of CTLA4 binding is greater than the first level of CTLA4 binding. In some embodiments wherein the masked antibody is an activatable antibody comprising a linkage moiety (LM) comprising at least a first cleavage site, access to CTLA4 by the activatable binding polypeptide is greater after cleavage within the cleavage site (e.g., by one or more proteases). In certain embodiments, an activatable antibody of the present disclosure comprises: (a) a masking unit (MU), (b) linkage unit (LU) comprising at least a first cleavage site, and (c) a target binding moiety (TBM).

In some embodiments, an activatable antibody of the present disclosure is generally considered to be an “activatable” binding polypeptide when binding affinity of the polypeptide to CTLA4 (e.g., human CTLA4) increases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more) after activation of the activatable antibody as compared to prior to activation of the activatable antibody (e.g., after activation by treatment with one or more proteases that cleave within the cleavage site, after activation by a change in pH (increased or decreased), after activation by a temperature shift (increased or decreased), after activation by being contacted with a second molecule (such as a small molecule), etc.). In some embodiments, an activatable antibody of the present disclosure is generally considered “activatable” if the EC₅₀ of the activatable antibody decreases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more) after “activation” (e.g., as measured by an ELISA or FACS assay; see the examples below) In some embodiments, an activatable antibody of the present disclosure is generally considered “activatable” if the EC₅₀ of the polypeptide decreases by at least about 2-fold after treatment with a protease that cleaves within the cleavage site (e.g., as measured by an ELISA or FACS assay; see the examples below).

In some embodiments, when the masking unit (MU) is bound to the target binding moiety (TBM) of the activatable antibody, the K, of the activatable antibody for CTLA4 is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more) times greater than when the masking unit (MU) is not bound to the target binding moiety (TBM) (e.g., after “activation” of the activatable antibody (such as after protease treatment to cleave within the cleavage site)) and/or than the K_{D} of the parental antibody for CTLA4. Methods of measuring affinity are known in the art, including, for example, by the methods described in the Examples below).

In some embodiments, when the masking unit is bound to the target binding moiety of the activatable antibody, the K_{D} of the activatable antibody for CTLA4 is reduced by at least about 25% (e.g., at least about 25%, at least about 30%, at least about 40%, at least about 50%, at least about 60%, at least about 70%, at least about 80%, at least about 90%, at least about 95%, at least about 99%) relative to when the masking unit is not bound to the target binding moiety (e.g., after “activation” of the activatable antibody (such as after protease treatment to cleave within the cleavage site)) and/or relative to the K_{D} of the parental antibody for CTLA4. Methods of measuring affinity are known in the art, including, for example, by the methods described in the Examples below).

In some embodiments, the masking unit sterically hinders binding of the activatable antibody to CTLA4 and/or allosterically hinders binding of the activatable antibody to CTLA4. In some embodiments, the masking unit does not comprise an amino acid sequence of a natural binding partner of the activatable antibody and/or parental antibody.

In some embodiments, the dissociation constant of the masking unit for the target binding moiety is greater than the dissociation constant for the activatable antibody for CTLA4 (when activated, e.g., when the cleavage site is cleaved). In some embodiments, the dissociation constant of the masking unit for the target binding moiety is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more) times greater than the dissociation constant for the activatable antibody for CTLA4 (when activated e.g., when the cleavage site is cleaved). In some embodiments, the dissociation constant of the masking unit for the target binding moiety is about equal to the dissociation constant for the activatable antibody for CTLA4 (when activated e.g., when the cleavage site is cleaved).

The anti-CTLA4 masked antibodies (e.g., activatable antibodies) described herein may be further modified. In some embodiments, the anti-CTLA4 masked antibodies (e.g., activatable antibodies) are linked to an additional molecular entity. Examples of additional molecular entities include pharmaceutical agents, peptides or proteins, detection agent or labels, and antibodies.

In some embodiments, an anti-CTLA4 masked antibody (e.g., activatable antibody) of the present disclosure is linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine³¹, indium¹¹¹, yttrium⁹⁰ and lutetium¹⁷⁷. Methods for linking a polypeptide to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies see e.g., Saito et al., Adv. Drug Deliv. Rev. 55: 199-215 (2003); Trail, et al., Cancer Immunol. Immunother. 52: 328-337 (2003); Payne, Cancer Cell 3: 207-212 (2003); Allen, Nat. Rev. Cancer 2: 750-763 (2002); Pastan and Kreitman, Curr. Opin. Investig. Drugs 3: 1089-1091 (2002); Senter and Springer (2001) Adv. Drug Deliv. Rev. 53: 247-264.

### V. Nucleic Acids, Vectors, Host Cells, and Recombinant Methods of Producing CTLA4 Antibodies and/or Masked Antibodies

Another aspect of the disclosure provides an isolated nucleic acid molecule that comprises a nucleotide sequence encoding an amino acid sequence of an anti-CTLA4 binding molecule (e.g., an antibody or masked antibody (e.g., activatable antibody)) provided herein. The amino acid sequence encoded by the nucleotide sequence may be any portion of an anti-CTLA4 antibody, such as a CDR, a sequence comprising one, two, or three CDRs, a variable region of a heavy chain, variable region of a light chain, or may be a full-length heavy chain or full length light chain. A nucleic acid of the disclosure can be, for example, DNA or RNA, and may or may not contain intronic sequences. Typically, the nucleic acid is a cDNA molecule.

In some embodiments, the disclosure provides an isolated nucleic acid molecule that comprises or consists of a nucleotide sequence encoding an amino acid sequence selected from the group consisting of: (1) amino acid sequence of an CDR-H1, CDR-H2, CDR-H3, CDR-L1, CDR-L2 and/or and CDR-L3 of an illustrative anti-CTLA4 antibody described herein; (2) a variable region of a heavy chain and/or variable region of a light chain of an illustrative anti-CTLA4 antibody described herein; or (3) a full length heavy chain or full length light chain of an illustrative anti-CTLA4 antibody.

In some embodiments, the nucleic acid molecule comprises or consists of a nucleotide sequence that encodes an amino acid sequence as set forth in any one of SEQ ID NOS: 104-117. Nucleic acids of the present disclosure may be obtained using any suitable molecular biology techniques. For antibodies expressed by hybridomas, cDNAs encoding the light and heavy chains of the antibody made by the hybridoma can be obtained by PCR amplification or cDNA cloning techniques. For antibodies obtained from an immunoglobulin gene library (e.g., using phage display techniques), the nucleic acid encoding the antibody can be recovered from the library.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al. (1991) NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art (see e.g., Kabat et al. (1991) NIH Publication No. 91-3242) and DNA fragments encompassing these regions can be obtained by standard PCR amplification. The light chain constant region can be a kappa or lambda constant region.

To create an scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (see e.g., Bird et al., Science 242: 423-426 (1988); Huston et al., Proc. Natl. Acad. Sci. USA 85: 5879-5883 (1988); and McCafferty et al., Nature 348: 552-554 (1990)).

The present disclosure further provides a vector that comprises a nucleic acid molecule described herein. In some embodiments, the vector is an expression vector or a display vector (e.g., a viral display vector, a bacterial display vector, a yeast display vector, an insect display vector, a mammalian display vector, etc.). The nucleic acid molecule may encode a portion of a light chain or heavy chain (such as a CDR or a HVR; a light or heavy chain variable region), a full-length light or heavy chain, polypeptide that comprises a portion or full-length of a heavy or light chain, or an amino acid sequence of an antibody derivative or antigen-binding fragment. In some embodiments, the vector is an expression vector useful for the expression of an anti-CTLA4 binding molecule, such as an anti-CTLA4 antibody, a masked antibody (e.g., activatable antibody), or an antigen binding fragment thereof. In some embodiments, provided herein are vectors, wherein a first vector comprises a polynucleotide sequence encoding a heavy chain variable region as described herein, and a second vector comprises a polynucleotide sequence encoding a light chain variable region as described herein. In some embodiments, a single vector comprises polynucleotides encoding a heavy chain variable region as described herein and a light chain variable region as described herein.

To express a binding molecule of the disclosure, DNAs encoding partial or full-length light and heavy chains are inserted into expression vectors such that the DNA molecules are operatively linked to transcriptional and translational control sequences. In this context, the term “operatively linked” means that an antibody gene is ligated into a vector such that transcriptional and translational control sequences within the vector serve their intended function of regulating the transcription and translation of the DNA molecule. The expression vector and expression control sequences are chosen to be compatible with the expression host cell used. The antibody light chain gene and the antibody heavy chain gene can be inserted into separate vectors, or both genes can be inserted into the same expression vector. The antibody genes are inserted into the expression vector by any suitable methods (e.g., ligation of complementary restriction sites on the antibody gene fragment and vector, or homologous recombination-based DNA ligation). The light and heavy chain variable regions of the anti-CTLA4 antibodies described herein can be used to create full-length anti-CTLA4 antibody genes of any antibody isotype and subclass by inserting them into expression vectors already encoding heavy chain constant and light chain constant regions of the desired isotype and subclass such that the V_{H} segment is operatively linked to the C_{H} segment (s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the anti-CTLA4 antibody sequences, the expression vectors of the disclosure typically carry regulatory sequences that control the expression of the anti-CTLA4 antibody sequences in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. (1988) Mol. Cell. Biol. 8: 466-472).

In addition to the antibody chain genes and regulatory sequences, the expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector (s) encoding the heavy and light chains is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the anti-CTLA4 antibodies of the disclosure in either prokaryotic or eukaryotic host cells, expression of anti-CTLA4 antibodies in eukaryotic cells, and typically mammalian host cells, is most typical.

The present disclosure further provides a host cell containing a nucleic acid molecule provided by the present disclosure. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Methods of introducing a recombinant nucleic acid into a host cell are known in the art, including, for example, by calcium phosphate transfection, DEAE, dextran mediated transfection, electroporation or phage infection.

Suitable prokaryotic hosts for transformation include E. coli, Bacillus subtilis, Salmonella typhimurium and various species within the genera Pseudomonas, Streptomyces, and Staphylococcus.

Suitable eukaryotic hosts for transformation include yeast, insect (e.g., S2 cells), and mammalian cells. Mammalian host cells for expressing a binding molecule of the disclosure include, for example, Chinese Hamster Ovary (CHO) cells (including dhfr-CHO cells, described in Urlaub and Chasin, Proc. Natl. Acad. Sci. USA 77: 4216-4220 (1980); Sharp, J. Mol. Biol. 159: 601-621 (1982)), NSO myeloma cells, COS cells, HEK293F cells, HEK293Tcells, and Sp2 cells. In particular, for use with NSO myeloma or CHO cells, another expression system is the GS (glutamine synthetase) gene expression system disclosed in WO 87/04462, WO 89/01036 and EP 338, 841. In some embodiments, anti-CTLA4 antibodies of the present disclosure are produced in CHO cells. In some embodiments, anti-CTLA4 antibodies of the present disclosure are modified, and do not include a C-terminal lysine residue (e.g., the C-terminal lysine residue of an antibody heavy chain described herein is removed (such as before or during antibody production)). When expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using any suitable protein purification methods known in the art (e.g., protein A chromatography and/or ion exchange chromatography).

### VI. Compositions

In other aspects, the present disclosure provides a composition containing an anti-CTLA4 binding molecule (e.g., an anti-CTLA4 antibody or masked antibody (e.g., activatable antibody)) provided by the disclosure. In one aspect, the composition is a pharmaceutical composition comprising an anti-CTLA4 binding molecule (e.g., an anti-CTLA4 antibody or masked antibody (e.g., activatable antibody)) and a pharmaceutically acceptable carrier. The compositions can be prepared by conventional methods known in the art.

The term “pharmaceutically acceptable carrier” refers to any inactive substance that is suitable for use in a formulation for the delivery of a binding molecule. A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an anti-CTLA4 binding molecule (e.g., an anti-CTLA4 antibody or masked antibody (e.g., activatable antibody)) is a sterile liquid, such as a solution, suspension, or dispersion, for injection or infusion. Sterile solutions can be prepared by incorporating the anti-CTLA4 antibody in the required amount in an appropriate carrier, followed by sterilization microfiltration. Generally, dispersions are prepared by incorporating the anti-CTLA4 binding molecule (e.g., anti-CTLA4 antibody or masked antibody (e.g., activatable antibody)) into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

In addition to the anti-CTLA4 binding molecule (e.g., anti-CTLA4 antibody or masked antibody (e.g., activatable antibody)), one or more additional therapeutic agents may be included in the composition. Examples of additional therapeutic agents are described herein below. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent which produces a therapeutic effect, but may be a lesser amount as well.

Any of the anti-CTLA4 binding molecules (e.g., anti-CTLA4 antibodies or masked antibodies (e.g., activatable antibodies)) and/or compositions (e.g., pharmaceutical compositions) described herein may be used in the preparation of a medicament (e.g., a medicament for use in treating or delaying progression of cancer in a subject in need thereof).

### VII. Use of the CTLA4 Binding Molecules and Pharmaceutical Compositions

The CTLA4 binding molecules (e.g., anti-CTLA4 antibodies or masked antibody (e.g., activatable antibody)) and pharmaceutical compositions provided by the present disclosure are useful for therapeutic, diagnostic, or other purposes, such as modulating an immune response, treating cancer, enhancing efficacy of other cancer therapy, enhancing vaccine efficacy, or treating autoimmune diseases. Thus, in other aspects, the present disclosure provides methods of using the anti-CTLA4 binding molecules (e.g., anti-CTLA4 antibodies or masked antibody (e.g., activatable antibodies)) or pharmaceutical compositions. In one aspect, the present disclosure provides a method of treating a disorder in a subject, which comprises administering to the subject in need of treatment an effective amount of a CTLA4 binding molecule (e.g., an anti-CTLA4 antibody or masked antibody (e.g., activatable antibody)) or composition provided by the present disclosure. In some embodiments, the subject is a mammal. In some embodiments, the subject is a human.

In some embodiments, provided herein is a method of treating or delaying progression of a disorder in a subject in need thereof, the method comprising administering to the subject an effective amount of an anti-CTLA4 antibody, an anti-CTLA4 antigen-biding fragment, an anti-CTLA4 masked antibody (e.g., an anti-CTLA4 activatable antibody), or pharmaceutical composition of the present disclosure. In some embodiments, the disorder is a cancer. A variety of cancers may be treated or prevented with a method, use, composition, or medicament provided by the present disclosure. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblastic, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraocular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In some embodiments, the cancer is a colorectal cancer. In some embodiments, the cancer is a lung cancer. In some embodiments, the cancer is a lymphoma. In certain embodiments, the method comprises administering to the subject an effective amount of the anti-CTLA4 antibody, the anti-CTLA4 masked antibody, or the pharmaceutical composition prior to a surgery or after a surgery to remove a solid tumor in the subject.

In some embodiments, the tumor is a cold tumor. In some embodiments, the cold tumor is a brain cancer (e.g., glioblastoma), a breast cancer, a head and neck cancer, an ovarian cancer, a pancreatic cancer, or a prostate cancer. In some embodiments, the cold tumor is selected from the group consisting of pancreatic cancer, ovarian cancer, and head and neck cancer. In certain embodiments, the cold tumor is pancreatic cancer. In certain embodiments, the cold tumor is ovarian cancer. In certain embodiments, the cold tumor is head and neck cancer. In some embodiments, the cold tumor is microsatellite stable colorectal cancer.

In some embodiments, provided herein is a method of reducing size of a solid tumor in a subject in need thereof, wherein the solid tumor has a size of about 400-1000 mm³ (e.g., between about 400 and about 1000 mm³, between about 400 and about 800 mm³, between about 400 and about 600 mm³, between about 400 and about 500 mm³ between about 500 and about 1000 mm³, between about 500 and about 700 mm³ between about 500 and about 600 mm³, between about 600 and about 1000 mm³ between about 600 and about 800 mm³, between about 700 and about 1000 mm³ between about 700 and about 900 mm³, between about 800 and about 1000 mm³, between about 800 and about 900 mm³, or between about 900 and about 1000 mm³), the method comprising administering to the subject an effective amount of an anti-CTLA4 antibody, an anti-CTLA4 antigen-biding fragment, an anti-CTLA4 masked antibody (e.g., an anti-CTLA4 activatable antibody), or pharmaceutical composition of the present disclosure. In some embodiments, the tumor is a colorectal tumor or a lung tumor. In certain embodiments, the method comprises administering to the subject an effective amount of the anti-CTLA4 antibody, the anti-CTLA4 masked antibody, or the pharmaceutical composition prior to a surgery or after a surgery to remove a solid tumor in the subject.

In some embodiments, provided herein is a method of reducing on-target off-tumor toxicity in a subject in need thereof, wherein the method comprises administering to the subject an effective amount of a masked anti-CTLA4 antibody (e.g., an activatable anti-CTLA4 antibody) of the present disclosure. In some embodiments, the subject has cancer. In some embodiments, the subject has a tumor. In some embodiments, the tumor is a colorectal tumor or a lung tumor. In some embodiments, the on-target off-tumor toxicity is diabetes. In certain embodiments, the on-target off-tumor toxicity is immune checkpoint inhibitor-associated diabetes (e.g., diabetes associated with the administration of an inhibitor of PD-1, such as an anti-PD-1 antibody). In some embodiments, the masked anti-CTLA4 antibody is administered after administration of an immune checkpoint inhibitor (e.g., an inhibitor of PD-1, such as an anti-PD-1 antibody). In further embodiments, the masked anti-CTLA4 antibody is administered concurrently with administration of an immune checkpoint inhibitor (e.g., an inhibitor of PD-1, such as an anti-PD-1 antibody). In some embodiments, administration of the masked anti-CTLA4 antibody (e.g., activatable anti-CTLA4 antibody) to the subject results in decreased frequency of symptoms of diabetes (e.g., immune checkpoint inhibitor-associated diabetes) in the subject (e.g., results in lower blood glucose levels or a slower increase in blood glucose levels) as compared to administration of a control antibody (e.g., a non-masked anti-CTLA4 antibody). In certain embodiments, the method comprises administration of an activatable anti-CTLA4 antibody comprising a masking unit (MU) and a target-binding moiety (TBM), wherein the administration results in decreased blood glucose levels as compared to administration of an anti-CTLA4 antibody comprising the same TBM but lacking the MU. In some embodiments, administration of the masked anti-CTLA4 antibody (e.g., activatable anti-CTLA4 antibody) results in symptoms of diabetes (e.g., elevated blood glucose levels) at a frequency of less than 60% (e.g., less than 60%, less than 50%, less than 40%, less than 30%, less than 20%, or less than 10%). Methods of measuring blood glucose, as well as healthy ranges of blood glucose for human and non-human animal subjects, are well known in the art.

In some embodiments, provided herein is a method of depleting Treg cells in a subject in need thereof, wherein the method comprises administering to the subject an effective amount of a masked anti-CTLA4 antibody (e.g., an activatable anti-CTLA4 antibody) of the present disclosure. In some embodiments, the subject has cancer. In some embodiments, the subject has a tumor. In some embodiments, the tumor is a colorectal tumor or a lung tumor. In some embodiments, the masked anti-CTLA4 antibody (e.g., activatable anti-CTLA4 antibody) has ADCC activity against a CTLA-4 expressing Treg cell. In certain embodiments wherein the masked anti-CTLA4 antibody (e.g., activatable anti-CTLA4 antibody) comprises an IgG1 Fc region comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the ADCC activity of the masked anti-CTLA4 antibody (e.g., activatable anti-CTLA4 antibody) is higher than that of a control antibody comprising a human IgG1 Fc region that does not comprise the substitutions. In certain embodiments, the ADCC activity of the masked anti-CTLA4 antibody (e.g., activatable anti-CTLA4 antibody) is higher than the ADCC activity of a control anti-CTLA4 antibody (e.g., ipilimumab). In some embodiments, administration of the masked anti-CTLA4 antibody (e.g., activatable anti-CTLA4 antibody) results in a reduction in the percentage of Tregs in T cells (e.g., CD4+ T cells or CD8+ T cells) in comparison to a control (e.g., an isotype control). In some embodiments, administration of the masked anti-CTLA4 antibody (e.g., activatable anti-CTLA4 antibody) results in a reduction in the percentage of Tregs in T cells (e.g., CD4+ T cells or CD8+ T cells) in tumor infiltrating lymphocytes (TILs) or peripheral blood mononuclear cells (PMBCs) in comparison to a control (e.g., an isotype control). In some embodiments, administration of the masked anti-CTLA4 antibody (e.g., activatable anti-CTLA4 antibody) results in a reduction in the percentage of Tregs in T cells (e.g., CD4+ T cells or CD8+ T cells) of at least about 0.5% (e.g., at least about 0.5%, at least about 1%, at least about 2%, at least about 3%, at least about 4%, at least about 5%, at least about 6%, at least about 8%, at least about 10%, at least about 12%, at least about 14%, at least about 16%, at least about 18%, at least about 20%, or at least about 25%). In some embodiments, administration of the masked anti-CTLA4 antibody (e.g., activatable anti-CTLA4 antibody) results in a reduction in the percentage of Tregs in T cells (e.g., CD4+ T cells or CD8+ T cells) of between about 0.5% and about 25% (e.g., between about 0.5% and 5%, between about 0.5% and 10%, between about 0.5% and about 15%, between about 0.5% and about 20%, between about 0.5% and about 25%, between about 5% and about 10%, between about 5% and about 15%, between about 5% and about 20%, between about 5% and about 25%, between about 10% and about 15%, between about 10% and about 20%, between about 10% and about 25%, between about 15% and about 20%, between about 15% and about 25%, or between about 20% and about 25%).

In some embodiments, provided herein is a method of treating cancer comprising administering an anti-CTLA4 antibody to a subject in need thereof, wherein the anti-CTLA4 antibody comprises (1) a first polypeptide and a second polypeptide each comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising an antibody light chain variable region (VL) comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and/or the second polypeptide comprise an IgG1 Fc domain. In some embodiments, the first polypeptide and/or the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and/or the second polypeptide each comprise an IgG1 Fc domain comprising a S239D substitution and/or an I332E substitution. In some embodiments, the anti-CTLA4 antibody is a masked antibody, wherein the third polypeptide and the fourth polypeptide each comprise from N- to C-terminus, a masking peptide comprising a masking unit (MU) and a linkage unit (LU), and the VL. In certain embodiments, the anti-CTLA4 antibody is an activatable antibody, wherein the third polypeptide and the fourth polypeptide each comprise from N- to C-terminus, a masking peptide comprising a masking unit (MU) and a linkage unit (LU), and the VL. In some embodiments, the LU comprises at least a first cleavage site. In some embodiments, the MU comprises an amino acid sequence selected from the group consisting of SEQ ID NOS: 146-151. In some embodiments, the linkage unit (LU) comprises an amino acid sequence selected from the group consisting of SEQ ID NOS: 99-103. In some embodiments, the cancer is a colorectal cancer. In some embodiments, the cancer is a lung cancer. In some embodiments, the cancer is a lymphoma.

In some embodiments, provided herein is a method of treating cancer comprising administering an anti-CTLA4 antibody to a subject in need thereof, wherein the anti-CTLA4 antibody comprises (1) a first polypeptide and a second polypeptide each comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising, from N- to C-terminus, (a) a masking peptide comprising a masking unit (MU) and a linkage unit comprising at least a first cleavage site, wherein the MU comprises an amino acid sequence selected from the group consisting of SEQ ID NOS: 146-151, and (b) an antibody light chain variable region (VL) comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In certain embodiments, the linkage unit (LU) comprises an amino acid sequence selected from the group consisting of SEQ ID NOS: 99-103. In some embodiments, the cancer is a colorectal cancer. In some embodiments, the cancer is a lung cancer. In some embodiments, the cancer is a lymphoma.

In some embodiments, provided herein is a method of treating cancer comprising administering an anti-CTLA4 antibody to a subject in need thereof, wherein the anti-CTLA4 antibody comprises (1) a first polypeptide and a second polypeptide each comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3, or a variant thereof comprising up to 5 (e.g., 1, 2, 3, 4, or 5) amino acid substitutions; and (2) and a third and a fourth polypeptide each comprising, from N- to C-terminus, (a) a masking peptide comprising a masking unit (MU) and a linkage unit (LU) comprising at least a first cleavage site, wherein the MU comprises the amino acid sequence of SEQ ID NO: 150, and the LU comprises the amino acid sequence of SEQ ID NO: 103; and (b) an antibody light chain variable region (VL) comprising a CDR-L1, comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In some embodiments, the first and second polypeptide comprise the amino acid sequence of SEQ ID NO: 105, or an amino acid sequence having at least 80% (e.g., at least 85%, 90%, 95%, 98%, or 99%; or 100%) sequence identity with SEQ ID NO: 105, and the third and fourth polypeptide comprise the amino acid sequence of SEQ ID NO: 121, or an amino acid sequence having at least 80% (e.g., at least 85%, 90%, 95%, 98%, or 99%; or 100%) sequence identity with SEQ ID NO: 121. In some embodiments, the cancer is a colorectal cancer. In some embodiments, the cancer is a lung cancer. In some embodiments, the cancer is a lymphoma.

In some embodiments of the methods described herein, the method further comprises administering to the subject an effective amount of at least one additional therapeutic agent. In some embodiments, the at least one additional therapeutic agent comprises viral gene therapy, immune checkpoint inhibitors, target therapies, radiation therapies, vaccination therapies, chemotherapies, or any combination thereof.

In some embodiments, the at least one additional therapeutic agent comprises an immune checkpoint inhibitor. In some embodiments, the immune checkpoint inhibitor is an inhibitor of PD-1. In certain embodiments, the inhibitor of PD-1 is an anti-PD-1 antibody. Any of the anti-PD-1 antibodies known in the art may be used in the present invention, including, but not limited to, Nivolumab, pembrolizumab, pidilizumab, BMS-936559, atezolizumab, Lambrolizumab, MK-3475, AMP-224, AMP-514. STI-A1110, TSR-042, 2E5 (CN107840887A), and biosimilars thereof. In some embodiments, the anti-PD-1 antibody is an anti-PD-1 antibody described in U.S. Pat. Pub. No. 2021/0122824, which is herein incorporated by reference in its entirety. In some embodiments, the anti-PD-1 antibody is a monoclonal antibody or a polyclonal antibody. In some embodiments, the anti-PD-1 antibody is an antigen-binding fragment selected from the group consisting of Fab, Fab′, F (ab′) 2, Fv, scFv, and other antigen-binding subsequences of the full-length anti-PD-1 antibody. In some embodiments, the anti-PD-1 antibody is a human, humanized, or chimeric antibody. In some embodiments, the anti-PD-1 antibody is a bispecific antibody, a multispecific antibody, a single domain antibody, a fusion protein comprising an antibody portion, or any other variants or derivatives thereof. In some embodiments, the inhibitor of PD-1 is a natural or engineered ligand of PD-1, such as PD-L1 or PD-L2. In some embodiments, the inhibitor of PD-1 is an inhibitor of the interaction between PD-1 and its ligand, for example, an inhibitor of PD-1/PD-L1 interaction or an inhibitor of PD-1/PD-L2 interaction. In some embodiments, the inhibitor of PD-1 is an inhibitor of a PD-1 ligand, such as an inhibitor of PD-L (e.g., anti-PD-L1 antibody) or an inhibitor of PD-L2 (e.g., anti-PD-L2 antibody). Any of the inhibitors of interaction between PD-1 and its ligand may be used in the present invention, see, for example, U.S. Pat. Nos. 7,709,214, 7,432,059, 7,722,868, 8,217,149, 8,383,796, and 9,102,725 and U.S. Pat. Pub. No. 2021/0122824.

In some embodiments, provided herein is a method of treating cancer comprising administering to a subject in need thereof a) an anti-CTLA4 antibody and b) an anti-PD-1 antibody, wherein anti-CTLA4 antibody comprises (1) a first polypeptide and a second polypeptide each comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising an antibody light chain variable region (VL) comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and/or the second polypeptide comprise an IgG1 Fc domain. In some embodiments, the first polypeptide and/or the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and/or the second polypeptide each comprise an IgG1 Fc domain comprising a S239D substitution and/or an I332E substitution. In some embodiments, the anti-CTLA4 antibody is a masked antibody, wherein the third polypeptide and the fourth polypeptide each comprise from N- to C-terminus, a masking peptide comprising a masking unit (MU) and a linkage unit (LU), and the VL. In certain embodiments, the anti-CTLA4 antibody is an activatable antibody, wherein the third polypeptide and the fourth polypeptide each comprise from N- to C-terminus, a masking peptide comprising a masking unit (MU) and a linkage unit (LU), and the VL. In some embodiments, the LU comprises at least a first cleavage site. In some embodiments, the MU comprises an amino acid sequence selected from the group consisting of SEQ ID NOS: 146-151. In some embodiments, the linkage unit (LU) comprises an amino acid sequence selected from the group consisting of SEQ ID NOS: 99-103. In some embodiments, the cancer is a colorectal cancer. In some embodiments, the anti-PD-1 antibody is selected from the group consisting of Nivolumab, pembrolizumab, pidilizumab, BMS-936559, atezolizumab, Lambrolizumab, MK-3475, AMP-224, AMP-514. STI-A1110, and TSR-042. In some embodiments, the anti-PD-1 antibody is an anti-PD-1 antibody described in U.S. Pat. Pub. No. 2021/0122824, which is herein incorporated by reference in its entirety.

In some embodiments, provided herein is a method of treating cancer comprising administering to a subject in need thereof a) an anti-CTLA4 antibody and b) an anti-PD-1 antibody, wherein anti-CTLA4 antibody comprises (1) a first polypeptide and a second polypeptide each comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising, from N- to C-terminus, (a) a masking peptide comprising a masking unit (MU) and a linkage unit (LU), wherein the MU comprises an amino acid sequence selected from the group consisting of SEQ ID NOS: 146-151, and (b) an antibody light chain variable region (VL) comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the LU comprises at least a first cleavage site. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In certain embodiments, the linkage unit (LU) comprises an amino acid sequence selected from the group consisting of SEQ ID NOS: 99-103. In some embodiments, the cancer is a colorectal cancer. In some embodiments, the anti-PD-1 antibody is selected from the group consisting of Nivolumab, pembrolizumab, pidilizumab, BMS-936559, atezolizumab, Lambrolizumab, MK-3475, AMP-224, AMP-514. STI-A1110, and TSR-042. In some embodiments, the anti-PD-1 antibody is an anti-PD-1 antibody described in U.S. Pat. Pub. No. 2021/0122824, which is herein incorporated by reference in its entirety.

In some embodiments, provided herein is a method of treating cancer comprising administering to a subject in need thereof a) an anti-CTLA4 antibody and b) an anti-PD-1 antibody, wherein anti-CTLA4 antibody comprises (1) a first polypeptide and a second polypeptide each comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3, or a variant thereof comprising up to 5 (e.g., 1, 2, 3, 4, or 5) amino acid substitutions; and (2) and a third and a fourth polypeptide each comprising, from N- to C-terminus, (a) a masking peptide comprising a masking unit (MU) and a linkage unit (LU) comprising a cleavage site, wherein the MU comprises the amino acid sequence of SEQ ID NO: 150, and the LU comprises the amino acid sequence of SEQ ID NO: 103; and (b) an antibody light chain variable region (VL) comprising a CDR-L1, comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In some embodiments, the first and second polypeptide comprise the amino acid sequence of SEQ ID NO: 105, or an amino acid sequence having at least 80% (e.g., at least 85%, 90%, 95%, 98%, or 99%; or 100%) sequence identity with SEQ ID NO: 105, and the third and fourth polypeptide comprise the amino acid sequence of SEQ ID NO: 121, or an amino acid sequence having at least 80% (e.g., at least 85%, 90%, 95%, 98%, or 99%; or 100%) sequence LGHQWLWy with SEQ ID NO: 121. In some embodiments, the cancer is a colorectal cancer. In some embodiments, the anti-PD-1 antibody is selected from the group consisting of Nivolumab, pembrolizumab, pidilizumab, BMS-936559, atezolizumab, Lambrolizumab, MK-3475, AMP-224, AMP-514. STI-A1110, and TSR-042. In some embodiments, the anti-PD-1 antibody is an anti-PD-1 antibody described in U.S. Pat. Pub. No. 2021/0122824, which is herein incorporated by reference in its entirety.

In some embodiments, the at least one additional therapeutic agent comprises a CD137 agonist. In some embodiments, the at least one additional therapeutic agent comprises an anti-CD137 antibody (e.g., an agonist anti-CD137 antibody). Any of the anti-CD137 antibodies known in the art may be used in the present invention, including, but not limited to, urelumab, utomilumab (PF-05082566), AG10131, AGEN2373, CTX-471, ATOR-1017, anti-CD137 bispecific antibodies, and biosimilars thereof. In some embodiments, the anti-CD137 antibody is an anti-CD137 antibody described in U.S. Pat. Pub. No. 2020/0369776, which is incorporated herein by reference in its entirety. In some embodiments, the anti-CD137 antibody is a monoclonal antibody or a polyclonal antibody. In some embodiments, the anti-CD137 antibody is an antigen-binding fragment selected from the group consisting of Fab, Fab′, F (ab′) 2, Fv, scFv, and other antigen-binding subsequences of the full-length anti-CD137 antibody. In some embodiments, the anti-CD137 antibody is a human, humanized, or chimeric antibody. In some embodiments, the anti-CD137 antibody is a bispecific antibody, a multispecific antibody, a single domain antibody, a fusion protein comprising an antibody portion, or any other variants or derivatives thereof. In some embodiments, the CD137 agonist is a natural or engineered ligand of CD137, such as CD137 ligand (CD137L).

In some embodiments, the at least one additional therapeutic agent comprises an anti-CD137 antibody. In some embodiments, the anti-CD137 antibody comprises a heavy chain variable region (VH) and a light chain variable region (VL), wherein a) the heavy chain variable region comprises an HVR-H1, an HVR-H2, and an HVR-H3, wherein the HVR-H1 comprises the amino acid sequence FSLSTGGVGVGWI (SEQ ID NO: 135) or a variant thereof comprising up to 5 (e.g., 1, 2, 3, 4, or 5) amino acid substitutions; the HVR-H2 comprises the amino acid sequence LALIDWADDKYYSPSLKSRL (SEQ ID NO: 136), or a variant thereof comprising up to 5 (e.g., 1, 2, 3, 4, or 5) amino acid substitutions; and/or the HVR-H3 comprises the amino acid sequence ARGGSDTVIGDWFAY (SEQ ID NO: 137), or a variant thereof comprising up to 5 (e.g., 1, 2, 3, 4, or 5) amino acid substitutions; and/or b) the light chain variable region comprises an HVR-L1, an HVR-L2, and an HVR-L3, wherein the HVR-L1 comprises the amino acid sequence RASQSIGSYLA (SEQ ID NO: 138), or a variant thereof comprising up to 5 (e.g., 1, 2, 3, 4, or 5) amino acid substitutions; the HVR-L2 comprises the amino acid sequence DASNLETGV (SEQ ID NO: 139), or a variant thereof comprising up to 5 (e.g., 1, 2, 3, 4, or 5) amino acid substitutions; and/or the HVR-L3 comprises the amino acid sequence YCQQGYYLWT (SEQ ID NO: 140), or a variant thereof comprising up to 5 (e.g., 1, 2, 3, 4, or 5) amino acid substitutions. In some embodiments, the anti-CD137 antibody comprises a heavy chain variable region (VH) and a light chain variable region (VL), wherein the VH comprises the amino acid sequence of SEQ ID NO: 141, or an amino acid sequence having at least 80% (e.g., at least 85%, 90%, 95%, 98%, or 99%; or 100%) sequence identity with SEQ ID NO: 141; and/or the VL comprises the amino acid sequence of SEQ ID NO: 142, or an amino acid sequence having at least 80% (e.g., at least 85%, 90%, 95%, 98%, or 99%; or 100%) sequence identity with SEQ ID NO: 142. In certain embodiments, the anti-CD137 antibody is AG10131.

### AG10131 Heavy Chain Variable Region (VH):

(SEQ ID NO: 141)
EVQLVESGGGLVQPGGSLRLSCAASGFSLSTGGVGVGWIRQAPGKG
LEWLALIDWADDKYYSPSLKSRLTISRDNSKNTLYLQLNSLRAEDT
AVYYCARGGSDTVIGDWFAYWGQGTLVTVSS

### AG10131 Light Chain Variable Region (VL):

(SEQ ID NO: 142)
DIQLTQSPSSLSASVGDRVTITCRASQSIGSYLAWYQQKPGKAPKL
LIYDASNLETGVPSRFSGSGSGTDFTLTISSLQPEDFATYYCQQGY
YLWTFGQGTKVEIKR

In some embodiments, provided herein is a method of treating cancer comprising administering to a subject in need thereof a) an anti-CTLA4 antibody and b) an anti-CD137 antibody, wherein anti-CTLA4 antibody comprises (1) a first polypeptide and a second polypeptide each comprising, from N- to C-terminus, an antibody heavy chain variable region (VH) and a human IgG1 Fc domain, wherein the human IgG1 Fe domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising an antibody light chain variable region (VL) comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and/or the second polypeptide comprise an IgG1 Fc domain. In some embodiments, the first polypeptide and/or the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and/or the second polypeptide each comprise an IgG1 Fc domain comprising a S239D substitution and/or an I332E substitution. In some embodiments, the anti-CTLA4 antibody is a masked antibody, wherein the third polypeptide and the fourth polypeptide each comprise from N- to C-terminus, a masking peptide comprising a masking unit (MU) and a linkage unit (LU), and the VL. In certain embodiments, the anti-CTLA4 antibody is an activatable antibody, wherein the third polypeptide and the fourth polypeptide each comprise from N- to C-terminus, a masking peptide comprising a masking unit (MU) and a linkage unit (LU), and the VL. In some embodiments, the MU comprises an amino acid sequence selected from the group consisting of SEQ ID NOS: 146-151. In some embodiments, the LU comprises at least a first cleavage site. In some embodiments, the LU comprises an amino acid sequence selected from the group consisting of SEQ ID NOS: 99-103. In some embodiments, the cancer is a colorectal cancer. In some embodiments, the anti-CD137 antibody comprises a heavy chain variable region (VH) and a light chain variable region (VL), wherein a) the heavy chain variable region comprises an HVR-H1, an HVR-H2, and an HVR-H3, wherein the HVR-H1 comprises the amino acid sequence of SEQ ID NO: 135, the HVR-H2 comprises the amino acid sequence of SEQ ID NO: 136, and the HVR-H3 comprises the amino acid sequence of SEQ ID NO: 137; and b) the light chain variable region comprises an HVR-L1, an HVR-L2, and an HVR-L3, wherein the HVR-L1 comprises the amino acid sequence of SEQ ID NO: 138, the HVR-L2 comprises the amino acid sequence of SEQ ID NO: 139, and the HVR-L3 comprises the amino acid sequence of SEQ ID NO: 140. In some embodiments, the anti-CD137 antibody comprises a VH and a VL, wherein the VH comprises the amino acid sequence of SEQ ID NO: 141 and the VL comprises the amino acid sequence of SEQ ID NO: 142. In some embodiments, the anti-CD137 antibody is selected from the group consisting of urelumab, utomilumab (PF-05082566), and AG10131. In certain embodiments, the anti-CD137 antibody is AG10131. In some embodiments, the anti-CD137 antibody is an anti-CD137 antibody described in U.S. Pat. Pub. No. 2020/0369776.

In some embodiments, provided herein is a method of treating cancer comprising administering to a subject in need thereof a) an anti-CTLA4 antibody and b) an anti-CD137 antibody, wherein anti-CTLA4 antibody comprises (1) a first polypeptide and a second polypeptide comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3; and (2) and a third and a fourth polypeptide each comprising, from N- to C-terminus, (a) a masking peptide comprising a masking unit (MU) and a linkage unit (LU), wherein the MU comprises an amino acid sequence selected from the group consisting of SEQ ID NOS: 146-151, and (b) an antibody light chain variable region (VL) comprising a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In some embodiments, the LU comprises at least a first cleavage site. In certain embodiments, the LU comprises an amino acid sequence selected from the group consisting of SEQ ID NOS: 99-103. In some embodiments, the cancer is a colorectal cancer. In some embodiments, the anti-CD137 antibody comprises a heavy chain variable region (VH) and a light chain variable region (VL), wherein a) the heavy chain variable region comprises an HVR-H1, an HVR-H2, and an HVR-H3, wherein the HVR-H1 comprises the amino acid sequence of SEQ ID NO: 135, the HVR-H2 comprises the amino acid sequence of SEQ ID NO: 136, and the HVR-H3 comprises the amino acid sequence of SEQ ID NO: 137; and b) the light chain variable region comprises an HVR-L1, an HVR-L2, and an HVR-L3, wherein the HVR-L1 comprises the amino acid sequence of SEQ ID NO: 138, the HVR-L2 comprises the amino acid sequence of SEQ ID NO: 139, and the HVR-L3 comprises the amino acid sequence of SEQ ID NO: 140. In some embodiments, the anti-CD137 antibody comprises a VH and a VL, wherein the VH comprises the amino acid sequence of SEQ ID NO: 141 and the VL comprises the amino acid sequence of SEQ ID NO: 142. In some embodiments, the anti-CD137 antibody is selected from the group consisting of urelumab, utomilumab (PF-05082566), and AG10131. In certain embodiments, the anti-CD137 antibody is AG10131. In some embodiments, the anti-CD137 antibody is an anti-CD137 antibody described in U.S. Pat. Pub. No. 2020/0369776.

In some embodiments, provided herein is a method of treating cancer comprising administering to a subject in need thereof a) an anti-CTLA4 antibody and b) an anti-CD137 antibody, wherein anti-CTLA4 antibody comprises (1) a first polypeptide and a second polypeptide comprising, from N- to C-terminus, an antibody heavy chain variable region (VH) and a human IgG1 Fc domain, wherein the human IgG1 Fc domains of the first and second polypeptide dimerize to form a human IgG1 Fc region, and wherein the VH comprises a CDR-H1 comprising the amino acid sequence of SEQ ID NO: 1; a CDR-H2 comprising the amino acid sequence of SEQ ID NO: 2; and a CDR-H3 comprising the amino acid sequence of SEQ ID NO: 3, or a variant thereof comprising up to 5 (e.g., 1, 2, 3, 4, or 5) amino acid substitutions; and (2) and a third and a fourth polypeptide each comprising, from N- to C-terminus, (a) a masking peptide comprising a masking unit (MU) and a linkage unit (LU) comprising a cleavage site, wherein the MU comprises the amino acid sequence of SEQ ID NO: 150, and the LU comprises the amino acid sequence of SEQ ID NO: 103; and (b) an antibody light chain variable region (VL) comprising a CDR-L1, comprising the amino acid sequence of SEQ ID NO: 4; a CDR-L2 comprising the amino acid sequence of SEQ ID NO: 5; and a CDR-L3 comprising the amino acid sequence of SEQ ID NO: 6. In some embodiments, the VH comprises the amino acid sequence of SEQ ID NO: 74, or an amino acid sequence having at least 80% (e.g., at least 85%, 90%, 95%, 98%, or 99%; or 100%) sequence identity with SEQ ID NO: 74, and/or the VL comprises the amino acid sequence of SEQ ID NO: 75, or an amino acid sequence having at least 80% (e.g., at least 85%, 90%, 95%, 98%, or 99%; or 100%) sequence identity with SEQ ID NO: 75. In some embodiments, the first polypeptide and second polypeptide are each heavy chains, and/or the third polypeptide and the fourth polypeptide are each light chains. In some embodiments, the first polypeptide and the second polypeptide each comprise an IgG1 Fc domain. In some embodiments, the first polypeptide and the second polypeptide each comprise an IgG1 Fc domain comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the first polypeptide and the second polypeptide each comprise an IgG1 Fc domain comprising a S239D substitution and an I332E substitution. In some embodiments, the first and second polypeptide comprise the amino acid sequence of SEQ ID NO: 105, or an amino acid sequence having at least 80% (e.g., at least 85%, 90%, 95%, 98%, or 99%; or 100%) sequence identity with SEQ ID NO: 105, and the third and fourth polypeptide comprise the amino acid sequence of SEQ ID NO: 121, or an amino acid sequence having at least 80% (e.g., at least 85%, 90%, 95%, 98%, or 99%; or 100%) sequence identity with SEQ ID NO: 121. In some embodiments, the cancer is a colorectal cancer. In some embodiments, the anti-CD137 antibody comprises a heavy chain variable region (VH) and a light chain variable region (VL), wherein a) the heavy chain variable region comprises an HVR-H1, an HVR-H2, and an HVR-H3, wherein the HVR-H1 comprises the amino acid sequence of SEQ ID NO: 135, the HVR-H2 comprises the amino acid sequence of SEQ ID NO: 136, and the HVR-H3 comprises the amino acid sequence of SEQ ID NO: 137; and b) the light chain variable region comprises an HVR-L1, an HVR-L2, and an HVR-L3, wherein the HVR-L1 comprises the amino acid sequence of SEQ ID NO: 138, the HVR-L2 comprises the amino acid sequence of SEQ ID NO: 139, and the HVR-L3 comprises the amino acid sequence of SEQ ID NO: 140. In some embodiments, the anti-CD137 antibody comprises a VH and a VL, wherein the VH comprises the amino acid sequence of SEQ ID NO: 141 and the VL comprises the amino acid sequence of SEQ ID NO: 142. In some embodiments, the anti-CD137 antibody is selected from the group consisting of urelumab, utomilumab (PF-05082566), and AG10131. In certain embodiments, the anti-CD137 antibody is AG10131. In some embodiments, the anti-CD137 antibody is an anti-CD137 antibody described in U.S. Pat. Pub. No. 2020/0369776.

In some embodiments, provided herein is a method inducing phagocytosis of cells expressing CTLA4, the method comprising contacting the cells expressing CTLA4 with an effective amount of an anti-CTLA4 antibody, an anti-CTLA4 antigen-biding fragment, an anti-CTLA4 masked antibody (e.g., an anti-CTLA4 activatable antibody). In some embodiments, the phagocytosis is antibody-dependent cellular phagocytosis (ADCP). In some embodiments of the method, the an anti-CTLA4 antibody, an anti-CTLA4 antigen-biding fragment, an anti-CTLA4 masked antibody (e.g., an anti-CTLA4 activatable antibody) comprises an IgG1 Fc domain, or a part thereof (e.g., a CH2 domain), comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution (e.g., i) an S298A substitution, an E333A substitution, and a K334A substitution; ii) an S298A substitution, an E333A substitution, a K334A substitution, and a K326A substitution; iii) an F243L substitution; iv) an F243L substitution, an R292P substitution, a Y300L substitution, and a P396L substitution; v) an F243L substitution, an R292P substitution, a Y300L substitution, a V305I substitution, and a P396L substitution; vi) an F243L substitution, an R292P substitution, a Y300L substitution, a L235V substitution, and a P396L substitution; vii) a S239D substitution, an I332E substitution, and an A330L substitution; or viii) a S239D substitution and an I332E substitution), wherein the residue numbering is according to EU numbering. In some embodiments, the anti-CTLA4 antibody, anti-CTLA4 antigen-biding fragment, or anti-CTLA4 masked antibody (e.g., an anti-CTLA4 activatable antibody) induces ADCP at a rate of at least 20% greater (e.g., at least 20%, at least 30%, at least 40%, at least 50%, at least 60%, at least 70%, at least 80%, at least 90%, at least 100%, at least 1.5 fold, or at least 2 fold greater) than an anti-CTLA4 antibody that does not comprise an IgG1 Fc domain, or a part thereof (e.g., a CH2 domain), comprising the substitutions. In some embodiments, the anti-CTLA4 antibody, anti-CTLA4 antigen-biding fragment, or anti-CTLA4 masked antibody (e.g., an anti-CTLA4 activatable antibody) induces ADCP at a rate of at least 20% greater (e.g., at least 20%, at least 30%, at least 40%, at least 50%, at least 60%, at least 70%, at least 80%, at least 90%, at least 100%, at least 1.5 fold, or at least 2 fold greater) than an anti-CTLA4 antibody that comprises an IgG1 Fc domain, or a part thereof (e.g., a CH2 domain), that does not comprise the substitutions. In certain embodiments, the anti-CTLA4 antibody, anti-CTLA4 antigen-biding fragment, or anti-CTLA4 masked antibody (e.g., an anti-CTLA4 activatable antibody) has a half maximal effective concentration (EC50) of less than 3 nM (e.g., less than 3 nM, less than 2 nM, less than 1.5 nM, less than 1 nM, less than 0.5 nM, less than 0.1 nM, or less than 0.05 nM) for inducing ADCP of cells expressing CTLA4.

In some embodiments of the foregoing methods, the anti-CTLA4 antibody induces ADCP effects by more than about 10% (e.g., induce ADCP by more than about 10%, more than about 15%, more than about 20%, more than about 25%, more than about 30%, more than about 35%, more than about 40%, etc.) relative to a control (e.g., an isotype control or ipilimumab). In certain embodiments wherein the anti-CTLA4 antibody comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibody induces ADCP effects by at a rate of at least about 10% (e.g., at least about 10%, at least about 20%, at least about 30%, at least about 40%, at least about 50%, at least about 60%, at least about 70%, at least about 80%, or at least about 90%) greater than an anti-CTLA4 antibody that comprises an Fc region that does not comprise the substitutions. In certain embodiments wherein the anti-CTLA4 antibody comprises an IgG1 Fc region (e.g., a human IgG1 Fc region) comprising one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering, the anti-CTLA4 antibody induces ADCP effects by at a rate of at least about 1.5 fold (e.g., at least about 1.5 fold, at least about 2 fold, at least about 2.5 fold, at least about 3 fold, at least about 4 fold, or at least about 5 fold) greater than an anti-CTLA4 antibody that comprises an Fc region that does not comprise the substitutions. In some embodiments, the IgG1 Fc region (e.g., human IgG1 Fc region) comprises two identical Fc domains. In certain embodiments, the IgG1 Fc region (e.g., human IgG1 Fc region) comprises two different (i.e., non-identical) Fc domains. Methods of measuring ADCP of antibodies and antigen binding fragments are also well known in the art. For example, to assess ADCP activity of a molecule of interest, an in vitro ADCP assay (see, e.g., Bracher et al., 2007, J. Immunol. Methods 323: 160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248: 167-82).

In another aspect, the present disclosure provides a method of enhancing an immune response in a subject, which comprises administering to the subject an effective amount of a CTLA4 binding molecule (e.g., an anti-CTLA4 antibody or masked antibody (e.g., activatable antibody)) or composition provided by the present disclosure. In some embodiments, the binding molecule is a CTLA4 antibody or antigen-binding fragment thereof and the subject is a human. In some embodiments, the binding molecule is a CTLA4 masked antibody (e.g., activatable antibody) and the subject is a human. The term “enhancing immune response” or its grammatical variations, means stimulating, evoking, increasing, improving, or augmenting any response of a subject's immune system. The immune response may be a cellular response (i.e. cell-mediated, such as cytotoxic T lymphocyte mediated) or a humoral response (i.e. antibody mediated), and may be a primary or secondary immune response. Examples of enhancement of immune response include activation of PBMCs and/or T cells (including increasing secretion of one or more cytokines such as IL-2 and/or IFNγ). The enhancement of immune response can be assessed using a number of in vitro or in vivo measurements known to those skilled in the art, including, but not limited to, cytotoxic T lymphocyte assays, release of cytokines, regression of tumors, survival of tumor bearing animals, antibody production, immune cell proliferation, expression of cell surface markers, and cytotoxicity. Typically, methods of the present disclosure enhance the immune response by a subject when compared to the immune response by an untreated subject or a subject not treated using the recited methods.

In practicing the therapeutic methods, the anti-CTLA4 binding molecules (e.g., anti-CTLA4 antibodies or masked antibodies (e.g., activatable antibodies)) may be administered alone as monotherapy, or administered in combination with one or more additional therapeutic agents or therapies. Thus, in another aspect, the present disclosure provides a combination therapy, which comprises an anti-CTLA4 binding molecule (e.g., an anti-CTLA4 antibody or masked antibody (e.g., activatable antibody)) in combination with one or more additional therapies or therapeutic agents for separate, sequential or simultaneous administration. The term “additional therapeutic agent” may refer to any therapeutic agent other than an anti-CTLA4 binding molecule (e.g., an anti-CTLA4 antibody or masked antibody (e.g., activatable antibody)) provided by the disclosure. In one particular aspect, the present disclosure provides a combination therapy for treating cancer in a subject, which comprises administering to the subject an effective amount of an anti-CTLA4 binding molecule (e.g., an anti-CTLA4 antibody or masked antibody (e.g., activatable antibody)) provided herein in combination with one or more additional therapeutic agents. In one embodiment, the subject is a mammal. In a further embodiment, the subject is a human.

The anti-CTLA4 binding molecules (e.g., anti-CTLA4 antibodies or masked antibodies (e.g., activatable antibodies)) and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The anti-CTLA4 binding molecules (e.g., anti-CTLA4 antibodies or masked antibodies (e.g., activatable antibodies)) and compositions of the present disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific anti-CTLA4 binding molecule (e.g., anti-CTLA4 antibody or masked antibody (e.g., activatable antibody)) being used, the rate of absorption desired, specific formulation or dosage form used, type or severity of the disorder being treated, the specific site of action, and conditions of the patient, and can be readily selected by a person skilled in the art.

The term “effective amount” of an anti-CTLA4 binding molecule (e.g., an anti-CTLA4 antibody or masked antibody (e.g., activatable antibody)) may refer to an amount that is effective for an intended therapeutic purpose. For example, in the context of enhancing an immune response, an “effective amount” may be any amount that is effective in stimulating, evoking, increasing, improving, or augmenting any response of a subject's immune system. In the context of treating a disease, an “effective amount” may be any amount that is sufficient to cause any desirable or beneficial effect in the subject being treated. Specifically, in the treatment of cancer, examples of desirable or beneficial effects include inhibition of further growth or spread of cancer cells, death of cancer cells, inhibition of reoccurrence of cancer, reduction of pain associated with the cancer, or improved survival of the subject. The therapeutically effective amount of an anti-CTLA4 binding molecule (e.g., an anti-CTLA4 antibody or masked antibody (e.g., activatable antibody)) usually ranges from about 0.001 to about 500 mg/kg, and more usually about 0.01 to about 100 mg/kg, of the body weight of the subject. The precise dosage level to be administered can be readily determined by a person skilled in the art and will depend on a number of factors, such as the type, and severity of the disorder to be treated, the particular anti-CTLA4 binding molecule (e.g., anti-CTLA4 antibody or masked antibody (e.g., activatable antibody)) employed, the route of administration, the time of administration, the duration of the treatment, the particular additional therapy employed, the age, sex, weight, condition, general health and prior medical history of the patient being treated, and like factors well known in the medical arts.

An anti-CTLA4 binding molecule (e.g., an anti-CTLA4 antibody or masked antibody (e.g., activatable antibody)) or composition is usually administered on multiple occasions. Intervals between single doses can be, for example, daily, weekly, monthly, every three months or yearly. An exemplary treatment regimen entails administration once per week, once every two weeks, once every three weeks, once every four weeks, once a month, once every three months or once every three to six months.

### VIII. Kits and Articles of Manufacture

In another aspect, provided herein is a kit comprising an anti-CTLA4 binding molecule (e.g., an anti-CTLA4 antibody or masked antibody (e.g., activatable antibody)) and/or composition of the present disclosure. In some embodiments, the kit further comprises a package insert comprising instructions for use of the anti-CTLA4 binding molecule (e.g., anti-CTLA4 antibody or masked antibody (e.g., activatable antibody)) and/or composition. In some embodiments, the kit further comprises one or more buffers, e.g., for storing, transferring, administering, or otherwise using the anti-CTLA4 binding molecule (e.g., anti-CTLA4 antibody or masked antibody (e.g., activatable antibody)) and/or composition. In some embodiments, the kit further comprises one or more containers for storing the anti-CTLA4 binding molecule (e.g., anti-CTLA4 antibody or masked antibody (e.g., activatable antibody)) and/or composition.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

### EXAMPLES

### Example 1. Construction of Activatable Antibodies Targeting CTLA4

A low copy number, CEN/ARS-based vector was used to express the target antibody (antibody TY21580, targeting human CTLA4) under the control of the inducible GAL1-10 promoter in the yeast S. cerevisiae. The surface display of scFvs was achieved through the Aga2 protein fused at its C-terminus under the control of the GAL1 promoter, similar to previously published arrangements (Boder and Wittrup (1997) Nat Biotechnol 15 (6): 553-7).

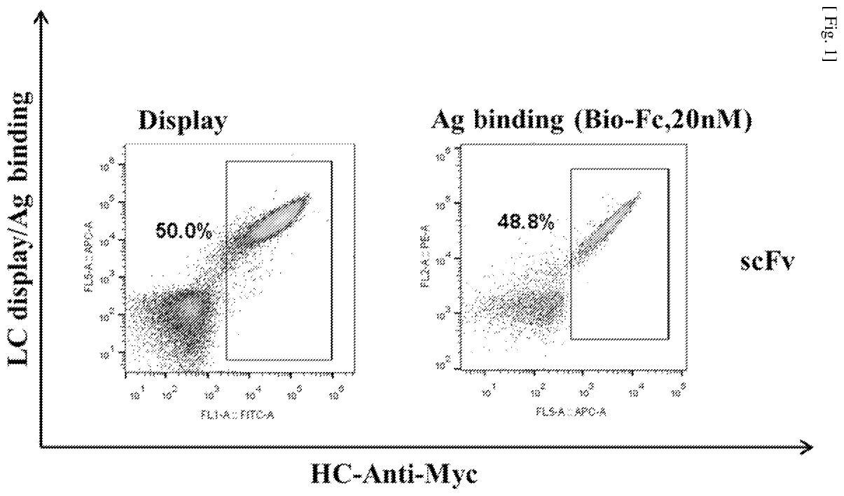

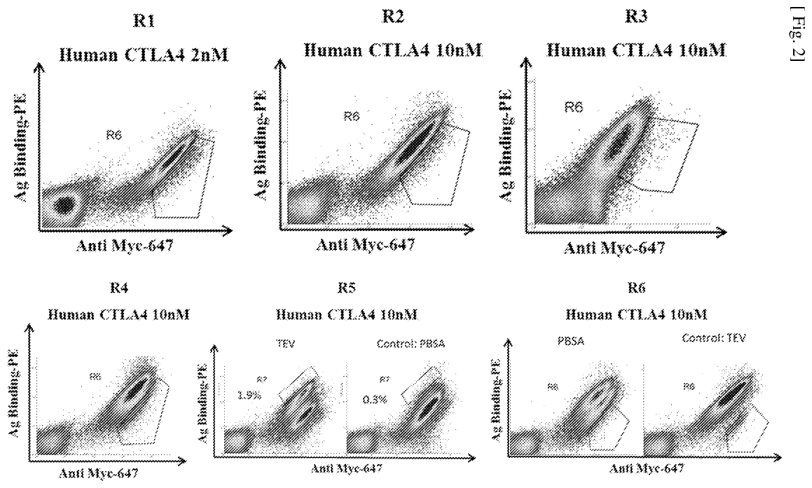

The surface display of the scFv was verified by staining with antibodies recognizing the fused affinity tag, and the functionality of the scFvs displayed on yeast was examined using biotinylated human CTLA4. Briefly, 48 hours after induction in galactose medium, yeast cells (1×10⁶) were harvested, washed once with PBSA buffer, and then incubated with 10 nM of biotinylated antigen for 1 hour at room temperature. The yeast cells were then washed twice with PBSA buffer, and incubated with PE conjugated streptavidin (1:500 dilution) (eBioscience #2-4317-87) for 30 minutes at 4° C. The yeast cells were then analyzed by flow cytometry. As shown in FIG. 1, scFvs targeting CTLA4 were successfully displayed on the yeast surface, and were capable of binding strongly to the antigens.

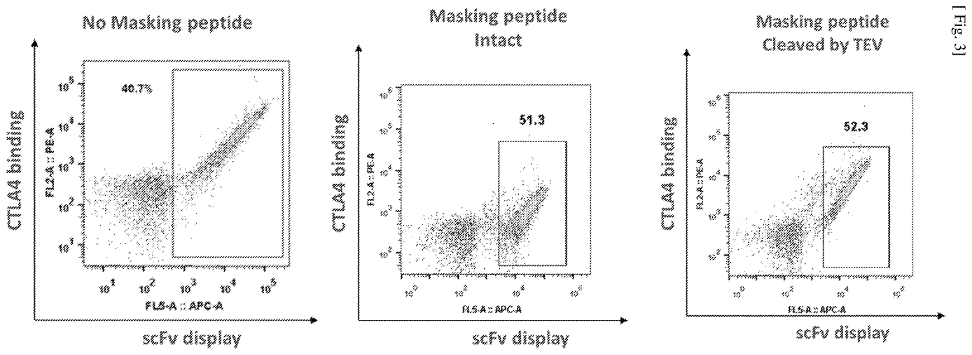

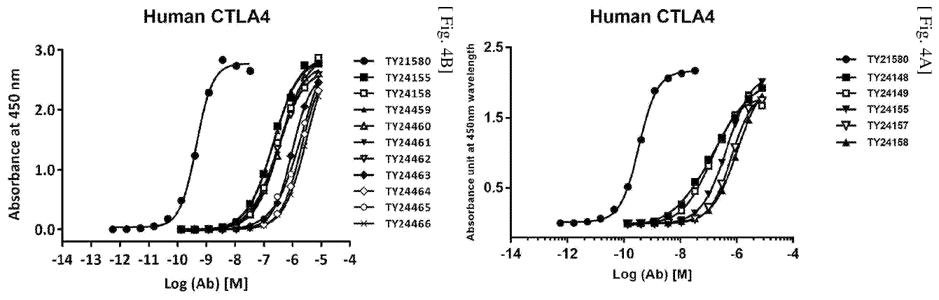

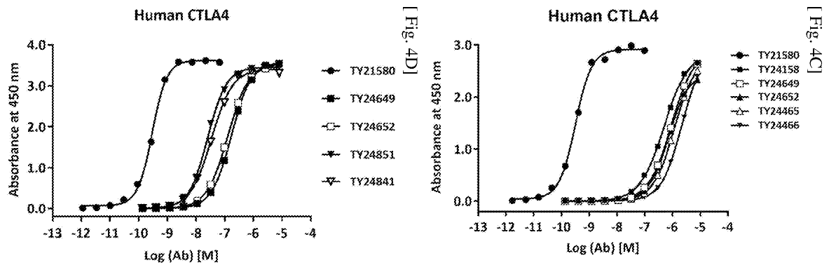

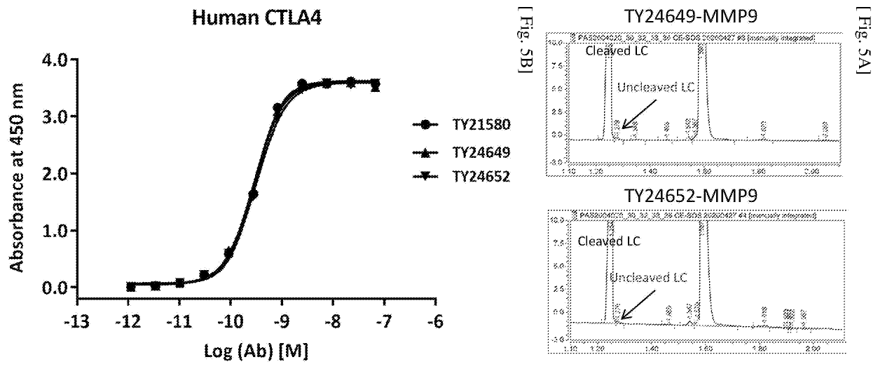

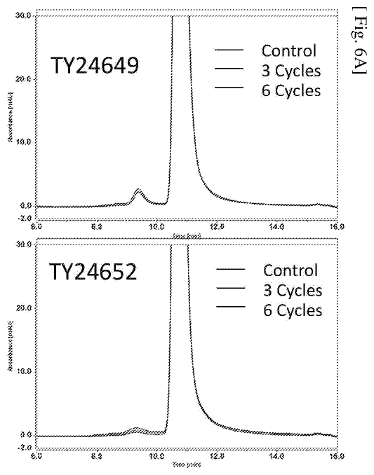

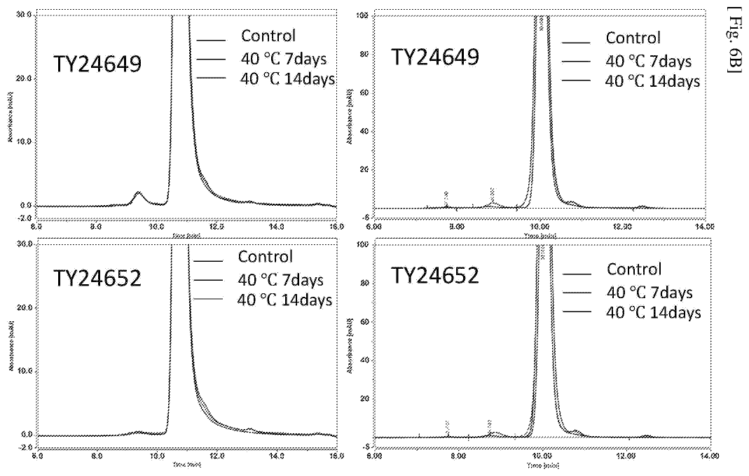

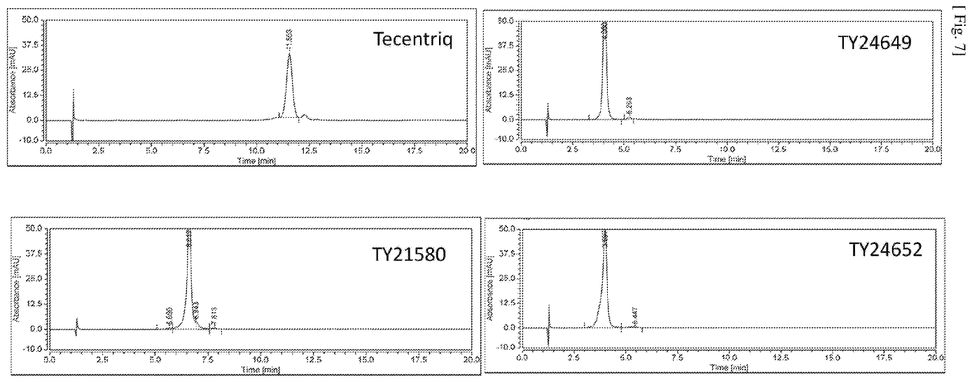

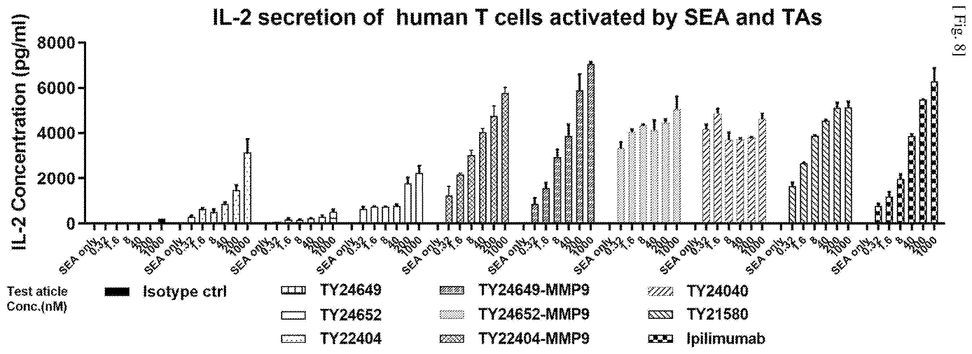

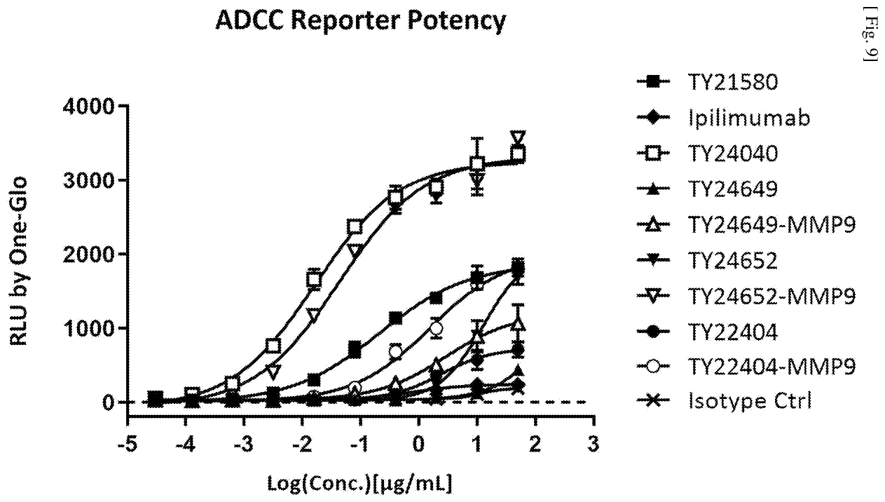

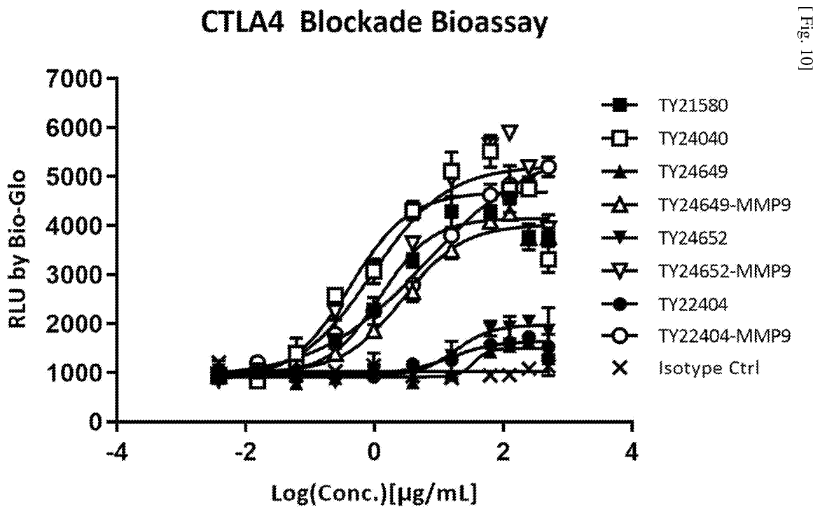

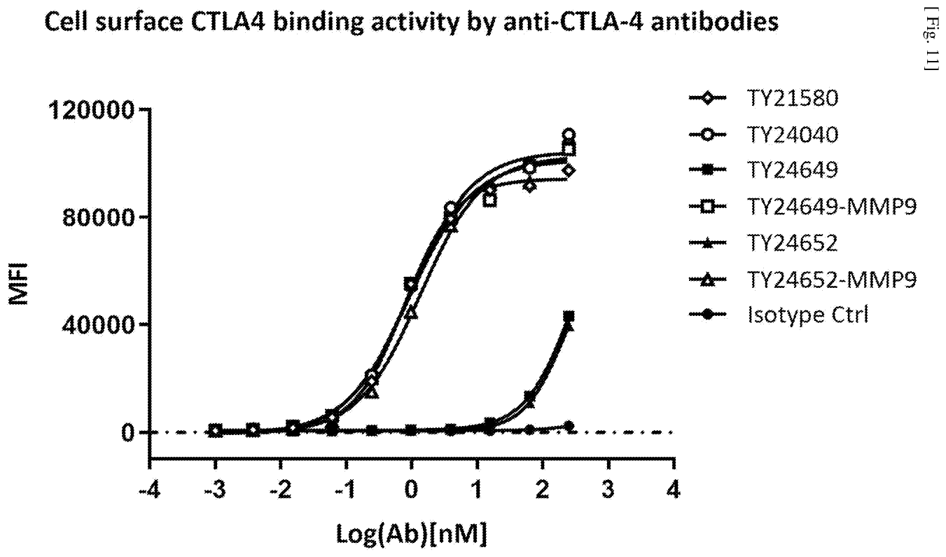

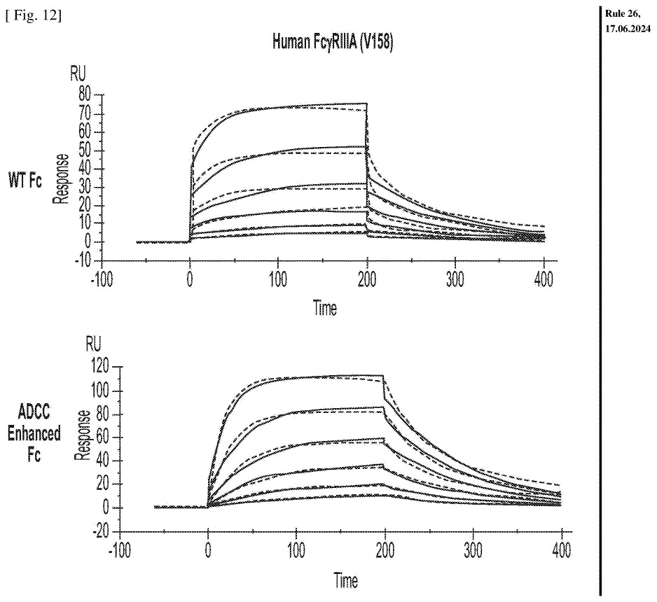

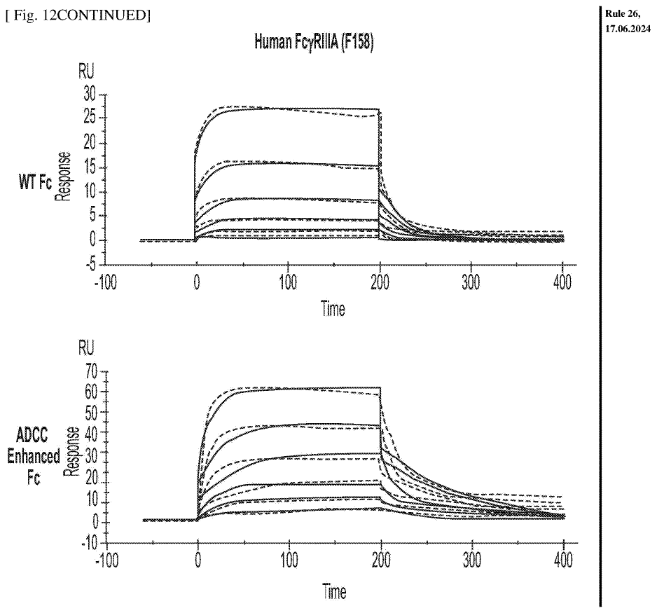

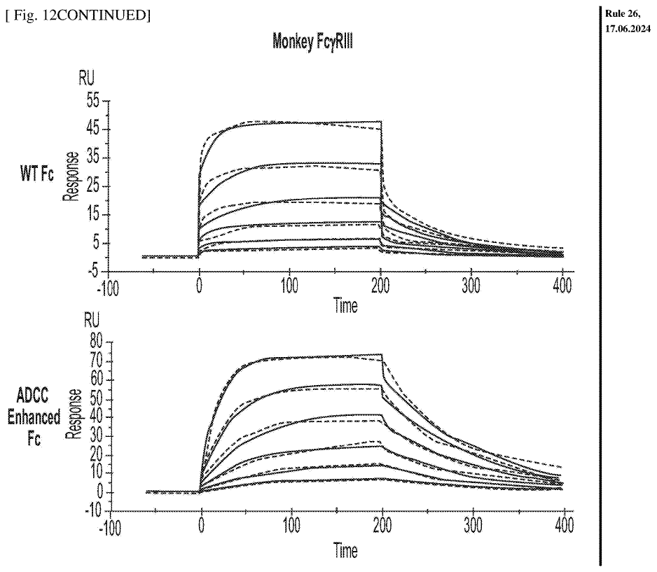

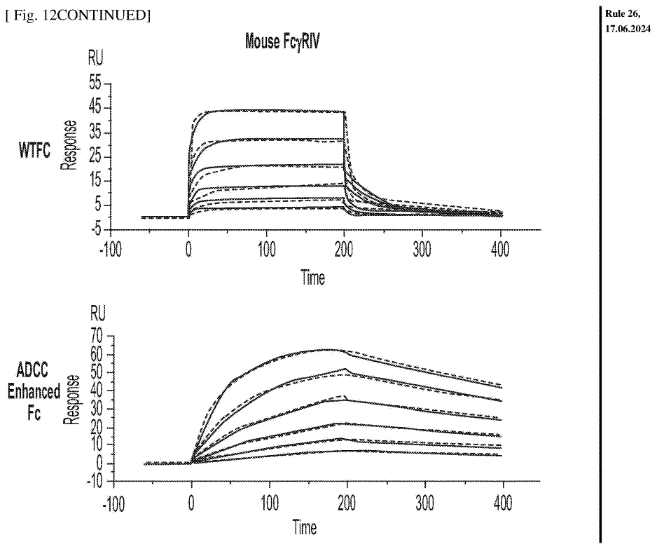

FACS-based screening of masking peptides against a CTLA4 antibody. A total of 1×10⁸ yeast cells from a CPL yeast library were used to screen for masking peptides against the target antibody. For each round of sorting through MoFlo XDP, yeast cells induced in galactose medium were harvested, washed once with PBSA buffer, and then incubated with 10 nM (decreased to 1 nM in the later rounds) of biotinylated antigen for 1 hour at room temperature. The yeast cells were then washed twice with PBSA buffer, and incubated with PE conjugated streptavidin (1:500 dilution) (eBioscience #2-4317-87) for 30 minutes at 4° C. After two more washes with PBSA buffer, the yeast cells were adjusted to 2-3 OD/mL, and subject to sorting. As shown in FIG. 2, in round 1, 2 nM of biotinylated CTLA4-hisFc was used, and the weak binders were enriched. Starting from the 2nd round of sorting, the concentration of the biotinylated CTLA4-hisFc was increased to 10 nM, and the weak binders were collected. The yeast cells from round 5, after growth in glucose medium, were induced in galactose medium and treated with TEV protease (6U/OD cell) (in-house) for 2 hours at 30° C., and the strong binders were purified. At the 6th round, fractions of the yeast cells were also treated with TEV in parallel, to verify the protease cleavage mediated activation of the target antibody. As shown in FIG. 2, it was apparent that TEV cleavage resulted in a dramatic increase of the population of cells that bound strongly to antigen, suggesting that the screening strategy was effective. The single clones from the 6th round of sorting were plated on selective media, and grown individually for further confirmation of cleavage mediated activated antigen binding.

As shown in FIG. 3, the selected CTLA4 activatable antibody clones in scFv format, exhibited little binding to antigen in the presence of masking peptide. However, binding to antigen was dramatically increased when the yeast cells were treated with TEV protease to remove the masking peptide. The incorporation of the TEV recognition site in the cleavage peptide, combined with the application of TEV protease to verify the selected clones, significantly increased the success rate of masking peptide selection.

To identify the masking peptide sequences, the shuttle plasmids were extracted from the selected yeast clones (Generay #GK2002-200), and transformed into competent E. coli cells. The plasmids were prepared, and the regions encoding the masking peptides were sequenced and aligned. As anticipated, these sequences could be separated into several groups, indicating clear enrichment through rounds of sorting. Masking peptide sequences, as well as additional masking peptide sequences are listed in Tables 5A-5F. Each masking peptide comprises an N-terminal unit, a masking unit, and a linkage unit. Some masking peptides comprise a linkage unit comprising, from N-terminus to C-terminus, a first cleavage site (C₁), a first linker (L₁), a second cleavage site (C₂), and a second linker (L₂) (C₁-L₁-C₂-L₂ configuration, Table 5A). Other masking peptides comprise a linkage unit comprising, from N-terminus to C-terminus, a first linker (L₁), a first cleavage site (C₁), and a second linker (L₂) (L₁-C₁-L₂ configuration, Table 5B). Still other of the making peptides comprise a linkage unit comprising only a linker and not comprising a cleavage site (Table 5C). For TY24800, TY24652 and, TY24851, mutations S239D/I332E were employed in Fc to improve ADCC.

TABLE 5A
Sequences of N-terminal unit, masking units,
linkers, and cleavage sites of antibodies
having C₁-L₁-C₂-L₂ configuration linkage units.

| | N- | SEQ | | SEQ | First | SEQ | First | SEQ | Second | SEQ | Second | SEQ |
|---|---|---|---|---|---|---|---|---|---|---|---|---|
| Sample | terminal | ID | | ID | Cleavage | ID | Linker | ID | Cleavage | ID | Linker | ID |
| ID | Unit | NO: | Masking Unit | NO: | Site (C₁) | NO: | (L₁) | NO: | Site (C₂) | NO: | (L₂) | NO: |
| TY24148 | EVGSY | 143 | VLCAKFPFCIT | 146 | SGRSA | 152 | GGGGT | 155 | PLGLAG | 154 | SGGS | 160 |
| TY24149 | EVGSY | 143 | PPCVGFYPYCKT | 147 | SGRSA | 152 | GGGGT | 155 | PLGLAG | 154 | SGGS | 160 |
| TY24155 | EVGSY | 143 | SSCKAPYPYCKG | 148 | SGRSA | 152 | GGGGT | 155 | PLGLAG | 154 | SGGS | 160 |
| TY24157 | EVGSY | 143 | ALCEPFATSVCRP | 149 | SGRSA | 152 | GGGGT | 155 | PLGLAG | 154 | SGGS | 160 |
| TY24158 | EVGSY | 143 | TLCEPFATSVCRP | 150 | SGRSA | 152 | GGGGT | 155 | PLGLAG | 154 | SGGS | 160 |

TABLE 5B
Sequences of N-terminal unit, masking units,
linkers, and cleavage sites of antibodies
having L₁-C₁-L₂ configuration linkage units.

| | N- | SEQ | | SEQ | | SEQ | First | SEQ | | SEQ |
|---|---|---|---|---|---|---|---|---|---|---|
| Sample | terminal | ID | | ID | First | ID | Cleavage | ID | Second | ID |
| ID | Unit | NO: | Masking Unit | NO: | Linker | NO: | Site | NO: | Linker | NO: |
| TY24460 | EVGSY | 143 | SSCKAPYPYCKG | 148 | SGGG | 157 | PLGLAG | 154 | GS | |
| TY24461 | EVGSY | 143 | SSCKAPYPYCKG | 148 | GGG | | PLGLAG | 154 | GS | |
| TY24462 | E | | SSCKAPYPYCKG | 148 | SGGG | 157 | PLGLAG | 154 | GS | |
| TY24464 | EVGSY | 143 | TLCEPFATSVCRP | 151 | SGGG | 157 | PLGLAG | 154 | GS | |
| TY24466 | E | | TLCEPFATSVCRP | 150 | SGGG | 157 | PLGLAG | 154 | GS | |
| TY24465 | EVGSY | 143 | TLCEPFATSVCRP | 150 | GGG | | PLGLAG | 154 | GS | |
| TY24800 | E | | TLCEPFATSVCRP | 150 | GGG | | PLGLAG | 154 | GS | |
| TY24649 | E | | TLCEPFATSVCRP | 150 | GGG | | PLGLAG | 154 | GGS | |
| TY24652 | E | | TLCEPFATSVCRP | 150 | GGG | | PLGLAG | 154 | GGS | |
| TY24459 | EVGSY | 143 | SSCKAPYPYCKG | 148 | SGRG | 153 | PLGLAG | 154 | GS | |
| TY24463 | EVGSY | 143 | TLCEPFATSVCRP | 151 | SGRG | 153 | PLGLAG | 154 | GS | |

TABLE 5C
Sequences of N-terminal unit, masking units,
and linkers of antibodies
having linkage units without cleavage sites.

| | N- | SEQ | | SEQ | | SEQ |
|---|---|---|---|---|---|---|
| Sample | terminal | ID | Masking | ID | | ID |
| ID | Unit | NO: | Unit | NO: | Linker | NO: |
| TY24851 | E | | TLCEPFA | 150 | GGGGSG | 159 |
| | | | TSVCRP | | GSGGGS | |
| TY24841 | E | | TLCEPFA | 150 | GGGGSG | 159 |
| | | | TSVCRP | | GSGGGS | |

TABLE 5D
N-terminal unit, masking unit, cleavage site,
and linker sequences by SEQ ID NO.

| | | SEQ |
|---|---|---|
| Sequence Type | Sequence | ID NO. |
| N-terminal unit | EVGSY | 143 |
| N-terminal unit | E | |
| Masking unit | VLCAKFPFCIT | 146 |
| Masking unit | PPCVGFYPYCKT | 147 |
| Masking unit | SSCKAPYPYCKG | 148 |
| Masking unit | ALCEPFATSVCRP | 149 |
| Masking unit | TLCEPFATSVCRP | 150 |
| Masking unit | TLCEPFATSVCRP | 151 |
| Cleavage site | SGRSA | 152 |
| Cleavage site | SGRG | 153 |
| Cleavage site | PLGLAG | 154 |
| Linker | GGGGT | 155 |
| Linker | GS | |
| Linker | SGGG | 157 |
| Linker | GGG | |
| Linker | GGGGSGGSGGGS | 159 |
| Linker | SGGS | 160 |
| Linker | GGGS | 161 |
| Linker | GGS | |

TABLE 5E
Sequences of combined N-terminal units
and masking units, and linkage units.

| Sample | Combined N-Terminal | | Linkage Unit | |
|---|---|---|---|---|
| ID | Unit and Masking Unit | | (cleavage sites and linkers) | |
| TY24148 | EVGSYVLCAKFPFCIT | SEQ ID NO: 92 | SGRSAGGGGTPLGLAGSGGS | SEQ ID NO: 99 |
| TY24149 | EVGSYPPCVGFYPYCKT | SEQ ID NO: 93 | SGRSAGGGGTPLGLAGSGGS | SEQ ID NO: 99 |
| TY24155 | EVGSYSSCKAPYPYCKG | SEQ ID NO: 94 | SGRSAGGGGTPLGLAGSGGS | SEQ ID NO: 99 |
| TY24157 | EVGSYALCEPFATSVCRP | SEQ ID NO: 95 | SGRSAGGGGTPLGLAGSGGS | SEQ ID NO: 99 |
| TY24158 | EVGSYTLCEPFATSVCRP | SEQ ID NO: 96 | SGRSAGGGGTPLGLAGSGGS | SEQ ID NO: 99 |
| TY24459 | EVGSYSSCKAPYPYCKG | SEQ ID NO: 94 | SGRGPLGLAGGS | SEQ ID NO: 100 |
| TY24460 | EVGSYSSCKAPYPYCKG | SEQ ID NO: 94 | SGGGPLGLAGGS | SEQ ID NO: 101 |
| TY24461 | EVGSYSSCKAPYPYCKG | SEQ ID NO: 94 | GGGPLGLAGGS | SEQ ID NO: 102 |
| TY24462 | ESSCKAPYPYCKG | SEQ ID NO: 97 | SGGGPLGLAGGS | SEQ ID NO: 101 |
| TY24463 | EVGSYTLCEPFATSVCRP | SEQ ID NO: 96 | SGRGPLGLAGGS | SEQ ID NO: 100 |
| TY24464 | EVGSYTLCEPFATSVCRP | SEQ ID NO: 96 | SGGGPLGLAGGS | SEQ ID NO: 101 |
| TY24466 | ETLCEPFATSVCRP | SEQ ID NO: 98 | SGGGPLGLAGGS | SEQ ID NO: 101 |
| TY24465 | EVGSYTLCEPFATSVCRP | SEQ ID NO: 96 | GGGPLGLAGGS | SEQ ID NO: 102 |
| TY24800 | ETLCEPFATSVCRP | SEQ ID NO: 98 | GGGPLGLAGGS | SEQ ID NO: 102 |
| TY24649 | ETLCEPFATSVCRP | SEQ ID NO: 98 | GGGPLGLAGGS | SEQ ID NO: 103 |
| TY24652 | ETLCEPFATSVCRP | SEQ ID NO: 98 | GGGPLGLAGGS | SEQ ID NO: 103 |
| TY24851 | ETLCEPFATSVCRP | SEQ ID NO: 98 | GGGGSGGSGGGS | SEQ ID NO: 159 |
| TY24841 | ETLCEPFATSVCRP | SEQ ID NO: 98 | GGGGSGGSGGGS | SEQ ID NO: 159 |

TABLE 5F
Sequences of full masking peptides.

| Sample | Masking Peptide | SEQ ID NO: |
|---|---|---|
| TY24148 | EVGSYVLCAKFPFCITSGRSAGGGGTPLGLAGSGGS | SEQ ID NO: 162 |
| TY24149 | EVGSYPPCVGFYPYCKTSGRSAGGGGTPLGLAGSGGS | SEQ ID NO: 163 |
| TY24155 | EVGSYSSCKAPYPYCKGSGRSAGGGGTPLGLAGSGGS | SEQ ID NO: 164 |
| TY24157 | EVGSYALCEPFATSVCRPSGRSAGGGGTPLGLAGSGGS | SEQ ID NO: 165 |
| TY24158 | EVGSYTLCEPFATSVCRPSGRSAGGGGTPLGLAGSGGS | SEQ ID NO: 166 |
| TY24459 | EVGSYSSCKAPYPYCKGSGRGPLGLAGGS | SEQ ID NO: 167 |
| TY24460 | EVGSYSSCKAPYPYCKGSGGGPLGLAGGS | SEQ ID NO: 168 |
| TY24461 | EVGSYSSCKAPYPYCKGGGGPLGLAGGS | SEQ ID NO: 169 |
| TY24462 | ESSCKAPYPYCKGSGGGPLGLAGGS | SEQ ID NO: 170 |
| TY24463 | EVGSYTLCEPFATSVCRPSGRGPLGLAGGS | SEQ ID NO: 171 |
| TY24464 | EVGSYTLCEPFATSVCRPSGGGPLGLAGGS | SEQ ID NO: 172 |
| TY24466 | ETLCEPFATSVCRPSGGGPLGLAGGS | SEQ ID NO: 173 |
| TY24465 | EVGSYTLCEPFATSVCRPGGGPLGLAGGS | SEQ ID NO: 174 |
| TY24800 | ETLCEPFATSVCRPGGGPLGLAGGS | SEQ ID NO: 175 |
| TY24649 | ETLCEPFATSVCRPGGGPLGLAGGGS | SEQ ID NO: 176 |
| TY24652 | ETLCEPFATSVCRPGGGPLGLAGGGS | SEQ ID NO: 176 |
| TY24851 | ETLCEPFATSVCRPGGGGSGGSGGGS | SEQ ID NO: 177 |
| TY24841 | ETLCEPFATSVCRPGGGGSGGSGGGS | SEQ ID NO: 177 |

IgG conversion and expression. The masking peptides listed in Table 5F were converted into IgG1s. Some of the IgGs contains only one MMP cleavage site. Two of the IgGs contain mutations in Fc to enhance the ADCC effects.

The heavy and light chains were cloned into the mammalian expression vector pCDNA3.3 (Thermo Fisher Scientific, cat #K830001) separately, and the masking peptides and the invariant cleavage peptide were fused to the N-terminus of the light chain in the same manner as displayed on yeast surface. The VH and VL sequences for the parental CTLA4 antibody TY21580 are listed below:

TY21580 anti-CTLA4 heavy chain variable region
(SEQ ID NO: 74):
EVQLVESGGGLVQPGGSLRLSCAASGYSISSGYHWSWIRQAPGKGLE
WLARIDWDDDKYYSTSLKSRLTISRDNSKNTLYLQLNSLRAEDTAVY
YCARSYVYFDYWGQGTLVTVSS
TY21580 anti-CTLA4 light chain variable region
(SEQ ID NO: 75):
DIQLTQSPSSLSASVGDRVTITCRASQSVRGRFLAWYQQKPGKAPKL
LIYDASNRATGIPSRFSGSGSGTDFTLTISSLQPEDFATYYCQQSSS
WPPTFGQGTKVEIKR

Pairs of plasmids were transiently transfected into HEK293F cells. After six days, the supernatants were harvested, cleared by centrifugation and filtration, and IgGs were purified with standard protein A affinity chromatography (MabSelect SuRe, GE Healthcare). The IgGs were eluted and neutralized, and buffer exchanged into 20 mM histidine, pH 5.5. Protein concentrations were determined by UV-spectrophotometry, and IgG purity was analyzed under denaturing, reducing and non-reducing conditions by SDS-PAGE or SEC-HPLC. Importantly, the expression levels of the activatable antibodies in HEK293 cells were similar to their parental antibody, and their purification yields after protein A resin were also similar, suggesting that the presence of the masking and cleavage peptides do not have a negative impact on antibody expression in mammalian cells.

### Example 2. Measurement of Masking Efficiency

Recombinant human CTLA4-hisFc was diluted to 1 μg/mL in PBS and coated on a Maxisorp plate at 4° C. overnight. Plates were blocked with PBS supplemented with 3% non-fat milk at 37° C. for 1 hour. After washing, 100 L of 3-fold serial dilutions of antibodies were added to each well. After incubation at 37° C. for 1 hour, plates were washed four times, and 100 μL HRP conjugated anti-human IgG (Fab specific) (1:6000 dilution) was added to each well. Plates were incubated at 37° C. for 1 hour, washed four times, and then 50 μL TMB substrate solution was added to each well, and the plate was incubated at room temperature. Absorbance at 450 nm was measured after the reactions were stopped with 50 μL H2SO4 per well. The EC50 was evaluated by fitting the ELISA data using the sigmoidal (four-parameter logistic equation) model of GraphPad Prism 6 software. Experiments for some activatable antibodies were performed twice or more, leading to two or more calculated masking efficiencies being obtained for each of these activatable antibodies. Masking efficiencies for each activatable antibody were calculated by dividing the EC50 for binding of the activatable antibody by the EC50 of the parental antibody (TY21580). As shown in FIGS. 4A-4D and Table 6, compared with the parental antibody, all of the activatable antibodies show dramatically reduced binding to its antigen, and the calculated masking efficiency ranged from 87 to 6976. Differences in masking efficiency likely resulted from variation in measurement and data fitting for the EC50 values, and the masking efficiency for each activatable antibody likely falls within the calculated ranges. These results indicated that multiple masking peptides identified from the CPLs maintained their masking efficiency when expressed in mammalian cells, and as part of a full IgG molecule.

TABLE 6
Activatable antibody ELISAs prior to protease cleavage

| Sample | EC50 | | | Masking |
|---|---|---|---|---|
| ID: | M: | nM: | R²: | efficiency: |

Data Batch 1

| | | | | |
|---|---|---|---|---|
| TY21580 | 3.44E−10 | 0.3444 | 0.9966 | 1.0 |
| TY24148 | 1.50E−07 | 150 | 0.9986 | 436 |
| TY24149 | 1.23E−07 | 122.5 | 0.9953 | 356 |
| TY24155 | 4.38E−07 | 437.7 | 0.9999 | 1271 |
| TY24157 | 6.34E−07 | 633.5 | 0.9985 | 1839 |
| TY24158 | 1.04E−06 | 1042 | 1 | 3026 |

Data Batch 2

| | | | | |
|---|---|---|---|---|
| TY21580 | 4.58E−10 | 0.4575 | 0.9963 | 1 |
| TY24155 | 2.10E−07 | 210.1 | 0.9982 | 459 |
| TY24158 | 3.54E−07 | 353.6 | 0.9959 | 773 |
| TY24459 | 2.09E−07 | 209.4 | 0.9995 | 458 |
| TY24460 | 3.39E−07 | 339 | 0.9988 | 741 |
| TY24461 | 3.10E−07 | 309.9 | 0.9994 | 677 |
| TY24462 | 3.14E−07 | 313.5 | 0.9996 | 685 |
| TY24463 | 9.96E−07 | 996 | 0.9998 | 2177 |
| TY24464 | 2.14E−06 | 2140 | 0.9999 | 4678 |
| TY24465 | 2.33E−06 | 2332 | 0.9982 | 5097 |
| TY24466 | 3.17E−06 | 3168 | 0.9996 | 6925 |

Data Batch 3

| | | | | |
|---|---|---|---|---|
| TY21580 | 3.20E−10 | 0.3198 | 0.9979 | 1 |
| TY24158 | 5.01E−07 | 501 | 0.9995 | 1566 |
| TY24649 | 9.27E−07 | 927 | 0.9999 | 2899 |
| TY24652 | 7.43E−07 | 743 | 0.9997 | 2324 |
| TY24465 | 1.20E−06 | 1199 | 1 | 3749 |
| TY24466 | 2.23E−06 | 2231 | 1 | 6976 |

Data Batch 4

| | | | | |
|---|---|---|---|---|
| TY21580 | 2.98E−10 | 0.298 | 0.9986 | 1 |
| TY24649 | 1.73E−07 | 173.2 | 0.9999 | 581 |
| TY24652 | 1.28E−07 | 128.7 | 1 | 432 |
| TY24851 | 2.58E−08 | 25.8 | 0.9993 | 87 |
| TY24841 | 3.49E−08 | 34.9 | 0.9991 | 117 |

### Example 3. Removal of the Masking Peptide Restores Antibody Activity

The purified activatable antibodies were treated with the proteases which recognize the cleavage sequences, and were then tested to determine whether removal of the masking peptide restored their activity. As an example, 20 g of TY24649 and TY24652 (0.5 mg/mL) was treated with 1 g of recombinant human MMP-9 (in-house) in reaction buffer (50 mM Tris, 10 mM CaCl₂), 150 mM NaCl, 0.05% Brij35 (w/v), pH 7.5). The reactions were carried out at 37° C. for 24 hours. The masking peptides were confirmed to be removed from the light chain by Reduced CE-SDS analysis FIG. 5A. The masking efficiency was then measured by ELISA as described above. As shown in FIG. 5B and Table 7, after removal of masking peptide, the activatable antibody became indistinguishable from the parental antibody in its binding to the antigen.

TABLE 7
Activatable antibody ELISAs after protease cleavage

| Sample ID | EC₅₀ (nM) | Masking efficiency |
|---|---|---|
| TY21580 | 0.298 | 1 |
| TY24649-MMP9 | 0.286 | 1 |
| TY24652-MMP9 | 0.313 | 1 |

### Example 4. Activatable Antibody Developability Profiles

For manufacturing purpose, it is critical that the discovered activatable antibodies have a good developability profile. Several different tests were performed with purified activatable antibodies that were expressed in mammalian cells. The activatable antibodies were adjusted to 1 mg/mL in 20 mM Histidine, pH 5.5, and antibody quality analysis was performed using analytical size-exclusion chromatography using a Thermo U3000 with a Thermo DAD detector and an XBridge BEH SEC column (7.8 mm×300 mm) (Waters). For each assay, 40 g of antibody was injected, and fractionation was performed at a flow rate of 0.7 mL/min in buffer (50 mM phosphate within 300 mM sodium chloride at pH 6.8). Two accelerated stress tests were conducted: incubation of the activatable antibodies at 40° C. for 28 days, and six cycles of freeze-thaw. The freeze-thaw tests were conducted by freezing 100 μL sample (1 mg/mL in 20 mM histidine, pH 5.5) at −80° C. for 30 minutes, followed by thawing at room temperature for 60 min.

As shown in FIGS. 6A-6B, all activatable antibodies remained stable, and exhibited little aggregation after storage 40° C. for 28 days. After six cycles of freeze-thaw, they show slight deterioration; however, the main monomer peak remained around 95%, indicating that these activatable antibodies were very stable under these accelerated stress tests. Without wishing to be bound by theory, it is worth noting that the activatable antibodies had not yet gone through an extensive buffer optimization process, and therefore, the stability of the activatable antibodies may be further improved with optimized buffer and excipient.

Besides, the hydrophobicity of the discovered activatable antibodies was tested, and compared to the parental antibody TY21580 through hydrophobic interaction chromatography using a Thermo U3000 with a Thermo DAD detector and a MAbPac HIC-10 (5 m, 1000 Å, 4.6×100 mm) column. For each assay, 10 g of antibody was injected, and fractionation was performed at a flow rate of 0.7 mL/min in a gradient buffer (mobile phase A: 1 M ammonium sulfate, 100 mM sodium phosphate, pH 7.0. Mobile phase B: 100 mM sodium phosphate, pH 7.0.0% B to 100% B in 15 min).

The results shown in FIG. 7 demonstrated that the activatable antibodies TY24649 and TY24652 show significantly reduced hydrophobicity than parental antibody TY21580, which was also much more hydrophilic than Tecentriq.

### Example 5. In Vitro Characterization of Activatable Antibodies Targeting CTLA4

### Enhancement of T Cell Activation Primed by SEA

Here in vitro biological activity of the anti-CTLA4 antibody and activatable antibodies vs Ipilimumab to enhance Staphylococcal enterotoxin A (SEA) peptide stimulated human T cell activation in the context of PBMCs, was evaluated in two experiments. SEA as a superantigen is known to activate a large fraction of human T cells by binding to MHCII from APC and T cell receptor (TCR) from T cells, and thus is chosen in this study to prime T cell activation.

In the first experiment, human PBMCs (peripheral blood mononuclear cells, 2.0×10⁵/well) were isolated from one healthy donor and stimulated with a sub-optimal concentration of the SEA peptide (50 ng/mL) and serially diluted concentrations of anti-CTLA4 antibody and activatable antibodies, Ipilimumab or an isotype control antibody. Replicate cell supernatants were collected after 4 days for measurement of IL-2 with ELISA as an endpoint for enhanced T cell activation. As demonstrated in FIG. 8, TY24649, TY24652 and TY22404 showed weak PBMC activation activity. After in vitro cleavage by recombinant MMP-9 their activity were recovered compared with their parental antibody TY21580 and TY24040 (i.e., TY21580 with the ADCC-enhancing substitutions S239D and I332E). TY24040 and TY24652-MMP9 show strong activity at very low dose level (0.32 nM), while other antibodies (TY21580, ipilimumab, TY22404-MMP9 and TY24649-MMP9) show dose dependent PBMC activation activity, indicating that the ADCC enhancing mutation can enhance the activity of this assay.

The second experiment was performed using the same method as the first experiment, except that PMBCs were isolated from three healthy donors (#SC12086W, #SC12096W and #SC12098W). As shown in Table 8, both TY24652-MMP9 and TY21580 enhanced SEA-induced IL-2 release in an exposure-dependent manner in PBMC cultures from all three donors, whereas the activatable TY24652 (uncleaved) and Isotype Ctrl had no such activity at concentrations up to 1000 nM. Of note, when compared with TY21580, TY24652-MMP9 was more active in enhancing T cell activation, as manifested by higher levels (e.g., 1.8-6.1-fold) of IL-2 release throughout the same tested concentrations for two donors. However, for the third donor (SC12098W), the maximal IL-2 release of TY21580 is a little higher than the TY24652-MMP9.

TABLE 8
EC50s of antibody-induced IL-2 release.

| | EC50 (nM) | | |
|---|---|---|---|
| | | | Fold Δ (TY21580/ |
| Donor | TY24652-MMP9 | TY21580 | TY26425-MMP9) |
| #SC12086W | 3.4 | 6.2 | 1.82 |
| #SC12096W | 4.26 | 24.03 | 5.64 |
| #SC12098W | 2.62 | 16.05 | 6.13 |
| Average | 3.43 | 15.43 | 4.5 |

### ADCC Reporter Activity

Next the reporter gene activity of antibody-dependent cell-mediated cytotoxicity (ADCC) of the anti-CTLA4 antibody and activatable antibodies vs Ipilimumab on their target cells (293F-CTLA4) was evaluated. The effector cells, Jurkat-NFAT-CD16, are engineered from the human T-lymphocyte Jurkat cell line. Jurkat cells naturally express a functional NFAT transcription factor, which is involved in the early signal events in ADCC. The target cells (2.0×10⁴) used here are 293F overexpressing CTLA4 target molecules. The ADCC reporter activity of the antibodies are assessed by co-incubation of 293F-CTLA4 target cells with effector cells (1.2×10⁵) in the presence and absence of the antibodies at different concentrations, followed by analysis of the luciferase. The IgG1 isotype control mAb is used as negative control for ADCC reporter activity. As shown in FIG. 9, the activatable antibodies showed several log lower ADCC activities than the parental antibody TY21580. After cleavage, the activity of TY24649-MMP9 and TY22404-MMP9 was partly recovered compared with TY21580, while the activity of TY24652-MMP9 was mostly recovered compared with TY24040.

### Ligand Blocking

The functional activity of the anti-CTLA4 antibodies and activatable antibodies to stimulate CD28 mediated downstream cell signaling by blocking CTLA4 sequestration of CD80 and CD86 ligands, was evaluated in two studies.

The first study employed the Promega CTLA4 blockade bioassay system, a bioluminescent cell-based assay, to assess the CTLA4 blocking function of the anti-CTLA4 mAbs. This assay system involves two genetically engineered cell lines: CTLA4 effector cells-Jurkat T cells expressing human CTLA-4 (1.0×10⁵/well) and a luciferase reporter driven by a IL-2 native promoter which responds to TCR/CD28 activation; and aAPC/Raji cells (2.0×10⁵)-Raji cells expressing cell surface protein designed to activate TCRs in an antigen-independent manner and endogenously expressing CTLA4 ligands CD80 and CD86. When the two cell types are co-cultured, CTLA-4 competes with CD28 for their shared ligands, CD80 and CD86, and thus inhibits CD28 pathway activation and promoter-mediated luminescence. Addition of the anti-CTLA4 antibody blocks the interaction of CTLA-4 with its ligands CD80 and CD86 and results in promoter-mediated luminescence. The bioluminescent signal can then be detected using the Bio-Glo™ Luciferase Assay System. As shown in FIG. 10, TY24649, TY24652 and TY22404 show weaker block activities than TY21580 and TY24040. After cleavage the blocking activities were mostly recovered. TY24040 and TY24652-MMP9 show similar blocking activities, which were higher than TY21580, TY22404 and TY24649-MMP9, indicating that the ADCC enhancing mutation can enhance the block activity.

In the second study, the effects of activatable antibody TY24652 (before the cleavage site is cleaved) and its activated form (after the cleavage site is cleaved) on the binding of CD80 or CD86 ligand to its receptor CTLA-4 was assessed by ELISA. Briefly, recombinant human CD80 or CD86 protein was coated onto ELISA microplates, to capture biotinylated CTLA-4 protein in the presence of serial dilutions of TY24652, TY24652-MMP9, or an isotype control antibody. After incubation and washing off unbound proteins, the interactions between CD80/86 and biotinylated CTLA-4 were then detected with an HRP-labeled Streptavidin, and subsequent chromogenic reaction with TMB substrate. Relative CTLA-4 and its ligand binding was monitored by reading the absorbance at 450 nm wavelength in the micro-plate reader. As shown in Table 9, when CD80 or CD86 ligand was immobilized onto plate, the activated (cleaved) form (TY24652-MMP9) completely and effectively blocked the CTLA-4 binding to CD80 and CD86 in a concentration-dependent manner, whereas the non-cleaved form TY24652 blocked very weakly the CTLA-4 binding to CD80 and CD86 only at high concentrations. As expected, the isotype control antibody had no blocking activity at all.

TABLE 9
Ligand blocking by activatable antibodies after
protease cleavage (determined by ELISA).

| | CTLA4/CD80 | | CTLA4/CD86 | |
|---|---|---|---|---|
| Antibody | IC50 (nM) | Fold Δ | IC50 (nM) | Fold Δ |
| TY24652 | >20000 | — | >4000 | — |
| TY24652-MMP9 | 279.7 | >72 | 17.49 | >229 |

### Example 6. Cell Surface Binding Activity

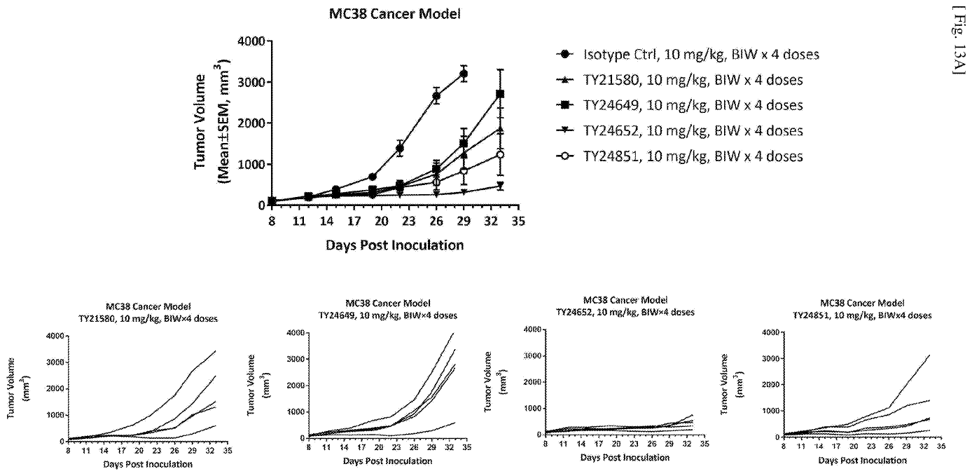

The target binding activity of the anti-CTLA4 antibodies and activatable antibodies was evaluated by flow cytometry with human CTLA-4 transfected 293F cells. 1.0×105 293F-hCTLA4 cells were incubated with serial diluted antibodies at 37° C. for 30 min. After wash, APC-labeled mouse anti-human IgG Fc 2nd antibody was added and incubated for another 30 min, followed by flow cytometry analysis. As shown in FIG. 11, the activatable antibodies TY24649, and TY24652 and TY22404 showed weak target binding activities at high concentrations, however, once cleaved by MMP9, the high target binding activities of these activatable anti-CTLA4 antibodies were fully recovered, at mean fluorescence intensities (MFI) similar to that observed with the parental TY21580 and TY24040 enhancing mutation.

### Example 7. Binding Affinities to Fcγ Receptors

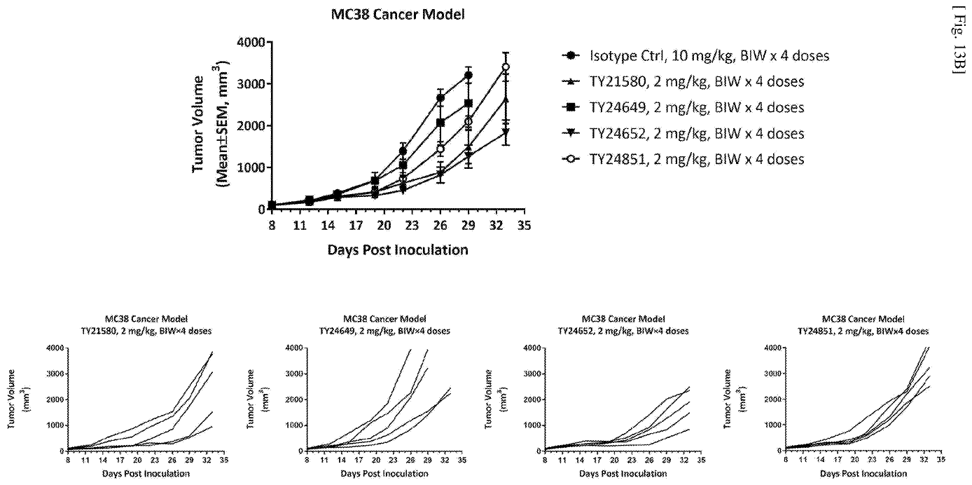

The binding affinities of the activated TY24649 with wild-type human IgG1 and the activated TY24652 with human ADCC enhancing mutation in the IgG1 Fc region, to the immobilized recombinant Fcg receptor proteins, were evaluated by Biacore assay. As shown in FIG. 12 and Table 10, compared to the activated TY24649 with wild-type human IgG1, the activated TY24652 with ADCC enhancing mutation exhibited much higher binding affinities to the CD16 proteins of human, monkey and mouse origins. These results provided mechanistic explanation for the enhanced ADCC activity of the activated TY24652.

TABLE 10
Binding affinities of TY24649 and TY24652 to FcγRIIIA

| | Human | Human | | |
|---|---|---|---|---|
| | FcγRIIIA | FcγRIIIA | Monkey | Mouse |
| Biacore K_{D} (nM) | (V158) | (F158) | FcγRIII | FcγRIV |
| Activated TY24649 | 150.4 | 426.3 | 117.4 | 94.7 |
| Activated TY24652 | 5.93 | 8.90 | 5.12 | 0.983 |
| Ratio | 25 | 48 | 23 | 95 |

### Example 8. In Vivo Characterization of Activatable Antibodies Targeting CTLA4

The anti-tumor activities of the activatable antibodies were next evaluated and compared with the anti-tumor activity of the parental antibody TY21580 in multiple syngeneic mouse tumor models, including an MC38 colorectal tumor model, a CT26 colorectal tumor models, and a Lewis lung tumor model.

### Anti-Tumor Efficacy in an MC38 Colorectal Tumor Model

In a first study, C57BL/6 mice (n=8 per group, female, 8-9 weeks old) were inoculated subcutaneously with MC38 (FDCC) murine colon cancer cells. When tumors were established (100 mm³), treatment began with isotype control antibody, parental antibody TY21580, or one of the two activatable antibodies TY24649 and TY24652, non-cleavable antibody TY24851, by intraperitoneal injection, twice a week. As shown in FIG. 13A tumor growth was monitored twice a week, the mean tumor volume±s.e.m. over time and individual tumor growth curves were assessed when 10 mg/kg of TY21580, TY24649, TY24652 and TY24851 was administered two times a week for 4 doses. As shown in FIG. 13B tumor growth was monitored twice a week, the mean tumor volume±s.e.m. over time and individual tumor growth curves were assessed when 2 mg/kg of TY21580, TY24649, TY24652 and TY24851 was administered two times a week for 4 doses. All tested antibodies show anti-tumor efficacy in MC38 colorectal tumor model. As shown in FIGS. 13A-B, TY24652 shows better efficacy than parental antibody TY21580 at both doses, TY24851 shows better efficacy than parental antibody TY21580 at high dose and comparable efficacy at low dose. While TY24649 shows comparable efficacy to the parental antibody TY21580 at high dose, but weaker efficacy at low dose.

In a second study, C57BL/6 mice (n=6 per group, female, 8-9 weeks old) were inoculated subcutaneously with MC38 (NTCC) murine colon cancer cells. When tumors were established (about 84 mm³), treatment began with a vehicle control, parental antibody TY21580, or the activatable antibody TY24652 at 0.05 mg/kg, 0.2 mg/kg and 1 mg/kg by intraperitoneal injection, twice a week. Tumor growth was monitored twice a week, the mean tumor volume±s.e.m. over time. As shown in FIG. 13C, TY21580 and TY24652 both showed dose dependent anti-tumor effect. TY21580 showed comparable efficacy with the TY24652 at 1 mg/kg and better efficacy than TY24652 at 0.05 mg/kg and 0.2 mg/kg.

### Anti-Tumor Efficacy in a CT26 Colorectal Tumor Model

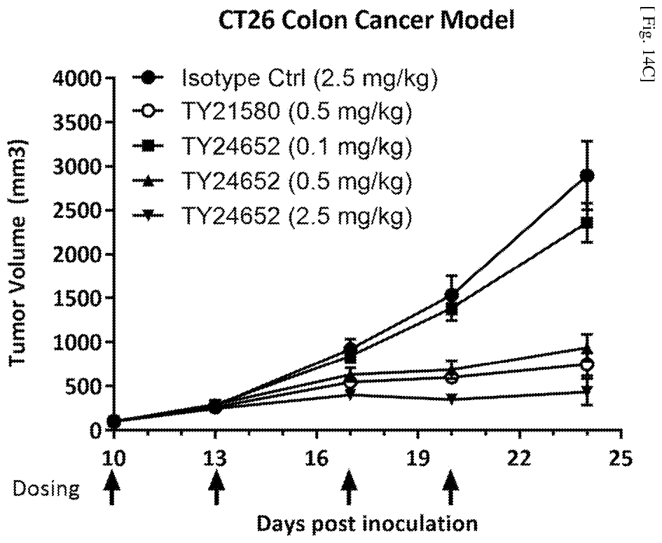

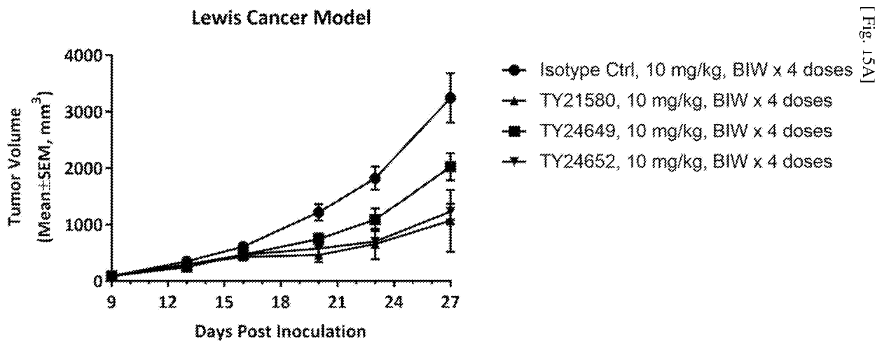

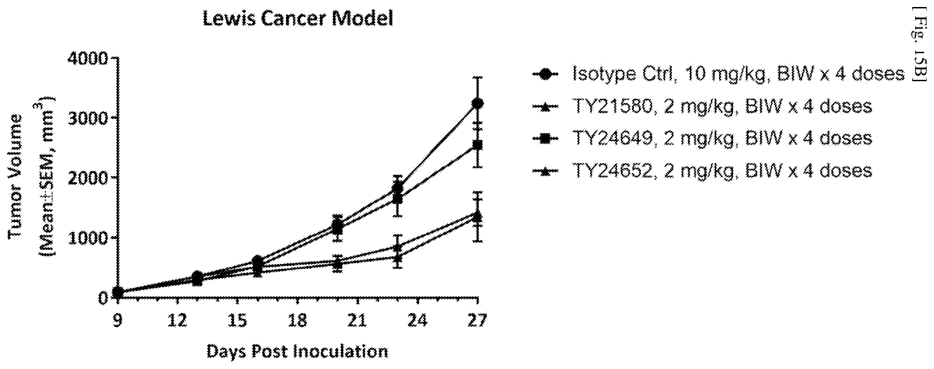

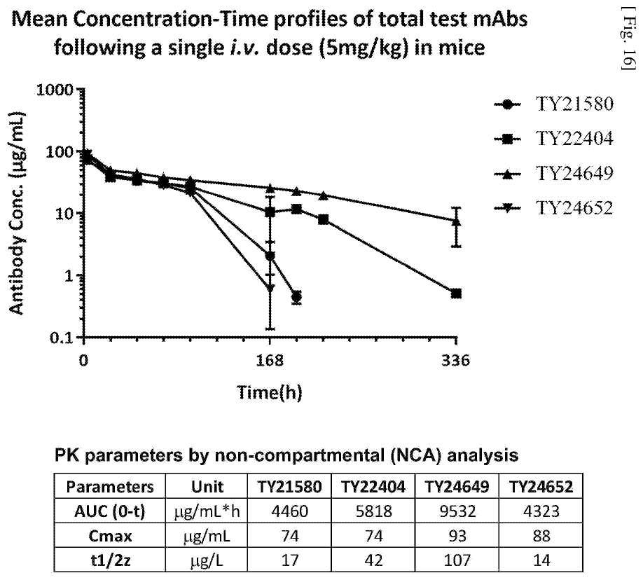

In a first study, BALB/c mice (n=8 per group, female, 6-7 weeks old) were inoculated subcutaneously with CT26 (Shanghai Institutes for Biological Sciences) murine colon cancer cells. When tumors were established (100 mm³), treatment began with isotype control antibody, parental antibody TY21580, or one of three activatable antibodies, non-cleavable antibody TY24851, at 5 mg/kg or 1 mg/kg by intraperitoneal injection, twice a week. Tumor growth was monitored twice a week and reported as the mean tumor volume±s.e.m. over time. As shown in FIGS. 14A-14B, all tested antibodies TY21580, TY21649 and TY24652 show anti-tumor efficacy in CT26 colorectal tumor model at both 5 mg/kg and 1 mg/kg. shows the similar efficacy as parental antibody TY21580 at both doses, while TY24649 TY24652 and TY24851 shows the comparable efficacy to the parental antibody TY21580 at high dose, but weaker efficacy at low dose.

In a second study, BALB/c mice (n=8 per group, female, 7-8 weeks old) were inoculated subcutaneously with CT26 (Shanghai Institutes for Biological Sciences) murine colon cancer cells. When tumors were established (103 mm³), treatment began with isotype control antibody, activatable antibody TY24652 at 0.1 mg/kg, 0.5 mg/kg and 2.5 mg/kg, or parental antibody TY21580 at 0.5 mg/kg by intraperitoneal injection, twice a week. Tumor growth was monitored twice a week and reported as the mean tumor volume±s.e.m. over time. As shown in FIG. 14C, TY24652 showed dose dependent anti-tumor effect and comparable efficacy with TY21580 at 0.5 mg/kg.

### Anti-Tumor Efficacy in a Lewis Lung Tumor Model

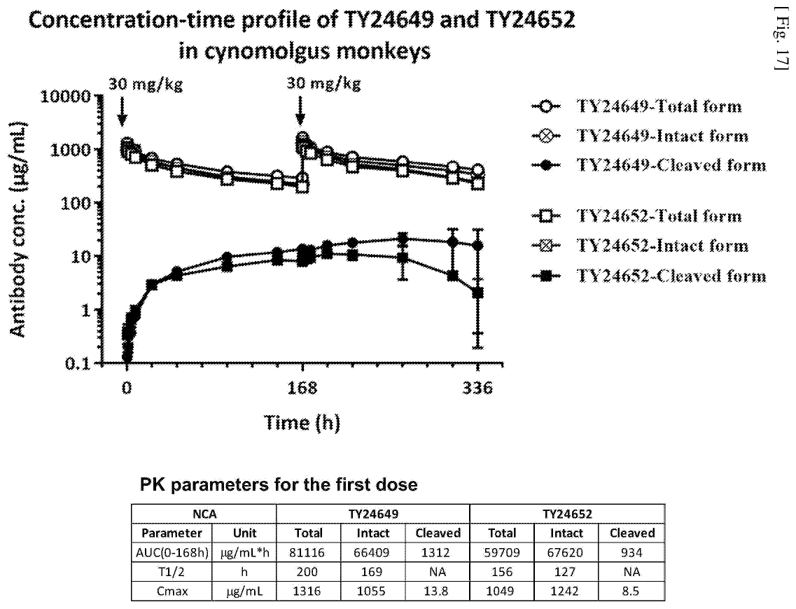

C57BL/6 mice (n=8 per group, female, 7-9 weeks old) were inoculated subcutaneously with Lewis (GuangZhou Jennio Biotech Co., Ltd) murine colon cancer cells. When tumors were established (91 mm³), treatment began with isotype control antibody, parental antibody TY21580, or one of the two activatable antibodies by intraperitoneal injection, twice a week. Tumor growth was monitored twice a week, the mean tumor volume±s.e.m. over time (FIGS. 15A-15B) were assessed. As shown in FIGS. 15A-15B, all tested antibodies TY21580, TY21649 and TY24652 show anti-tumor efficacy in a Lewis lung tumor model at both 10 mg/kg and 2 mg/kg. TY24652 shows the similar efficacy as parental antibody TY21580 at both doses, while TY24649 shows weaker efficacy at both doses.

### Example 9. Pharmacokinetics (PK) in Mice

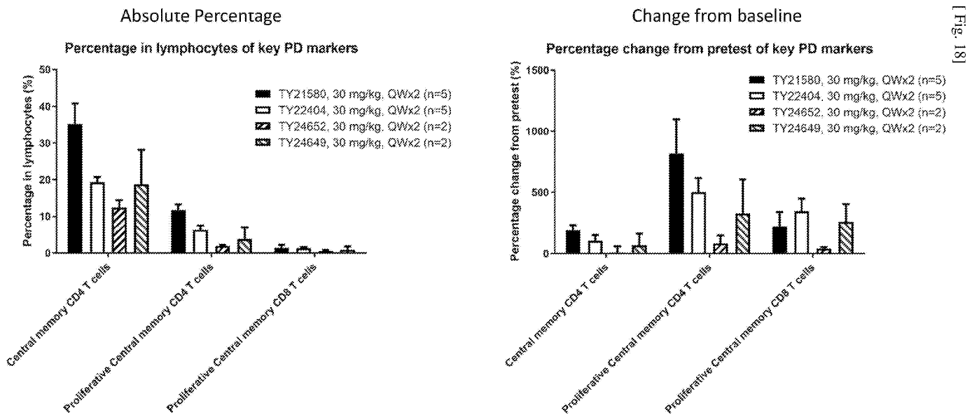

A pharmacokinetics study was conducted in BALB/c female mice at about eight weeks of age. Four mice per group were intraperitoneally injected with the test article at 5 mg/kg. Blood samples (—50 μl per sample) were collected at 3, 24, 48, 72, 96, 168, 192, 216 and 336 hours post-dosing. Serum concentrations of each test antibody were determined by ELISA, in which anti-human IgG Fc was used for capture, and HRP-labeled anti-human IgG (Fab specific) antibody (Sigma) was used for detection (FIG. 16). As compared to the data collected for parental antibody TY21580, activatable antibodies TY24649 had a slower clearance time, longer half-life and higher systemic exposure. With ADCC enhancing mutation, TY24652 showed similar PK profile as the parental antibody TY21580 in mice.

### Example 10. Pharmacokinetics (PK) in Cynomolgus Monkeys

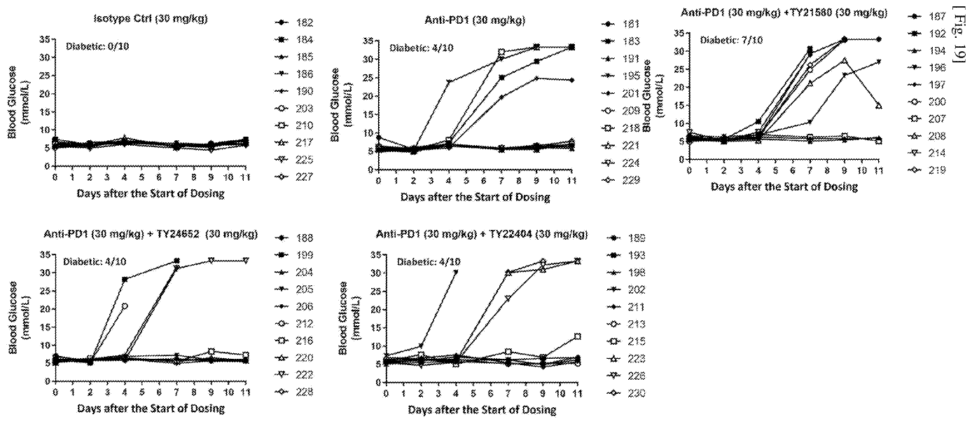

To evaluate the pharmacokinetics of the activatable antibodies on peripheral lymphocyte. Cynomolgus monkeys were i. v. administered with TY24649 and TY24652 weekly at 30 mg/kg for 2 doses. Peripheral plasma samples were collected before and during the treatments at different time points. Plasma concentrations of these antibodies were determined by ELISA assays. The total form antibodies were measure by ELISA assay with anti-human IgG, the intact form antibodies were measured by ELISA with a specific anti-idiotype antibody recognizing the masking peptide, and the cleaved form antibodies were measure by ELISA with a specific anti-idiotype antibody recognizing the active antibodies. As shown in FIG. 17, TY24649 and TY24652 showed similar PK profile in cynomolgus monkeys. The majority of these two antibodies exhibited as intact forms in circulation, with very low levels (<2%) of the cleaved forms compared to the total forms in AUC0-168 h accumulated over time.

### Example 11. Pharmacodynamic Activity in Cynomolgus Monkeys

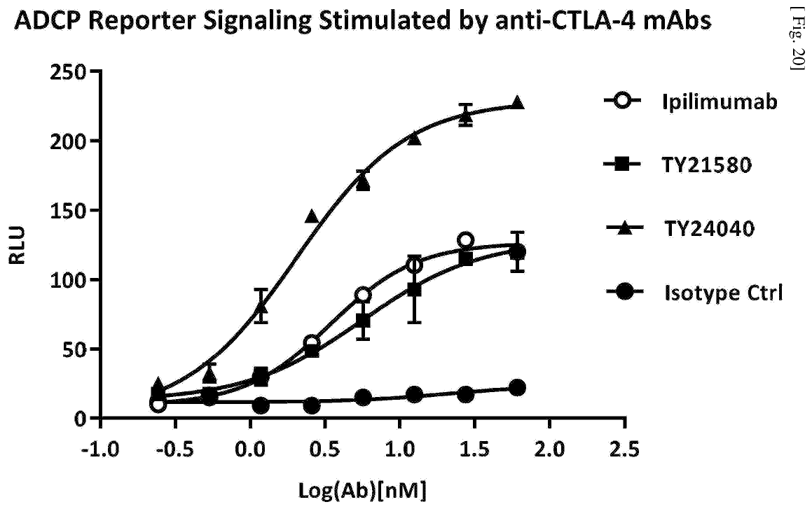

To evaluate the pharmacodynamic activities of the anti-CTLA4 antibodies and activatable antibodies on peripheral lymphocyte. Cynomolgus monkeys were i. v. administered with TY21580, TY22404, TY24649 and TY24652 weekly at 30 mg/kg for 2 doses. Peripheral blood samples were collected before and during the treatments, subtypes of lymphocytes were examined by flow cytometry with specific markers. Percentages of the central memory CD4+ T cells (CD45+CD3+CD4+CD8-CD28+CD95+), proliferative (Ki67+) central memory CD4+ T cells, and proliferative (Ki67+) central memory CD8+ T cells (CD45+CD3+CD4-CD8+CD28+CD95+), were significantly affected on Day 14 post-dose by the anti-CTLA4 antibodies. As shown in FIG. 18, TY21580 showed the highest stimulatory activity on these lymphocyte subtypes, followed by TY22404, TY24649 and TY24652.

### Example 12. TOX Study in NOD Mice

To evaluate the diabetes induction toxicity of the anti-CTLA-4 antibodies, female NOD/LtJ mice at 5-week-old age (n=10 per group) were i. p. administered with a single dose (30 mg/kg) of a mouse cross-reactive anti-PD-1 alone, or together with 30 mg/kg of parental TY21580, or the activatable antibodies TY22404, or TY24652. An isotype control antibody was also included. Following the treatment, the levels of blood glucose were monitored over time for each individual mouse. As shown in FIG. 19, anti-PD-1 treatment alone can induce diabetes with 4 of 10 mice exhibited dramatically increased blood glucose levels. The parental anti-CTLA4 antibody TY21580 further enhanced diabetes induction frequency when combined with anti-PD-1, with 7 of 10 mice exhibited increased blood glucose levels. However, both the activatable TY22404 and TY24652 showed no further increased diabetes incidence when combined with anti-PD-1, suggesting that the activatable TY22404 and TY24652 could have better safety profile when combined with anti-PD-1 in terms of diabetes inducing side effect.

### Example 13. Enhanced ADCP Signaling Activity of Fc-Engineered Anti-CTLA-4 Antibody

The reporter gene activity of antibody-dependent cellular phagocytosis (ADCP) of the anti-CTLA-4 mAbs TY21580 and TY24040 vs. Ipilimumab on their target cells (293F-CTLA-4) was evaluated. The effector cells, Jurkat-NFAT-CD32a, are engineered from the human T-lymphocyte Jurkat cell line. Jurkat cells naturally express a functional NFAT transcription factor, which is involved in the early signal events in ADCP. The target cells (1.0×10⁵) used here are 293F overexpressing CTLA-4 target molecules. The ADCP reporter activity of the anti-CTLA-4 mAbs are assessed by co-incubation of 293F-CTLA-4 target cells with effector cells (1.0×10⁵) in the presence and absence of the anti-CTLA-4 mAbs at different concentrations, followed by analysis of the luciferase. The IgG1 isotype control mAb is used as negative control for ADCP reporter activity.

As shown in FIG. 20 and Table 11, TY24040 antibody showed better ADCP reporter activity, compared with TY21580 and Ipilimumab. The isotype control in this assay showed no detectable ADCP activity.

TABLE 11
ADCP reporter activity of anti-CTLA-4 antibodies.

| Antibody | EC50 (nM) | Span | Total Area |
|---|---|---|---|
| Ipilimumab | 3.441 | 117.5 | 169.2 |
| TY21580 | 5.597 | 116.3 | 151.9 |
| TY24040 | 2.04 | 227 | 334.9 |

### Example 14. Binding Affinity of TY24652 and its Activated Form, TY21580 and Ipilimumab to CTLA-4 Across Species

Here binding kinetics and affinity of TY24652, TY24652-MMP9, TY21580 and Ipilimumab to the recombinant CTLA-4 proteins of human, cynomolgus monkey, mouse and rat origins were determined by surface plasmon resonance (SPR) separately. Briefly, the TY24652, MMP9-cleaved (activated) TY24652, TY21580, and Ipilimumab antibodies were immobilized onto the Biacore sensor chip through chip-coupled anti-human IgG (Fc) antibody. Serial dilutions of recombinant CTLA-4 proteins of different species origins were sequentially loaded and flowed through the chip to measure the kinetics of CTLA-4 antigen association (kₐ), followed by a buffer flow-through phase for dissociation kinetics (k_{d}) measurement. The binding affinity (K_{D}) is then calculated with k_{d}/k_{d}.

As shown in Table 12, the anti-CTLA-4 activatable antibody TY24652 can bind to the recombinant CTLA-4 proteins of human, cynomolgus monkey, mouse and rat, but with low affinities (K_{D}s ranging from 32.27 to 59.5 nM). While its activated form (TY24652-MMP9) binds to the recombinant CTLA-4 proteins of all four species, with comparable high affinities (K_{D}s ranging from 0.44 to 0.69 nM), which are close to its parental antibody TY21580 (K_{D}s ranging from 0.37 to 0.78 nM). Of note, the activated form, TY24652-MMP9, shows ~3-fold higher binding affinity to human CTLA-4 than the clinically approved anti-CTLA-4 antibody Ipilimumab. These results demonstrate that the antigen-binding activity is masked in the intact (non-cleaved) TY24652 activatable antibody, while cleaving off the masking peptide by protease MMP9 activates TY24652 and rescues its potent binding activity to its target CTLA-4 proteins with over 60-fold increased affinities.

TABLE 12
Binding affinity to recombinant CTLA-4
across species (as determined by SPR).

| Antigen | Antibody | K_{D} (nM) | Fold | kₐ (1/Ms) | k_{d} (1/Ms) |
|---|---|---|---|---|---|
| Human | TY24652 | 32.49 | — | 1.03E+05 | 3.35E−03 |
| CTLA-4 | TY24652-MMP9 | 0.51 | 63.7 | 1.02E+06 | 5.17E−04 |
| | TY21580 | 0.44 | 73.8 | 1.15E+06 | 5.02E−04 |
| | Ipilimumab | 1.7 | 19.1 | 3.22E+05 | 5.48E−04 |
| Monkey | TY24652 | 32.27 | — | 1.13E+05 | 3.66E−03 |
| CTLA-4 | TY24652-MMP9 | 0.44 | 73.3 | 8.45E+05 | 3.72E−04 |
| | TY21580 | 0.37 | 87.2 | 9.61E+05 | 3.51E−04 |
| | Ipilimumab | 1.18 | 27.3 | 3.74E+05 | 4.40E−04 |
| Mouse | TY24652 | 49.4 | — | 1.46E+05 | 7.19E−03 |
| CTLA-4 | TY24652-MMP9 | 0.63 | 78.4 | 9.08E+05 | 5.72E−04 |
| | TY21580 | 0.5 | 98.8 | 1.07E+06 | 5.36E−04 |
| Rat | TY24652 | 59.5 | — | 1.64E+05 | 9.73E−03 |
| CTLA-4 | TY24652-MMP9 | 0.69 | 86.2 | 5.07E+05 | 3.52E−04 |
| | TY21580 | 0.78 | 76.3 | 5.39E+05 | 4.19E−04 |

### Example 15. Enhanced Antitumor Efficacy by TY24652 in Combination Therapies

TY24652 in Combination with an Anti-CD137 Antibody

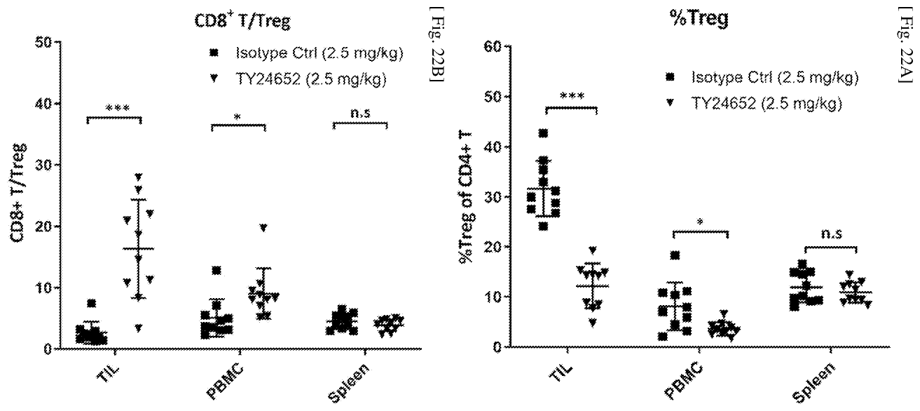

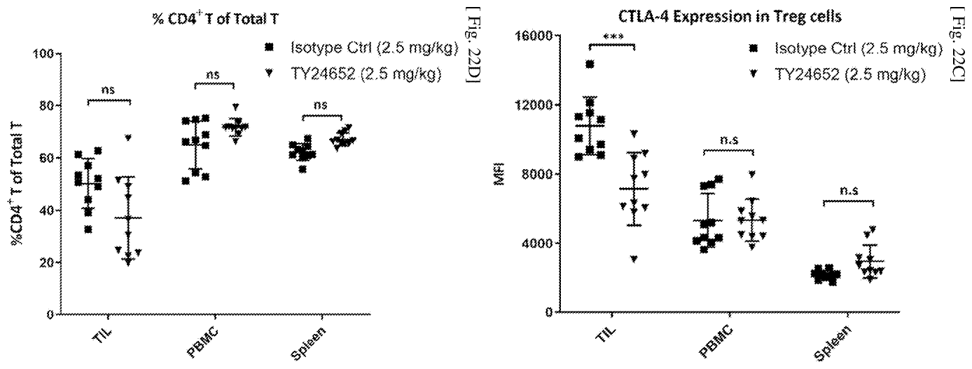

The efficacy of a combination of TY24652 and the anti-CD137 antibody AG10131 to inhibit tumor growth in a MC38 colon tumor model was evaluated. C57BL/6 mice (n=8 per group, female, 7-8 weeks old) were inoculated subcutaneously with MC38 (NTCC) murine colon cancer cells. When tumors were established (88 mm³), treatment began with a vehicle control, TY24652 (0.2 mg/kg), AG10131 (10 mg/kg) or the combination, by intraperitoneal injection, twice a week. Tumor growth was monitored twice a week and reported as the mean tumor volume±s.e.m. over time. As shown in FIG. 21A, TY24652 and AG10131 showed partial efficacy as single agent, with tumor growth inhibition at 66% and 75% on Day 31 respectively. The combination of TY24652 and AG106 resulted in significantly enhanced antitumor efficacy, with a tumor growth inhibition at 97%.

TY24652 in Combination with an Anti-PD-1 Antibody

The efficacy of a combination of TY24652 and an anti-PD-1 antibody (2E5, CN107840887A) to inhibit tumor growth in a MC38 colon tumor model was evaluated. BALB/c mice (n=8 per group, female, 8-9 weeks old) were inoculated subcutaneously with CT26 (SIBS) murine colon cancer cells. When tumors were established (84 mm³), treatment began with Vehicle, TY24652 (0.5 mg/kg), the anti-PD1 antibody (5 mg/kg), or the combination, by intraperitoneal injection, twice a week. Tumor growth was monitored twice a week and reported as the mean tumor volume±s.e.m. over time. As shown in FIG. 21B, TY24652 and the anti-PD1 antibody showed partial efficacy as single agent, with TGI at 58% and 42% on Day 22 respectively. The combination of TY24652 and anti-PD1 resulted in significantly enhanced antitumor efficacy, with a TGI at 75%.

### Example 16. Treg Depletion by Anti-CTLA4 Activatable Antibody a CT26 Colon Tumor Model

The depletion of Treg cells by activatable antibody TY24652 was evaluated. BALB/c mice (n=10 per group, female, 7-8 weeks old) were inoculated subcutaneously with CT26 (SIBS) murine colon cancer cells. When tumors were established (141 mm³), treatment began with isotype control antibody and TY24652 at 2.5 mg/kg, by intraperitoneal injection, twice a week. The mice were sacrificed on Day 5 after start of dosing and tumors, blood and spleen were collected for the analysis of the effect of TY24652 to tumor-infiltrating lymphocytes (TILs) and peripheral lymphocytes.

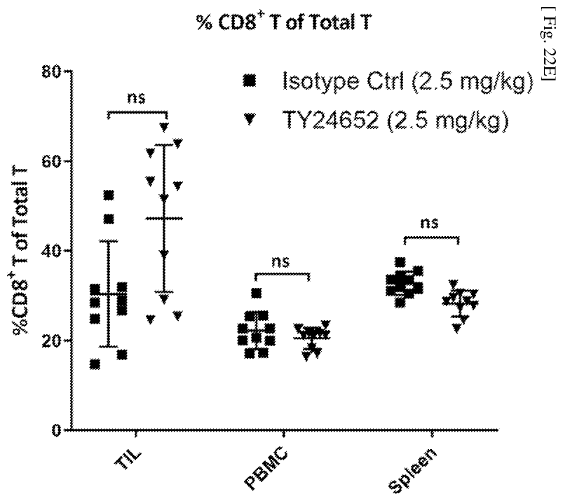

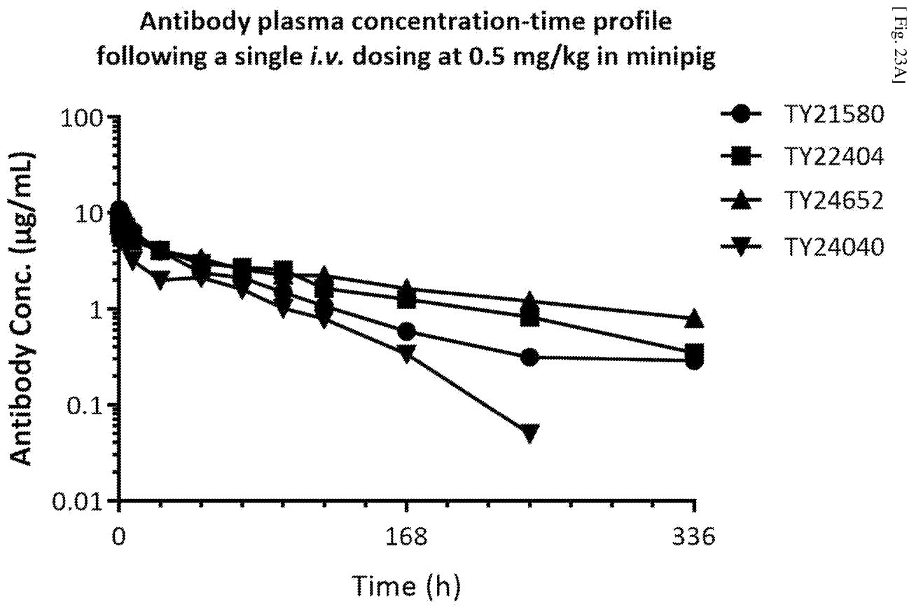

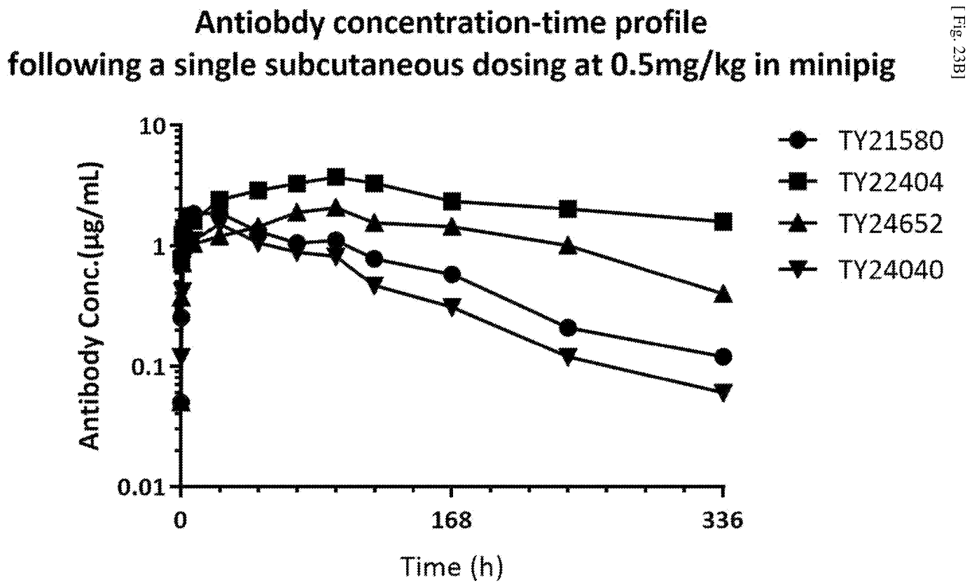

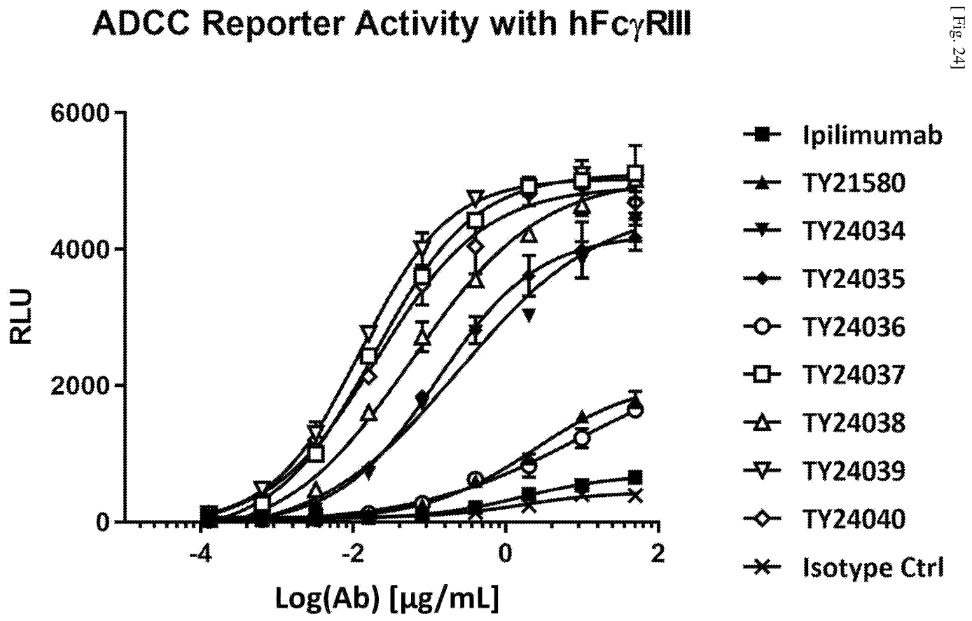

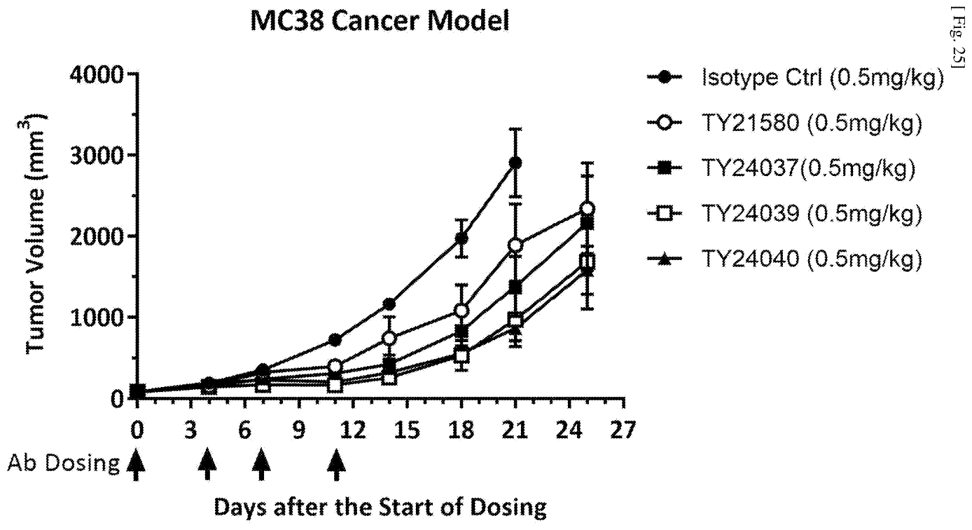

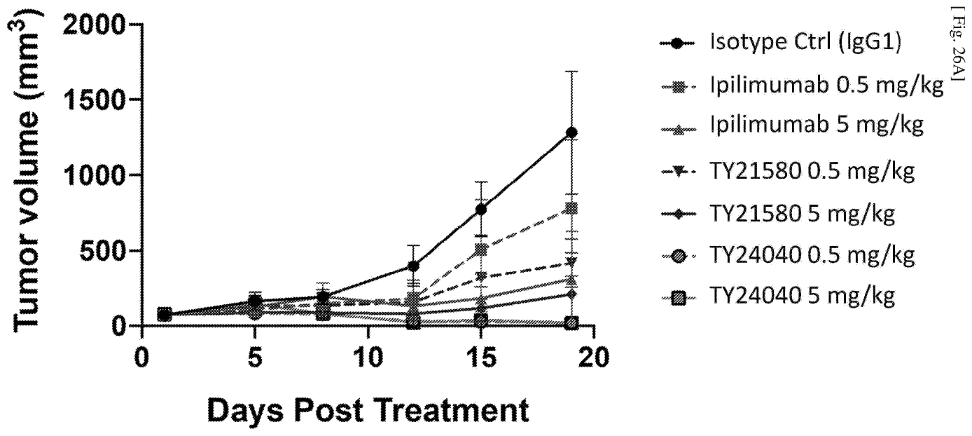

As shown in FIG. 22A, TY24652 significantly reduced the percentage of Tregs in CD4+ T cells comparing with Isotype Ctrl in TILs (12.2% vs 31.7%, P<0.001) and to a lesser extent in PBMCs (3.6% vs 8.2%, P<0.05). However, TY24652 did not influence the percentage of Tregs in CD4+ T cells in spleen (10.9% vs 12.0%, P>0.05; FIG. 22A). TY24652 also significantly increased the mean CD8+T/Treg ratio comparing with Isotype Ctrl in TILs (16.3 vs 2.7, P<0.001) and to a lesser extent in PBMCs (9.0 vs 5.1, P<0.05; FIG. 22B). However, TY24652 did not influence the CD8+T/Treg cell ratio in Spleen (3.9 vs 4.5, P>0.05; FIG. 22B). TY24652 significantly decreased CTLA-4 expression level of Tregs in TILs (FIG. 22C). However, the CTLA-4 expression level of Tregs in PBMCs and spleen were not influenced (FIG. 22C). The percentage of CD4+ and CD8+ T cells in TILs, PBMCs and spleen were not influenced by TY24652 treatment (FIGS. 22D and 22E).

### Example 17. Pharmacokinetics (PK) in Minipig

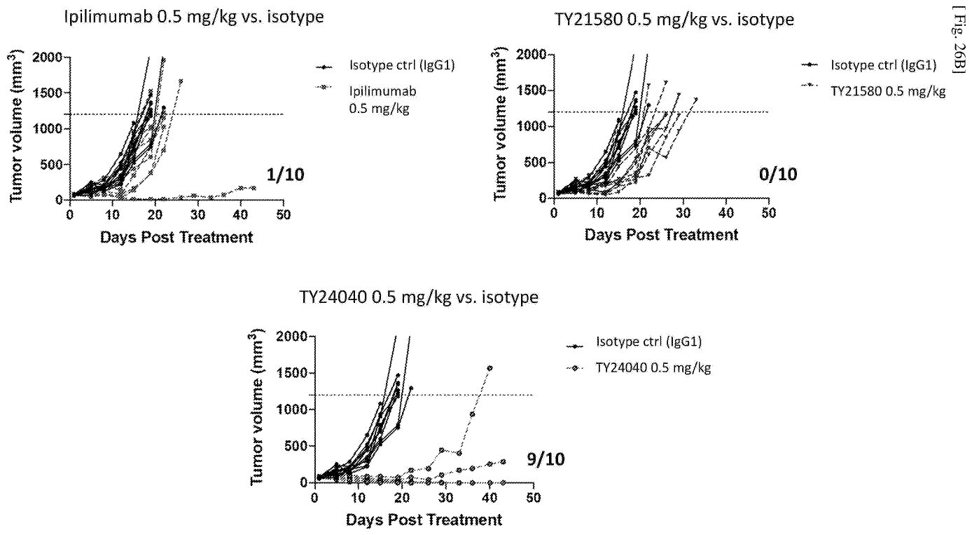

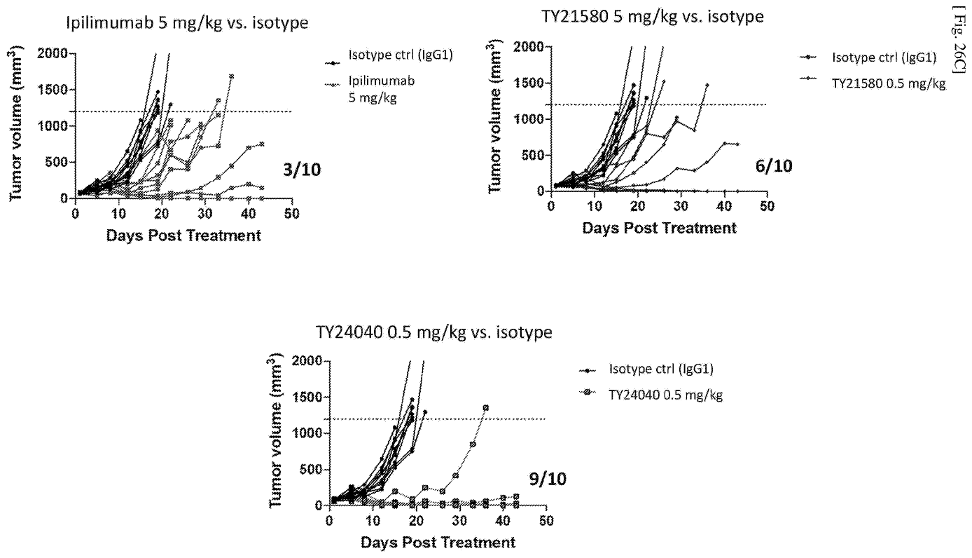

The pharmacokinetics (PK) of the anti-CTLA4 antibodies TY21580, TY22404, TY24652, and TY24040 were evaluated in a minipig model. Minipigs received a single injection intravenously or subcutaneously with the antibody at a dose of 0.5 mg/kg. The parameters and results of the PK analysis is shown in Table 13, and the plasma concentration time profiles of the antibodies are shown in FIGS. 23A-23B. For total drug PK, the SC bioavailability (F %) of TY21580, TY22404, TY24652 and TY24040 is estimated to be ~56%, >100%, ~61% and ~61%, respectively. For intact drug PK, the SC F % of TY22404 and TY24652 is estimated to be ~85% and ~52%, respectively. Based on intact drug, Tmax after SC dosing is 8-24 hours (TY21580), 48-72 hours (TY22404), 72-120 hours (TY24652), and 8-24 hours (TY24040). Cleaved form was measurable for TY22404, which accounted for ~28% AUC of total drug.

TABLE 13
Pharmacokinetic parameters of anti-CTLA4 antibodies in minipig.

| | | | | | | | | | Cleaved/ |
|---|---|---|---|---|---|---|---|---|---|
| | Dose | | Cₘₐₓ | Tₘₐₓ | T_{1/2} | AUCₗₐₛₜ | AUC_{inf} | F | Total |
| AB | Route | Format | (μg/mL) | (h) | (h) | (μg/mL) | (μg/mL) | (%) | (AUC %) |
| TY21580 | IV | Total | 10.9 | — | 82.9 | 460 | 467 | — | — |
| | SC | Total | 2.04 | 8-24 | 220 | 259 | 282 | 56.4 | — |
| TY22404 | IV | Total | 8.17 | — | 108 | 650 | 664 | — | — |
| | | Intact | 8.96 | — | 64.4 | 418 | 425 | — | — |
| | | Cleaved | 0.58 | 96.0 | 151 | 181 | 200 | — | 27.9 |
| | SC | Total | 3.77 | 72-96 | 176 | 1093 | 1170 | 168 | — |
| | | Intact | 1.88 | 48-72 | 84.0 | 354 | 364 | 84.6 | — |
| | | Cleaved | 0.89 | 168-240 | 76.1 | 311 | 325 | — | 28.4 |
| TY24652 | IV | Total | 7.67 | — | 120 | 777 | 803 | — | — |
| | | Intact | 8.86 | — | 116 | 681 | 702 | — | — |
| | SC | Total | 2.13 | 96-120 | 124 | 470 | 487 | 60.5 | — |
| | | Intact | 1.88 | 72-120 | 106 | 354 | 364 | 51.9 | — |
| TY24040 | IV | Total | 5.84 | — | 35.9 | 260 | 264 | | |
| | SC | Total | 1.52 | 8-24 | 70.9 | 159 | 165 | 61.1 | |

### Example 18. ADCC Reporter Activity of Anti-CTLA4 Antibodies with IgG1 Fc Mutations

Here the reporter gene activity of antibody-dependent cell-mediated cytotoxicity (ADCC) of the anti-CTLA-4 mAbs with various Fc mutations vs Ipilimumab and its wild type antibodies on their target cells (293F-CTLA-4) was evaluated. Various IgG1 Fc mutations were introduced into TY21580 to generate the antibodies described in Table 14. The effector cells, Jurkat-NFAT-CD16, are engineered from the human T-lymphocyte Jurkat cell line. Jurkat cells naturally express a functional NFAT transcription factor, which is involved in the early signal events in ADCC. The target cells used here are 293F overexpressing CTLA-4 target molecules. The ADCC reporter activity of the anti-CTLA-4 mAbs are assessed by co-incubation of 293F-CTLA-4 target cells (2.0×104 per well) with effector cells (1.2×10⁵ per well) in the presence or absence of the anti-CTLA-4 mAbs in a series of sequential dilutions (50-0.000128 g/mL). After 6-hours incubation, 100 μL of One-Glo reagent was added to the cells, and the cells were lysed for 10 min. Supernatants (100 μL) were collected for luminescence measurements using a SpectraMax i3x plate reader. The IgG1 isotype control mAb is used as negative control for ADCC reporter activity.

TABLE 14
ADCC reporter activity of anti-CTLA4
antibodies with IgG1 Fc mutations

| IgG ID | Fc Mutations |
|---|---|
| TY24034 | IgG1-S298A/E333A/K334A |
| TY24035 | IgG1-S298A/E333A/K334A/K326A |
| TY24036 | IgG1-F243L |
| TY24037 | IgG1-F243L/R292P/Y300L/V305I/P396L |
| TY24038 | IgG1-F243L/R292P/Y300L/L235V/P396L |
| TY24039 | IgG1-S239D/I332E/A330L |
| TY24040 | IgG1-S239D/I332E |

As shown in FIG. 24, the ADCC enhancing mutants TY24037, TY24038, TY24039, and TY24040 exhibited much stronger ADCC reporter gene activities (about 4-10-folds), compared with wild type antibodies TY21580. The Ipilimumab showed much lower ADCC potency, even compared with wild type antibody TY2158 (Fc WT).

### Example 19. Tumor Growth Inhibition by Anti-CTLA4 Antibodies with IgG1 Fc Mutations

Here the tumor suppression activity of the anti-CTLA-4 mAbs with various Fc mutations vs Ipilimumab and its wild type antibodies on was evaluated in an MC38 cancer model. C57BL/6 mice (n=7 per group, female, 7-8 weeks old) were inoculated subcutaneously with MC38 (NTCC) murine colon cancer cells. When tumors were established (about 82 mm³), treatment began with a isotype control, parental antibody TY21580, or ADCC enhanced antibodies TY24037, TY24039 and TY24040 at 0.5 mg/kg by intraperitoneal injection, twice weekly for a total of 4 doses. Tumor growth was monitored twice a week, and data were represented as mean tumor volume±s.e.m. over time.

As shown in FIG. 25, TY21580 and TY24037 show slight inhibitory effect on tumor growth at 0.5 mg/kg (P>0.05), and their TGI were 35% and 53%, respectively. TY24039 and TY24040 had significant inhibition on tumor growth at 0.5 mg/kg (P<0.01) with TGI of 67% and 71%, respectively.

### Example 20. In Vivo Characterization of an Unmasked Anti-CTLA4 Antibody TY24040 with ADCC-Enhancing IgG1 Fc Mutations

Anti-CTLA4 antibodies TY21580 (wild-type human IgG1 Fc region) and TY24040 (human IgG1 Fc region with S239D/I332E substitutions) were further evaluated in comparison to commercially available ipilimumab and an isotype control in an MC38 cancer model.

### A. Anti-Tumor Efficacy in an MC38 Cancer Model

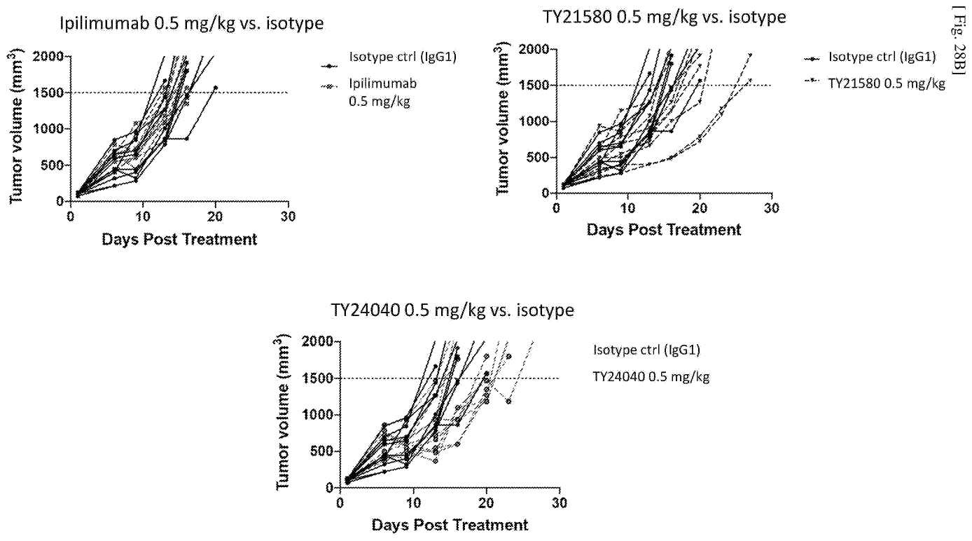

A first study was performed using the method described in Example 20, except that there were 10 mice per group, each antibody was administered at both 0.5 mg/kg and 5 mg/kg, and administration of the antibodies was initiated when the tumors reached a volume of about 75 mm³. The mice were sacrificed if tumor volume exceeded 1, 250 mm³. As shown in FIG. 26A-26C, both TY21580 and TY24040 showed greater tumor growth suppression than ipilimumab at both 0.5 mg/kg and 5 mg/kg doses, with TY24040 being the most effective. At 0.5 mg/kg, only 1 out of 10 mice treated with ipilimumab survived beyond day 40 of the study, and none of the mice treated with TY21580 survived to 40 days. By contrast, 9 out of 10 mice treated with 0.5 mg/kg TY24040 survived beyond day 40, and the tenth mouse survived beyond day 30 of the study (FIG. 26B). Similarly, at 5 mg/kg, 9 out of 10 mice treated with TY24040 survived beyond day 40, as compared to 6 out of 10 for TY21580 and 3 out of 10 for ipilimumab (FIG. 26C).

### B. Treg Depletion in an MC38 Cancer Model

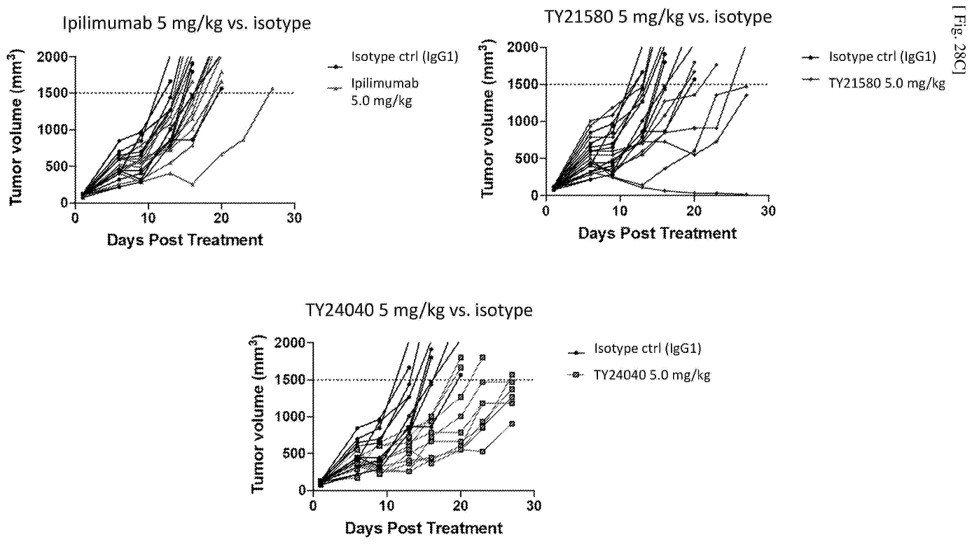

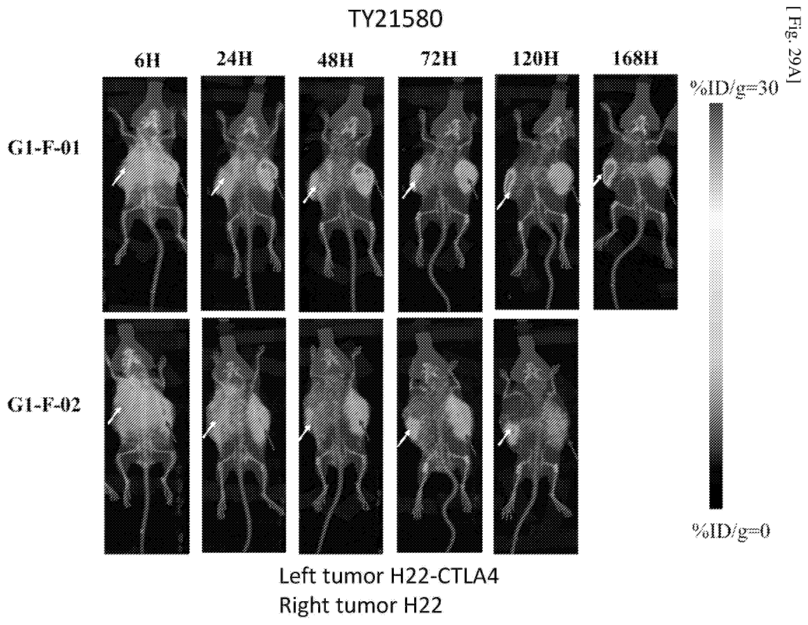

Five mice from each group of the study in Example 21A were sacrificed, and their tumors were collected for the analysis of the efficacy of the antibodies to induce Treg depletion. As shown in FIG. 27A, both TY21580 and TY24040 showed a significantly lower percent of CD4+ T cells in the tumor microenvironment than both the isotype control and ipilimumab. As shown in FIG. 27B, both TY21580 and TY24040 showed a lower number of Tregs per million live cells than ipilimumab and a significantly lower number of Tregs per million live cells than the isotype control.

C. Anti-Tumor Efficacy in an MC38 Cancer Model with Large, Established Tumors

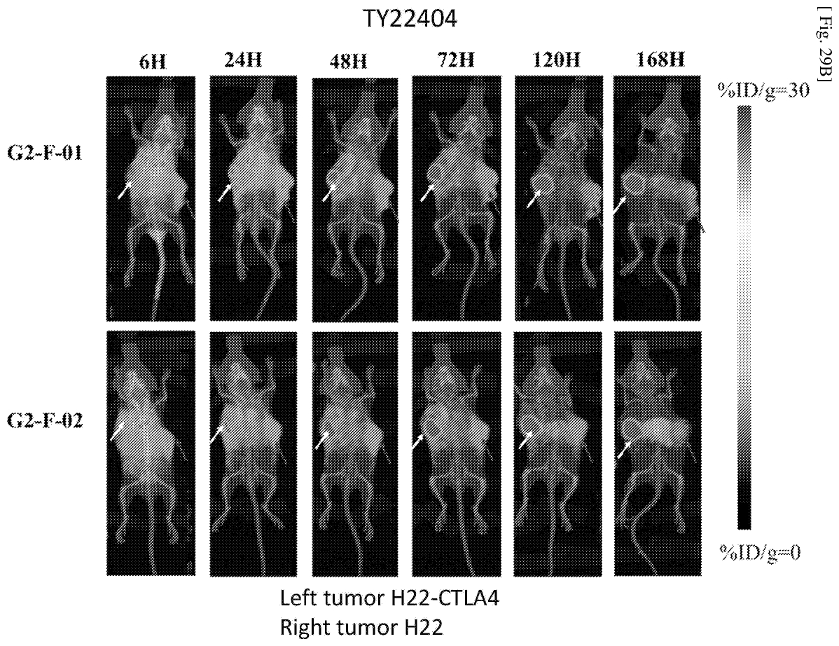

A second study was performed using the same method described in Example 21A, except that administration of the antibodies was initiated when the tumors had reached a volume of about 300 mm³, representing treatment of large, established tumors. The mice were sacrificed if tumor volume exceeded 1, 500 mm³. As shown in FIGS. 28A-28C, at both dosages, TY24040 generated the strongest tumor growth inhibition in large, established tumors, as compared to TY21580 and ipilimumab. Notably, at the 5 mg/kg dosage, none of the ipilimumab-treated mice maintained a tumor volume of less than 1, 500 mm³ at the end of the study, while 3 of the mice treated with TY21580 and 5 of the mice treated with TY24040 maintained a tumor volume of less than 1, 500 mm³ at the end of the study (FIG. 28C).

### Example 21. Tumor Accumulation of Antibodies Targeting CTLA4

The biodistribution of anti-CTLA4 antibodies was assessed using antibodies radiolabeled with ⁸⁹Zr. Radiolabeled antibodies were injected into mice with two hepatocellular carcinoma tumors; tumors on the right side of the animals were established using H22 cancer cells, and tumors on the left side of the animals were established using H22 cancer cells expressing CTLA4. Animals were dosed according to Table 15. Biodistribution of antibodies was analyzed using PET/CT imaging 6, 24, 48, 72, 120, and 168 hours following antibody injection.

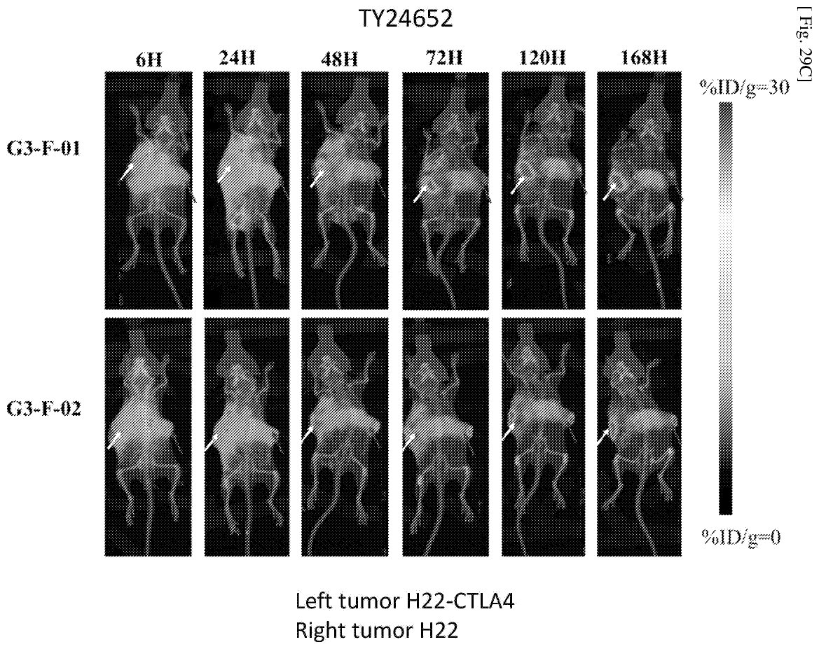

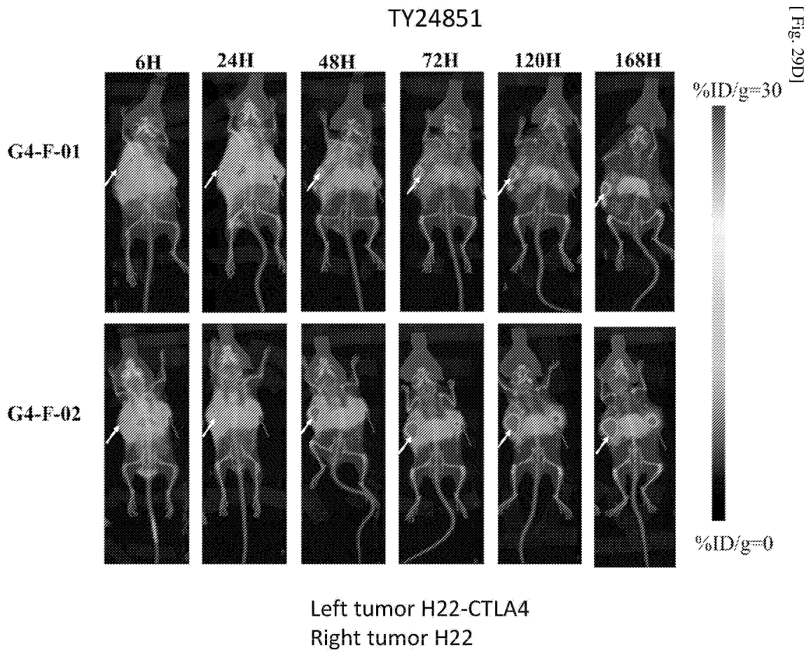

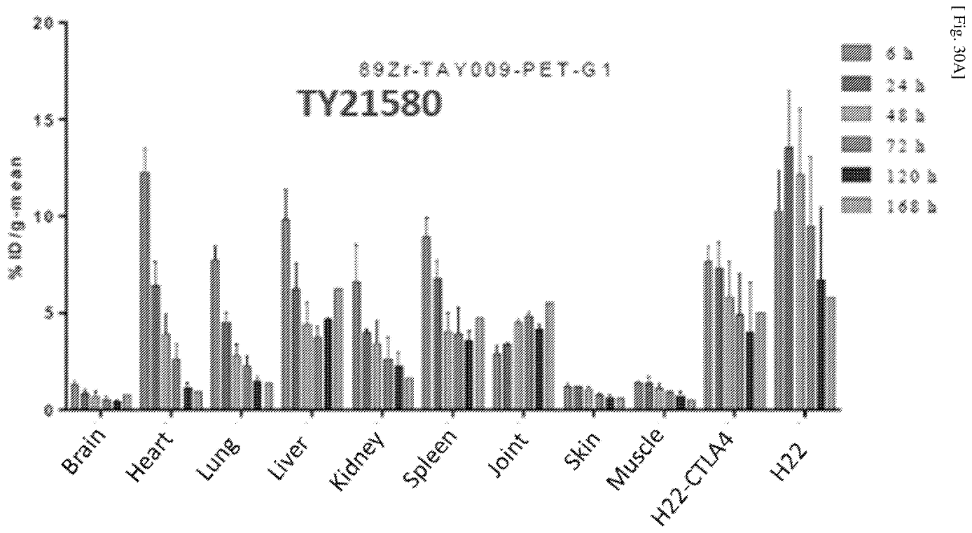

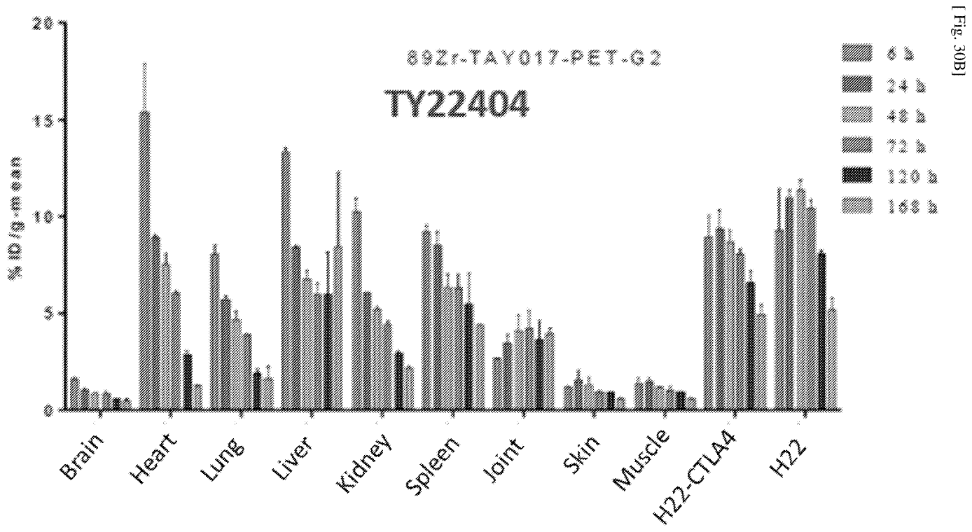

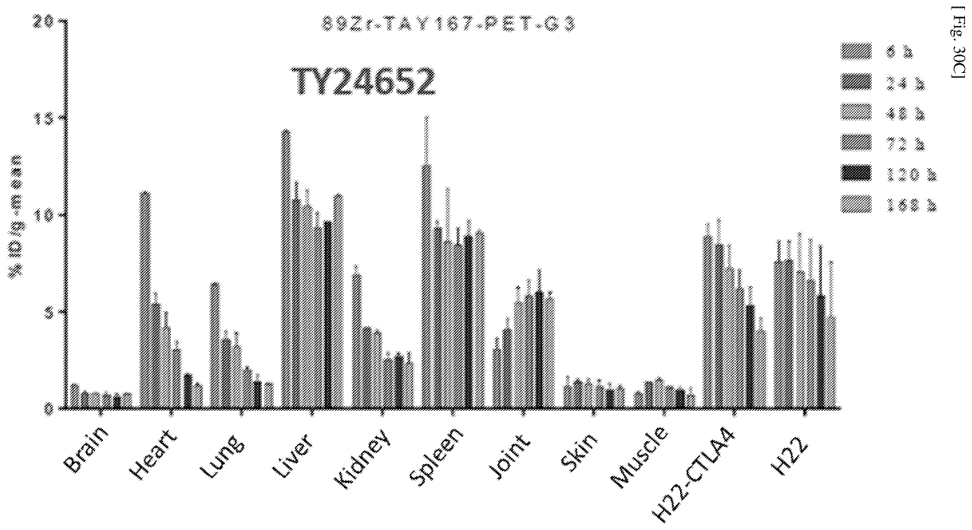

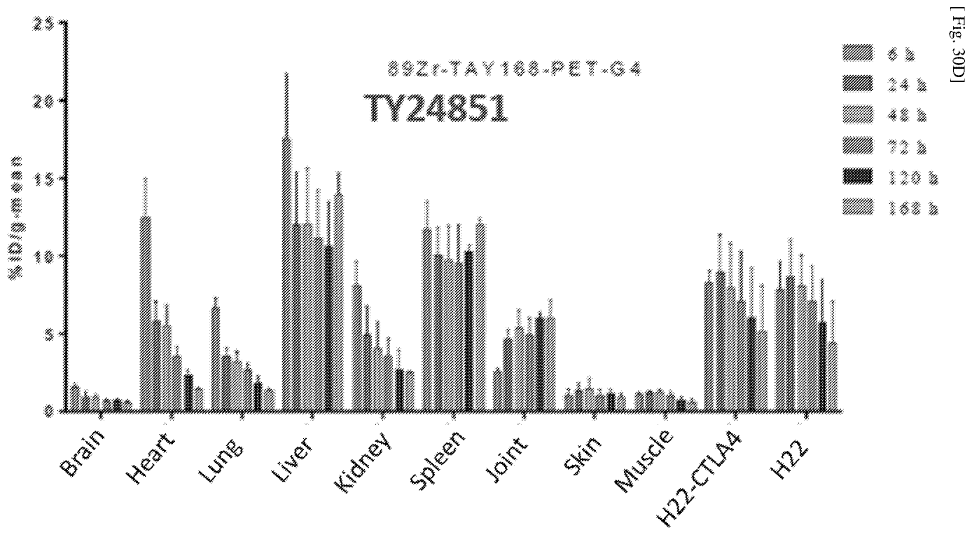

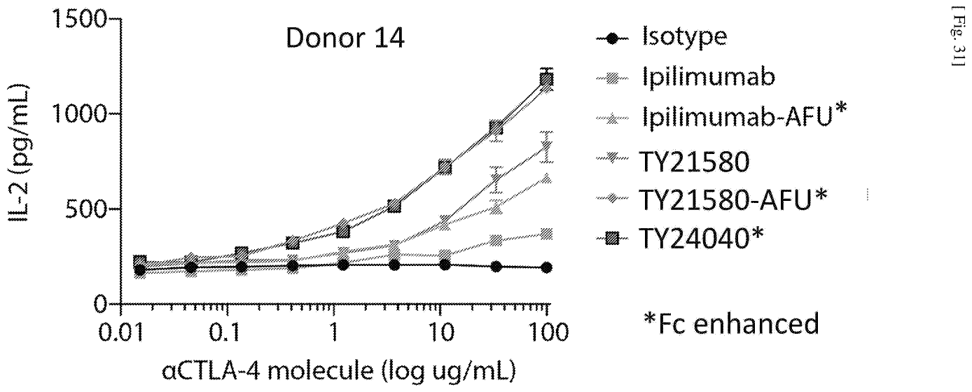

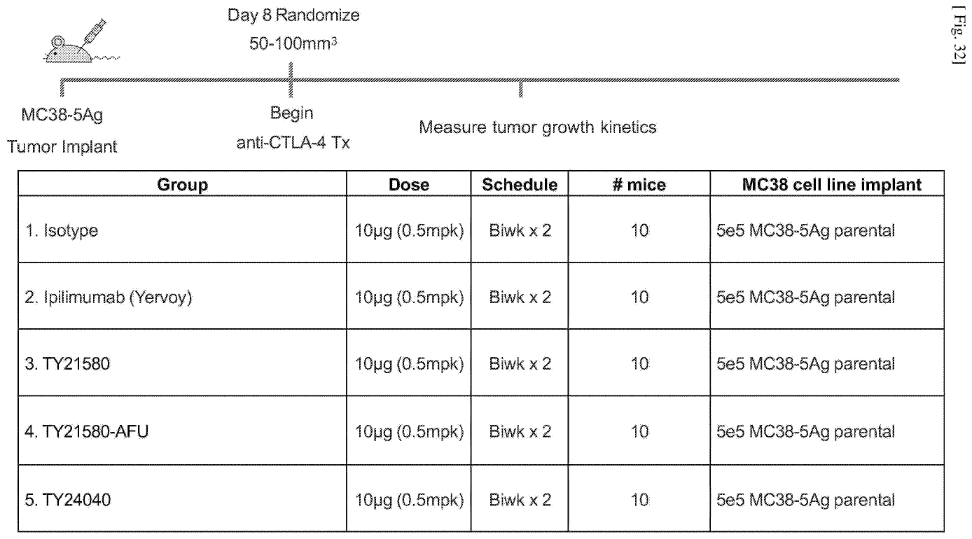

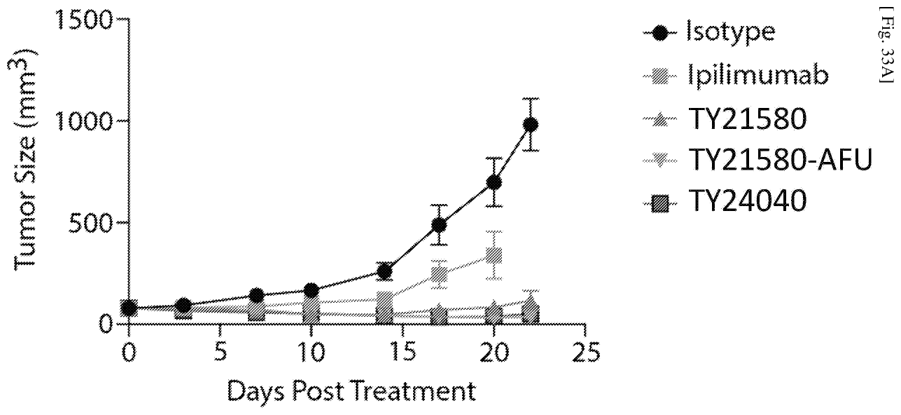

As shown in FIGS. 29-30, radioactivity signals concentrate in tumors, as well as tissues with abundant blood flow. As shown in FIGS. 29A and 30A, TY21580 peaks in H22-CTLA4 tumors 6 hours following injection, but peaks in H22 tumors after 24 hours. Similar trends are also observed for the other antibodies (FIGS. 29-30). The PET/CT scans shown in FIG. 29A-29D suggest that antibody concentrations are greater in H22-CTLA4 than in H22 at 168 hours post injection, suggesting that the masked antibodies can also be enriched in the target-expressing tumors with time.

TABLE 15
Dosage of radiolabeled anti-CTLA4 antibodies

| | | | Specific | Radioactive dosage | | Antibody dosage | |
|---|---|---|---|---|---|---|---|
| | Animal | | radioactivity | (uCi) | | (mg/kg) | |
| Group | ID | Antibody | (mCi/mg) | Individual | Mean | Individual | Mean |
| G1 | G1-F-01 | TY21580 | 1.24 | 88.8 | 88.5 | 4.4 | 4.3 |
| | G1-F-02 | | | 88.7 | | 4.2 | |
| G2 | G2-F-01 | TY22404 | 1.14 | 83.6 | 84.1 | 4.4 | 4.6 |
| | G2-F-02 | | | 84.6 | | 4.7 | |
| G3 | G3-F-01 | TY24652 | 1.15 | 90.4 | 89.3 | 4.3 | 4.3 |
| | G3-F-02 | | | 88.3 | | 4.3 | |
| G4 | G4-F-01 | TY24851 | 1.13 | 96.7 | 97.3 | 4.4 | 5.1 |
| | G4-F-02 | | | 97.9 | | 5.9 | |

### Example 22. In Vitro and In Vivo Characterization of an Unmasked Anti-CTLA4 Antibody TY24040 with ADCC-Enhancing IgG1 Fc Mutations

Anti-CTLA4 antibodies TY21580 (wild-type human IgG1 Fc region), TY21580-AFU and TY24040 (human IgG1 Fc region with S239D/I332E substitutions) were further evaluated in comparison to commercially available ipilimumab and an isotype control in cell culture and in an MC38 cancer model.

### A. Enhancement of T Cell Activation Primed by SEA

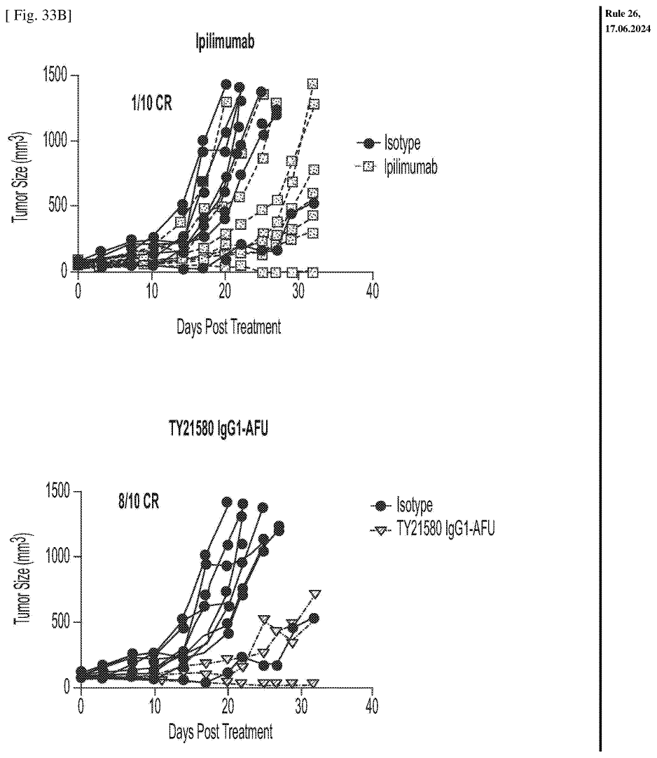

The experiment was performed as described in Example 5. As demonstrated in FIG. 31, parental antibody TY21580, and the variants TY21580-AFU and TY24040 (i.e., TY21580 with the ADCC-enhancing substitutions S239D and I332E) showed greater dose dependent PBMC activation activity compared to the isotype control, ipilimumab, and ipilimumab-AFU (a Fc-enhanced variant), indicating that ADCC enhancing mutations can further enhance the antibody's effectiveness as measured by this assay.

### B. Anti-Tumor Efficacy in an MC38 Cancer Model

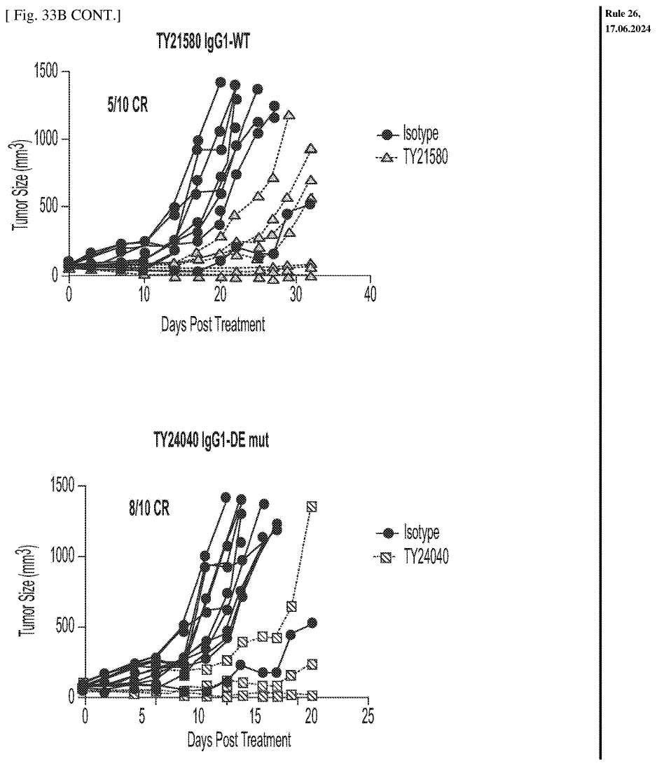

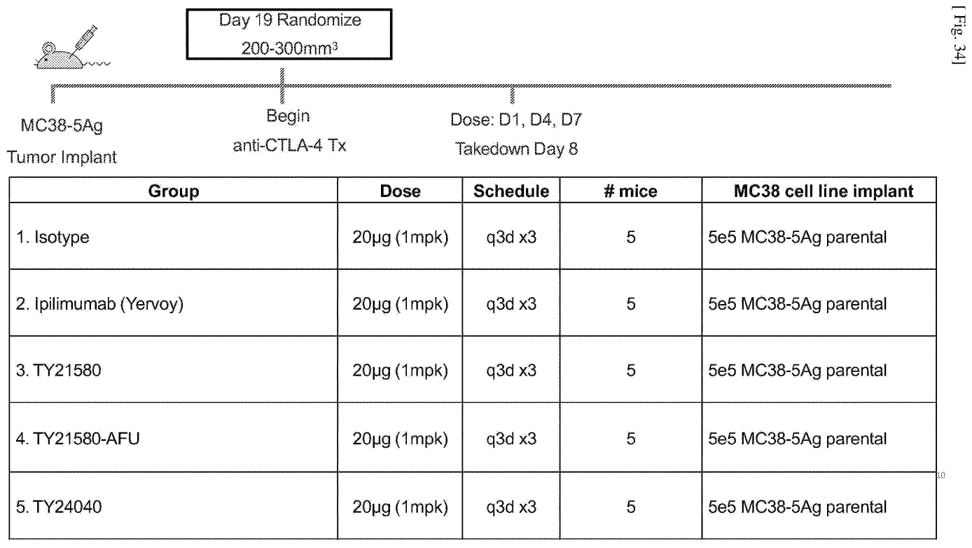

The study was performed using the method described in Example 20, except that there were 10 mice per group, each antibody was administered at 0.5 mg/kg and administration of the antibodies was initiated when the tumors reached a volume of about 50-100 mm³ (FIG. 32). The mice were sacrificed if tumor volume exceeded 1, 250 mm³. As shown in FIG. 33A, TY21580, TY21580-AFU and TY24040 showed greater tumor growth suppression than ipilimumab and the isotype control at 0.5 mg/kg, with TY24040 being the most effective. At 0.5 mg/kg, only 1 out of 10 mice treated with ipilimumab survived beyond day 31 of the study, 5 out of 10 mice treated with TY21580 survived beyond 31 days, and 8 out of 10 mice treated with TY21580-AFU survived beyond 31 days. 8 out of 10 mice treated (FIG. 32B).

### C. Treg Depletion in an MC38 Cancer Model

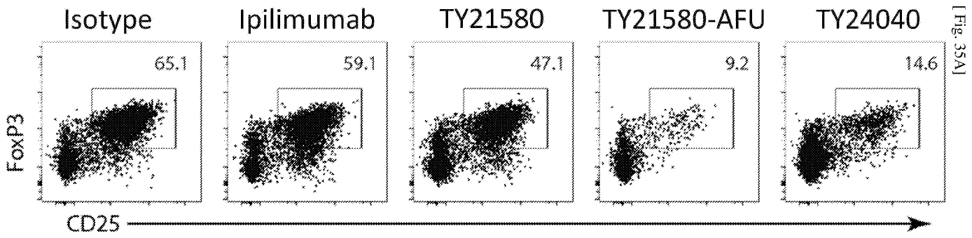

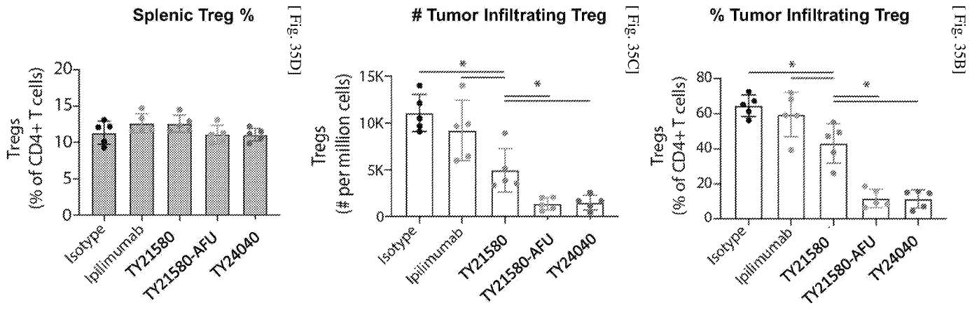

Five mice from each group of the study as described in FIG. 34 were sacrificed, and their tumors were collected for the analysis of the efficacy of the antibodies to induce Treg depletion. As shown in FIG. 35A, TY21580-AFU and TY24040 showed a significantly lower FoxP3 positive CD4+ T cells in the tumor microenvironment than both the isotype control and ipilimumab. TY21580 showed a lower percentage of Treg cells in the population of CD4+ T cells compared to ipilimumab and the isotype control, but TY21580-AFU and TY24040 demonstrated an even greater reduction in Treg cells (FIG. 35B). As shown in FIG. 35C, TY21580-AFU and TY24040 showed a lower number of Tregs per million live cells than ipilimumab and a significantly lower number of Tregs per million live cells than the isotype control. FIG. 35D shows that the Tregs found in splenic T cells were largely unchanged, indicating that the effect in Treg depletion in tumors was due to the antibody.

1. An isolated antibody that binds to human CTLA4, wherein the antibody comprises a heavy chain comprising a heavy chain variable region (VH) and a light chain comprising a light chain variable region (VL), wherein the heavy chain comprises a heavy chain constant region comprising a human IgG1 Fc region, wherein the heavy chain constant region comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering.

2. The antibody of claim 1, wherein
1) the VH comprises a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3, and the VL comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
2) the VH comprises a CDR-H1 comprising the amino acid sequence of SEQ ID NO: 12, a CDR-H2 comprising the amino acid sequence of SEQ ID NO: 13, a CDR-H3 comprising the amino acid sequence of SEQ ID NO: 14, and the VL comprises a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17;
3) the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 53, and the VL comprises a CDR-L1 comprising the amino acid sequence of SEQ ID NO: 54, a CDR-L2 comprising the amino acid sequence of SEQ ID NO: 16, and a CDR-L3 comprising the amino acid sequence of SEQ ID NO: 55; or
4) the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 46, and the VL comprises a CDR-L1 comprising the amino acid sequence of SEQ ID NO: 47, a CDR-L2 comprising the amino acid sequence of SEQ ID NO: 5, and a CDR-L3 comprising the amino acid sequence of SEQ ID NO: 48.

3. The isolated antibody of claim 1 or 2, wherein: 1) the VH comprises the amino acid sequence of SEQ ID NO: 74, and/or the VL comprises the amino acid sequence of SEQ ID NO: 75; 2) the VH comprises the amino acid sequence of SEQ ID NO: 66, and/or the VL comprises the amino acid sequence of SEQ ID NO: 67; 3) the VH comprises the amino acid sequence of SEQ ID NO: 84, and/or the VL comprises the amino acid sequence of SEQ ID NO: 85; or 4) the VH comprises the amino acid sequence of SEQ ID NO: 80, and/or the VL comprises the amino acid sequence of SEQ ID NO: 81.

4. The isolated antibody of any one of claims 1-3, wherein the heavy chain comprises the amino acid sequence of SEQ ID NO: 91, and the light chain comprises the amino acid sequence of SEQ ID NO: 90.

5. The isolated antibody of any one of claims 1-3, wherein the heavy chain comprises the amino acid sequence of SEQ ID NO: 91 without the C-terminal lysine, and the light chain comprises the amino acid sequence of SEQ ID NO: 90

6. The isolated antibody of any one of claims 1-5, wherein the antibody comprises two heavy chains and two light chains, wherein each human IgG1 Fc domain of the heavy chains comprises:
i) an S298A substitution, an E333A substitution, and a K334A substitution;
ii) an S298A substitution, an E333A substitution, a K334A substitution, and a K326A substitution;
iii) an F243L substitution;
iv) an F243L substitution, an R292P substitution, a Y300L substitution, and a P396L substitution;
v) an F243L substitution, an R292P substitution, a Y300L substitution, a V305I substitution, and a P396L substitution;
vi) an F243L substitution, an R292P substitution, a Y300L substitution, a L235V substitution, and a P396L substitution;
vii) a S239D substitution, an I332E substitution, and an A330L substitution; or
viii) a S239D substitution and an I332E substitution.

7. The isolated antibody of claim 6, wherein each human IgG1 Fc domain comprises a S239D substitution and an I332E substitution.

8. The isolated antibody of claim 6, wherein each human IgG1 Fc domain comprises a S239D substitution, an I332E substitution, and an A330L substitution.

9. The isolated antibody of claim 6, wherein each human IgG1 Fc domain comprises an F243L substitution, an R292P substitution, a Y300L substitution, a V305I substitution, and a P396L substitution.

10. The isolated antibody of any one of claims 1-9, wherein the isolated antibody induces antibody-dependent cell cytotoxicity (ADCC) against a CTLA4-expressing human cell or a human Treg cell, wherein the ADCC activity of the isolated antibody is higher than that of a control antibody comprising a human IgG1 Fc region that does not comprise the substitutions, and/or wherein the ADCC activity of the isolated antibody is higher than the ADCC activity of ipilimumab.

11. A masked antibody that binds to human CTLA4, comprising a masking peptide (MP) and an antibody, wherein the antibody comprises a heavy chain comprising a heavy chain variable region (VH) and a light chain comprising a light chain variable region (VL), wherein the MP is linked to the N-terminal of the VH or VL, wherein the MP comprises, from N-terminus to C-terminus, a masking unit (MU) and a linkage unit (LU), wherein the MU comprises an amino acid sequence selected from the list consisting of SEQ ID NOs: 146-151, and wherein
1) the VH comprises a CDR-H1 comprising the amino acid sequence of SEQ ID NO: 1, a CDR-H2 comprising the amino acid sequence of SEQ ID NO: 2, a CDR-H3 comprising the amino acid sequence of SEQ ID NO: 3, and the VL comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
2) the VH comprises a CDR-H1 comprising the amino acid sequence of SEQ ID NO: 12, a CDR-H2 comprising the amino acid sequence of SEQ ID NO: 13, a CDR-H3 comprising the amino acid sequence of SEQ ID NO: 14, and the VL comprises a CDR-L1 comprising the amino acid sequence of SEQ ID NO: 15, a CDR-L2 comprising the amino acid sequence of SEQ ID NO: 16, and a CDR-L3 comprising the amino acid sequence of SEQ ID NO: 17;
3) the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 53, and the VL comprises a CDR-L1 comprising the amino acid sequence of SEQ ID NO: 54, a CDR-L2 comprising the amino acid sequence of SEQ ID NO: 16, and a CDR-L3 comprising the amino acid sequence of SEQ ID NO: 55; or
4) the VH comprises a CDR-H1 comprising the amino acid sequence of SEQ ID NO: 44, a CDR-H2 comprising the amino acid sequence of SEQ ID NO: 45, a CDR-H3 comprising the amino acid sequence of SEQ ID NO: 46, and the VL comprises a CDR-L1 comprising the amino acid sequence of SEQ ID NO: 47, a CDR-L2 comprising the amino acid sequence of SEQ ID NO: 5, and a CDR-L3 comprising the amino acid sequence of SEQ ID NO: 48.

12. The masked antibody of claim 11, wherein the MP further comprises an N-terminal unit (NU) linked to the N-terminal of the MU.

13. The masked antibody of claim 12, wherein the N-terminal unit is about 1-10 amino acid residues long.

14. The masked antibody of claim 12, wherein the N-terminal unit comprises an amino acid sequence selected from the group consisting of SEQ ID NOs: 143-145.

15. The masked antibody of any one of claims 11-14, wherein the LU does not comprise a cleavage site.

16. The masked antibody of claim 15, wherein the LU comprises a linker.

17. The masked antibody of claim 16 wherein the linker comprises an amino acid sequence selected from the group consisting of SEQ ID NOs: 155-161.

18. The masked antibody of claim 16 wherein the linker comprises the amino acid sequence of SEQ ID NO: 159.

19. The masked antibody of any one of claims 11-14, wherein the masked antibody is an activatable antibody.

20. The masked antibody of claim 19, wherein the LU comprises at least a first cleavage site (C₁).

21. The masked antibody of claim 20, wherein the first cleavage site (C₁) is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

22. The masked antibody of claim 20 or 21, wherein the first cleavage site (C₁) comprises an amino acid sequence selected from the group consisting of SEQ ID NOs: 152-154.

23. The masked antibody of any one of claims 20-22, wherein the LU further comprises a second cleavage site (C₂).

24. The masked antibody of claim 23, wherein the second cleavage (C₂) site is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

25. The masked antibody of claim 23 or 24, wherein the second cleavage site (C₂) comprises an amino acid sequence selected from the group consisting of SEQ ID NOs: 152-154.

26. The masked antibody of any one of claims 23-25, wherein the first and second cleavage sites are different.

27. The masked antibody of any one of claims 19-26, wherein the LU further comprises a first linker.

28. The masked antibody of claim 27, wherein the first linker comprises an amino acid sequence selected from the group consisting of SEQ ID NOs: 155-161.

29. The masked antibody of claim 27 or 28, wherein the LU further comprises a second linker (L₂).

30. The masked antibody of claim 29, wherein the second linker comprises an amino acid sequence selected from the group consisting of SEQ ID NOs: 155-161.

31. The masked antibody of any one of claims 27-30, wherein the LU comprises, from N-terminus to C-terminus:
1) the first cleavage site (C₁), the first linker (L₁), the second cleavage site (C₂), and the second linker (L₂); or
2) the first linker (L₁), the first cleavage site (C₁), and the second linker (L₂).

32. The masked antibody of any one of claims 11-31, wherein the LU comprises an amino acid sequence selected from the group consisting of SEQ ID NOs: 99-103.

33. The masked antibody of any one of claims 11-31, wherein
the MU comprises the amino acid sequence of SEQ ID NO: 146, and the LU comprises the amino acid sequence of SEQ ID NO: 99;
the MU comprises the amino acid sequence of SEQ ID NO: 147, and the LU comprises the amino acid sequence of SEQ ID NO: 99;
the MU comprises the amino acid sequence of SEQ ID NO: 148, and the LU comprises the amino acid sequence of SEQ ID NO: 99;
the MU comprises the amino acid sequence of SEQ ID NO: 149, and the LU comprises the amino acid sequence of SEQ ID NO: 99;
the MU comprises the amino acid sequence of SEQ ID NO: 150, and the LU comprises the amino acid sequence of SEQ ID NO: 99;
the MU comprises the amino acid sequence of SEQ ID NO: 148, and the LU comprises the amino acid sequence of SEQ ID NO: 100;
the MU comprises the amino acid sequence of SEQ ID NO: 148, and the LU comprises the amino acid sequence of SEQ ID NO: 101;
the MU comprises the amino acid sequence of SEQ ID NO: 148, and the LU comprises the amino acid sequence of SEQ ID NO: 102;
the MU comprises the amino acid sequence of SEQ ID NO: 148, and the LU comprises the amino acid sequence of SEQ ID NO: 101;
the MU comprises the amino acid sequence of SEQ ID NO: 151, and the LU comprises the amino acid sequence of SEQ ID NO: 100;
the MU comprises the amino acid sequence of SEQ ID NO: 151, and the LU comprises the amino acid sequence of SEQ ID NO: 101;
the MU comprises the amino acid sequence of SEQ ID NO: 150, and the LU comprises the amino acid sequence of SEQ ID NO: 101;
the MU comprises the amino acid sequence of SEQ ID NO: 150, and the LU comprises the amino acid sequence of SEQ ID NO: 102;
the MU comprises the amino acid sequence of SEQ ID NO: 150, and the LU comprises the amino acid sequence of SEQ ID NO: 102;
the MU comprises the amino acid sequence of SEQ ID NO: 150, and the LU comprises the amino acid sequence of SEQ ID NO: 103; or
the MU comprises the amino acid sequence of SEQ ID NO: 150, and the LU comprises the amino acid sequence of SEQ ID NO: 103.

34. The masked antibody of any one of claims 11-33, wherein the masking peptide comprises an amino acid sequence selected from the group consisting of SEQ ID NOs: 162-177.

35. The masked antibody of any one of claims 11-34, wherein 1) the VH comprises the amino acid sequence of SEQ ID NO: 74, and/or the VL comprises the amino acid sequence of SEQ ID NO: 75; 2) the VH comprises the amino acid sequence of SEQ ID NO: 66, and/or the VL comprises the amino acid sequence of SEQ ID NO: 67; 3) the VH comprises the amino acid sequence of SEQ ID NO: 84, and/or the VL comprises the amino acid sequence of SEQ ID NO: 85; or 4) the VH comprises the amino acid sequence of SEQ ID NO: 80, and/or the VL comprises the amino acid sequence of SEQ ID NO: 81.

36. The masked antibody of any one of claims 11-35, wherein the masked antibody is a Fab fragment, an scFv, a F (ab′)₂ fragment, or an Fv fragment.

37. The masked antibody of any one of claims 11-35, wherein the antibody comprises two heavy chains and two light chains, wherein each human IgG1 Fc domain of the heavy chain comprises one or more substitutions selected from the group consisting of a L235V substitution, a S239D substitution, an F243L substitution, an R292P substitution, an S298A substitution, a Y300L substitution, a V305I substitution, a K326A substitution, an A330L substitution, an I332E substitution, an E333A substitution, a K334A substitution, and a P396L substitution, wherein the residue numbering is according to EU numbering.

38. The masked antibody of claim 37, wherein each human IgG1 Fc domain comprises:
i) an S298A substitution, an E333A substitution, and a K334A substitution;
ii) an S298A substitution, an E333A substitution, a K334A substitution, and a K326A substitution;
iii) an F243L substitution;
iv) an F243L substitution, an R292P substitution, a Y300L substitution, and a P396L substitution;
v) an F243L substitution, an R292P substitution, a Y300L substitution, a V305I substitution, and a P396L substitution;
vi) an F243L substitution, an R292P substitution, a Y300L substitution, a L235V substitution, and a P396L substitution;
vii) a S239D substitution, an I332E substitution, and an A330L substitution; or
viii) a S239D substitution and an I332E substitution.

39. The masked antibody of claim 38, wherein each human IgG1 Fc domain comprises a S239D substitution and an I332E substitution.

40. The masked antibody of claim 38, wherein each human IgG1 Fc domain comprises a S239D substitution, an I332E substitution, and an A330L substitution.

41. The masked antibody of claim 38, wherein each human IgG1 Fc domain comprises an F243L substitution, an R292P substitution, a Y300L substitution, a V305I substitution, and a P396L substitution.

42. The masked antibody of any one of claims 37-41, wherein the heavy chains each comprise an amino acid sequence selected from the group consisting of SEQ ID NOS: 104-105, and the light chains each comprise an amino acid sequence selected from the group consisting of SEQ ID NOS: 106-121.

43. The masked antibody of claim 42, wherein the heavy chains each comprise the amino acid sequence of SEQ ID NO: 105, and the light chains each comprise the amino acid sequence of SEQ ID NO: 121.

44. The masked antibody of any one of claims 37-43, wherein the masked antibody induces antibody-dependent cell cytotoxicity (ADCC) against a CTLA4-expressing human cell or a human Treg cell, wherein the ADCC activity of the isolated antibody is higher than that of a control antibody comprising a human IgG1 Fc region that do not comprise the substitutions, and/or wherein the ADCC activity of the isolated antibody is higher than the ADCC activity of ipilimumab.

45. An isolated polynucleotide encoding one or more polypeptide chains of the isolated antibody of any one of claims 1-10, or the masked antibody of any one of claims 11-44.

46. A vector comprising the polynucleotide of claim 45.

47. The vector of claim 46, wherein the vector is an expression vector and/or a display vector.

48. A host cell comprising the polynucleotide or vector of any one of claims 45-47.

49. The host cell of claim 48, wherein the host cell is a eukaryotic cell.

50. The host cell of claim 48 or 49, wherein the host cell is a Chinese Hamster Ovary (CHO) cell.

51. A method of making an antibody or masked antibody comprising culturing the host cell of any one of claims 48-50 under conditions suitable for producing the antibody or activatable antibody.

52. The method of claim 51, further comprising recovering the antibody or masked antibody produced by the cell.

53. A pharmaceutical composition comprising the antibody of any one of claims 1-10, or the masked antibody of any one of claims 11-44, and a pharmaceutically acceptable carrier.

54. A method of treating or delaying progression of a cancer in a subject in need thereof, the method comprising administering to the subject an effective amount of the antibody of any one of claims 1-10, the masked antibody of any one of claims 11-44, or the pharmaceutical composition of claim 53.

55. A method of reducing size of a solid tumor in a subject in need thereof, wherein the solid tumor has a size of about 400-1000 mm³, the method comprises administering to the subject an effective amount of the antibody of any one of claims 1-10, the masked antibody of any one of claims 11-44, or the pharmaceutical composition of claim 53.

56. A method of reducing on-target off-tumor toxicity in a subject having a cancer in need thereof, wherein the method comprises administering to the subject an effective amount of the masked antibody of any one of claims 11-44.

57. The method of any one of claims 54-56, wherein the tumor is a colorectal tumor or a lung tumor.

58. The method of any one of claims 54-57, wherein the tumor is a cold tumor.

59. The method of claim 58, wherein the cold tumor is selected from the group consisting of pancreatic cancer, ovarian cancer, and head and neck cancer.

60. The method of any one of claims 54-59, further comprising administering to the subject an effective amount of at least one additional therapeutic agent.

61. The method of claim 60, wherein the at least one additional therapeutic agent is selected from the group consisting of viral gene therapy, immune checkpoint inhibitors, target therapies, radiation therapies, vaccination therapies, and chemotherapies.

62. The method of claim 61, wherein the at least one additional therapeutic agent is an immune checkpoint inhibitor.

63. The method of claim 62, wherein the at least one additional therapeutic agent is an inhibitor of PD-1.

64. The method of any one of claims 54-63, wherein the method comprises administering to the subject an effective amount of the antibody, the masked antibody, or the pharmaceutical composition prior to a surgery or after a surgery to remove a solid tumor in the subject.

65. A method inducing phagocytosis of cells expressing CTLA4, the method comprising contacting the cells expressing CTLA4 with an effective amount of the antibody of any one of claims 1-10, or the masked antibody of any one of claims 11-44.

66. The method of claim 65, wherein the phagocytosis is antibody-dependent cellular phagocytosis (ADCP).

67. The method of any of claims 65-66, wherein the antibody, or the masked antibody, has a half maximal effective concentration (EC50) of less than 3 nM for inducing ADCP of cells expressing CTLA4.

68. A method for depleting regulatory T cells, wherein the method comprises administering to the subject an effective amount of the antibody of any one of claims 1-10 or the pharmaceutical composition of claim 53.

69. The masked antibody of any one of claims 11-35, wherein the antibody comprises an a human IgG1 Fc region, and wherein the human IgG1 Fc region is afucosylated.
